(19) United States
(12) Patent Application Publication
(10) Pub. No.: US 2025/0248207 A1
(43) Pub. Date: Jul. 31, 2025

(54) LIGHT-EMITTING ELEMENT, NITROGEN-CONTAINING COMPOUND FOR LIGHT-EMITTING ELEMENT, AND DISPLAY DEVICE INCLUDING THE LIGHT-EMITTING ELEMENT

(71) Applicant: Samsung Display Co., Ltd., Yongin-si (KR)

(72) Inventors: DONGJUN KIM, Yongin-si (KR); HANKYU PAK, Yongin-si (KR); HYUNBIN PARK, Yongin-si (KR); JINYOUNG YUN, Yongin-si (KR); SANGHYUN HAN, Yongin-si (KR)

(21) Appl. No.: 18/961,217

(22) Filed: Nov. 26, 2024

(30) Foreign Application Priority Data
Jan. 31, 2024 (KR) 10-2024-0015285

(51) Int. Cl. H10K 50/16; C09K 11/06; H10K 50/115; H10K 85/60

(52) U.S. Cl. CPC H10K 50/16; C09K 11/06; H10K 50/115; H10K 85/654; C09K 2211/1059

(57) ABSTRACT

A light-emitting element including a first electrode, a second electrode on the first electrode, and at least one functional layer between the first electrode and the second electrode is provided. The functional layer includes a nitrogen-containing compound represented by Formula 1.

### CROSS-REFERENCE TO RELATED APPLICATION

This application claims priority to and the benefit of Korean Patent Application No. 10-2024-0015285, filed on Jan. 31, 2024, in the Korean Intellectual Property Office, the entire content of which is incorporated herein by reference.

### BACKGROUND

### 1. Field

The present disclosure herein relates to a light-emitting element, a nitrogen-containing compound used in the light-emitting element, and a display device including the light-emitting element.

### 2. Description of the Related Art

Recently, organic electroluminescence display devices as image display devices have been actively developed. Unlike liquid crystal display devices, an organic electroluminescence display device is so-called “self-luminous” type or kind display devices that function by recombing, in an emission layer of the organic electroluminescence display device, holes and electrons provided or injected, respectively, from a first electrode and a second electrode of the organic electroluminescence display device. Subsequently, a light-emitting material of the emission layer emits light to achieve or implement display (e.g., of an image).

In applying a light-emitting element to display devices, improvements in luminous efficiency, and long lifespan are desired or required, and the development of materials for a light-emitting element capable of stably achieving these improvements is continuously desired or required.

For example, in order to achieve the light-emitting element having relatively high efficiency and long lifespan, a material for an electron transport region having excellent or suitable charge transport properties and stability is under development or being pursued.

### SUMMARY

One or more aspects of embodiments of the present disclosure are directed toward a light-emitting element having improved luminous efficiency and element lifespan.

One or more aspects of embodiments of the present disclosure are directed toward a nitrogen-containing compound capable of improving luminous efficiency and element of the light-emitting element.

One or more aspects of embodiments of the present disclosure are directed toward a display device having excellent or suitable display quality by including the light-emitting element having improved luminous efficiency and lifespan.

Additional aspects will be set forth in part in the description which follows and, in part, will be apparent from the description, or may be learned by practice of the presented embodiments of the disclosure.

One or more embodiments of the present disclosure provides a light-emitting element including a first electrode; a second electrode arranged on the first electrode; and at least one functional layer arranged between the first electrode and the second electrode, and including a nitrogen-containing compound represented by Formula 1.

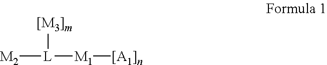

In Formula 1, A₁ is -(L₁)ₐ₁M₄, L₁ may be a substituted or unsubstituted arylene group having 6 to 60 ring-forming carbons, or a substituted or unsubstituted heteroarylene group having 2 to 60 ring-forming carbons, M₂ to M₄ may each independently be a substituted or unsubstituted triazine group, a1 may be an integer of 1 to 3, m and n may each independently be 0 or 1, a sum of m and n may be 1, if (e.g., when) n is 0, (then) M₁ may be a substituted or unsubstituted triazine group, if (e.g., when) n is 1, M₁ may be a substituted or unsubstituted divalent triazine group, if (e.g., when) m is 0, L may be represented by -(L₂)ₐ₂-, L₂ may be a substituted or unsubstituted arylene group having 6 to 60 ring-forming carbons, or a substituted or unsubstituted heteroarylene group having 2 to 60 ring-forming carbons, and a2 may be an integer of 1 to 3, if (e.g., when) m is 1, L may be a substituted or unsubstituted trivalent aryl group having 6 to 60 ring-forming carbons, or a substituted or unsubstituted trivalent heteroaryl group having 2 to 60 ring-forming carbons, at least one selected from among M₁ to M₄ includes, as a substituent, an alkyl group having 1 to 30 carbons, or a substituted or unsubstituted cycloalkyl group having 3 to 30 ring-forming carbons, and in Formula 1, cases where each of L, L₁, and L₂ includes a substituted or unsubstituted carbazole linker are excluded (e.g., each of L, L₁, and L₂ does not comprise a or any substituted or unsubstituted carbazole linker).

In one or more embodiments, the at least one functional layer may include an emission layer; a hole transport region arranged between the first electrode and the emission layer; and an electron transport region arranged between the emission layer and the second electrode, wherein the electron transport region includes a nitrogen-containing compound represented by Formula 1.

In one or more embodiments, the electron transport region may include an electron transport layer arranged on the emission layer; and an electron injection layer arranged on the electron transport layer, wherein the electron transport layer includes a nitrogen-containing compound, represented by Formula 1.

In one or more embodiments, an adjacent layer to the emission layer (e.g., a layer adjacent to the emission layer) selected from among a plurality of layers included in the electron transport region may include a nitrogen-containing compound represented by Formula 1.

In one or more embodiments, at least one selected from among M₁ to M₄ may be substituted with a substituted or unsubstituted methyl group, a substituted or unsubstituted isopropyl group, a substituted or unsubstituted t-butyl group, a substituted or unsubstituted cyclohexyl group, a substituted or unsubstituted bicycloheptanyl group, or a substituted or unsubstituted adamantyl group.

In one or more embodiments, L₁ and L₂ may each independently be a substituted or unsubstituted divalent phenyl group, a substituted or unsubstituted divalent biphenyl group, or a substituted or unsubstituted divalent naphthyl group, and L may be a substituted or unsubstituted trivalent phenyl group, a substituted or unsubstituted trivalent biphenyl group, or a substituted or unsubstituted trivalent naphthyl group.

In one or more embodiments, the nitrogen-containing compound represented by Formula 1 may be represented by Formula 2-1 or Formula 2-2.

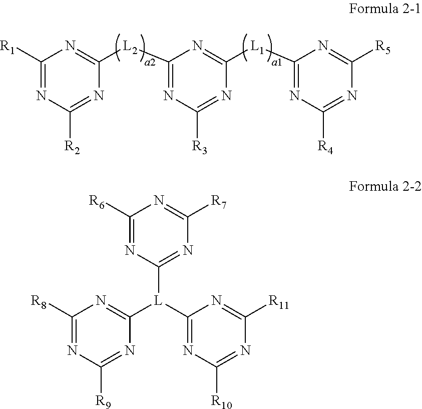

In Formula 2-1 and Formula 2-2, R₁ to R₁₁ may each independently be a hydrogen atom, a deuterium atom, a halogen atom, a nitro group, a cyano group, a substituted or unsubstituted alkyl group having 1 to 30 carbons, a substituted or unsubstituted cycloalkyl group having 3 to 30 ring-forming carbons, a substituted or unsubstituted aryl group having 6 to 60 ring-forming carbons, or a substituted or unsubstituted heteroaryl group having 2 to 60 ring-forming carbons, in Formula 2-1, at least one selected from among R₁ to R₅ may be a substituted or unsubstituted alkyl group having 1 to 30 carbons, or a substituted or unsubstituted cycloalkyl group having 3 to 30 ring-forming carbons, and in Formula 2-2, at least one selected from among R₆ to R₁₁ may be a substituted or unsubstituted alkyl group having 1 to 30 carbons, or a substituted or unsubstituted cycloalkyl group having 3 to 30 ring-forming carbons.

In Formula 2-1 and Formula 2-2, L, L₁, L₂, a1, and a2 may be as defined in Formula 1.

In one or more embodiments, the nitrogen-containing compound represented by Formula 2-1 may be represented by any one selected from among Formula 3-1 to Formula 3-3.

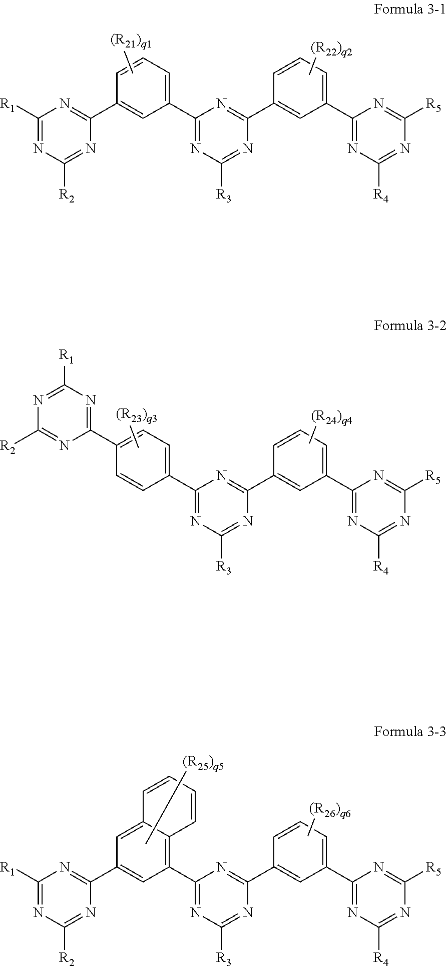

In Formula 3-1 to Formula 3-3, R₂₁ to R₂₆ may each independently be a hydrogen atom, a deuterium atom, a halogen atom, a cyano group, a nitro group, a substituted or unsubstituted alkyl group having 1 to 30 carbons, a substituted or unsubstituted aryl group having 6 to 60 ring-forming carbons, or a substituted or unsubstituted heteroaryl group having 2 to 60 ring-forming carbons, q1 to q4, and q6 may each independently be an integer of 0 to 4, q5 may be an integer of 0 to 6.

In Formula 3-1 to Formula 3-3, R₁ to R₅ may be as defined in Formula 2-1.

In one or more embodiments, the nitrogen-containing compound represented by Formula 2-2 may be represented by Formula 4.

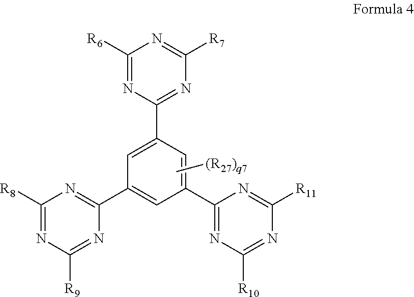

In Formula 4, R₂₇ may be a hydrogen atom, a deuterium atom, a halogen atom, a cyano group, a nitro group, a substituted or unsubstituted alkyl group having 1 to 20 carbons, a substituted or unsubstituted aryl group having 6 to 60 ring-forming carbons, or a substituted or unsubstituted heteroaryl group having 2 to 60 ring-forming carbons, q7 may be an integer of 0 to 3.

In Formula 4, R₆ to R₁₁ may be as defined in Formula 2-2.

In one or more embodiments, in Formula 2-1, at least one selected from among R₁ to R₅ may be a substituted or unsubstituted alkyl group having 1 to 10 carbons, and the rest (e.g., any remaining selected from among R₁ to R₅) may each independently be a substituted or unsubstituted phenyl group, or a substituted or unsubstituted biphenyl group; and in Formula 2-2, at least one selected from among R₆ to R₁₁ may be a substituted or unsubstituted alkyl group having 1 to 10 carbons, and the rest (e.g., any remaining selected from among R₆ to R₁₁) may each independently be a substituted or unsubstituted phenyl group, or a substituted or unsubstituted biphenyl group.

In one or more embodiments, L₁ and L₂ may each independently be represented by any one selected from among Formula L-1 to L-9, and L may be represented by Formula L-10.

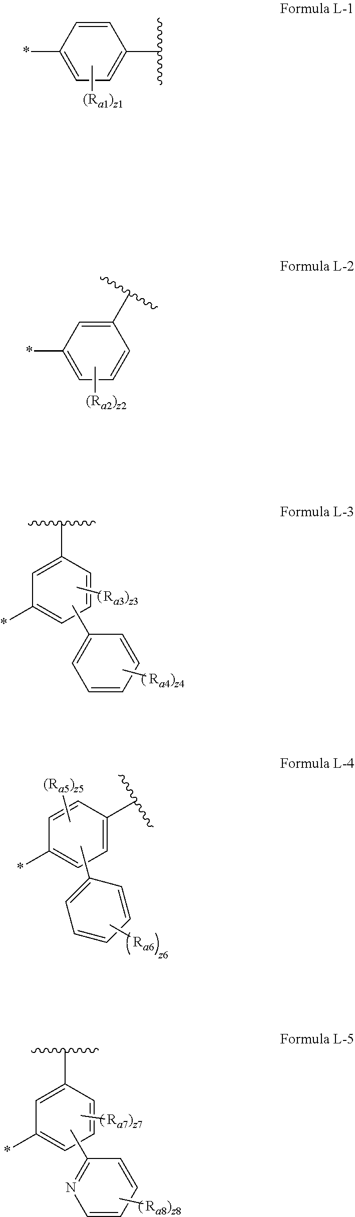

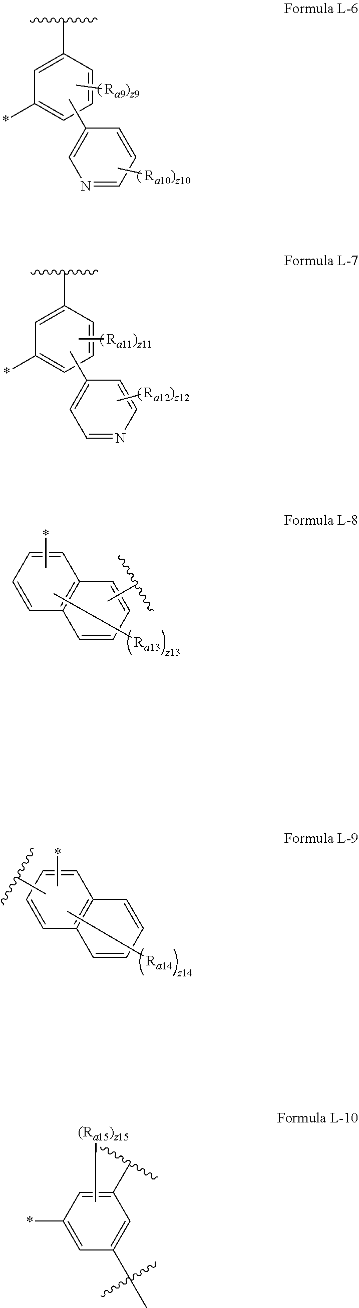

In Formula L-1 to L-10, Rₐ₁ to Rₐ₁₅ may each independently be a hydrogen atom, a deuterium atom, a halogen atom, a cyano group, a substituted or unsubstituted phosphine oxide group, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons, z1, z2, z8, z10, and z12 are each may be independently an integer of 0 to 4, z3, z5, z7, z9, z11, and z15 may each independently be an integer of 0 to 3, z4 and z6 may each independently be an integer of 0 to 5, and z13 and z14 may each independently be an integer of 0 to 6.

In one or more embodiments, the nitrogen-containing compound according to one or more embodiments of the disclosure may be represented by Formula 1.

In one or more embodiments of the disclosure, a display device includes a base layer; a circuit layer arranged on the base layer, and a display element layer arranged on the circuit layer and including a light-emitting element, wherein the light-emitting element includes a first electrode, a second electrode arranged on the first electrode, an emission layer arranged between the first electrode and the second electrode, and at least one functional layer arranged between the first electrode and the emission layer and including a nitrogen-containing compound represented by Formula 1

In one or more embodiments, the light-emitting element may further include a capping layer arranged on the second electrode, and a refractive index of the capping layer for light in a wavelength range of about 550 nm to about 660 nanometer (nm) may be at least about 1.6.

In one or more embodiments, the display device may further include an optical control layer arranged on the display element layer, the optical control layer including a quantum dot, wherein the light-emitting element may be to emit a first color light, and the optical control layer may include: a first optical control part that converts the first color light to a second color light in a longer wavelength range than (e.g., having a wavelength range longer than the wavelength range of) the first color light; a second optical control part that converts the first color light to a third color light in a longer wavelength range than (that of each of) the first color light and the second color light (e.g., having a wavelength range longer than the wavelength range of each of the first color light and the wavelength range of the second color light); and a third optical control part that transmits the first color light.

### BRIEF DESCRIPTION OF THE DRAWINGS

The accompanying drawings are included to provide a further understanding of the present disclosure, and are incorporated in and constitute a part of this specification. The drawings illustrate example embodiments of the present disclosure and, together with the description, serve to explain principles of the present disclosure. In the drawings:

FIG. 1 is a plan view of a display device according to one or more embodiments of the present disclosure;

FIG. 2 is a cross-sectional view of a display device according to one or more embodiments of the present disclosure;

FIG. 3 is a cross-sectional view schematically illustrating a light-emitting element according to one or more embodiments of the present disclosure;

FIG. 4 is a cross-sectional view schematically illustrating a light-emitting element according to one or more embodiments of the present disclosure;

FIG. 5 is a cross-sectional view schematically illustrating a light-emitting element according to one or more embodiments of the present disclosure;

FIG. 6 is a cross-sectional view schematically illustrating a light-emitting element according to one or more embodiments of the present disclosure;

FIG. 7 and FIG. 8 are each a cross-sectional view of a display device according to one or more embodiments of the present disclosure;

FIG. 9 is a cross-sectional view illustrating a display device according to one or more embodiments of the present disclosure;

FIG. 10 is a cross-sectional view illustrating a display device according to one or more embodiments of the present disclosure; and

FIG. 11 is a view illustrating a vehicle in which a display device according to one or more embodiments is arranged.

### DETAILED DESCRIPTION

The present disclosure may be modified in one or more suitable manners and have many forms, and thus specific embodiments will be exemplified in the drawings and described in more detail in the detailed description of present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are used for referring to like elements. In the accompanying drawings, the dimensions of each structure are exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the present disclosure. As used herein, the singular forms, “a,” “an,” and “the” are intended to include the plural forms as well, unless the context clearly indicates otherwise.

In the present application, it will be understood that the terms “include,” “includes,” “including,” “comprise,” “comprises”, “comprising,” “has,” “having,” “have,” and/or the like specify the presence of features, numbers, steps, operations, component, parts, and/or one or more (e.g., any suitable) combinations thereof disclosed in the specification, but do not exclude the possibility of presence or addition of one or more other features, numbers, steps, operations, component, parts, and/or one or more (e.g., any suitable) combinations thereof.

In the present application, if (e.g., when) a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary to this, if (e.g., when) a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that if (e.g., when) a part is referred to as being “on” another part, it can be arranged above the other part, or arranged under the other part as well.

For example, the terms, such as “lower”, “above”, “upper” and/or the like, are utilized herein for ease of description to describe one element's relation to another element(s) as illustrated in the drawings. The terms are relative concepts and are described based on the directions indicated in the drawings. It will be understood that the terms have a relative concept and are described on the basis of the orientation depicted in the drawings. It will be understood that the spatially relative terms are intended to encompass different orientations of the device in use or operation in addition to the orientation depicted in the drawings. For example, if the device in the drawings is turned over, elements described as “beneath” other elements or features would then be oriented “above” or “over” the other elements or features. Thus, the term “beneath” may encompass both an orientation of above and below. The device may be otherwise oriented (rotated 90 degrees or at other orientations), and the spatially relative descriptors used herein should be interpreted accordingly.

As utilized herein, the terms “use,” “using,” and “used” may be considered synonymous with the terms “utilize,” “utilizing,” and “utilized,” respectively.

As utilized herein, expressions such as “at least one of,” “one of,” “selected from,” and “selected from among,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

The term “and/or” includes all combinations of one or more of the associated listed elements.

As utilized herein, the term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Unless otherwise defined, all terms (including chemical, technical and scientific terms) utilized herein have the same meaning as commonly understood by one of ordinary skill in the art to which this present disclosure belongs. It will be further understood that terms, such as those defined in commonly utilized dictionaries, should be interpreted as having a meaning that is consistent with their meaning in the context of the relevant art and will not be interpreted in an idealized or overly formal sense unless expressly so defined herein.

As utilized herein, the phrase “consisting essentially of” refers to that any additional components will not materially affect the chemical, physical, optical, or electrical properties of the semiconductor film.

As utilized herein, the phrase “on a plane,” or “plan view,” refers to viewing a target portion from the top, and the phrase “on a cross-section” refers to viewing a cross-section formed by vertically cutting a target portion from the side.

In present disclosure, “not include a or any ‘component’” “exclude a or any ‘component’”, “‘component’-free”, and/or the like refers to that the “component” not being added, selected or utilized as a component in the composition, but the “component” of less than a suitable amount may still be included due to other impurities and/or external factor.

### Definitions

In the specification, the term “substituted or unsubstituted” may refer to substituted or unsubstituted with at least one substituent selected from among the group including (e.g., consisting of) a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified herein may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but one or more embodiments of the present disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but one or more embodiments of the present disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but one or more embodiments of the present disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 50, 6 to 40, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but one or more embodiments of the present disclosure is not limited thereto.

In the specification, the fluorenyl group may be substituted, and two substituents may be bonded to each other to form a spiro structure. Examples of the substituted fluorenyl group are as follows. However, one or more embodiments of the present disclosure is not limited thereto.

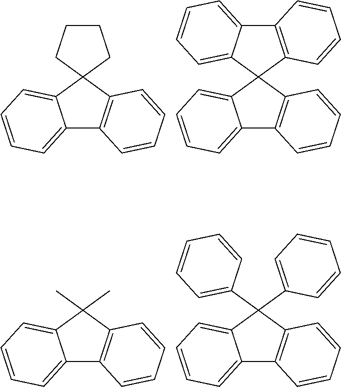

The heterocyclic group herein refers to any functional group or substituent derived from a ring containing at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si or S as a heteroatom. If the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 60, 2 to 50, 2 to 40, 2 to 30, 2 to 20, or 2 to 10.

In the specification, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but one or more embodiments of the present disclosure is not limited thereto.

In the specification, the heteroaryl group may contain at least one of B, O, N, P, Si, or S as a heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 60, 2 to 50, 2 to 40, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but one or more embodiments of the present disclosure is not limited thereto.

In the specification, the description of the aryl group may be applied to an arylene group except that the arylene group is a divalent group. The description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but one or more embodiments of the present disclosure is not limited thereto.

In the specification, the germanium group includes an alkylgermanium group and an arylgermanium group. Examples of germanium group may include a trimethylgermanium group, triethylgermanium group, a t-butyldimethylgermanium group, a vinyldimethylgermanium group, a propyldimethylgermanium group, a triphenylgermanium group, a tribiphenylgermanium group, a dipenylgermanium group, a phenyl germanium group, and/or the like, but one or more embodiments of the present disclosure is not limited thereto.

In the specification, the number of ring-forming carbon atoms in the carbonyl group is not specifically limited, but may be 1 to 40, 1 to 30, or 1 to 20. For example, the carbonyl group may have the following structures, but one or more embodiments of the present disclosure is not limited thereto.

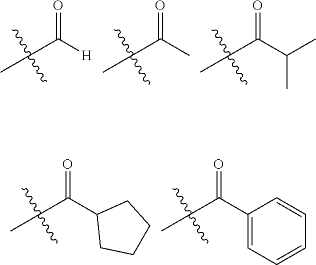

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined herein. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but one or more embodiments of the present disclosure is not limited thereto.

In the specification, an oxy group may refer to that an oxygen atom is bonded to the alkyl group or the aryl group as defined herein. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but one or more embodiments of the present disclosure is not limited thereto.

The boron group herein may refer to that a boron atom is bonded to the alkyl group or the aryl group as defined herein. The boron group includes an alkyl boron group and an aryl boron group. Examples of the boron group may include a dimethylboron group, a trimethylboron group, a t-butyldimethylboron group, a diphenylboron group, a phenylboron group, and/or the like, but one or more embodiments of the present disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and/or the like, but one or more embodiments of the present disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In one or more embodiments, in the specification,

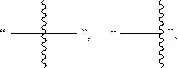

and “-*” refer to a position to be connected.

Hereinafter, embodiments of the present disclosure will be described with reference to the accompanying drawings.

### Display Device

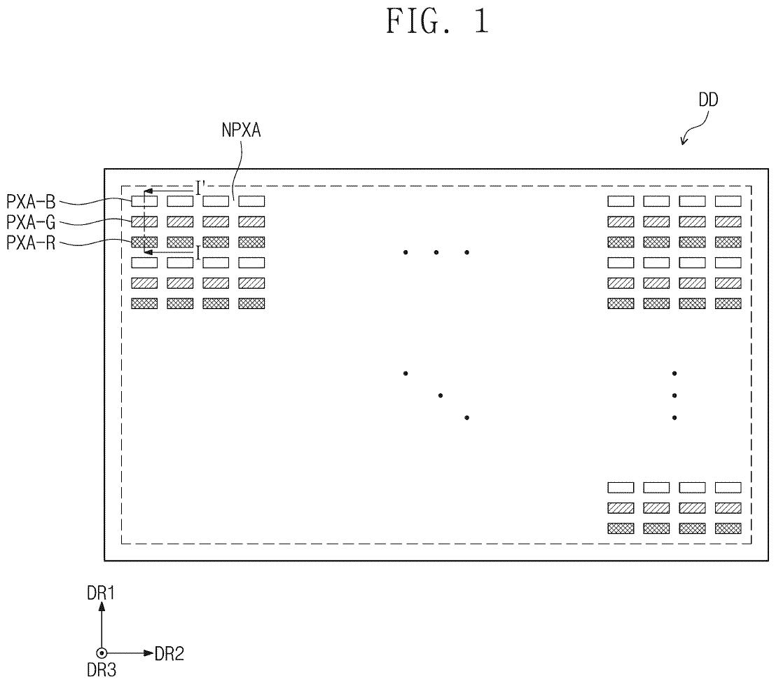

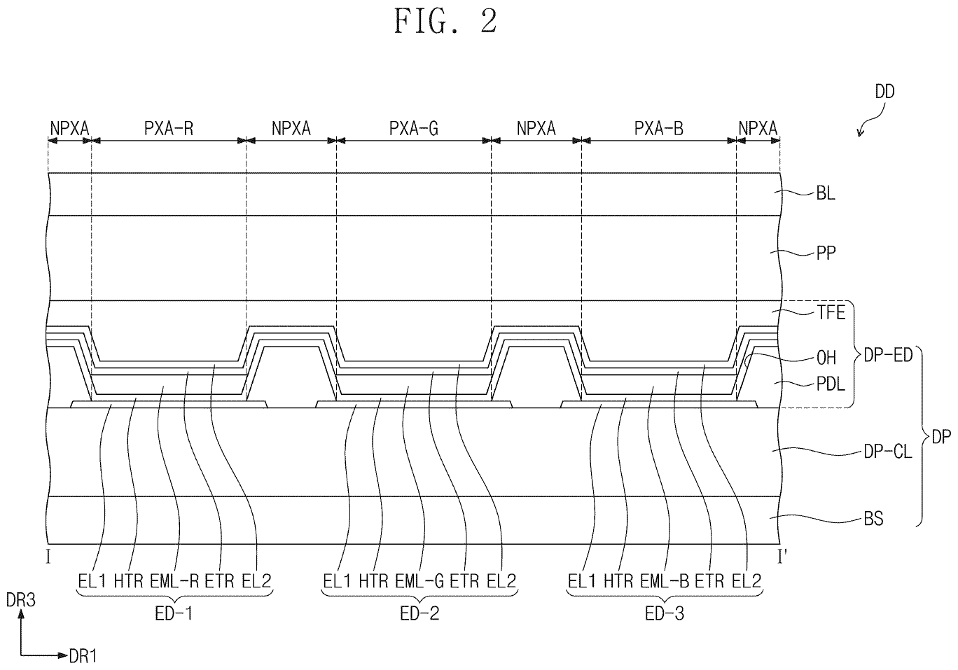

FIG. 1 is a plan view illustrating one or more embodiments of a display device DD. FIG. 2 is a cross-sectional view of the display device DD of the embodiment. FIG. 2 is a cross-sectional view illustrating a part taken along the line I-I′ of FIG. 1.

The display device DD may include a display panel DP and an optical layer PP arranged on the display panel DP. The display panel DP includes light-emitting elements ED-1, ED-2, and ED-3. The display device DD may include a plurality of light-emitting elements ED-1, ED-2, and ED-3. The optical layer PP may be arranged on the display panel DP to control reflected light in the display panel DP due to external light. The optical layer PP may include, for example, a polarization layer or a color filter layer. In one or more embodiments, unlike the configuration illustrated in the drawing, the optical layer PP may not be provided from the display device DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, one or more embodiments of the disclosure is not limited thereto, and the base substrate BL may be an inorganic layer, an organic layer, or a composite material layer. In some embodiments, unlike the configuration illustrated, in one or more embodiments, the base substrate BL may not be provided.

The display device DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light-emitting elements ED-1, ED-2, and ED-3 arranged between portions of the pixel defining film PDL, and an encapsulation layer TFE arranged on the light-emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, one or more embodiments is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light-emitting elements ED-1, ED-2, and ED-3 of the display device layer DP-ED.

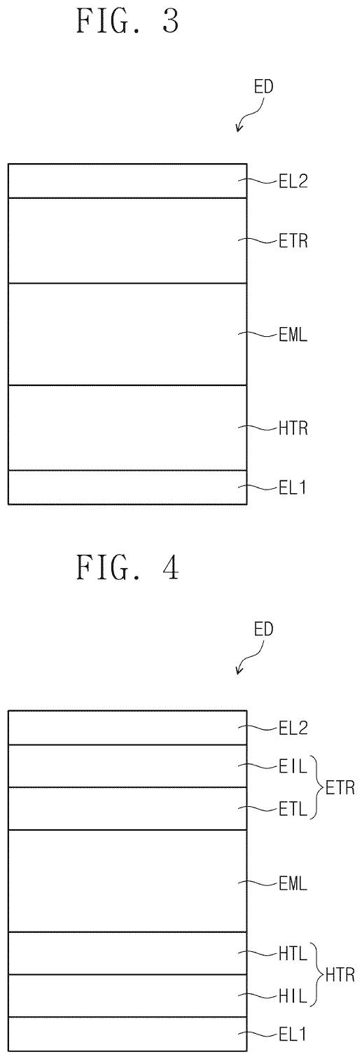

Each of the light-emitting elements ED-1, ED-2, and ED-3 may have a structure of each light-emitting element ED of embodiments according to FIGS. 3 to 6, as described in more detail elsewhere herein. Each of the light-emitting elements ED-1, ED-2, and ED-3 may include a first electrode EL1, a hole transport region HTR, emission layers EML-R, EML-G, and EML-B, an electron transport region ETR, and a second electrode EL2.

FIG. 2 illustrates one or more embodiments in which the emission layers EML-R, EML-G, and EML-B of the light-emitting elements ED-1, ED-2, and ED-3 are arranged in openings OH defined in the pixel defining film PDL, and the hole transport region HTR, the electron transport region ETR, and the second electrode EL2 are provided as a common layer in the entire light-emitting elements ED-1, ED-2, and ED-3. However, one or more embodiments of the disclosure is not limited thereto, and unlike the configuration illustrated in FIG. 2, the hole transport region HTR and the electron transport region ETR in one or more embodiments may be provided by being patterned inside the openings OH defined in the pixel defining film PDL. For example, the hole transport region HTR, the emission layers EML-R, EML-G, and EML-B, and the electron transport region ETR of the light-emitting elements ED-1, ED-2, and ED-3 in one or more embodiments may be provided by being patterned in an inkjet printing method.

The encapsulation layer TFE may cover the light-emitting elements ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display device layer DP-ED from moisture and/or oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but one or more embodiments of the disclosure is not particularly limited thereto. The encapsulation-organic film may include an acrylic-based compound, an epoxy-based compound, and/or the like. The encapsulation-organic film may include a photopolymerizable organic material, but one or more embodiments of the disclosure is not particularly limited thereto.

The encapsulation layer TFE may be arranged on the second electrode EL2 and may be arranged filling the opening OH.

Referring to FIGS. 1 and 2, the display device DD may include one or more non-light emitting region(s) NPXA and also include light emitting regions PXA-R, PXA-G, and PXA-B. The light emitting regions PXA-R, PXA-G, and PXA-B may be regions in which light generated by the respective light-emitting elements ED-1, ED-2, and ED-3 are emitted. The light emitting regions PXA-R, PXA-G, and PXA-B may be spaced and/or apart (e.g., spaced apart or separated) from each other on a plane (e.g., in a plan view).

Each of the light emitting regions PXA-R, PXA-G, and PXA-B may be a region divided (i.e., defined) by the pixel defining film PDL. The non-light emitting areas NPXA may be areas between the adjacent light emitting areas PXA-R, PXA-G, and PXA-B, which correspond to the pixel defining film PDL. In one or more embodiments, in the specification, the light emitting regions PXA-R, PXA-G, and PXA-B may respectively correspond to pixels. The pixel defining film PDL may divide the light-emitting elements ED-1, ED-2, and ED-3. The emission layers EML-R, EML-G, and EML-B of the light-emitting elements ED-1, ED-2, and ED-3 may be arranged in openings OH defined in the pixel defining film PDL and separated from each other.

The light emitting regions PXA-R, PXA-G, and PXA-B may be divided into a plurality of groups according to the color of light generated from the light-emitting elements ED-1, ED-2, and ED-3. In the display device DD of one or more embodiments illustrated in FIGS. 1 and 2, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display device DD according to one or more embodiments, the plurality of light-emitting elements ED-1, ED-2 and ED-3 may be to emit light beams having wavelengths different from each other. For example, in one or more embodiments, the display device DD may include a first light-emitting element ED-1 that emits red light, a second light-emitting element ED-2 that emits green light, and a third light-emitting element ED-3 that emits blue light. For example, the red light emitting region PXA-R, the green light emitting region PXA-G, and the blue light emitting region PXA-B of the display device DD may correspond to the first light-emitting element ED-1, the second light-emitting element ED-2, and the third light-emitting element ED-3, respectively.

However, one or more embodiments of the disclosure is not limited thereto, and the first to third light-emitting elements ED-1, ED-2, and ED-3 may be to emit light beams in substantially the same wavelength range or at least one light-emitting element may be to emit a light beam in a wavelength range different from the others. For example, the first to third light-emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 1, the plurality of red light emitting regions PXA-R, may be arranged with each other along a second directional axis DR2, the plurality of green light emitting regions PXA-G may be arranged with each other along the second directional axis DR2, and the plurality of blue light emitting regions PXA-B each may be arranged along the second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but one or more embodiments of the disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas if (e.g., when) viewed on a plane defined by the first directional axis DR1 and the second directional axis DR2.

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, the arrangement form of the light emitting regions PXA-R, PXA-G, and PXA-B may be a pentile (PENTILE™) arrangement form or a diamond (Diamond Pixel™) arrangement form, (PENTILE™ and Diamond Pixel™ are registered trademarks owned by Samsung Display Co., Ltd.).

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but one or more embodiments of the disclosure is not limited thereto.

Hereinafter, FIG. 3 to FIG. 6 are cross-sectional views schematically showing light-emitting elements according to one or more embodiments. The light-emitting element ED according to one or more embodiments may include a first electrode EL1, a second electrode EL2 oppositely arranged to the first electrode EL1, and at least one functional layer arranged between the first electrode EL1 and the second electrode EL2. The light-emitting element ED of one or more embodiments may include a fused polycyclic compound of one or more embodiments, which will be explained later, in the at least one functional layer.

The light-emitting element ED may include a hole transport region HTR, an emission layer EML, an electron transport region ETR, and/or the like, stacked in order, as the at least one functional layer. Referring to FIG. 3, the light-emitting element ED of one or more embodiments may include a first electrode EL1, a hole transport region HTR, an emission layer EML, an electron transport region ETR, and a second electrode EL2, stacked in order.

The light-emitting element ED according to one or more embodiments may include a nitrogen-containing compound of one or more embodiments, which will be explained later, in the electron transport region ETR between the first electrode EL1 and the second electrode EL2. However, one or more embodiments of the disclosure is not limited thereto. In some embodiments to the electron transport region ETR, the light-emitting element ED according to one or more embodiments may include a nitrogen-containing compound of one or more embodiments, which will be explained later, in the light-emitting layer EML or the hole transport retion HTR which are a plurality of functional layer between the first electrode EL1 and the second electrode EL2, or may include an amine compound of one or more embodiments, which will be explained later, in the capping layer CPL on the second electrode EL2.

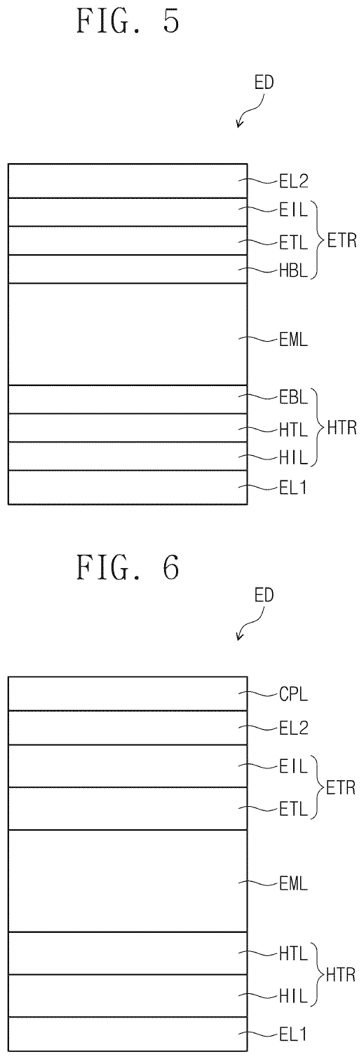

Compared with FIG. 3, FIG. 4 illustrates a cross-sectional view of a light-emitting element ED of one or more embodiments, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light-emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light-emitting element ED of one or more embodiments including a capping layer CPL arranged on a second electrode EL2.

The light-emitting element ED according to one or more embodiments may include a nitrogen-containing compound of one or more embodiments, which will be explained later, in the electron transport region ETR. In the light-emitting element ED according to one or more embodiments, at least one among the electron injection layer EIL, the electron transport layer ETL, and the hole blocking layer HBL may include a nitrogen-containing compound of one or more embodiments. In one or more embodiments, a layer adjacent to the light-emitting layer among the plurality of layers included in the electron transport region ETR may contain a nitrogen-containing compound of one or more embodiments represented by Formula 1.

The first electrode EL1 has conductivity (e.g., is a conductor). The first electrode EL1 may be formed of a metal material, a metal alloy, or a conductive compound. The first electrode EL1 may be an anode or a cathode. However, one or more embodiments of the disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among among Ag, Mg, Cu, Al, Pt, Pd, Au, Ni, Nd, Ir, Cr, Li, Ca, LiF, Mo, Ti, W, In, Sn, and Zn, a compound of two or more selected from among among these, a mixture of two or more selected from among among these, or an oxide thereof.

If the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In one or more embodiments, the first electrode EL1 may have a multilayer structure including a reflective film or a transflective film formed of the herein-described materials, and a transparent conductive film formed of ITO, IZO, ZnO, ITZO, and/or the like. For example, the first electrode EL1 may have a three-layer structure of ITO/Ag/ITO, but one or more embodiments of the disclosure is not limited thereto. In some embodiments, one or more embodiments of the disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 angstrom (Å) to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission-auxiliary layer, or an electron blocking layer EBL. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but one or more embodiments of the disclosure is not limited thereto.

The hole transport region HTR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include a compound represented by Formula H-1:

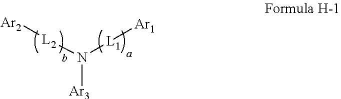

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a or b is an integer of 2 or greater, (then) a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In one or more embodiments, the compound represented by Formula H-2 may be a diamine compound in which at least one among Ar₁ to Ar₃ includes the amine group as a substituent. In some embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one selected from among the compounds in Compound Group H. However, the compounds listed in Compound Group H are examples, and the compounds represented by Formula H-1 are not limited to those represented by Compound Group H:

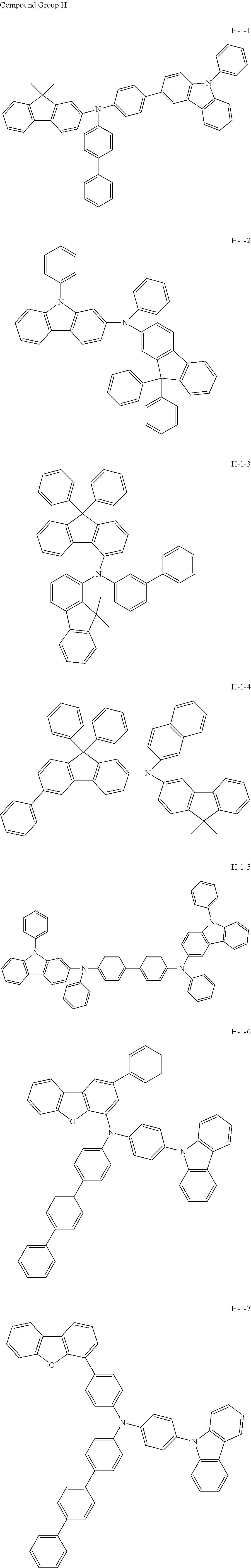

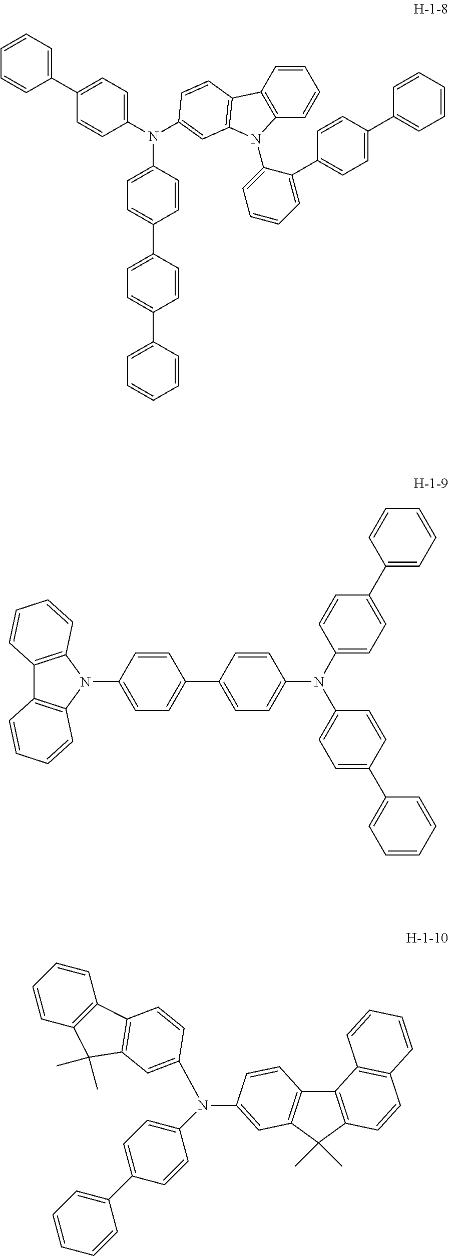 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

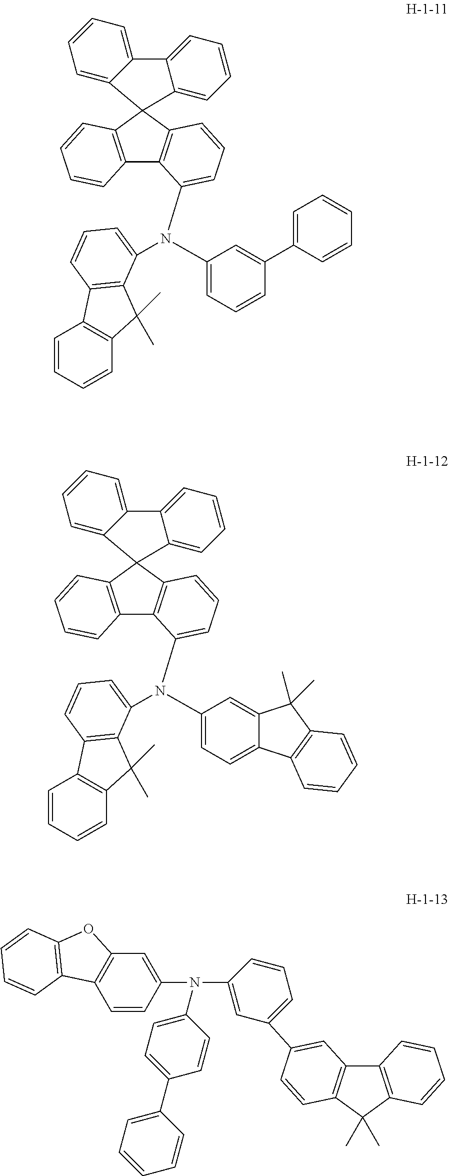 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

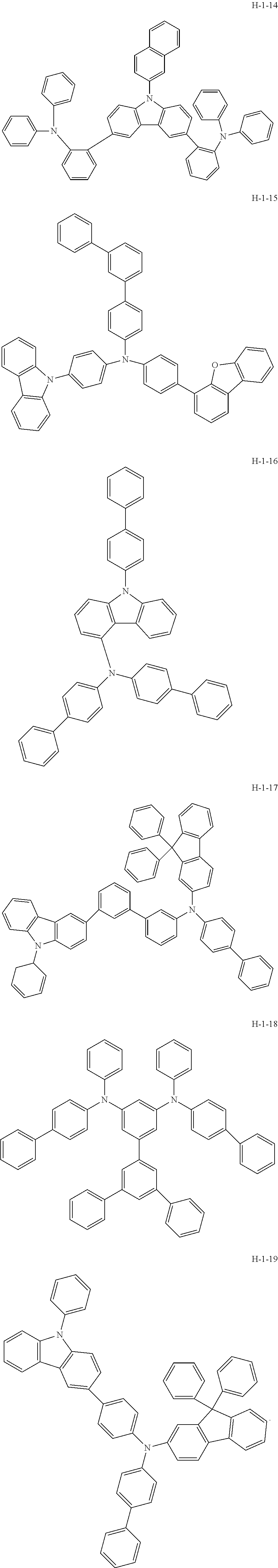

The hole transport region HTR may include a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), and/or the like.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the herein-described compounds of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, if (e.g., when) the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the herein-described ranges, satisfactory hole transport properties may be achieved without a substantial increase in driving voltage.

The hole transport region HTR may further include a charge generating material to increase conductivity in addition to the herein-described materials. The charge generating material may be dispersed uniformly (e.g., substantially uniformly) or non-uniformly (e.g., substantially non-uniformly) in the hole transport region HTR. The charge generating material may be, for example, a p-dopant. The p-dopant may include at least one of a halogenated metal compound, a quinone derivative, a metal oxide, or a cyano group-containing compound, but one or more embodiments of the disclosure is not limited thereto. For example, the p-dopant may include a metal halide compound such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f: 2′,3′-h]quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but one or more embodiments of the disclosure is not limited thereto.

As described herein, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be used as a material to be included in the buffer layer. The electron blocking layer EBL is a layer that serves to prevent or reduce the electron injection from the electron transport region ETR to the hole transport region HTR.

The light emitting layer EML is provided on the hole transport region HTR. The light emitting layer EML may have a thickness of about 100 Å to about 1000 Å, or about 100 Å to about 300 Å. The light emitting layer EML may have a single-layered structure having a single layer formed of a single material, a single-layered structure having a single layer formed of a plurality of different materials, or a multi-layered structure having a plurality of layers formed of a plurality of different materials.

In the light-emitting element ED of one or more embodiments, the light-emitting layer EML may include anthracene derivatives, pyrene derivatives, fluoranthene derivatives, chrysene derivatives, dihydrobenzanthracene derivatives, or triphenylene derivatives. For example, the light-emitting layer EML may include anthracene derivatives or pyrene derivatives.

In the light-emitting element ED of one or more embodiments illustrated in FIG. 3 to FIG. 6, the light emitting layer EML may include a host and a dopant.

The emission layer EML may include a first compound represented by any one selected from among Formula F-a to Formula F-c. The compounds represented by Formula F-a to Formula F-c may be used as a fluorescent dopant material. For example, the compound represented by Formula F-c may be used as a fluorescence dopant material in the emission layer EML.

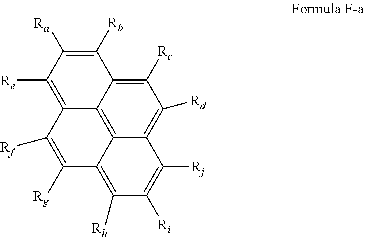

In Formula F-a, two selected from among Rₐ to Rⱼ may each independently be substituted with *—NAr₁Ar₂. Any remaining selected from among Rₐ to Rⱼ not substituted with *—NAr₁Ar₂ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *-NA₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including O or S as a ring-forming atom.

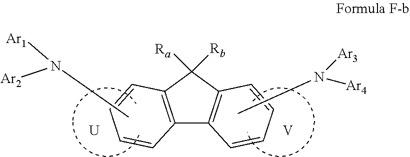

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms. At least one among Ar₁ to Ar₄ may be a heteroaryl group including O or S as a ring-forming atom.

In Formula F-b, the number of rings represented by U and V may each independently be 0 or 1. For example, in Formula F-b, if (e.g., when) the number of U or V is 1, one ring may form a fused ring at the designated part by U or V, and if (e.g., when) the number of U or V is 0, a ring may not be present at the designated part by U or V. For example, if (e.g., when) the number of U is 0, and the number of V is 1, or if (e.g., when) the number of U is 1, and the number of V is 0, a fused ring having the fluorene core of Formula F-b may be a ring compound with four rings. In some embodiments, if (e.g., when) the number of both (e.g., simultaneously) U and V is 0, the fused ring of Formula F-b may be a ring compound with three rings. In some embodiments, if (e.g., when) the number of both (e.g., simultaneously) U and V is 1, a fused ring having the fluorene core of Formula F-b may be a ring compound with five rings.

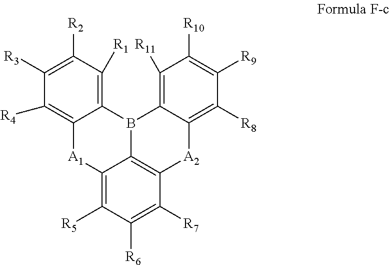

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may each independently be combined with the substituents of an adjacent ring to form a fused ring. For example, if (e.g., when) A₁ and A₂ are each independently NRₘ, A₁ may be combined with R₄ or R₅ to form a ring. In some embodiments, A₂ may be combined with R₇ or R₈ to form a ring.

The emission layer EML may include a compound represented by Formula E-1. The compound represented by Formula E-1 may be used as a fluorescence host material.

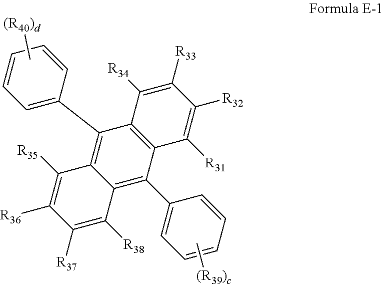

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In one or more embodiments, each of R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one selected from among Compound E1 to Compound E19.

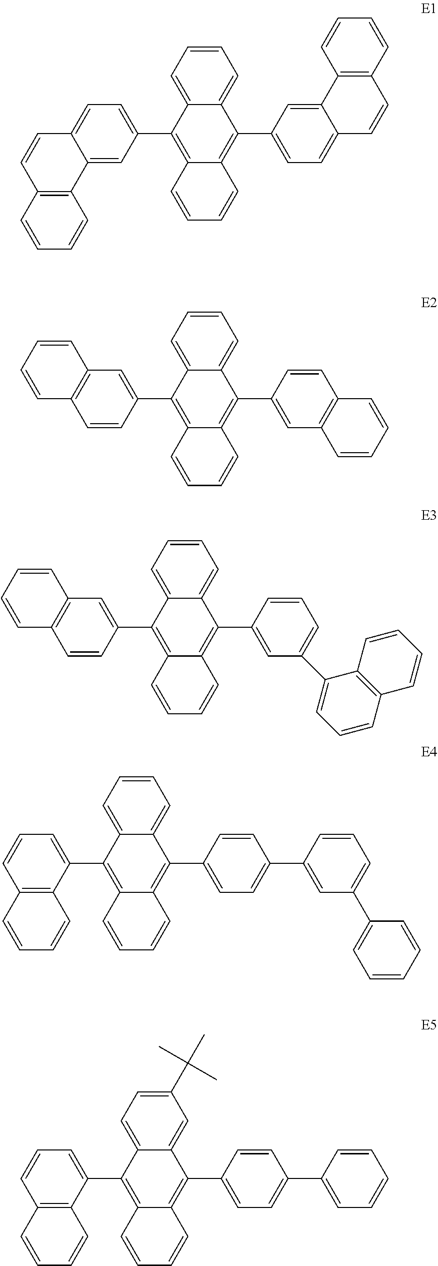

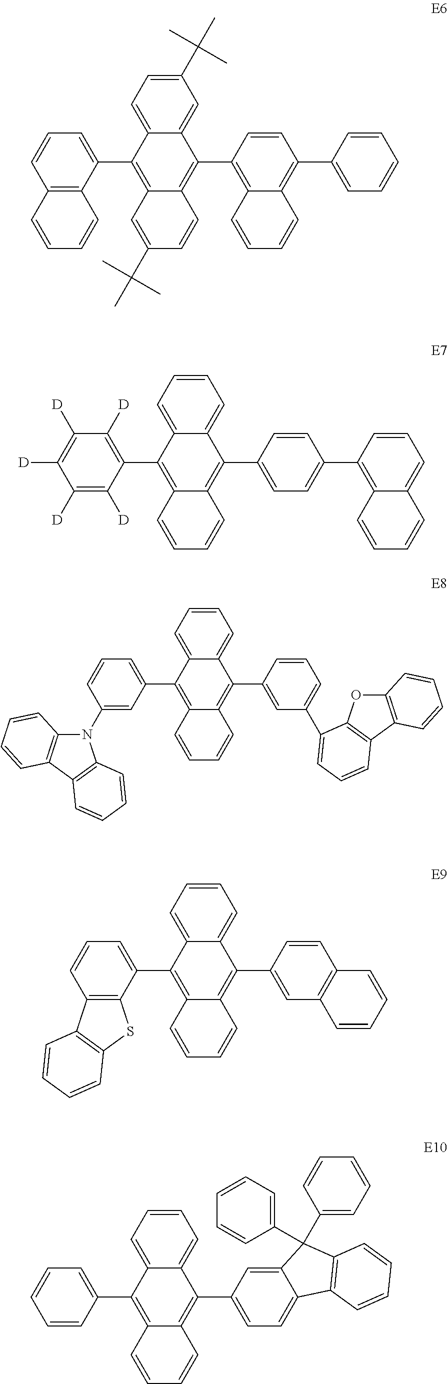

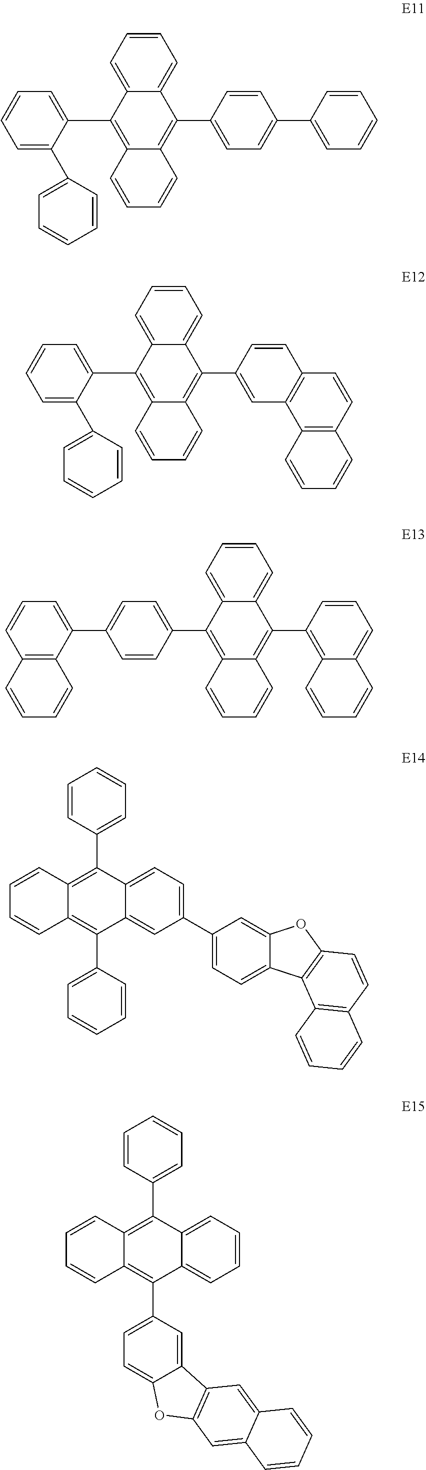

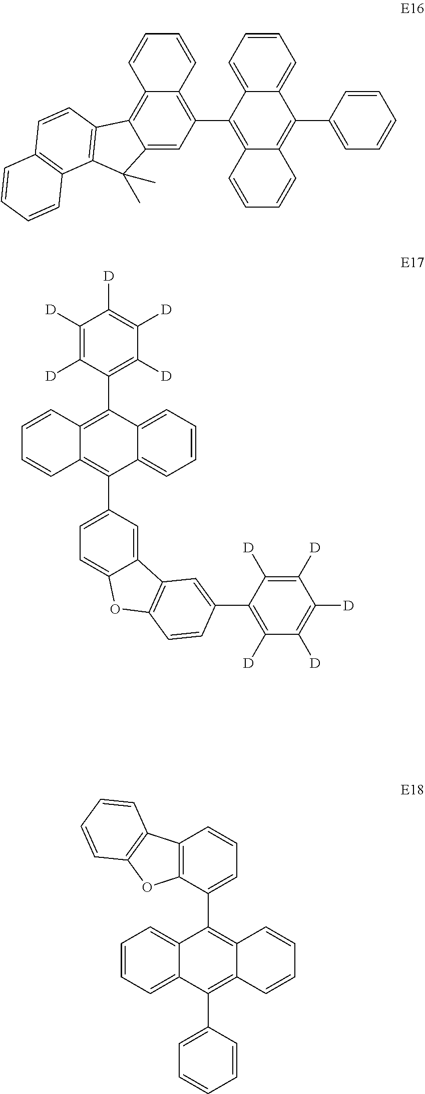

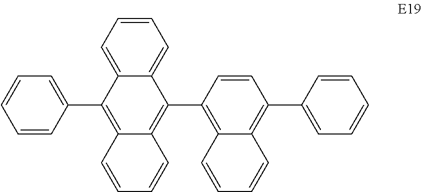 [SMILES: c1ccc(-c2ccc(-c3c4ccccc4c(-c4ccccc4)c4ccccc34)c3ccccc23)cc1]

In one or more embodiments, the emission layer EML may include the first compound represented by any one selected from among Formula F-1 to Formula F-c, and at least one of (e.g., selected from among) the second compound represented by Formula HT-1, the third compound represented by Formula ET-1, or the fourth compound represented by Formula D-1.

In one or more embodiments, the second compound may be used as a hole transport host material of the emission layer EML.

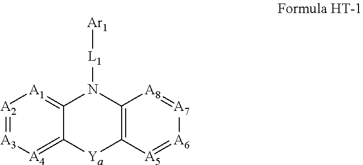

In Formula HT-1, A₁ to A₈ may each independently be N or CR₅₁. For example, A₁ to A₈ may each be (e.g., all) CR₅₁. In one or more embodiments, any one selected from among A₁ to A₈ may be N, and any remaining selected from among A₁ to A₈ may be CR₅₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, and/or the like, but one or more embodiments of the disclosure is not limited thereto.

In Formula HT-1, Yₐ may be a direct linkage, CR₅₂R₅₃, or SiR₅₄R₅₅. For example, two benzene rings connected with the nitrogen atom of Formula HT-1 may be connected via a direct linkage,

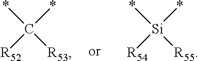

In Formula HT-1, if Yₐ is a direct linkage, the second compound represented by Formula HT-1 may include a carbazole moiety.

In Formula HT-1, Ar₁ may be a substituted or unsubstituted aryl group of 6 to 30 ring-forming carbon atoms, or a substituted or unsubstituted heteroaryl group of 2 to 30 ring-forming carbon atoms. For example, Ar₁ may be a substituted or unsubstituted carbazole group, a substituted or unsubstituted dibenzofuran group, a substituted or unsubstituted dibenzothiophene group, or a substituted or unsubstituted biphenyl group, but one or more embodiments of the disclosure is not limited thereto.

In Formula HT-1, R₅₁ to R₅₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 ring-forming carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, each of R₅₁ to R₅₅ may be combined with an adjacent group to form a ring. For example, R₅₁ to R₅₅ may each independently be a hydrogen atom or a deuterium atom. R₅₁ to R₅₅ may each independently be an unsubstituted methyl group or an unsubstituted phenyl group.

In one or more embodiments, the second compound represented by Formula HT-1 may be represented by any one selected from among the compounds represented in Compound Group 2. The emission layer EML may include at least one selected from among the compounds represented in Compound Group 2 as a hole transport host material.

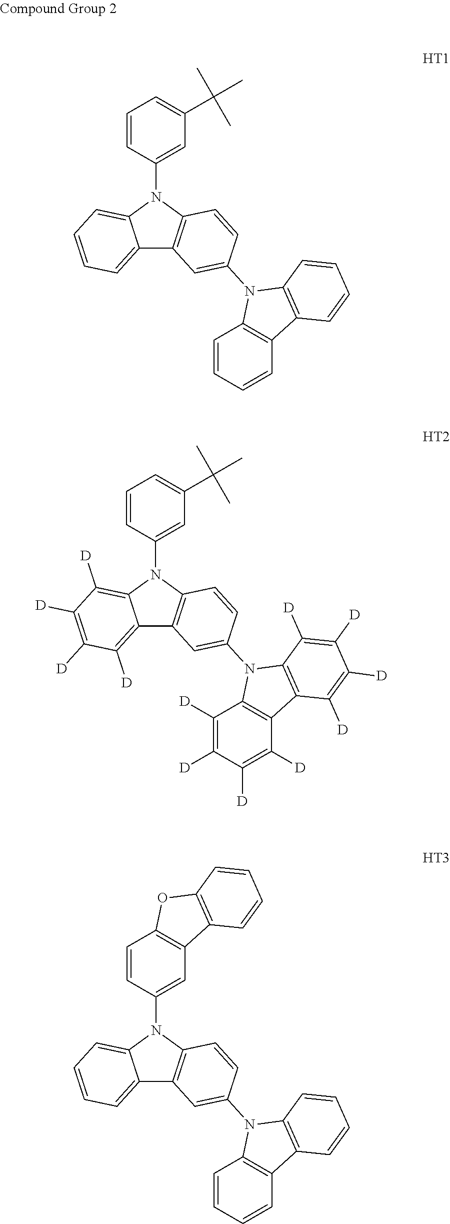 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

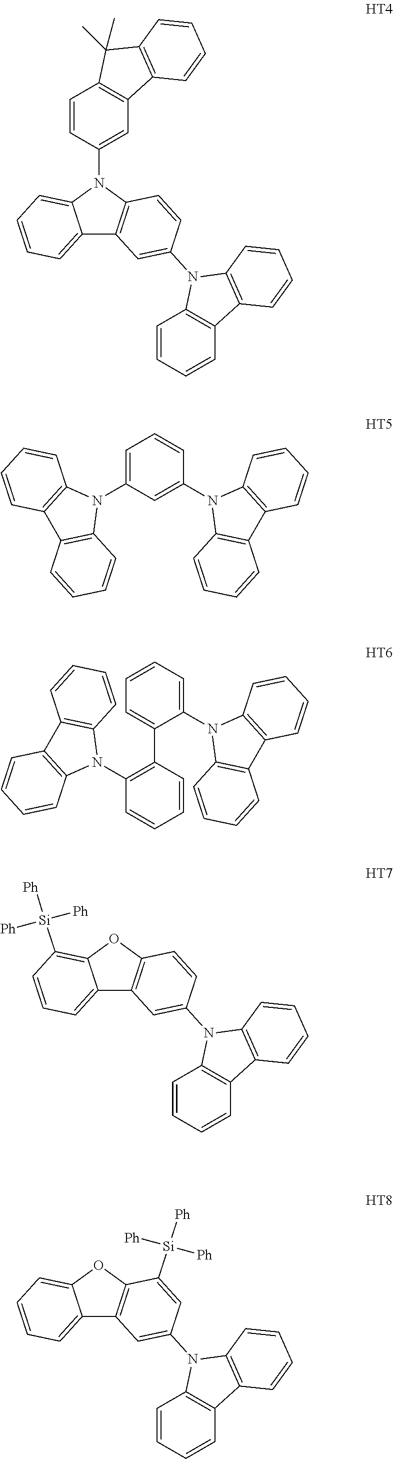 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

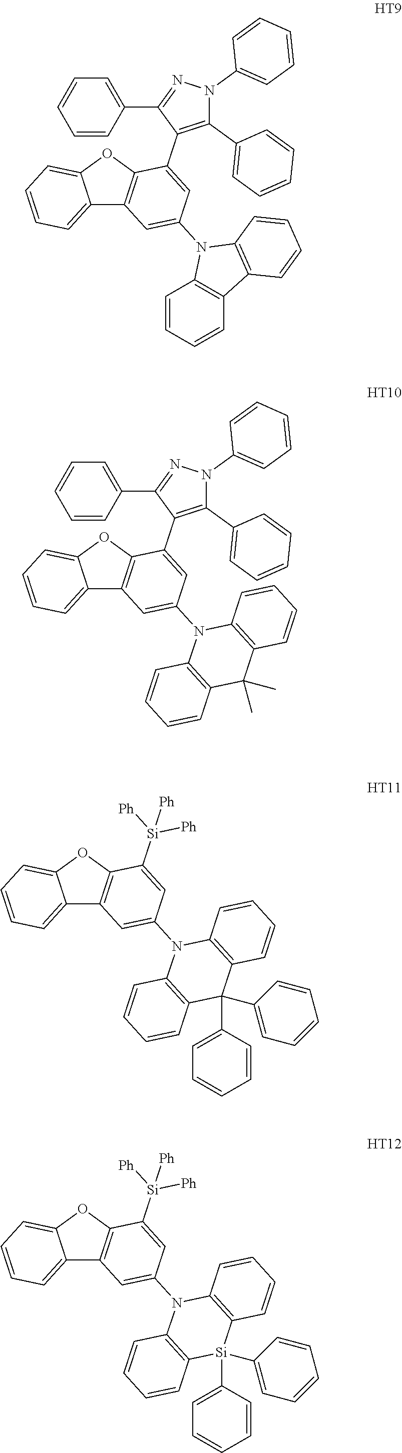 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

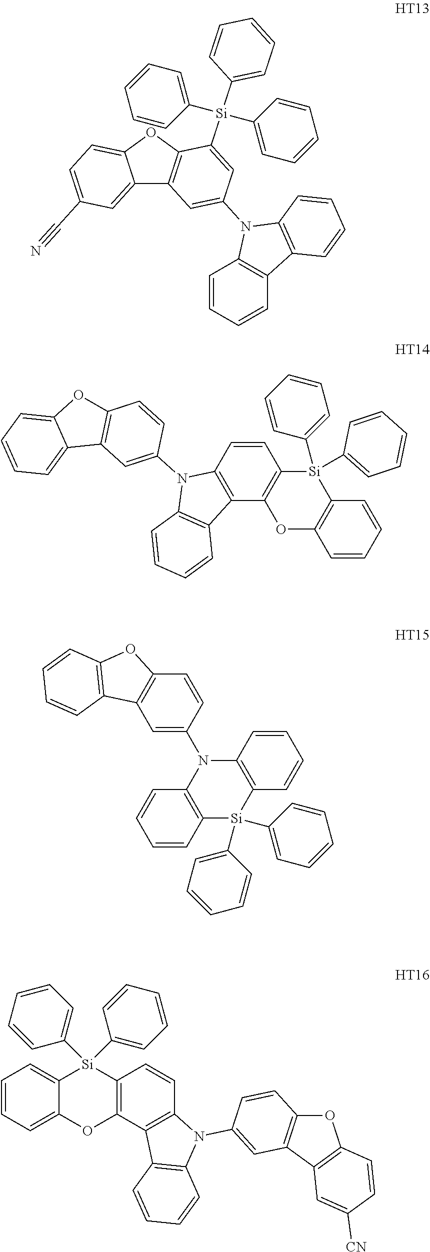 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

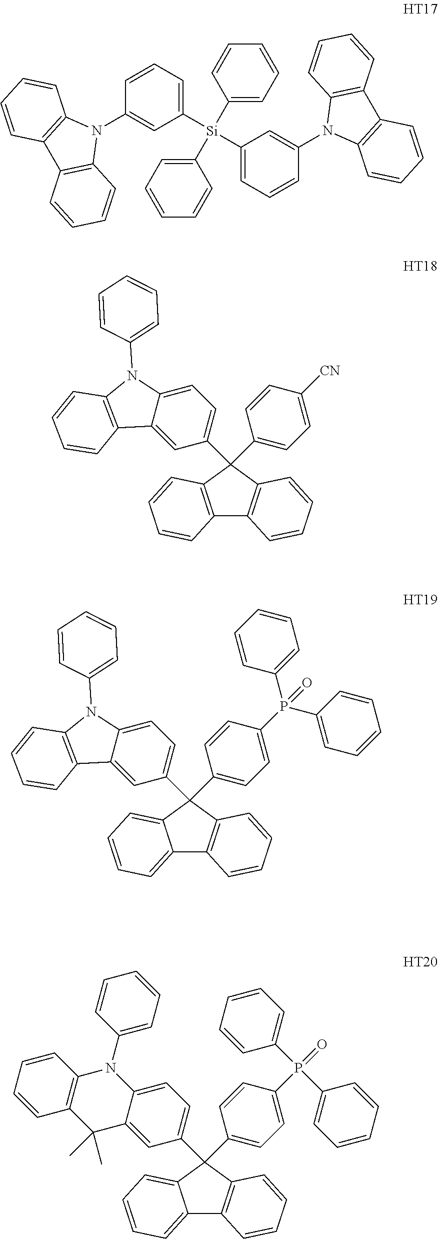 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

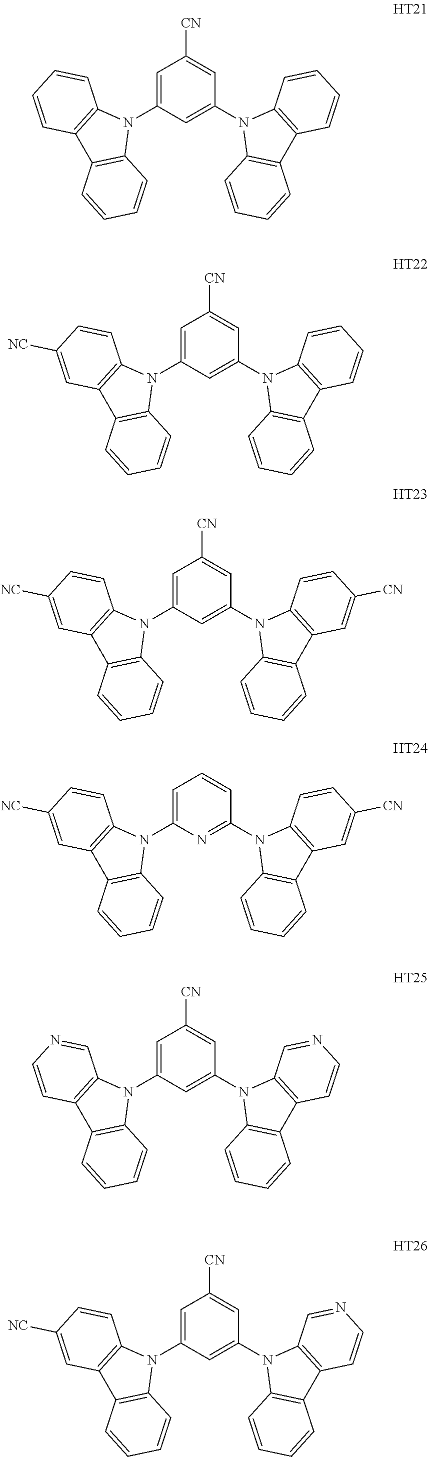 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

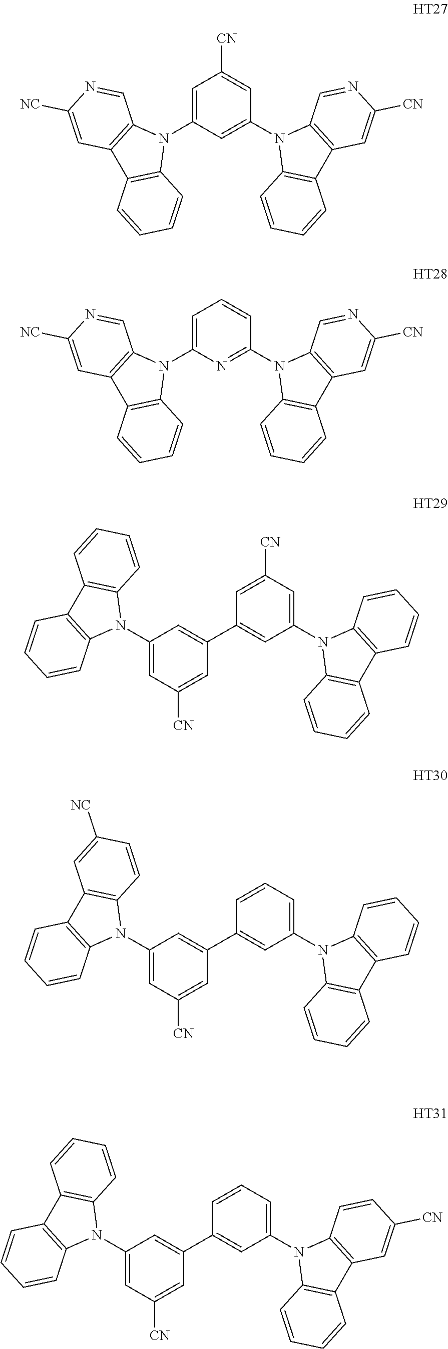 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

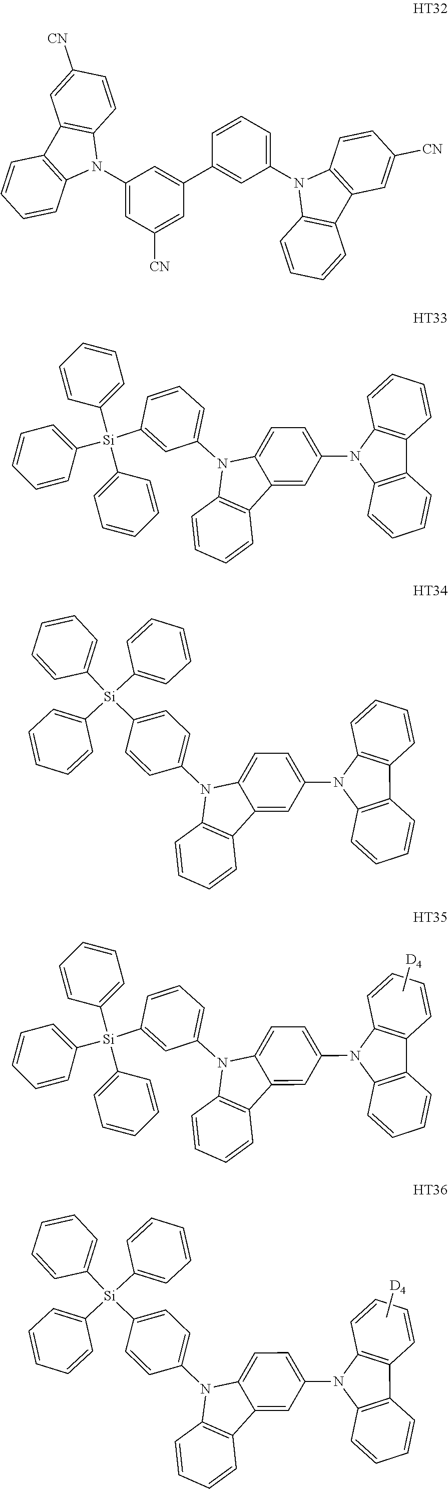

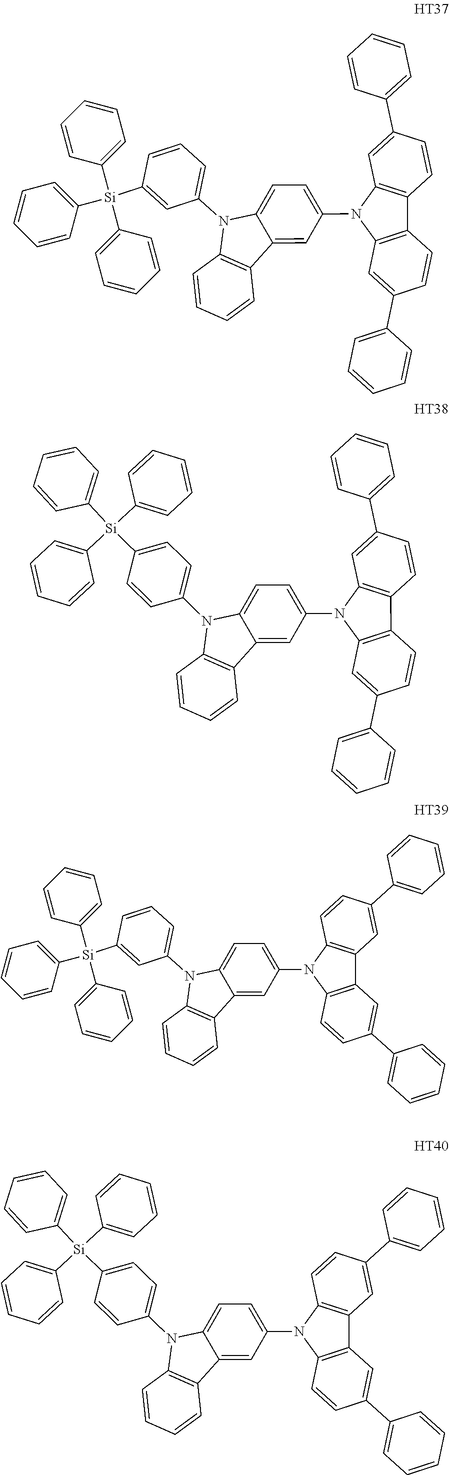

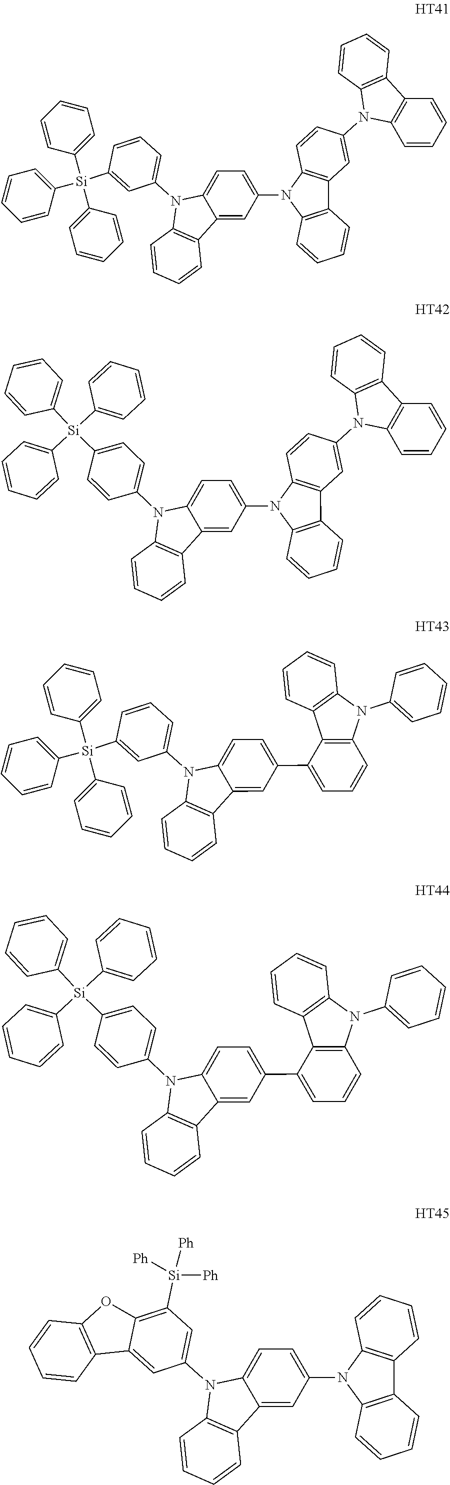

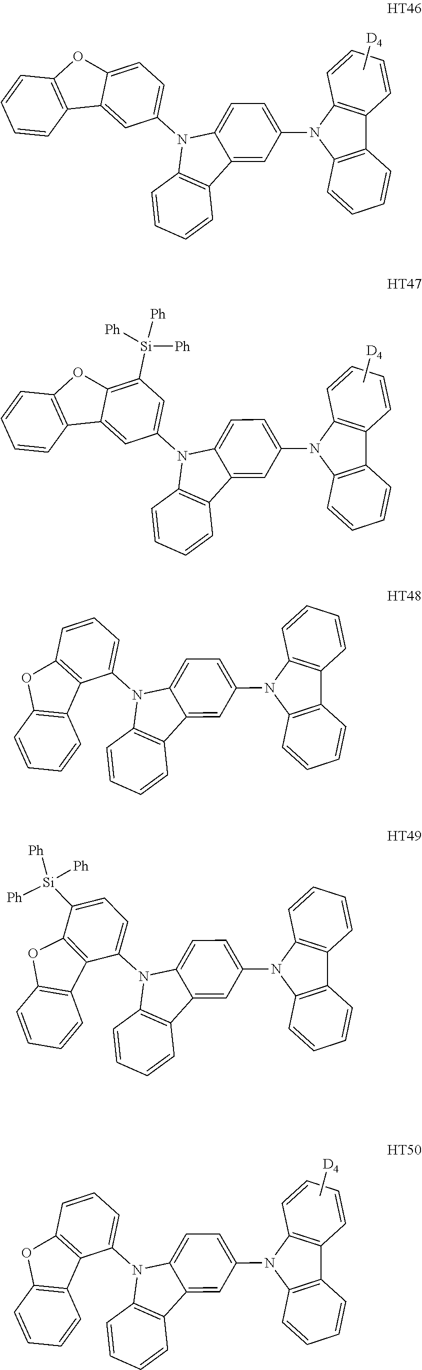

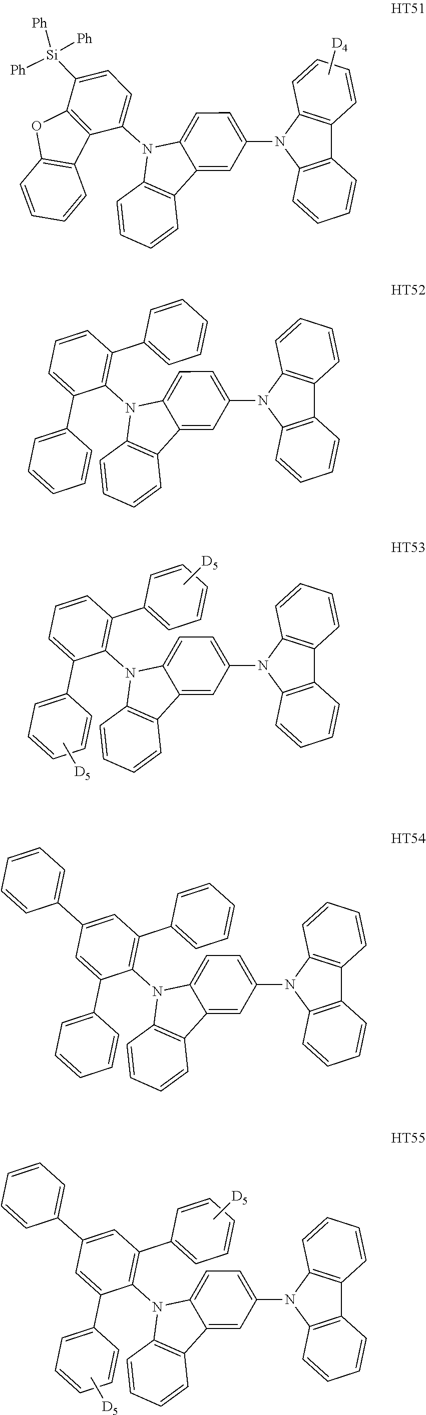

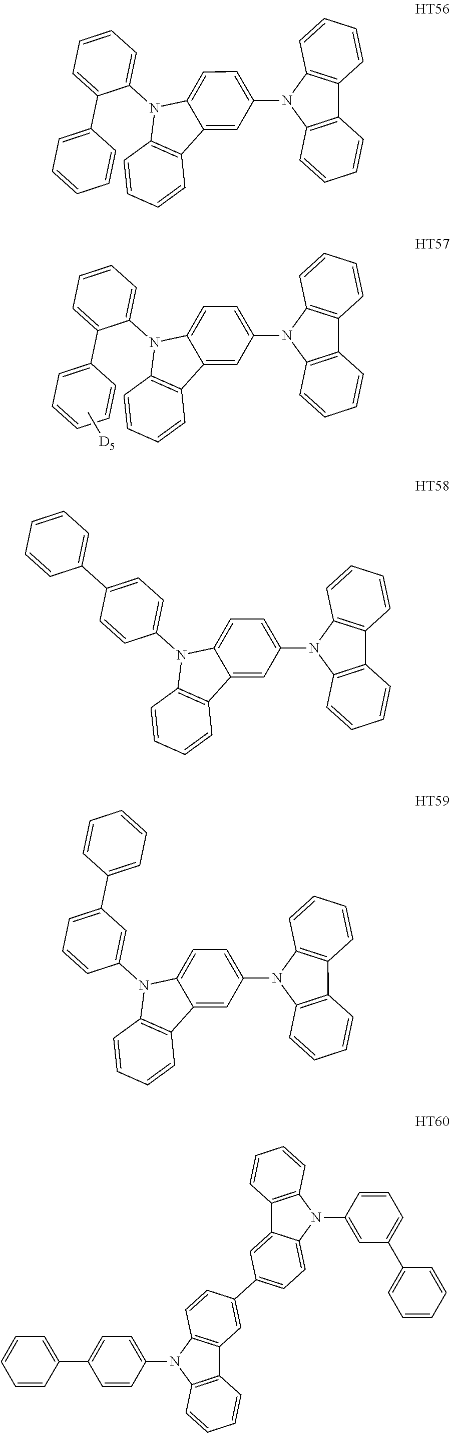

In the particular compounds suggested in Compound Group 2, “D” refers to a deuterium atom, and “Ph” refers to an unsubstituted phenyl group.

In one or more embodiments, the emission layer EML may include a third compound represented by Formula ET-1. For example, the third compound may be used as the electron transport host material of the emission layer EML.

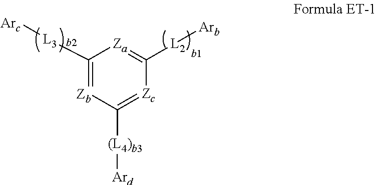

In Formula ET-1, at least one selected from among Zₐ to Z_{c} is N, and the remainder are CR₅₆. For example, any one selected from among Zₐ to Z_{c} may be N, and the remaining two may each independently be CR₅₆. In this case, the third compound represented by Formula ET-1 may include a pyridine moiety. In one or more embodiments, two selected from among Zₐ to Z_{c} may be N, and the remaining one may be CR₅₆. In this case, the third compound represented by Formula ET-1 may include a pyrimidine moiety. In one or more embodiments, Zₐ to Z_{c} may each be (e.g., all) N. In this case, the third compound represented by Formula ET-1 may include a triazine moiety.

In Formula ET-1, R₅₆ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula ET-1, b1 to b3 may each independently be an integer of 0 to 10.

In Formula ET-1, Ar_{b} to Ar_{d} may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar_{b} to Ara may be substituted or unsubstituted phenyl groups or substituted or unsubstituted carbazole groups.

In Formula ET-1, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if b1 to b3 are integers of 2 or more, L₂ to L₄ may each independently be a substituted or unsubstituted arylene group of 6 to 30 ring-forming carbon atoms, or a substituted or unsubstituted heteroarylene group of 2 to 30 ring-forming carbon atoms.

In one or more embodiments, the third compound may be represented by any one selected from among the compounds in Compound Group 3. The light-emitting element ED of one or more embodiments may include any one selected from among the compounds in Compound Group 3.

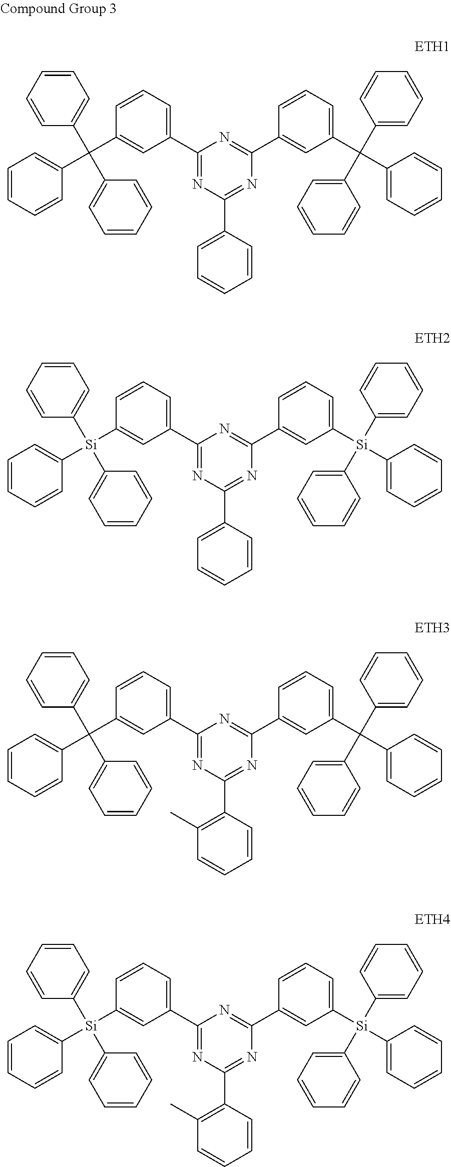 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

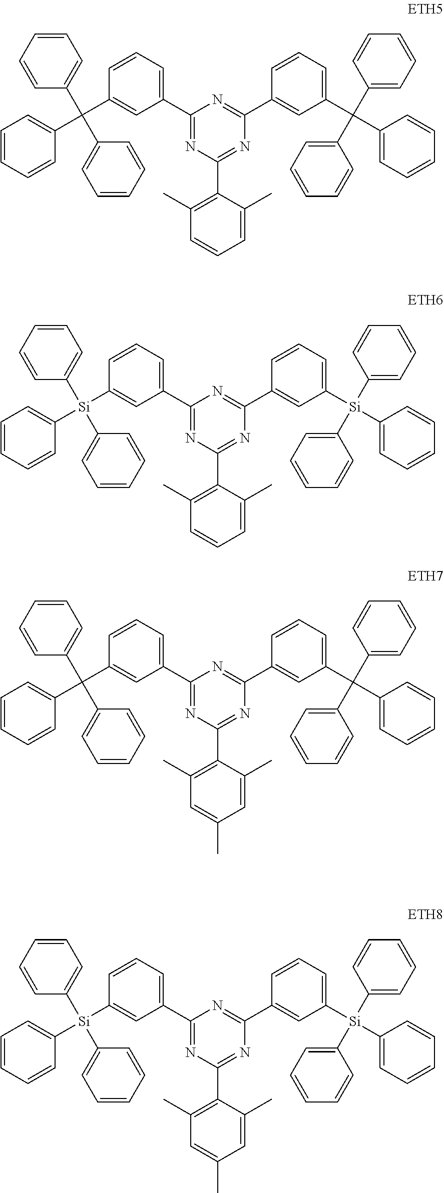 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

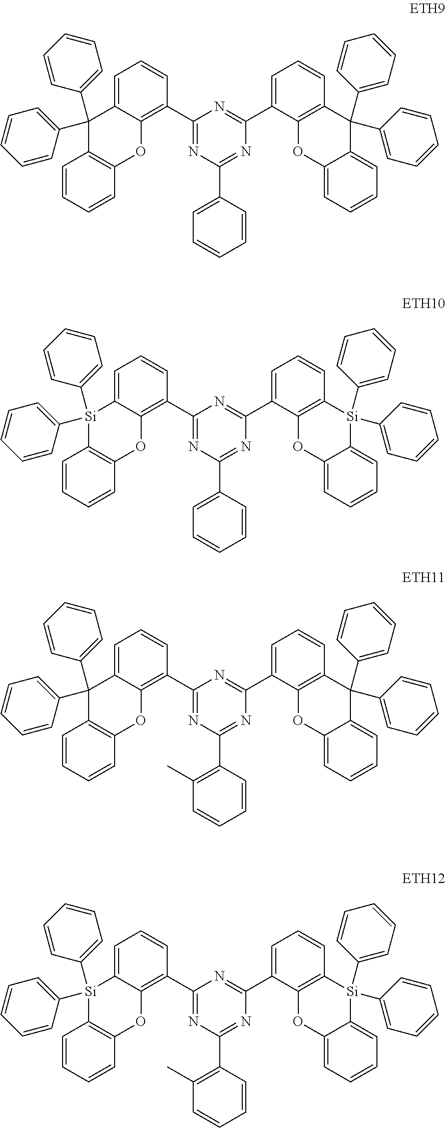 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

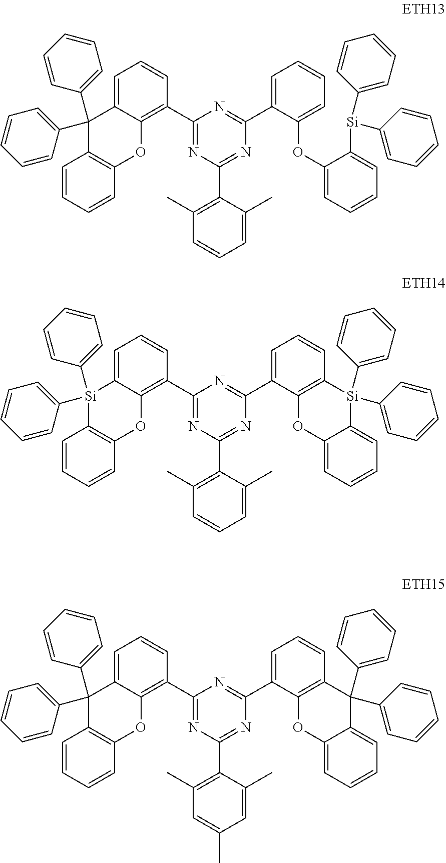

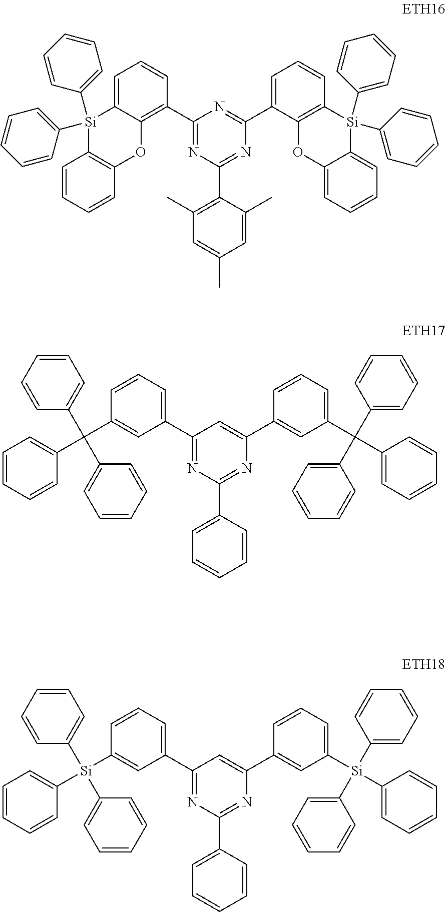 [SMILES: Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1 ; c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1]

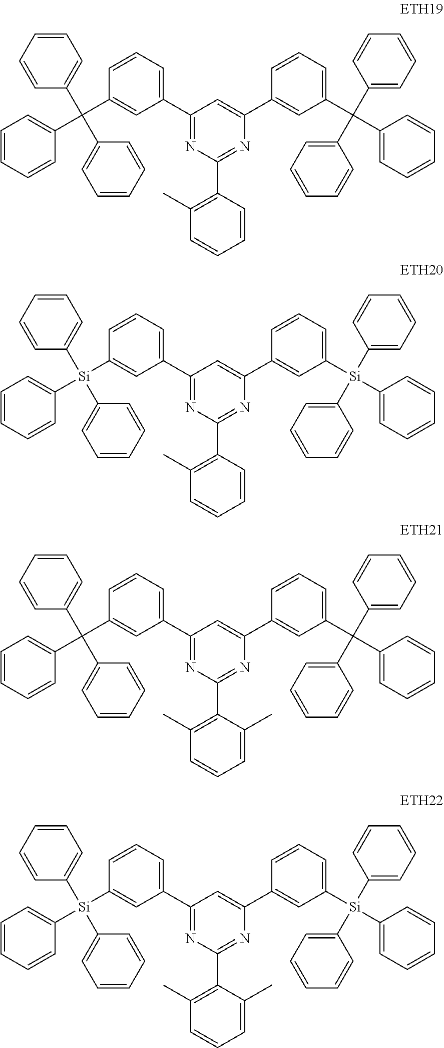 [SMILES: Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

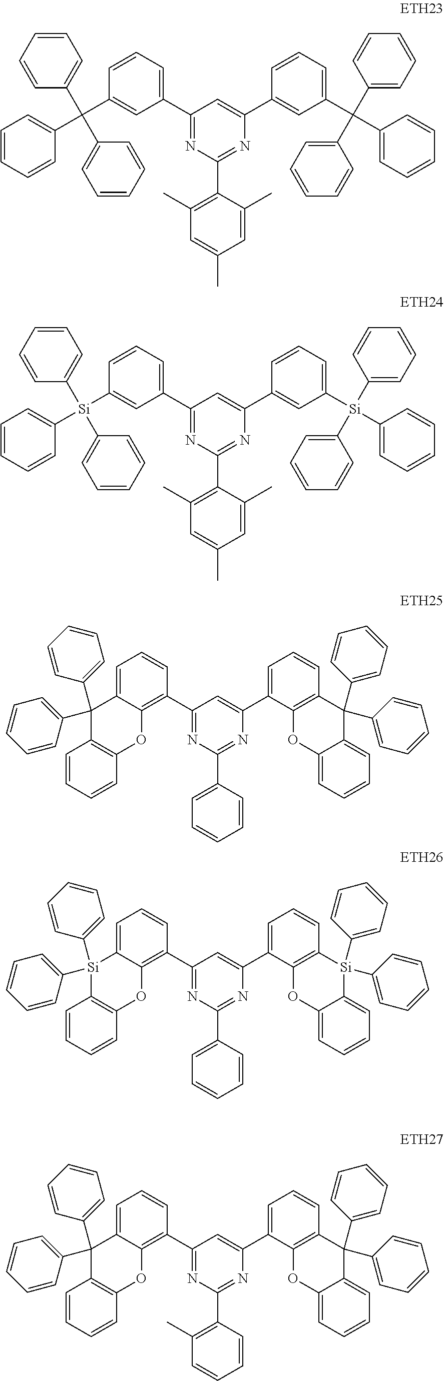

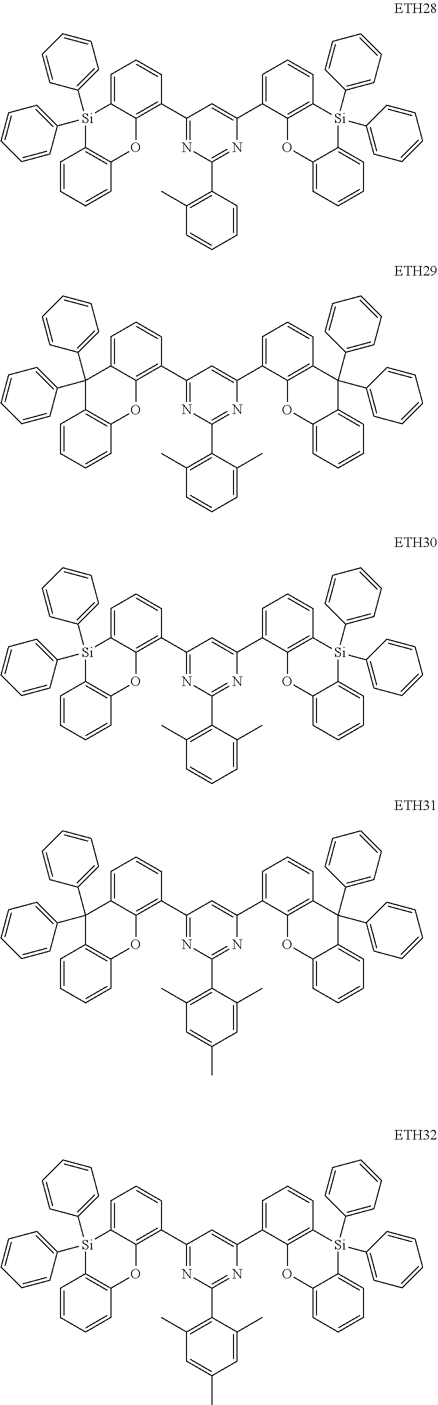

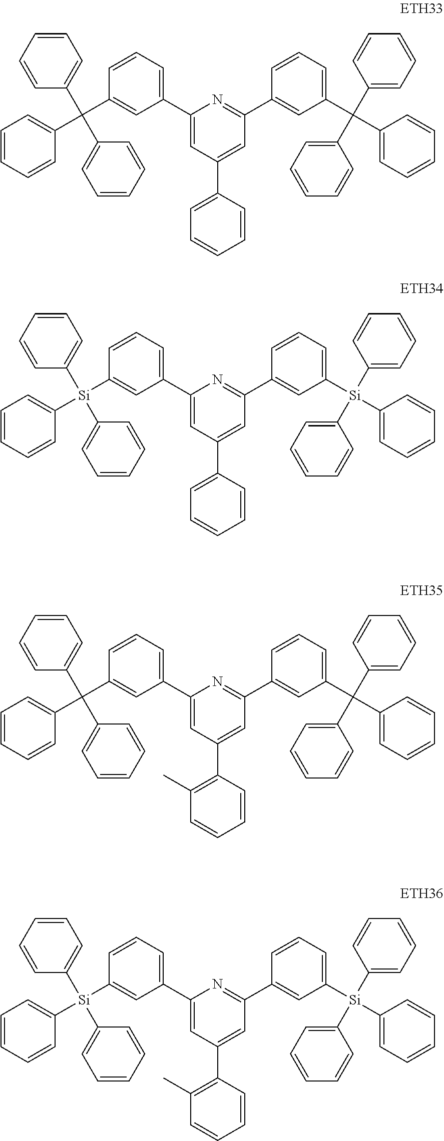 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

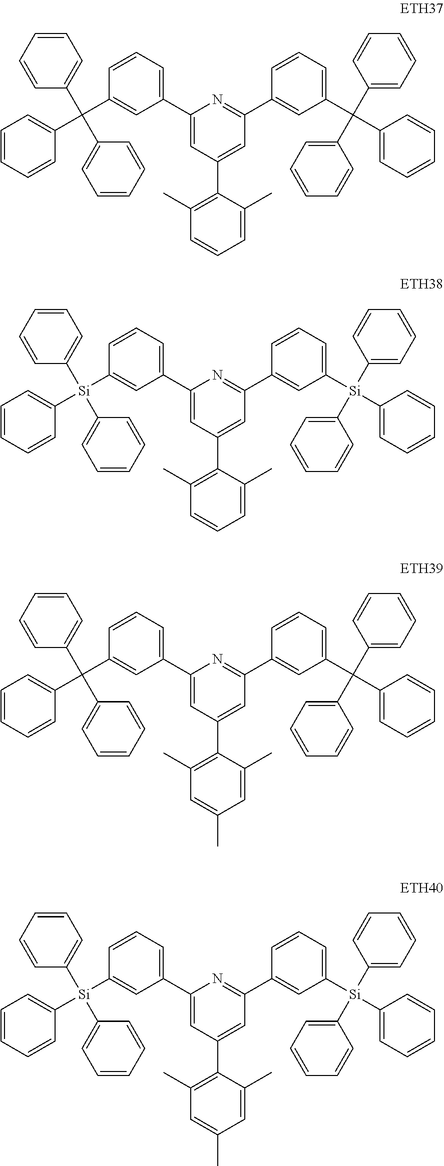 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

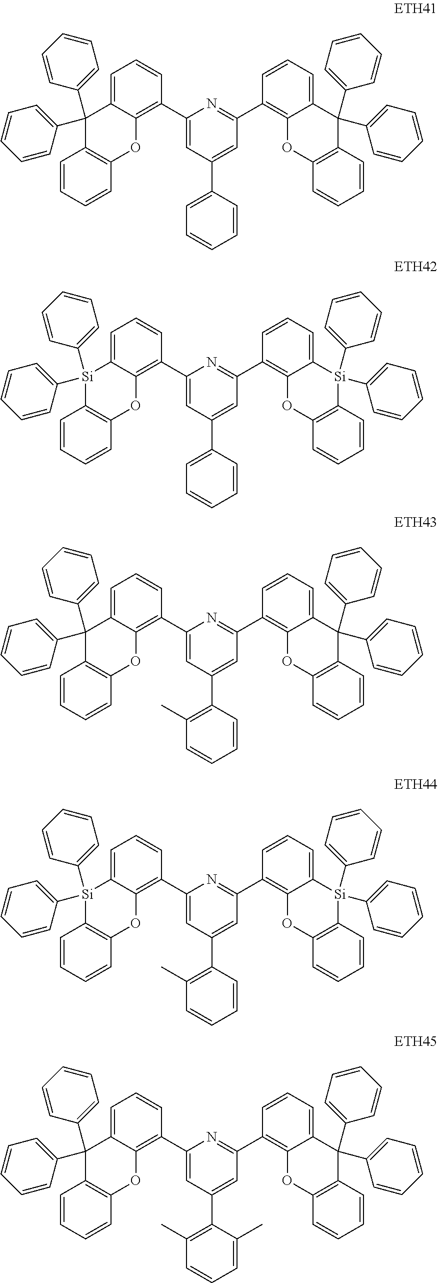

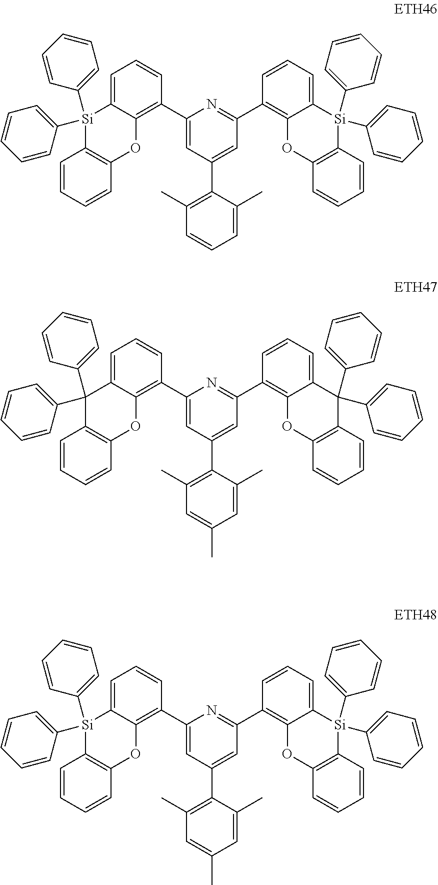 [SMILES: Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

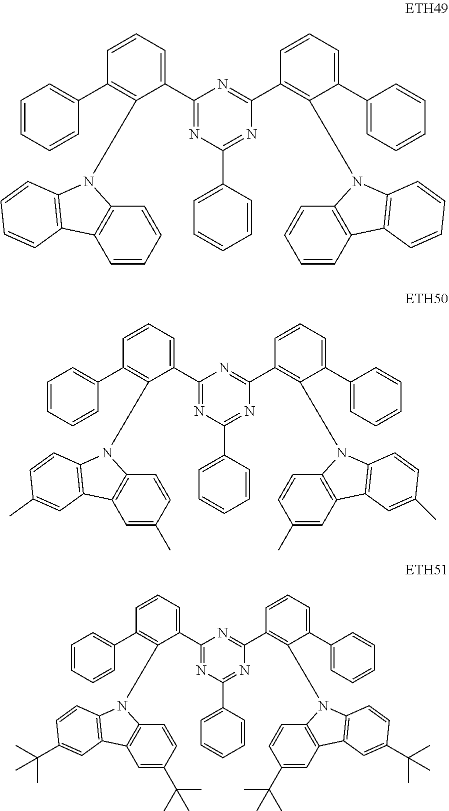

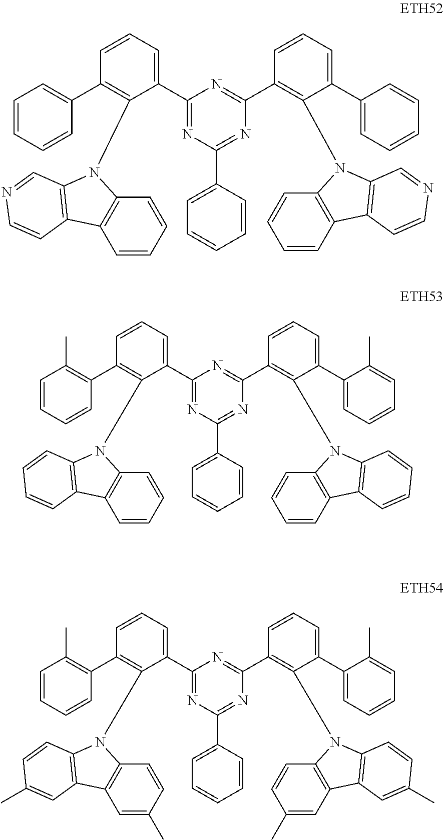

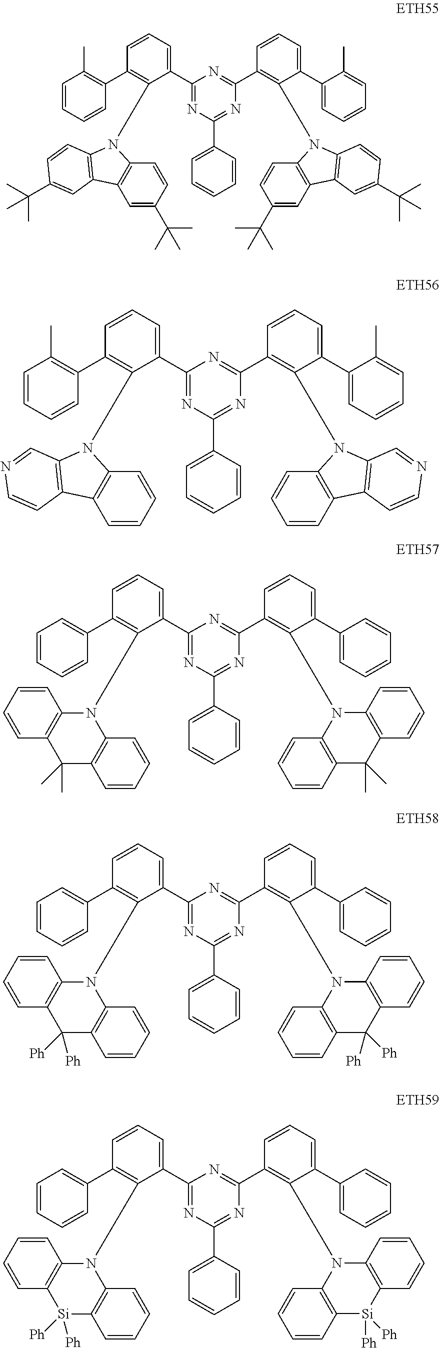

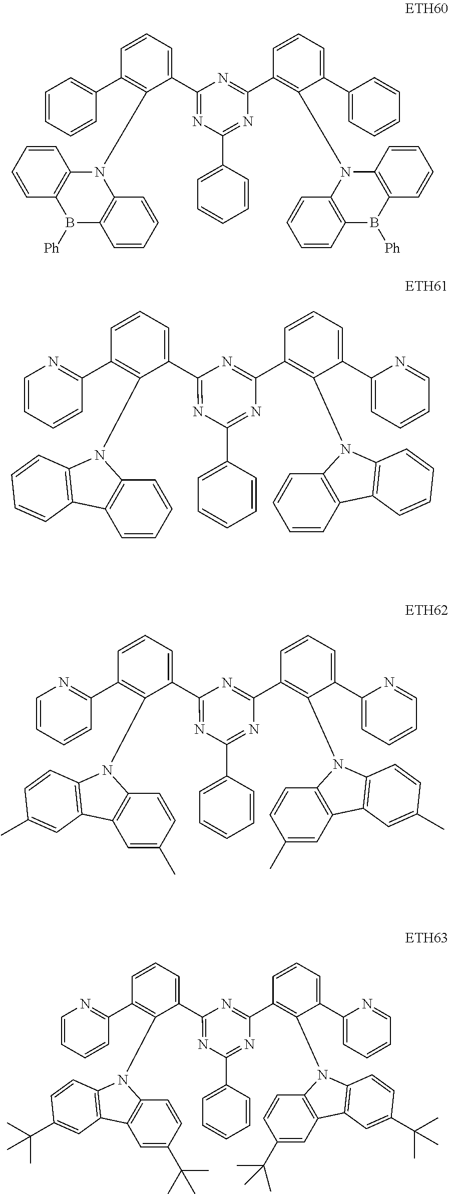

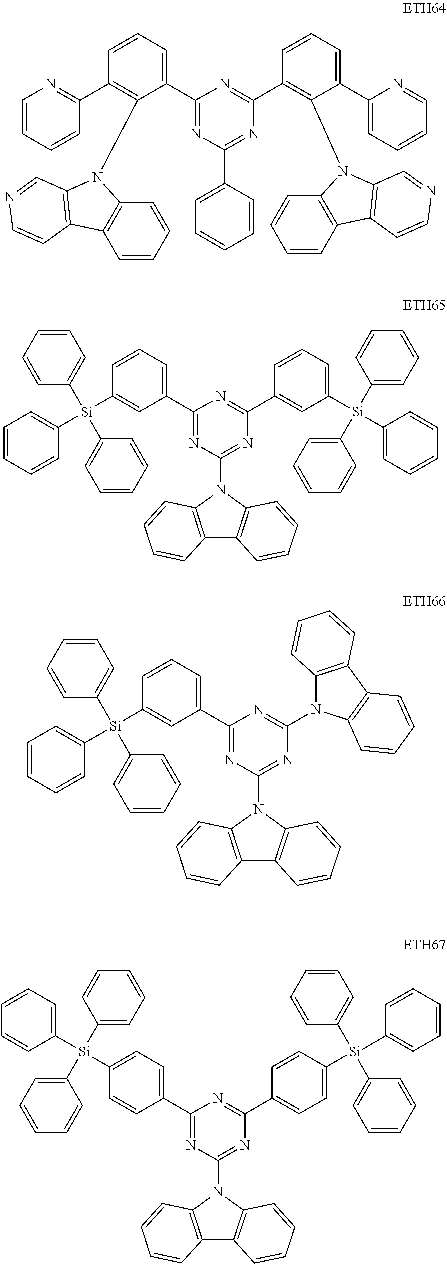 [SMILES: c1ccc(-c2nc(-c3cccc(-c4ccccn4)c3-n3c4ccccc4c4ccncc43)nc(-c3cccc(-c4ccccn4)c3-n3c4ccccc4c4ccncc43)n2)cc1 ; c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1]

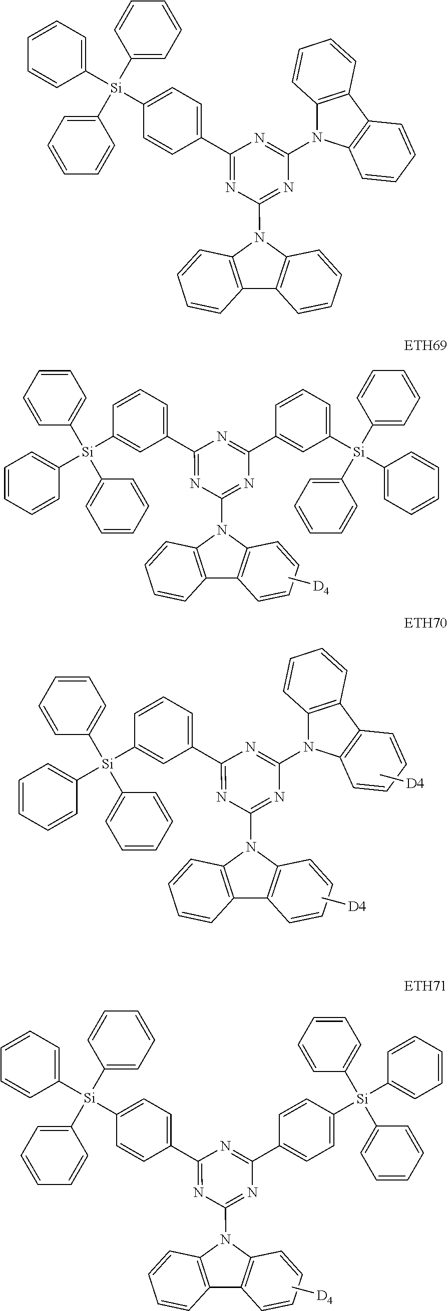

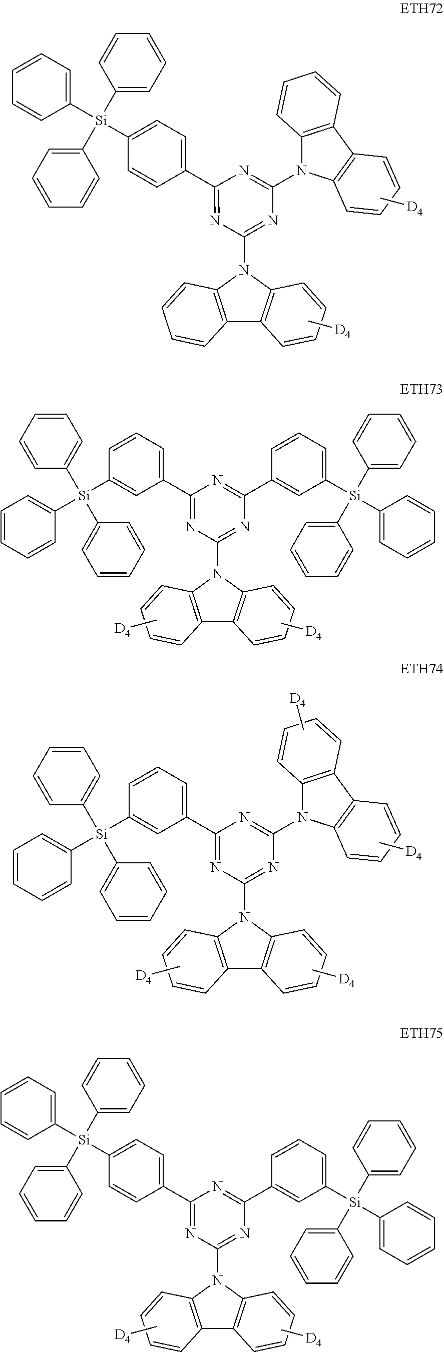

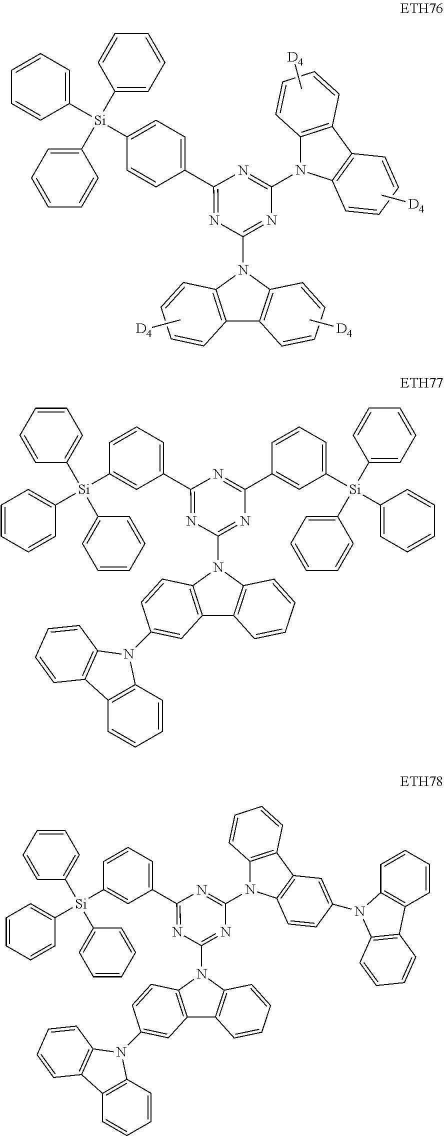

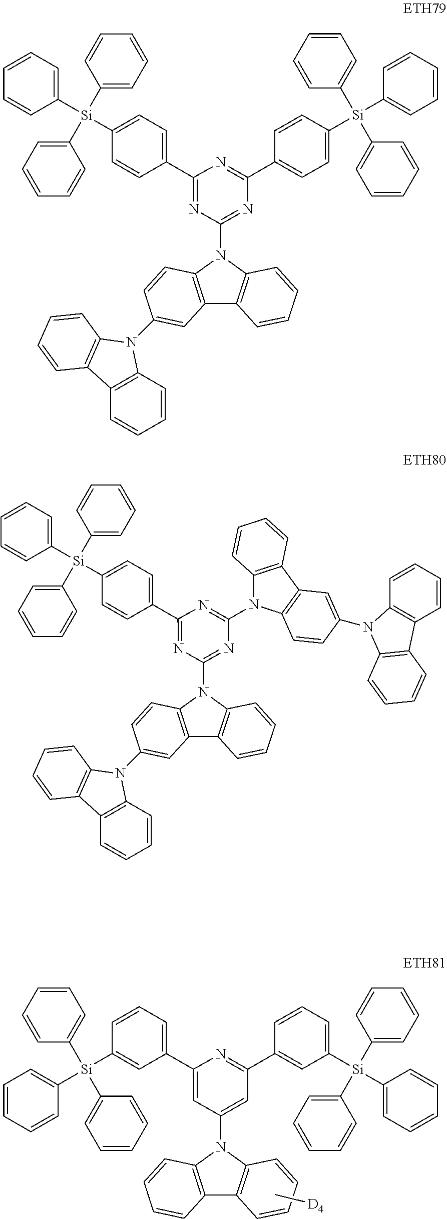

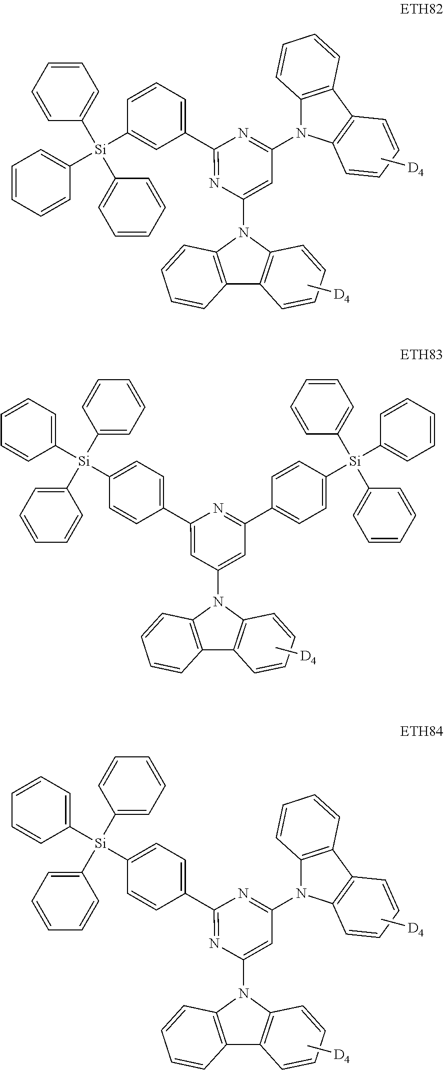

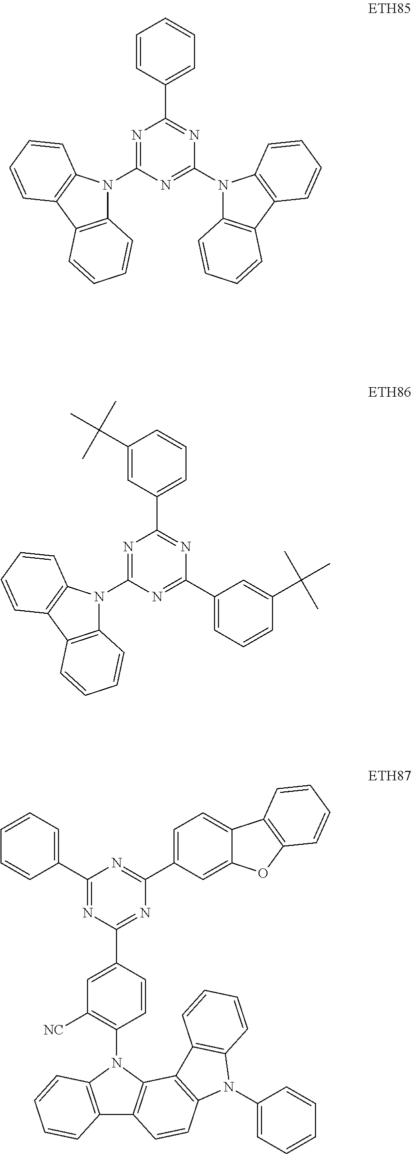 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1 ; N#Cc1cc(-c2nc(-c3ccccc3)nc(-c3ccc4c(c3)oc3ccccc34)n2)ccc1-n1c2ccccc2c2ccc3c(c4ccccc4n3-c3ccccc3)c21]

In the particular compounds suggested in Compound Group 3, “D” refers to a deuterium atom, and “Ph” refers to an unsubstituted phenyl group.

The emission layer EML includes the second compound and the third compound, and the second compound and the third compound may form an exciplex. In the emission layer EML, the exciplex may be formed by a hole transport host and an electron transport host. In this case, the triplet energy of the exciplex formed by the hole transport host and the electron transport host may correspond to an energy level difference between the lowest unoccupied molecular orbital (LUMO) energy level of the electron transport host and the highest occupied molecular orbital (HOMO) energy level of the hole transport host.

For example, the absolute value of the triplet energy level (T1) of the exciplex formed by the hole transport host and the electron transport host may be about 2.4 electron volt (eV) to about 3.0 eV. In some embodiments, the triplet energy of the exciplex may be smaller than the energy gap of each host material. The exciplex may have a triplet energy of about 3.0 eV or less, which is the energy gap between the hole transport host and the electron transport host.

In one or more embodiments, the emission layer EML may include the fourth compound in addition to the first compound to the third compound. The fourth compound may be used as a phosphorescence sensitizer of the emission layer EML. Energy transfer from the fourth compound to the first compound may occur to emit light.

For example, the emission layer EML may include an organometallic complex including platinum (Pt) as a central metal atom and ligands bonded to the central metal atom, as the fourth compound. In the light-emitting element ED of one or more embodiments, the emission layer EML may include a compound represented by Formula D-1 as the fourth compound. In one or more embodiments, the fourth compound in the present specification may be referred to as an “organometallic compound.”

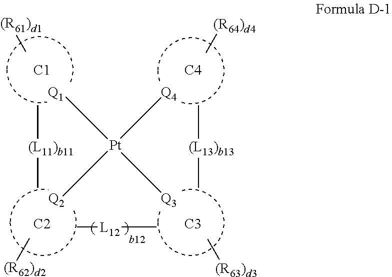

In Formula D-1, Q₁ to Q₄ may each independently be C or N.

In Formula D-1, C1 to C4 may each independently be a substituted or unsubstituted hydrocarbon ring of 5 to 30 ring-forming carbon atoms, or a substituted or unsubstituted heterocycle of 2 to 30 ring-forming carbon atoms.

In Formula D-1, L₁₁ to L₁₃ may each independently be a direct linkage, *—O—*, *—S—*,

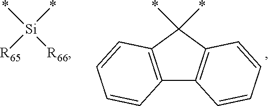

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “-*” refers to a part connected with C₁ to C₄.

In Formula D-1, b11 to b13 may each independently be 0 or 1. If b11 is 0, C1 and C2 may not be connected to each other. If b12 is 0, C2 and C3 may not be connected to each other. If b13 is 0, C3 and C4 may not be connected to each other.

In Formula D-1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 ring-forming carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, each of R₆₁ to R₆₆ may be connected with an adjacent group to form a ring. R₆₁ to R₆₆ may each independently be a substituted or unsubstituted methyl group or a substituted or unsubstituted t-butyl group.

In Formula D-1, d1 to d4 may each independently be an integer of 0 to 4. In Formula D-1, if (e.g., when) d1 to d4 are 0, the fourth compound may be unsubstituted with R₆₁ to R₆₄, respectively. Cases where d1 to d4 are 4, and R₆₁ to R₆₄ are hydrogen atoms, may be the same as cases where d1 to d4 are 0, respectively. If (e.g., when) d1 to d4 are integers of 2 or more, each of multiple R₆₁ to R₆₄ may be substantially the same, or at least one selected from among each of multiple R₆₁ to R₆₄ may be different.

In Formula D-1, C1 to C4 may each independently be a substituted or unsubstituted hydrocarbon ring, or a substituted or unsubstituted heterocycle, represented by any one selected from among C-1 to C-4.

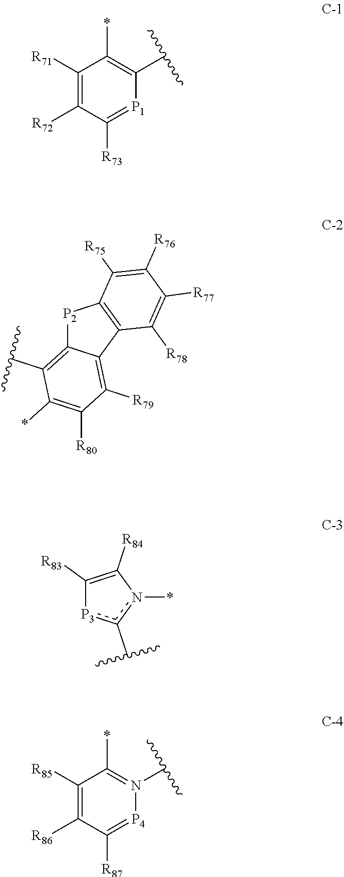

In C-1 to C-4, P₁ may be C—* or CR₇₄, P₂ may be N—* or NR₈₁, P₃ may be N—* or NR₈₂, and P₄ may be C—* or CR₈₈. R₇₁ to R₈₈ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In C-1 to C-4,

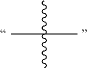

is a part connected with a Pt central metal atom, and “-*” corresponds to a part connected with an adjacent ring group (C1 to C4) or a linker (L₁₁ to L₁₃).

The emission layer EML of one or more embodiments may include the first compound which is a fused polycyclic compound, and at least one selected from among the second compound to the fourth compound. For example, the emission layer EML may include the first compound, the second compound and the third compound. In the emission layer EML, the second compound and the third compound may form an exciplex, and energy transfer from the exciplex to the first compound may occur to emit light.

In some embodiments, the emission layer EML may include the first compound, the second compound, the third compound and the fourth compound. In the emission layer EML, the second compound and the third compound may form an exciplex, and energy transfer from the exciplex to the fourth compound and the first compound may occur to emit light. In one or more embodiments, the fourth compound may be a sensitizer. In the light-emitting element ED of one or more embodiments, the fourth compound included in the emission layer EML may function as a sensitizer and may play the role of transferring energy from the host to the first compound which is a light emitting dopant. For example, the fourth compound which plays the role of an auxiliary dopant may accelerate energy transfer to the first compound which is the light emitting dopant and may increase the emission ratio of the first compound. Accordingly, the emission layer EML of one or more embodiments may improve emission efficiency. In some embodiments, if the energy transfer to the first compound increases, excitons formed in the emission layer EML may not be accumulated in the emission layer EML but may rapidly emit light, and the deterioration of an element may be reduced. Accordingly, the lifetime of the light-emitting element ED of one or more embodiments may increase.

The light-emitting element ED of one or more embodiments may include each (e.g., any or all) of the first compound, the second compound, the third compound and the fourth compound, and the emission layer EML may include the combination of two host materials and two dopant materials. In the light-emitting element ED of one or more embodiments, the emission layer EML may include the second compound and the third compound, which are two different hosts, the first compound which emits delayed fluorescence, and the fourth compound including an organometallic complex, concurrently (e.g., simultaneously), and may show excellent or suitable emission efficiency properties.

In one or more embodiments, the fourth compound represented by Formula D-1 may be represented by at least one selected from among the compounds represented in Compound Group 4. The emission layer EML may include at least one selected from among the compounds represented in Compound Group 4 as a sensitizer material.

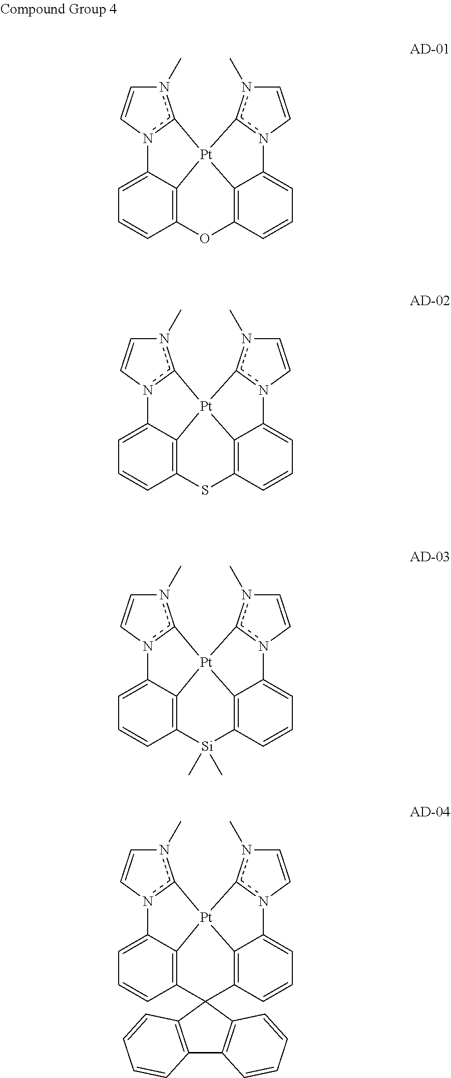

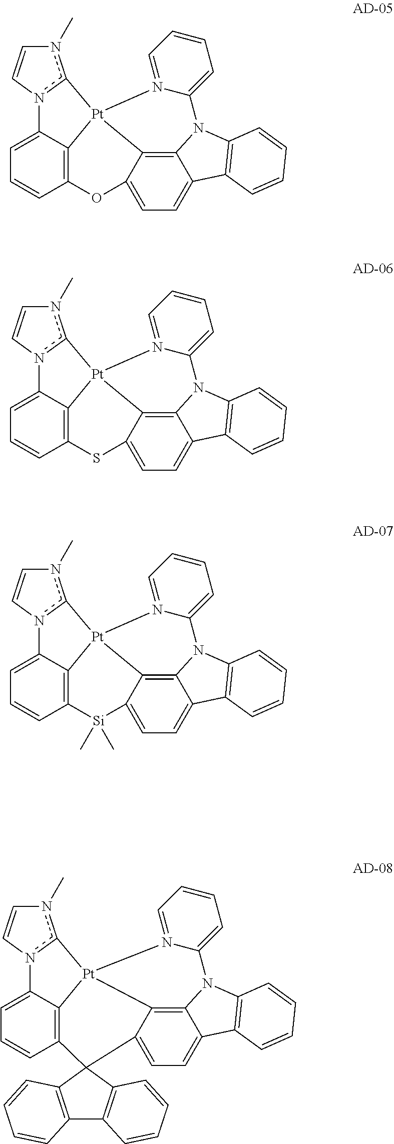

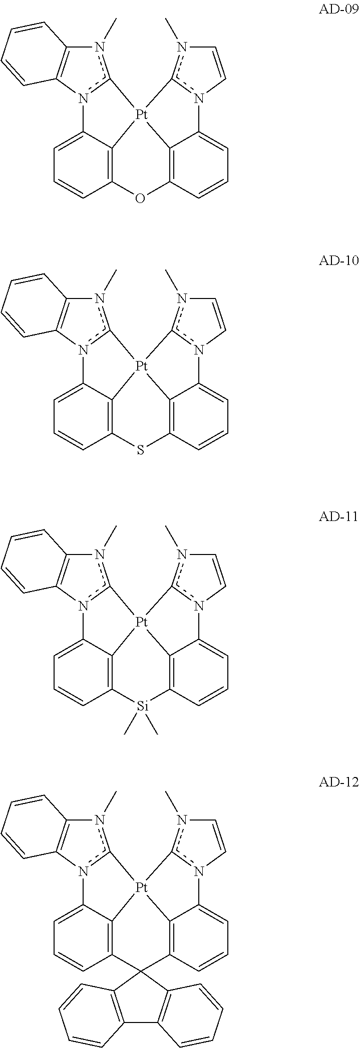

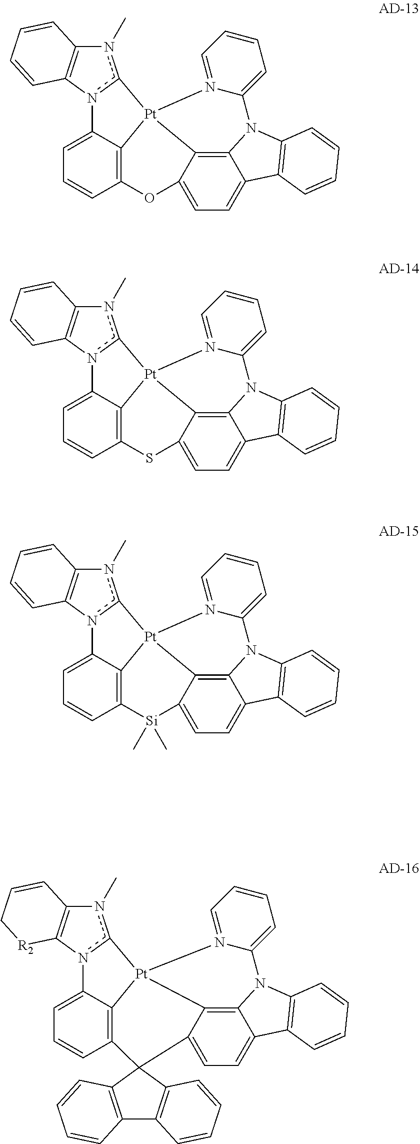

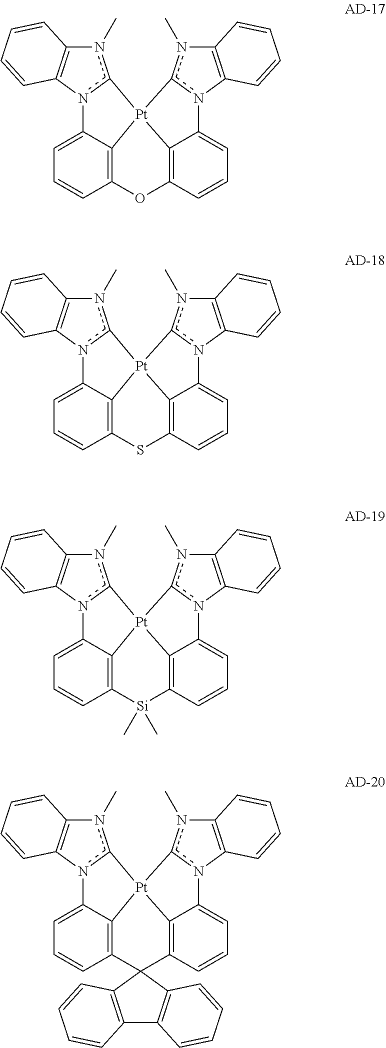

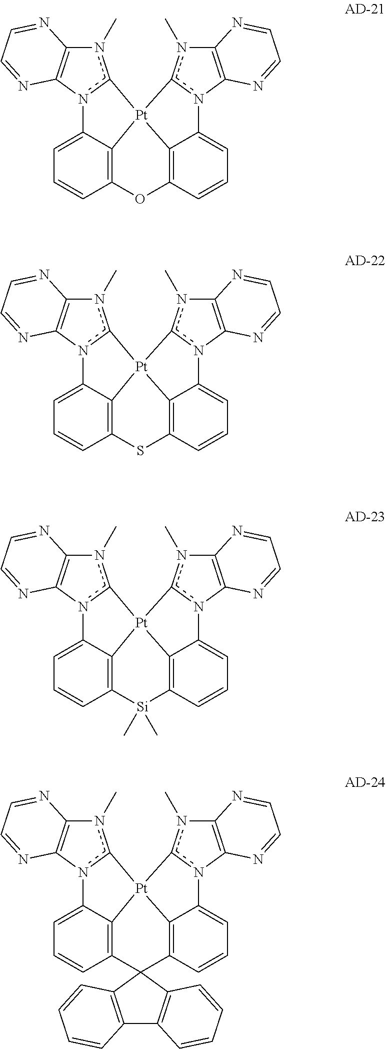

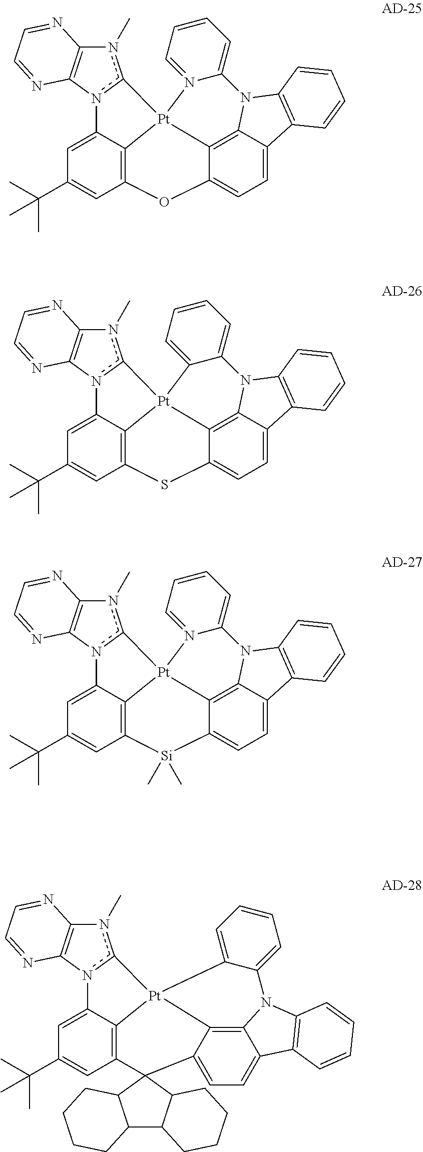

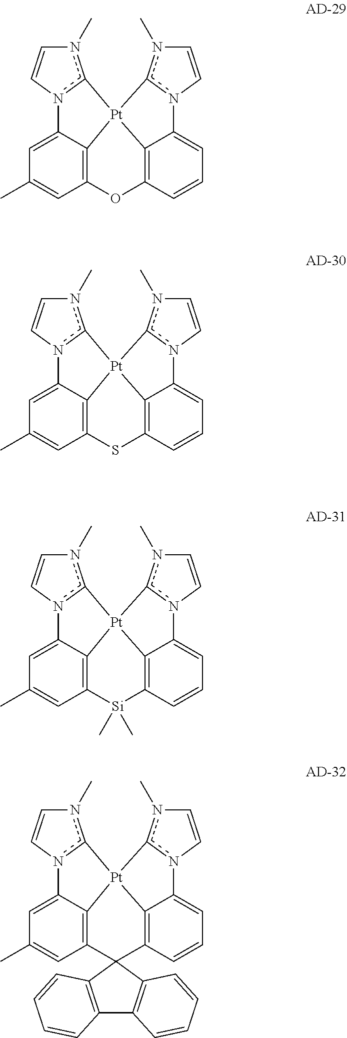

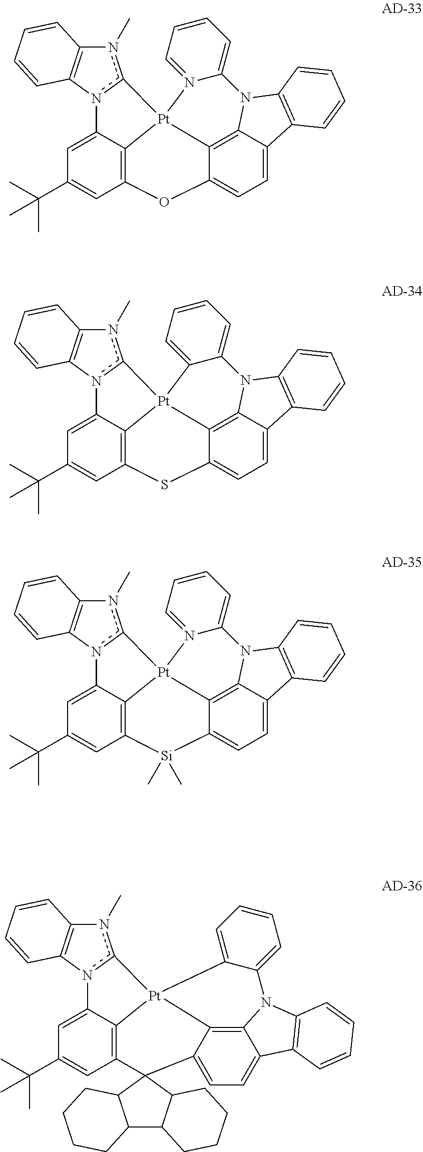

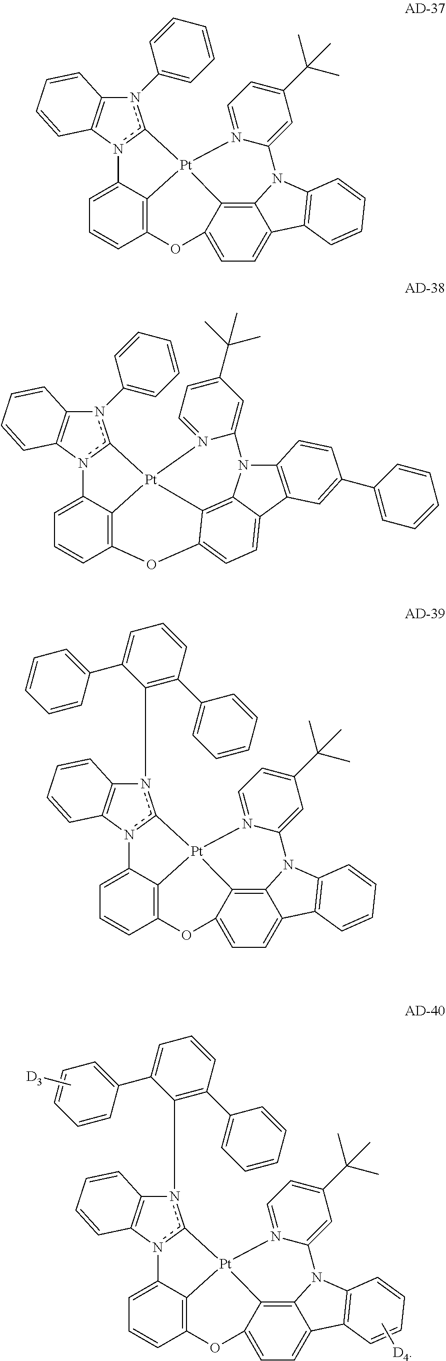

In the particular compounds suggested in Compound Group 4, “D” refers to a deuterium atom.

In one or more embodiments, the light-emitting element ED of one or more embodiments may include multiple emission layers. Multiple emission layers may be stacked in order and provided, and for example, a light-emitting element ED including multiple emission layers may be to emit white light. The light-emitting element including multiple emission layers may be a light-emitting element of a tandem structure. If the light-emitting element ED includes multiple emission layers, at least one emission layer EML may include the first compound represented by Formula 1 of one or more embodiments. In some embodiments, if the light-emitting element ED includes multiple emission layers, at least one emission layer EML may include all of the first compound, the second compound, the third compound, and the fourth compound.

In the light-emitting element ED of one or more embodiments, if (e.g., when) the emission layer EML includes all of the first compound, the second compound, the third compound, and the fourth compound, the amount of the first compound may be about 0.1 wt % to about 5 wt % based on the total weight of the first compound, the second compound, the third compound, and the fourth compound. However, one or more embodiments of the disclosure is not limited thereto. If (e.g., when) the amount of the first compound satisfies the herein-described ratio, energy transfer from the second compound and the third compound to the first compound may increase, and accordingly, the emission efficiency and device lifetime may increase.

In the emission layer EML, the total amount of the second compound and the third compound may be the remaining amount excluding the amount of the first compound and the fourth compound. For example, the total amount of the second compound and the third compound may be about 65 wt % to about 95 wt % based on the total weight of the first compound, the second compound, the third compound, and the fourth compound.

In the total amount of the second compound and the third compound, the weight ratio of the second compound and the third compound may be about 3:7 to about 7:3.

If (e.g., when) the total amount of the second compound and the third compound satisfies the herein-described ratio, charge balance properties in the emission layer EML may be improved, and emission efficiency and device lifetime may be improved. If (e.g., when) the total amount of the second compound and the third compound deviates from the herein-described ratio range, charge balance in the emission layer EML may be broken, emission efficiency may be degraded, and the device may be easily deteriorated.

If (e.g., when) the emission layer EML includes the fourth compound, the amount of the fourth compound may be about 4 wt % to 30 wt % based on the total weight of the first compound, the second compound, the third compound and the fourth compound in the emission layer EML. However, one or more embodiments of the disclosure is not limited thereto. If (e.g., when) the amount of the fourth compound satisfies the herein-described amount, energy transfer from a host to the first compound that is a light emitting dopant may increase, and emission ratio may be improved. Accordingly, the emission efficiency of the emission layer EML may be improved. If (e.g., when) the amount ratio of the first compound, the second compound, the third compound and the fourth compound, included in the emission layer EML satisfies the herein-described amount ratio, excellent or suitable emission efficiency and long lifetime may be achieved.

In one or more embodiments, the emission layer EML may include a compound represented by Formula E-2a or Formula E-2b. The compound represented by Formula E-2a or Formula E-2b may be used as a phosphorescence host material.

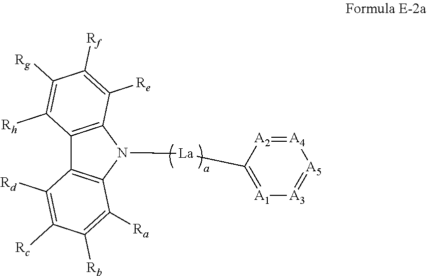

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) “a” is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₈ may each independently be N or CRi. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Each of Rₐ to Rᵢ may be combined with an adjacent group to form a hydrocarbon ring or a heterocycle including N, O, S, and/or the like. as a ring-forming atom.

In one or more embodiments, in Formula E-2a, two or three selected from among A₁ to A₅ may be N, and any remaining selected from among A₁ to A₅ may be CRᵢ.

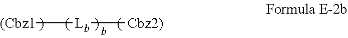

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” may be an integer of 0 to 10, and if (e.g., when) “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds listed in Compound Group E-2 are only illustrations, and the compound represented by Formula E-2a or Formula E-2b is not limited to the compounds represented in Compound Group E-2.

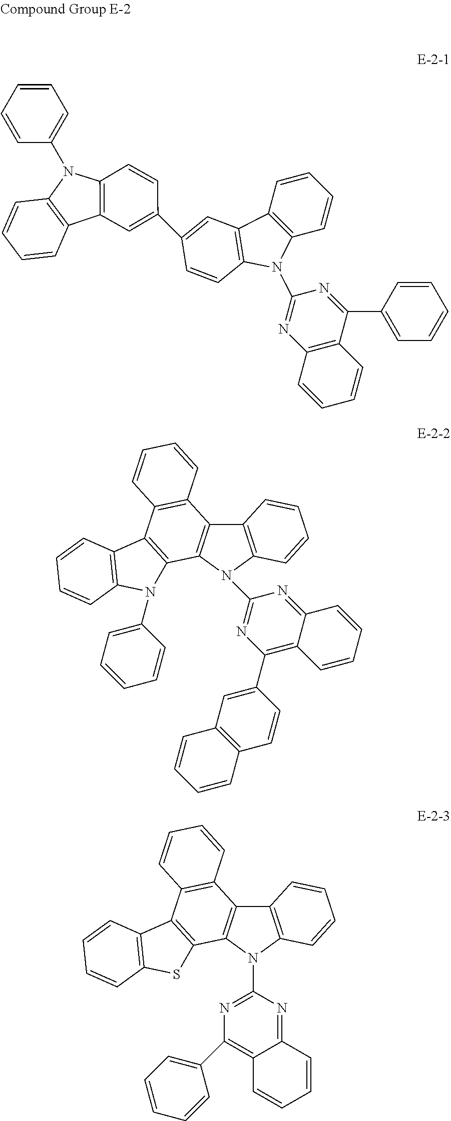

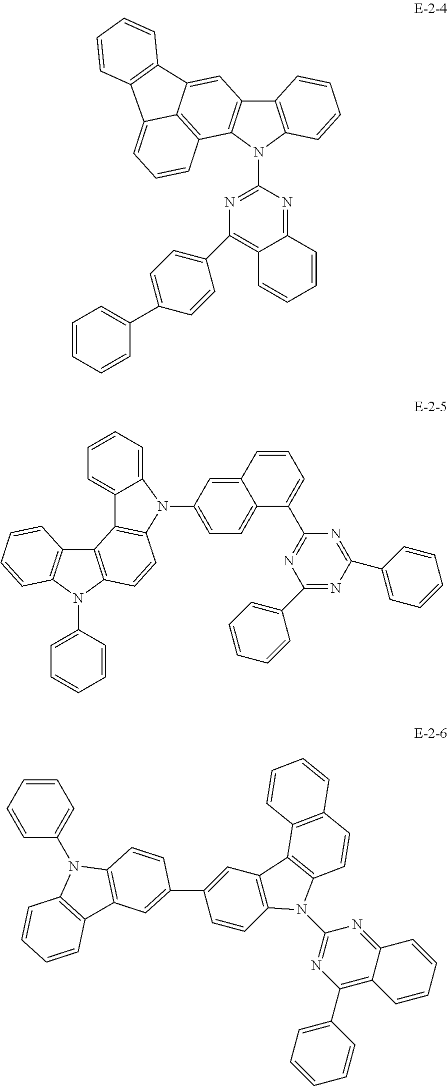 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

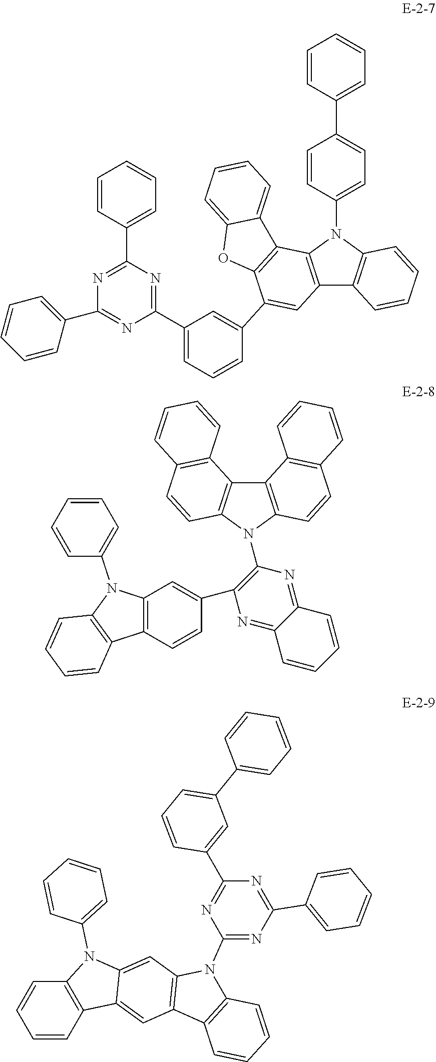

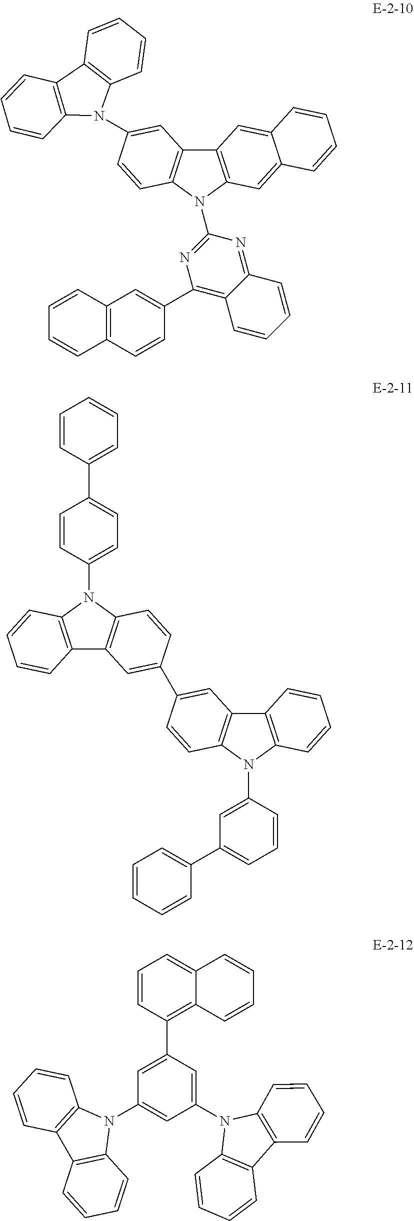

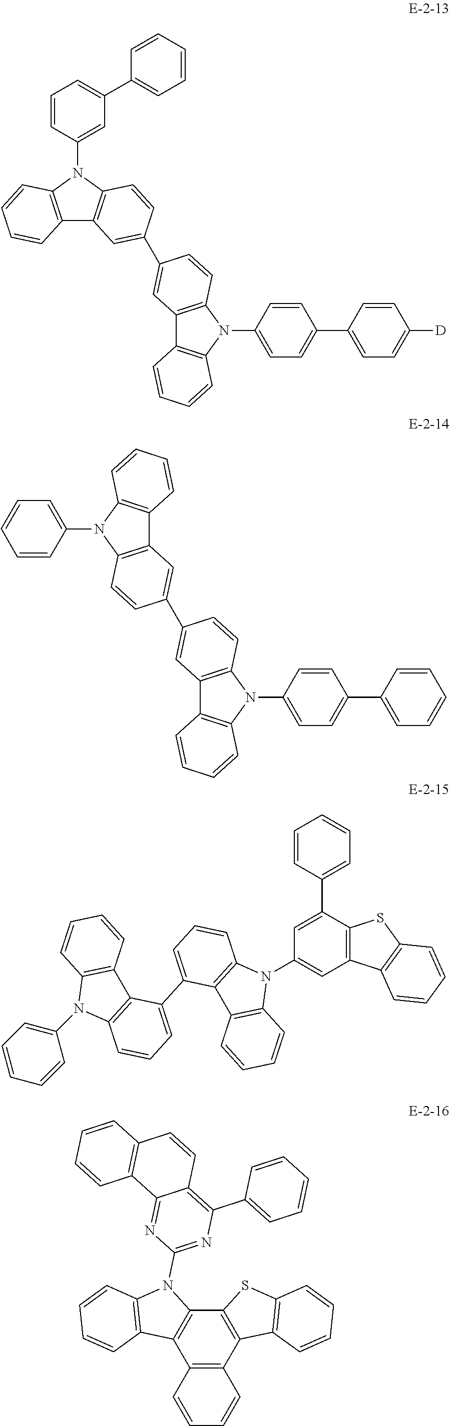 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1]

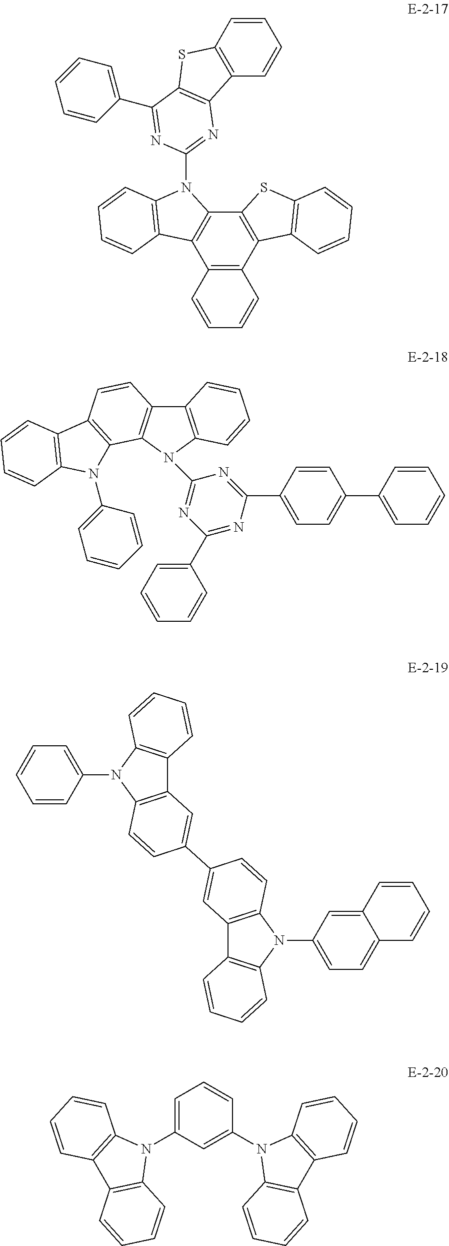 [SMILES: c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1]

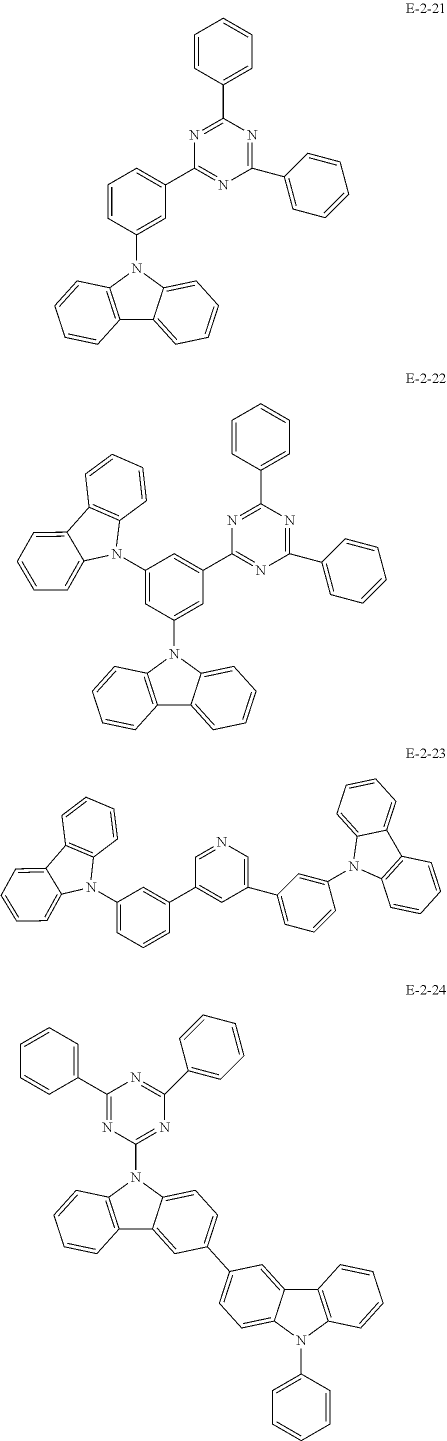 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-suitable in the art as a host material. For example, the emission layer EML may include as a host material, at least one of (e.g., selected from among) bis(4-(9H-carbazol-9-yl)phenyl) diphenylsilane (BCPDS), (4-(1-(4-(diphenylamino)phenyl) cyclohexyl)phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl) dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the disclosure is not limited thereto. For example, tris(8-hydroxyquinolino) aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO3), octaphenylcyclotetra siloxane (DPSiO4), and/or the like. may be used as the host material.

The emission layer EML may include a compound represented by Formula M-a. The compound represented by Formula M-a may be used as a phosphorescence dopant material.

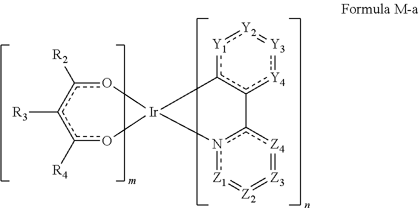

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” may be 0 or 1, and “n” may be 2 or 3. In Formula M-a, if (e.g., when) “m” is 0, “n” is 3, and if (e.g., when) “m” is 1, “n” is 2.

The compound represented by Formula M-a may be used as a phosphorescence dopant.

The compound represented by Formula M-a may be represented by any one selected from among Compounds M-a1 to M-a25. However, Compounds M-a1 to M-a25 are illustrations, and the compound represented by Formula M-a is not limited to the compounds represented by Compounds M-a1 to M-a25.

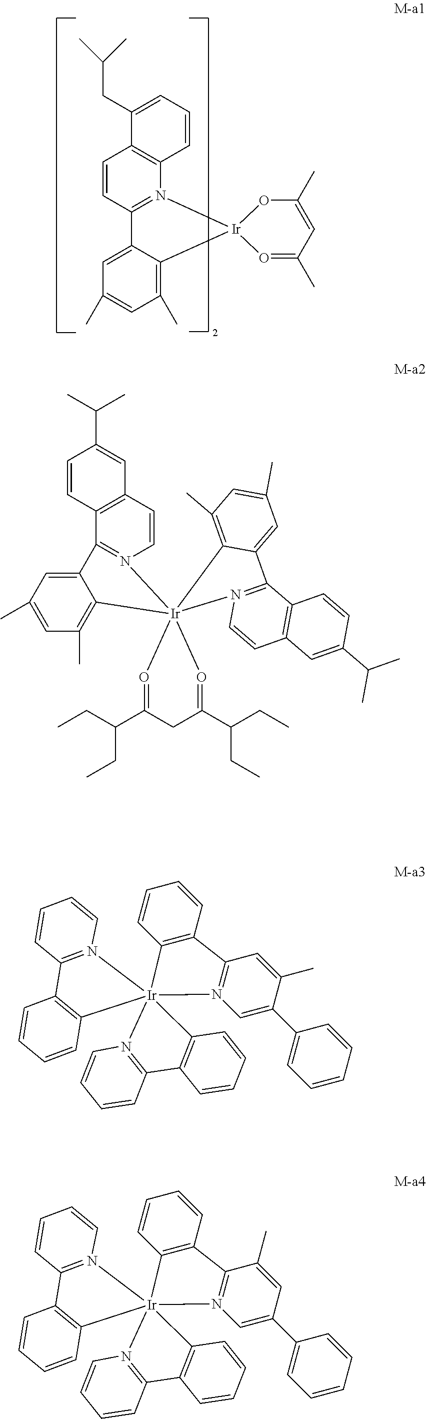

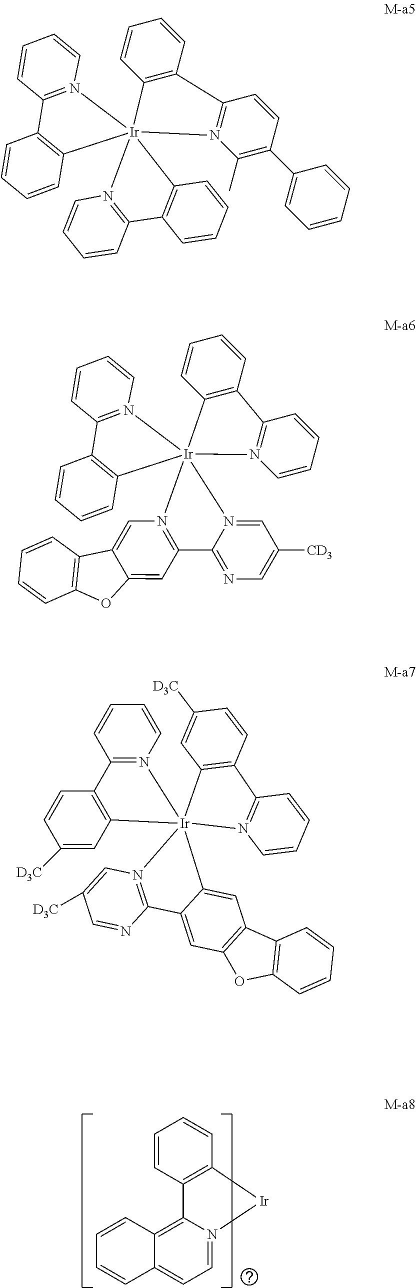

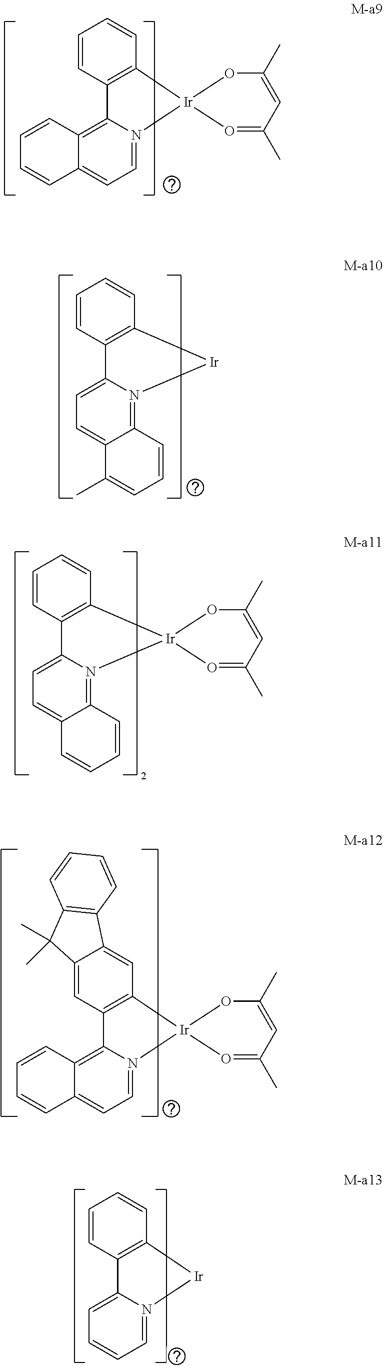

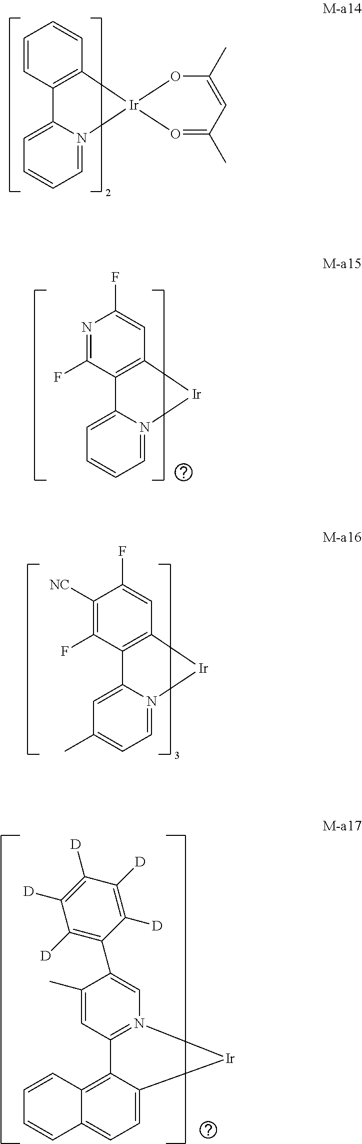

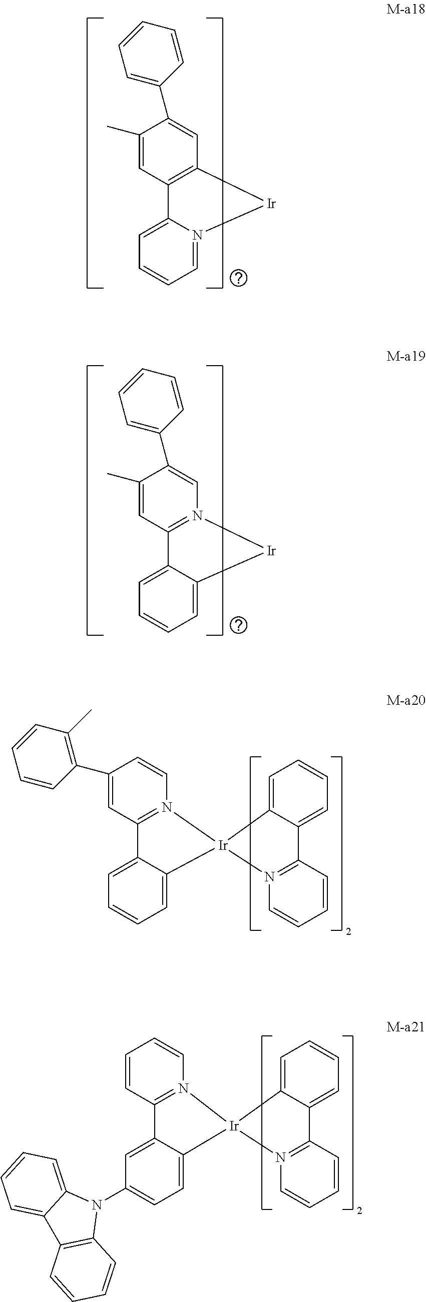

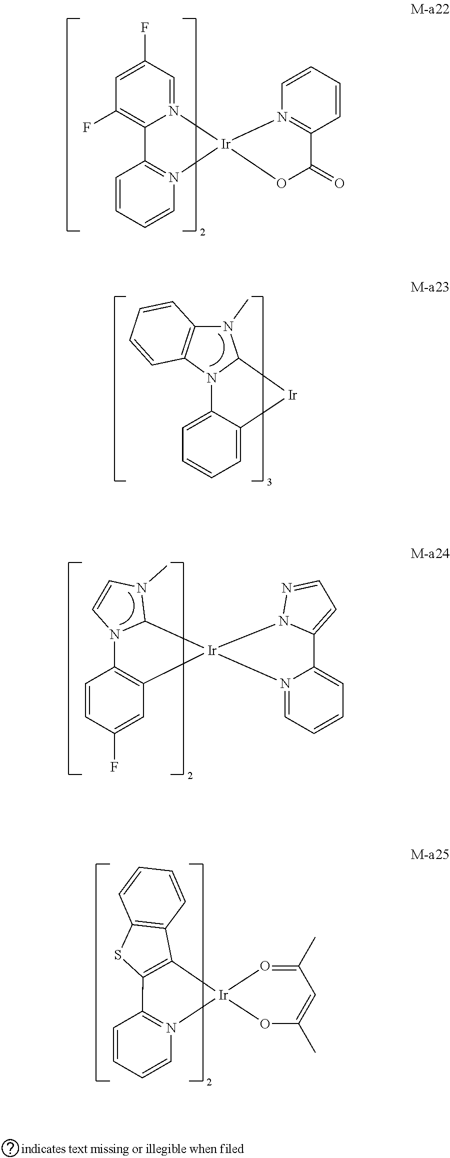

In one or more embodiments, the emission layer EML may include as a suitable dopant material, styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino) pyrene), and/or the like.

The emission layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). for example, iridium(III) bis(4,6-difluorophenylpyridinato-N,C2′) picolinate (Flrpic), bis(2,4-difluorophenylpyridinato)-tetrakis(1-pyrazolyl) borate iridium(III) (Fir6), or platinum octaethyl porphyrin (PtOEP) may be used as the phosphorescence dopant. However, one or more embodiments of the disclosure is not limited thereto.

The emission layer EML may include a quantum dot material.

In the description, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling the element ratio in the quantum dot compound.

The diameter of the quantum dot may be, for example, about 1 nanometer (nm) to about 10 nm. In the present disclosure, when quantum dot, quantum dots, or quantum dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size

The quantum dot may be synthesized by a chemical bath deposition, a metal organic chemical vapor deposition, a molecular beam epitaxy or a similar process therewith.

The chemical bath deposition is a method of mixing an organic solvent and a precursor material and then, growing a quantum dot particle crystal. During growing the crystal, the organic solvent may naturally play the role of a dispersant which is coordinated on the surface of the quantum dot crystal and may control the growth of the crystal. Accordingly, the chemical bath deposition is more advantageous if (e.g., when) compared to a vapor deposition method including a metal organic chemical vapor deposition (MOCVD) and molecular beam epitaxy (MBE), and the growth of the quantum dot particle may be controlled or selected through a low-cost process.

The emission layer EML may include a quantum dot material. The core of the quantum dot may be selected from among a II-VI group compound, a III-VI group compound, a I-III-VI group compound, a III-V group compound, a III-II-V group compound, a IV-VI group compound, a IV group element, a IV group compound, and/or one or more (e.g., any suitable) combinations thereof.

The Group II-VI compound may be selected from among the group including (e.g., consisting of) a binary compound selected from among the group including (e.g., consisting of) CdSe, CdTe, CdS, ZnS, ZnSe, ZnTe, ZnO, HgS, HgSe, HgTe, MgSe, MgS, and/or a (e.g., any suitable) mixture thereof; a ternary compound selected from among the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or a (e.g., any suitable) mixture thereof; and a quaternary compound selected from among the group including (e.g., consisting of) HgZnTeS, CdZnSeS, CdZnSeTe, CdZnSTe, CdHgSeS, CdHgSeTe, CdHgSTe, HgZnSeS, HgZnSeTe, and/or a (e.g., any suitable) mixture thereof. In one or more embodiments, the Group II-VI compound may further include a Group I metal and/or a Group IV element. The Group I-II-VI compound may be selected from among CuSnS or CuZnS, and the Group II-IV-VI compound may be selected from among ZnSnS and/or the like. The Group I-II-IV-VI compound may be selected from among quaternary compounds selected from among the group including (e.g., consisting of) Cu₂ZnSnS₂, Cu₂ZnSnS₄, Cu₂ZnSnSe₄, Ag₂ZnSnS₂, and/or a (e.g., any suitable) mixture thereof.

The III-VI group compound may include a binary compound such as In₂S₃, and In₂Se₃, a ternary compound such as InGaS₃, and InGaSe₃, or arbitrary combinations thereof.

The I-III-VI group compound may be selected from among a ternary compound selected from among the group including (e.g., consisting of) AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The III-V group compound may be selected from among the group including (e.g., consisting of) a binary compound selected from among the group including (e.g., consisting of) GaN, GaP, GaAs, GaSb, AlN, AlP, AlAs, AlSb, InN, InP, InAs, InSb, and mixtures thereof, a ternary compound selected from among the group including (e.g., consisting of) GaNP, GaNAs, GaNSb, GaPAs, GaPSb, AlNP, AlNAs, AlNSb, AlPAS, AlPSb, InGaP, InAlP, InNP, InNAs, InNSb, InPAs, InPSb, and mixtures thereof, and a quaternary compound selected from among the group including (e.g., consisting of) GaAlNP, GaAlNAs, GaAlNSb, GaAlPAs, GaAlPSb, GalnNP, GalnNAs, GalnNSb, GalnPAs, GalnPSb, InAlNP, InAlNAs, InAlNSb, InAlPAs, InAlPSb, and mixtures thereof. In one or more embodiments, the III-V group compound may further include a II group metal. For example, InZnP, and/or the like. may be selected as a Ill-II-V group compound.

The Group IV-VI compound may be selected from among the group including (e.g., consisting of) a binary compound selected from among the group including (e.g., consisting of) SnS, SnSe, SnTe, PbS, PbSe, PbTe, and/or a (e.g., any suitable) mixture thereof, a ternary compound selected from among the group including (e.g., consisting of) SnSeS, SnSeTe, SnSTe, PbSeS, PbSeTe, PbSTe, SnPbS, SnPbSe, SnPbTe, and/or a (e.g., any suitable) mixture thereof, and a quaternary compound selected from among the group including (e.g., consisting of) SnPbSSe, SnPbSeTe, SnPbSTe, and/or a (e.g., any suitable) mixture thereof.

The Group II-IV-V compound may be selected from among a ternary compound selected from among the group including (e.g., consisting of) ZnSnP, ZnSnP₂, ZnSnAs₂, ZnGeP₂, ZnGeAs₂, CdSnP₂, and CdGeP₂ and/or a (e.g., any suitable) mixture thereof.

The Group IV element may be selected from among the group including (e.g., consisting of) Si, Ge, and/or a (e.g., any suitable) mixture thereof. The Group IV compound may be a binary compound selected from among the group including (e.g., consisting of) SiC, SiGe, and/or a (e.g., any suitable) mixture thereof.

Each element included in the multi-element compound such as the binary compound, ternary compound, and quaternary compound may be present in particles at a substantially uniform concentration or a non-substantially uniform concentration. For example, the Formula herein indicates the types (kinds) of elements included in a compound, and element ratios in the compound may be different. For example, AgInGaS₂ may indicate AgInₓGa₁₋ₓS₂ (x is a real number between 0 and 1).

In this case, the binary compound, the ternary compound or the quaternary compound may be present at substantially uniform concentration in a particle or may be present at a partially different concentration distribution state in substantially the same particle. In some embodiments, a core/shell structure in which one quantum dot wraps another quantum dot may be possible. The interface of the core and the shell may have a concentration gradient in which the concentration of an element present in the shell is decreased toward the center.

In one or more embodiments, the quantum dot may have the herein-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, and/or one or more (e.g., any suitable) combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but one or more embodiments of the disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but one or more embodiments of the disclosure is not limited thereto.

The quantum dot may have a full width of half maximum (FWHM) of emission wavelength spectrum of about 45 nm or less, about 40 nm or less, or about 30 nm or less. Within this range, color purity or color reproducibility may be improved. In some embodiments, light emitted via such quantum dot is emitted in all directions, and light view angle properties may be improved.

In some embodiments, the shape of the quantum dot may be generally used shapes in the art, without specific limitation. More particularly, the shape of spherical, pyramidal, multi-arm, or cubic nanoparticle, nanotube, nanowire, nanofiber, nanoplate particle, and/or the like. may be used.

As the size of the quantum dot or the ratio of elements in the quantum dot compound is regulated, the energy band gap may be accordingly controlled or selected to obtain light of one or more suitable wavelengths from the quantum dot emission layer. Therefore, by using the quantum dots as described herein (using quantum dots of different sizes or having different element ratios in the quantum dot compound), a light-emitting element emitting light of one or more suitable wavelengths may be obtained. For example, the size of the quantum dots or the ratio of elements in the quantum dot compound may be regulated to emit red, green, and/or blue light. In some embodiments, the quantum dots may be configured to emit white light by combining light of one or more suitable colors.

In the light-emitting element ED according to one or more embodiments illustrated in FIG. 3 to FIG. 6, an electron transport region ETR may be provided on an emission layer EML. The electron transport region ETR may include at least one among a hole-blocking layer HBL, an electron transport layer ETL, and an electron injection layer EIL, but one or more embodiments of the disclosure is not limited thereto.

The electron transport region ETR may have a single layer made of a single material, a single layer made of different materials, or may have a multi-layered structure having a plurality of layers made of a plurality of different materials.

For example, the electron transport region ETR may have a single-layered structure of an electron injection layer EIL or an electron transport layer ETL, or may have a single-layered structure made of an electron injection material and an electron transport material. In some embodiments, the electron transport region ETR may have a single-layered structure made of a plurality of different materials or may have a structure of an electron transport layer ETL/electron injection layer EIL, a hole-blocking layer HBL/electron transport layer ETL/electron injection layer EIL, which are sequentially stacked from the emission layer EML, but one or more embodiments of the disclosure is not limited thereto. The electron transport region ETR may have a thickness of, for example, about 1000 Å to about 1500 Å.

The electron transport region ETR may be formed by using one or more suitable methods such as a vacuum deposition method, a spin coating method, a casting method, the Langmuir-Blodgett (LB) method, an inkjet printing method, a laser printing method, and a laser induced thermal imaging (LITI) method.

The light-emitting element ED according to one or more embodiments may contain a nitrogen-containing compound according to one or more embodiments in the electron transport region ETR. In the light-emitting element ED according to one or more embodiments, the electron transport region ETR may include an electron injection layer EIL and an electron transport layer ETL, and the electron transport layer ETL may contain a nitrogen-containing compound according to one or more embodiments. The nitrogen-containing compound according to one or more embodiments may be included in a layer, among layers included in the electron transport region ETR, adjacent to the emission layer EML.

The nitrogen-containing compound according to one or more embodiments may include three triazine moieties and a first substituent that is connected to at least one among the three triazine moieties. In one or more embodiments, the first substituent may be a substituted or unsubstituted alkyl group having 1 to 30 carbons, or a cycloalkyl group having 3 to 30 ring-forming carbons. In the nitrogen-containing compound according to one or more embodiments, the first substituent may be singular or plural.

The three triazine moieties included in the nitrogen-containing compound according to one or more embodiments may be connected to each other via a linker. The three triazine moieties included in the nitrogen-containing compound according to one or more embodiments may be connected via one linker. In some embodiments, the three triazine moieties included in the nitrogen-containing compound according to one or more embodiments may be connected via two linkers. For example, among the three triazine moieties included in the nitrogen-containing compound, a first triazine moiety and a second triazine moiety according to one or more embodiments may be connected via a first linker, and the first triazine moiety and a third triazine moiety may be connected via a second linker.

Because the three triazine moieties are connected via a linker and the nitrogen-containing compound according to one or more embodiments includes the first substituent connected to at least one among the three triazine moieties, the nitrogen-containing compound according to one or more embodiments may have excellent or suitable electrical stability and high charge-transporting ability, and high glass transition temperature thereof prevents crystallization, thereby exhibiting excellent or suitable chemical stability. Therefore, if (e.g., when) the nitrogen-containing compound according to one or more embodiments is applied to the electron transport region ETR of the light-emitting element ED, the light-emitting element may exhibit excellent or suitable luminous efficiency and improved lifespan characteristics.

### Nitrogen-Containing Compound

The nitrogen-containing compound according to one or more embodiments may be represented by Formula 1.

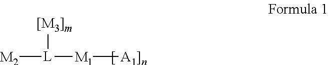

The nitrogen-containing compound represented by Formula 1 includes first to third triazine moieties and a first substituent linked to at least one selected from among the first to third triazine moieties. In one or more embodiments, in Formula 1, a substituent which is represented by M₁ may correspond to the first triazine moiety, a substituent which is represented by M₂ may correspond to the second triazine moiety, and substituents represented by M₃ and M₄ may each correspond to the third triazine moiety. In some embodiments, in Formula 1, L, L₁, and L₂ may each correspond to a linker of the herein-described nitrogen-containing compound.

In Formula 1, A₁ may be -(L₁)ₐ₁M₄.

In Formula 1, L₁ may be a substituted or unsubstituted arylene group having 6 to 60 ring-forming carbons, or a substituted or unsubstituted heteroarylene group having 2 to 60 ring-forming carbons. In one or more embodiments, L₁ may be a substituted or unsubstituted arylene group having 6 to 30 ring-forming carbons. For example, L₁ may be a substituted or unsubstituted phenylene group, a substituted or unsubstituted divalent biphenyl group, or a substituted or unsubstituted divalent naphthyl group. In one or more embodiments, L₁ may correspond to the second linker of the herein-described nitrogen-containing compound.

In Formula 1, M₂ to M₄ may each independently be a substituted or unsubstituted triazine group.

In Formula 1, a1 may be an integer of 1 to 3. For example, a1 may be 1.

In Formula 1, m and n may each independently be 0 or 1.

In Formula 1, the sum of m and n may be 1. For example, if (e.g., when) m is 0, n may be 1, and if (e.g., when) n is 0, m may be 1.

In Formula 1, if (e.g., when) n is 0, M₁ may be a substituted or unsubstituted triazine group.

In Formula 1, if (e.g., when) n is 1, M₁ may be a substituted or unsubstituted divalent triazine group.

In Formula 1, m is 0, L may be represented by -(L₂)ₐ₂-.

In Formula 1, L₂ may be a substituted or unsubstituted arylene group having 6 to 60 ring-forming carbons, or a substituted or unsubstituted heteroarylene group having 2 to 60 ring-forming carbons. In one or more embodiments, L₂ may be a substituted or unsubstituted arylene group having 6 to 30 ring-forming carbons. For example, L₂ may be a substituted or unsubstituted phenylene group, a substituted or unsubstituted divalent biphenyl group, or a substituted or unsubstituted divalent naphthyl group. In one or more embodiments, L₂ may correspond to the first linker of the herein-described nitrogen-containing compound.

In Formula 1, a2 may be an integer of 1 to 3. For example, a2 may be 1.

In Formula 1, if (e.g., when) m is 1, L may be a substituted or unsubstituted trivalent aryl group having 6 to 60 ring-forming carbons, or a substituted or unsubstituted trivalent heteroaryl group having 2 to 60 ring-forming carbons. In one or more embodiments, L may be a substituted or unsubstituted trivalent aryl group having 6 to 30 ring-forming carbons. For example, L may be a substituted or unsubstituted trivalent phenyl group.

In Formula 1, at least one selected from among M₁ to M₄ includes a substituted or unsubstituted alkyl group having 1 to 30 carbons, or a substituted or unsubstituted cycloalkyl group having 3 to 30 ring-forming carbons as a substituent. More specifically, if (e.g., when) m is 0 and n is 1, at least one selected from among M₁, M₂, and M₄ includes a substituted or unsubstituted alkyl group having 1 to 30 carbons, or a substituted or unsubstituted cycloalkyl group having 3 to 30 ring-forming carbons as a substituent. In one or more embodiments, if (e.g., when) m is 1 and n is 0, at least one selected from among M₁ to M₃ includes a substituted or unsubstituted alkyl group having 1 to 30 carbons or a substituted or unsubstituted cycloalkyl group having 3 to 30 ring-forming carbons as a substituent.

In one or more embodiments, at least one selected from among M₁ to M₄ may be substituted with a substituted or unsubstituted methyl group, a substituted or unsubstituted isopropyl group, a substituted or unsubstituted t-butyl group, a substituted or unsubstituted cyclohexyl group, a substituted or unsubstituted bicycloheptanyl group, or a substituted or unsubstituted adamantyl group. More specifically, if m is 0 and n is 1, at least one among M₁, M₂, and M₄ may be substituted with a substituted or unsubstituted methyl group, a substituted or unsubstituted isopropyl group, a substituted or unsubstituted t-butyl group, a substituted or unsubstituted cyclohexyl group, a substituted or unsubstituted bicycloheptanyl group, or a substituted or unsubstituted adamantyl group. In one or more embodiments, if (e.g., when) m is 1 and n is 0, at least one selected from among M₁ to M₃ may be substituted with a substituted or unsubstituted methyl group, a substituted or unsubstituted isopropyl group, a substituted or unsubstituted t-butyl group, a substituted or unsubstituted cyclohexyl group, a substituted or unsubstituted bicycloheptanyl group, or a substituted or unsubstituted adamantyl group.

In Formula 1, cases where each of L, L₁, and L₂ includes a substituted or unsubstituted carbazole linker are excluded (e.g., each of L, L₁, and La do not comprise a substituted or unsubstituted carbazole linker). For example, the nitrogen-containing compound according to one or more embodiments represented by Formula 1 may include no substituted or unsubstituted carbazole linker, as a linker that connects a triazine moiety. For example, if (e.g., when) m is 0 and n is 1 in Formula 1, (then) cases where each of L₁ and L₂ is a substituted or unsubstituted divalent carbazole group are excluded, and if (e.g., when) m is 1 and n is 0 in Formula 1, (then) a case where L is a substituted or unsubstituted trivalent carbazole group is excluded. If (e.g., when) L, L₁, and L₂, which correspond to linkers in the nitrogen-containing compound according to one or more embodiments represented by Formula 1, each includes a carbazole linker, (then) the charge-transporting properties are reduced, which may act as a disadvantage in luminous efficiency and lifespan characteristics. In the present disclosure, because the cases where each of L, L₁, and L₂, each corresponding to a linker in the nitrogen-containing compound according to one or more embodiments represented by Formula 1, includes a carbazole linker are excluded, the charge-transporting ability may be improved, and thus improvements in luminous efficiency and lifespan characteristics of the light-emitting element ED may be exhibited.

In one or more embodiments, L₁ and L₂ may each independently be a substituted or unsubstituted divalent phenyl group, a substituted or unsubstituted divalent biphenyl group, or a substituted or unsubstituted divalent naphthyl group, and L may be a substituted or unsubstituted trivalent phenyl group, a substituted or unsubstituted trivalent biphenyl group, or a substituted or unsubstituted trivalent naphthyl group.

In one or more embodiments, the nitrogen-containing compound represented by Formula 1 may be represented by Formula 2-1 or Formula 2-2.

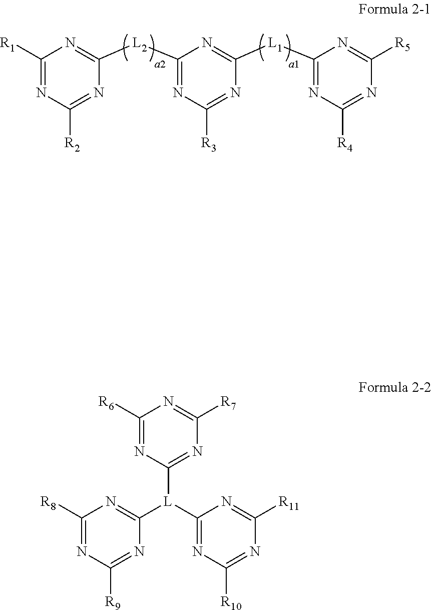

Formula 2-1 and Formula 2-2 each represent cases where m and n are specified in Formula 1. Formula 2-1 represents a case where m is 0 and n is 1 in Formula 1, and Formula 2-2 represents a case where m is 1 and n is 0 in Formula 1.

In Formula 2-1 and Formula 2-2, R₁ to R₁₁ may each independently be a hydrogen atom, a deuterium atom, a halogen atom, a nitro group, a cyano group, a substituted or unsubstituted alkyl group having 1 to 30 carbons, a substituted or unsubstituted cycloalkyl group having 3 to 30 ring-forming carbons, a substituted or unsubstituted aryl group having 6 to 60 ring-forming carbons, or a substituted or unsubstituted heteroaryl group having 2 to 60 ring-forming carbons. In one or more embodiments, R₁ to R₁₁ may be a substituted or unsubstituted alkyl group having 1 to 20 carbons, or a substituted or unsubstituted aryl group having 6 to 30 ring-forming carbons. For example, R₁ to R₁₁ may each independently be a substituted or unsubstituted methyl group, a substituted or unsubstituted isopropyl group, a substituted or unsubstituted t-butyl group, a substituted or unsubstituted cyclohexyl group, a substituted or unsubstituted bicycloheptanyl group, a substituted or unsubstituted adamantyl group, a substituted or unsubstituted pyridine group, a substituted or unsubstituted phenyl group, or a substituted or unsubstituted biphenyl group.

At least one selected from among R₁ to R₅ in Formula 2-1 may be a substituted or unsubstituted alkyl group having 1 to 30 carbons, or a substituted or unsubstituted cycloalkyl group having 3 to 30 ring-forming carbons. In one or more embodiments, at least one selected from among R₁ to R₅ in Formula 2-1 may be a substituted or unsubstituted alkyl group having 1 to 10 carbons, or a substituted or unsubstituted cycloalkyl group having 3 to 30 ring-forming carbons, and the remaining may each independently be a substituted or unsubstituted phenyl group, or a substituted or unsubstituted biphenyl group.

At least one selected from among R₆ to R₁₁ in Formula 2-2 may be a substituted or unsubstituted alkyl group having 1 to 30 carbons, or a substituted or unsubstituted cycloalkyl group having 3 to 30 ring-forming carbons. In one or more embodiments, at least one selected from among R₆ to R₁₁ in Formula 2-2 may be a substituted or unsubstituted alkyl group having 1 to 10 carbons, or a substituted or unsubstituted cycloalkyl group having 3 to 30 ring-forming carbons, and the remaining may each independently be a substituted or unsubstituted phenyl group, or a substituted or unsubstituted biphenyl group.

In Formula 2-1 and Formula 2-2, the descriptions of L, L₁, L₂, a1, and a2 described in Formula 1 may be similarly applied.

In one or more embodiments, the nitrogen-containing compound represented by Formula 2-1 may be represented by any one selected from among Formula 3-1 to Formula 3-3.

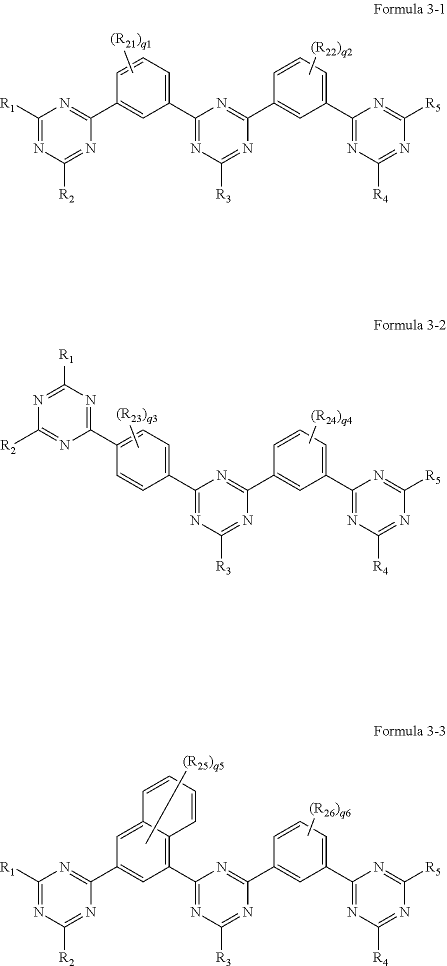

Formula 3-1 to Formula 3-3 each represent cases where the types (kinds) and connection positions of L₁ and L₂ are specified in Formula 2-1.

In Formula 3-1 to Formula 3-3, R₂₁ to R₂₆ may each independently be a hydrogen atom, a deuterium atom, a halogen atom, a cyano group, a nitro group, a substituted or unsubstituted phosphine oxide group, a substituted or unsubstituted alkyl group having 1 to 30 carbons, a substituted or unsubstituted aryl group having 6 to 60 ring-forming carbons, or a substituted or unsubstituted heteroaryl group having 2 to 60 ring-forming carbons. For example, R₂₁ to R₂₆ may each independently be a hydrogen atom, a halogen atom, a cyano group, a substituted or unsubstituted phosphine oxide group, a substituted or unsubstituted phenyl group, or a substituted or unsubstituted pyridine group.

In Formula 3-1 to Formula 3-3, q1 to q4, and q6 may each independently be an integer of 0 to 4, and q5 is an integer of 0 to 6. If q1 to q6 are each 0, the nitrogen-containing compound according to one or more embodiments may be unsubstituted with each of R₂₁ to R₂₆. Cases where q1 to q4, and q6 are each 4 and R₂₁ to R₂₄, and R₂₆ are all hydrogen atoms may be the same as cases where q1 to q4, and q6 are each 0. A case where q5 is 6 and all R₂₅s are hydrogen atoms may be the same as the case where q5 is 0. If q1 to q6 are each an integer of 2 or greater, all R₂₁s to R₂₆s provided in plural may be the same, or at least one among a plurality of R₂₁s to R₂₆s may be different.

In Formula 3-1 to Formula 3-3, the descriptions of R₁ to R₅ described in Formula 2-1 may be similarly applied.

In one or more embodiments, the nitrogen-containing compound represented by Formula 2-2 may be represented by Formula 4.

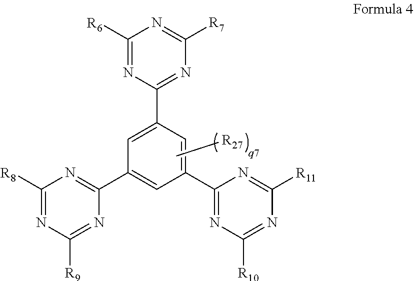

Formula 4 represents a case where the type or kind and connection position of L in Formula 2-2 is specified.

In Formula 4, R₂₇ may be a hydrogen atom, a deuterium atom, a halogen atom, a cyano group, a nitro group, a substituted or unsubstituted phosphine oxide group, a substituted or unsubstituted alkyl group having 1 to 20 carbons, a substituted or unsubstituted aryl group having 6 to 60 ring-forming carbons, or a substituted or unsubstituted heteroaryl group having 2 to 60 ring-forming carbons. For example, R₂₇ may be a hydrogen atom, a halogen atom, a cyano group, or a substituted or unsubstituted phosphine oxide group.

In Formula 4, q7 may be an integer of 0 to 3. If (e.g., when) q7 is 0, the nitrogen-containing compound according to one or more embodiments may be unsubstituted with R₂₇. A case where q7 is 3 and R₂₇s are all hydrogen atoms in Formula 4 may be the same as the case where q7 is 0 in Formula 4. If q7 is an integer of 2 or greater, R₂₇s provided in plural may be all the same, or at least one among the plurality of R₂₇s may be different.

In Formula 4, the descriptions of R₆ to R₁₁ described in Formula 2-2 may be similarly applied.

In one or more embodiments, L₁ and L₂ in Formula 1 may each independently be represented by any one selected from among Formula L-1 to Formula L-9, and L may be represented by Formula L-10.

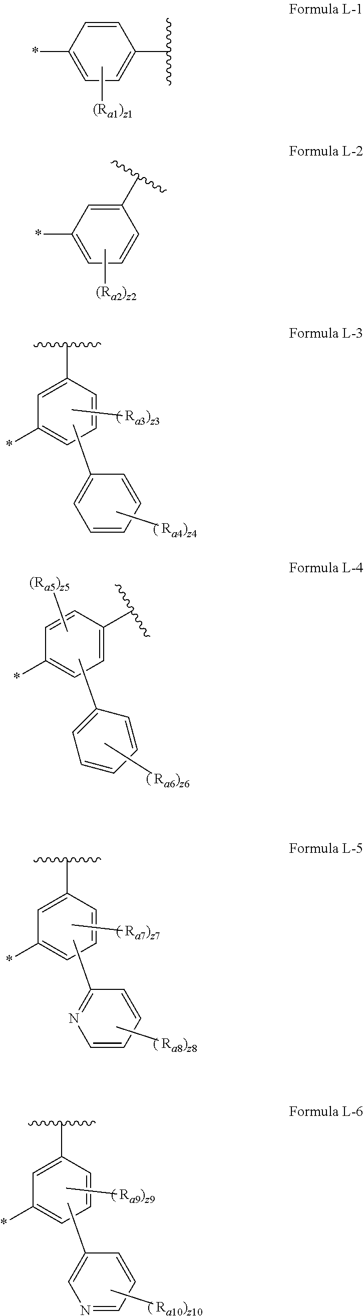

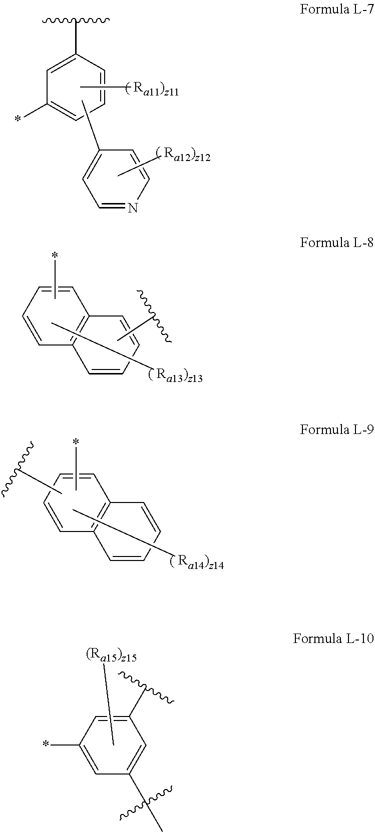

In Formula L-1 to Formula L-10, Rₐ₁ to Rₐ₁₅ may each independently be a hydrogen atom, a deuterium atom, a halogen atom, a cyano group, a substituted or unsubstituted phosphine oxide group, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons. For example, Rₐ₁ to Rₐ₁₅ may be a hydrogen atom, a halogen atom, a cyano group, or a substituted or unsubstituted phosphine oxide group.

In Formula L-1 to Formula L-10, z1, z2, z8, z10, and z12 may each independently be an integer of 0 to 4, z3, z5, z7, z9, z11, and z15 may each independently be an integer of 0 to 3, z4 and z6 may each independently be an integer of 0 to 5, and z13 and z14 may each independently be an integer of 0 to 6. If (e.g., when) z1 to z15 are each 0, the nitrogen-containing compound according to one or more embodiments may be unsubstituted with each of Rₐ₁ to Rₐ₁₅. Cases where z1, z2, z10, and z12 are each 4 and all Rₐ₁, Rₐ₂, Rₐ₈, Rₐ₁₀, and Rₐ₁₂ are hydrogen atoms may be the same as the cases where z1, z2, z8, z10, and z12 are each 0. Cases where z3, z5, z7, z9, z11, and z15 are each 3, and all Rₐ₃, Rₐ₅, Rₐ₇, Rₐ₉, Rₐ₁₁, and Rₐ₁₅ are hydrogen atoms may be the same as the cases where z3, z5, z7, z9, z11, and z15 are each 0. Cases where z4, and z6 are each 5, and both (e.g., simultaneously) Rₐ₄, and Rₐ₆ are hydrogen atoms may be the same as the cases where z4, and z6 are each 0. Cases where z13, and z14 are each 6, and both (e.g., simultaneously) Rₐ₁₃, and Rₐ₁₄ are hydrogen atoms may be the same as the cases where z13, and z14 are each 0. If (e.g., when) z1 to z15 are each an integer of 2 or greater, each of Rₐ₁s to Rₐ₁₅s provided in plural may be the same or at least one among the plurality of Rₐ₁s to Rₐ₁₅s may be different.

The nitrogen-containing compound according to one or more embodiments may be any one selected from among compounds present in Compound Group 1. At least one functional layer included in the light-emitting element ED according to one or more embodiments may include at least one nitrogen-containing compound selected from among the compounds present in Compound Group 1. The light-emitting element ED according to one or more embodiments may include at least one nitrogen-containing compound selected from among the compounds present in Compound Group 1 in the electron transport region ETR.

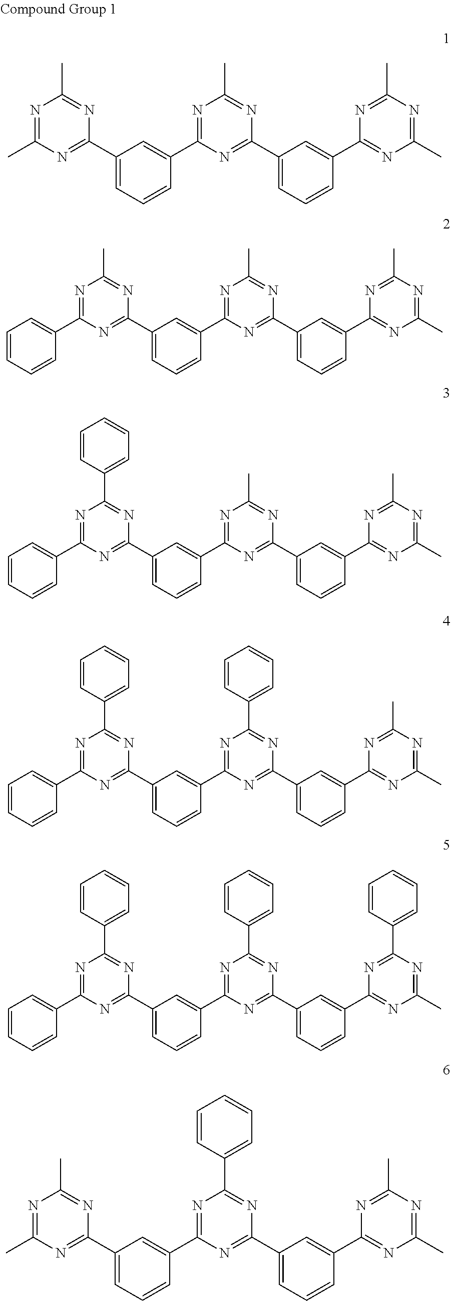

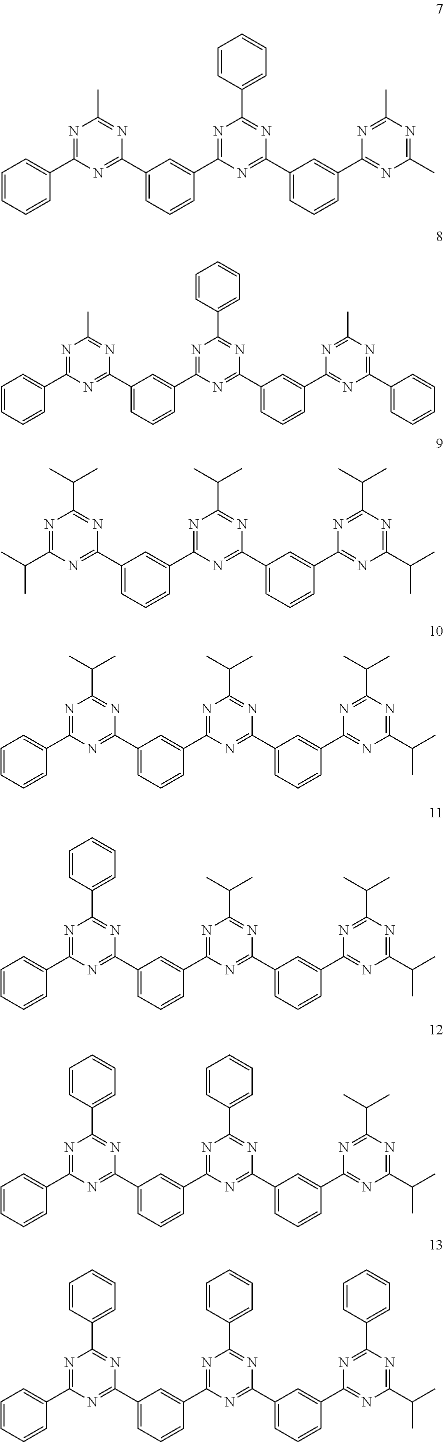

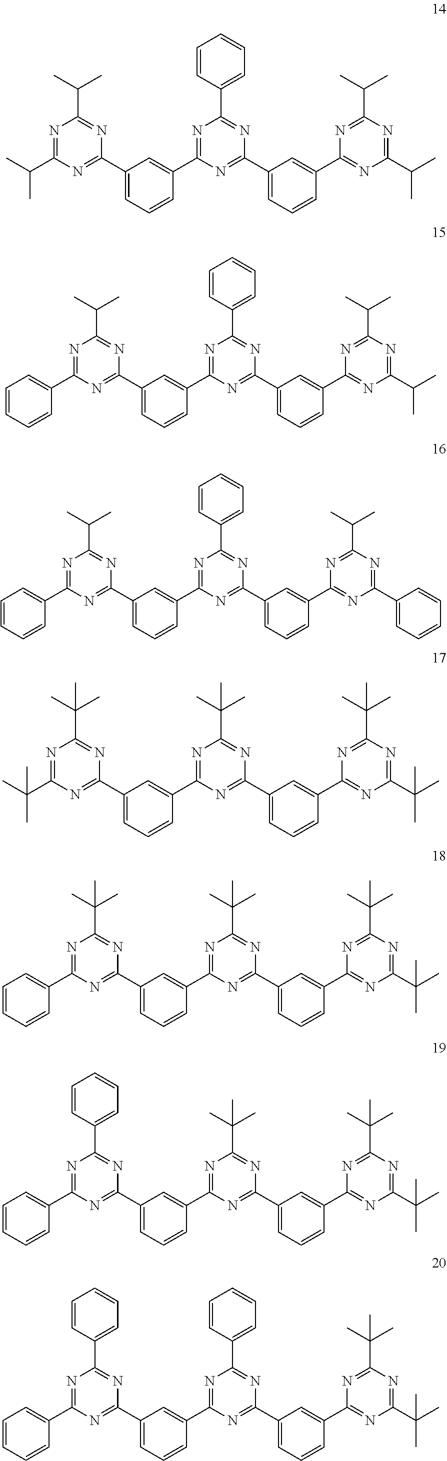

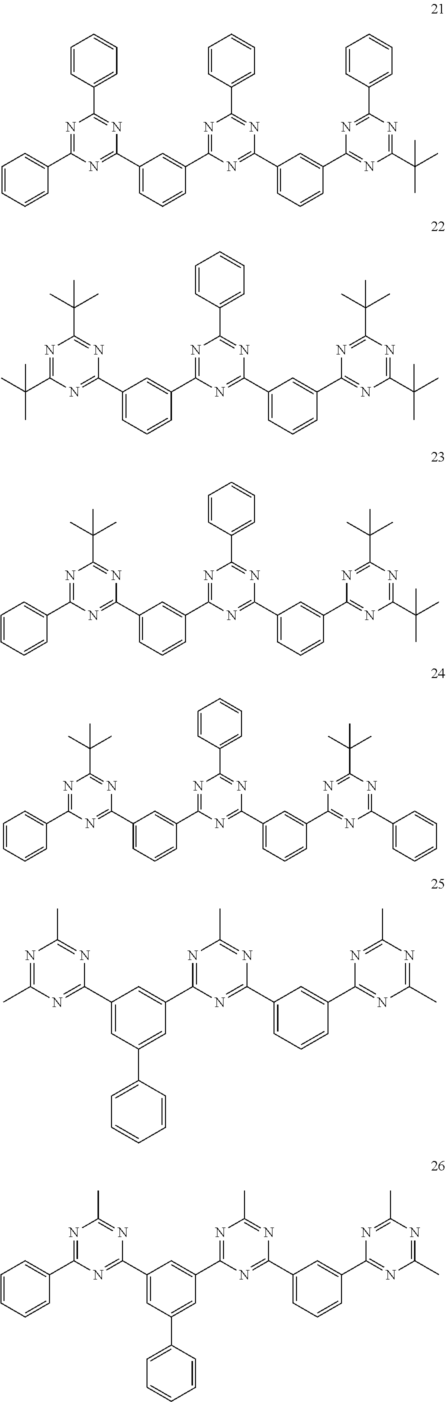

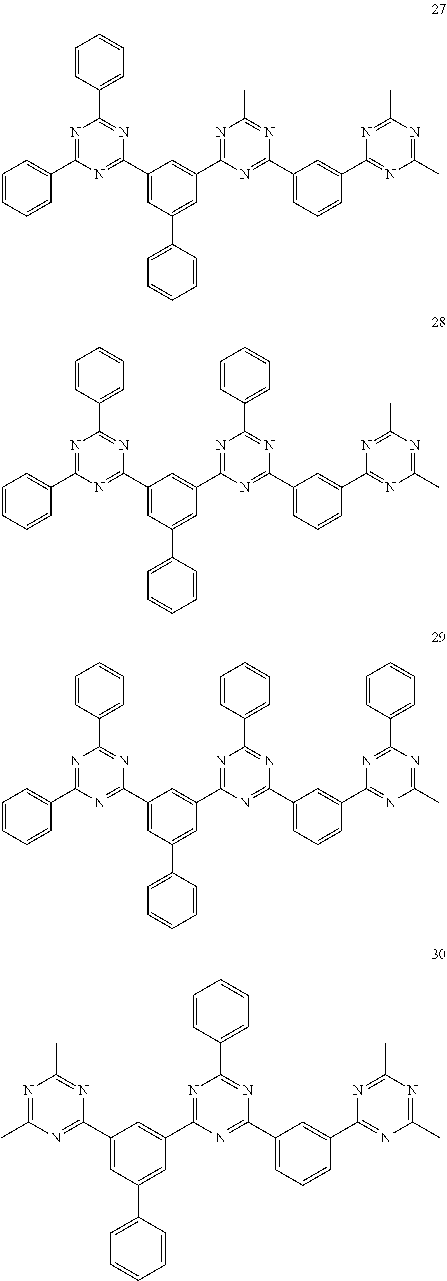

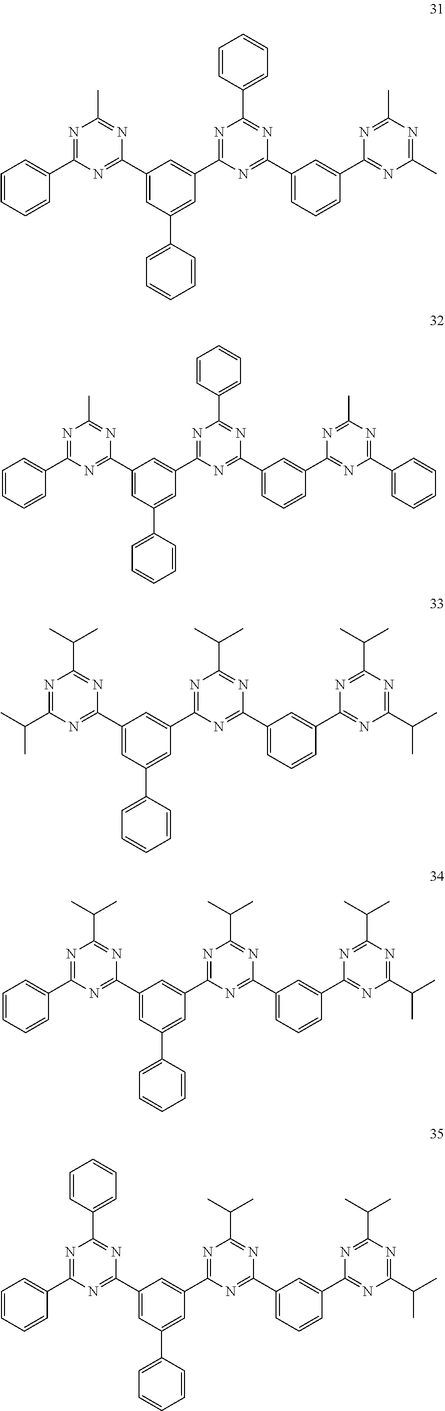

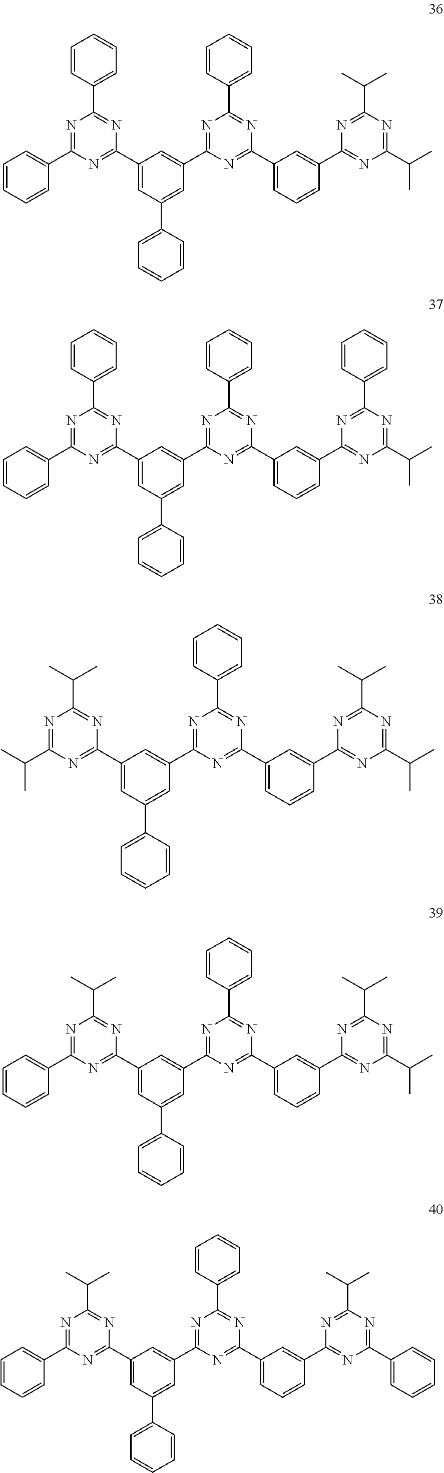

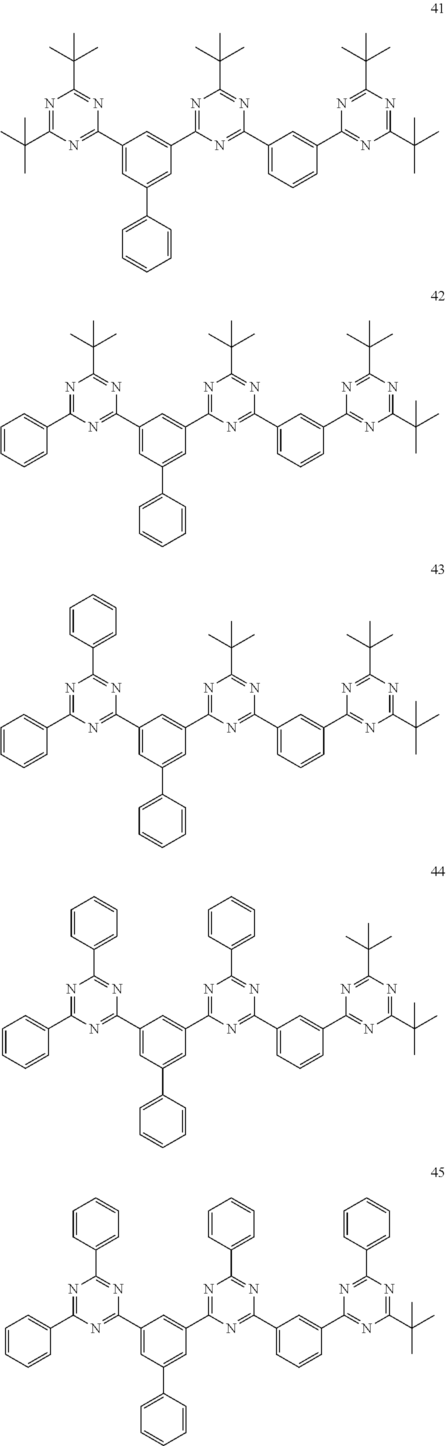

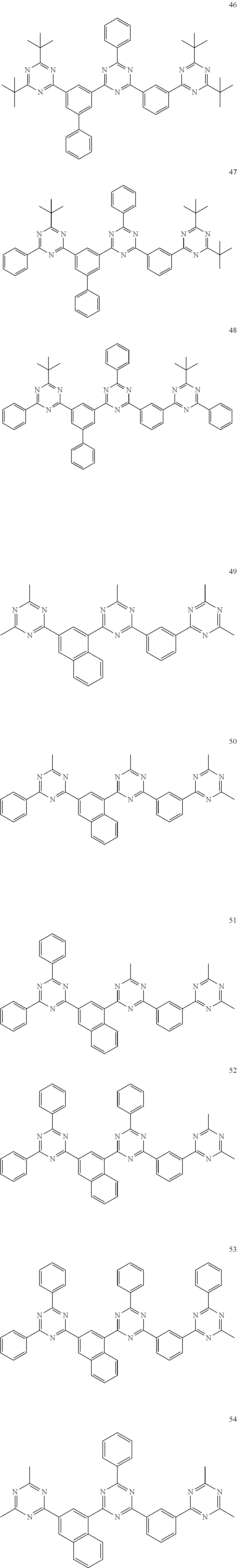

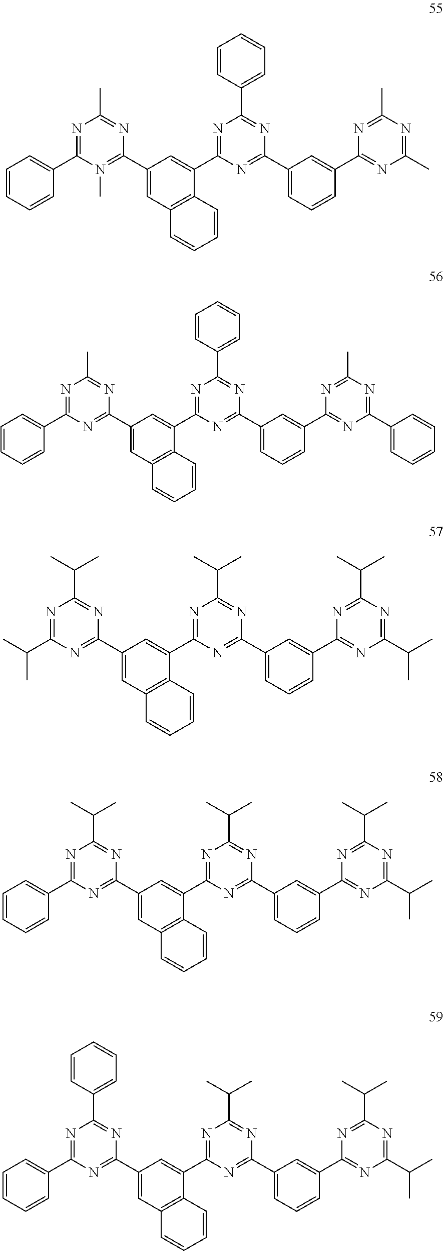

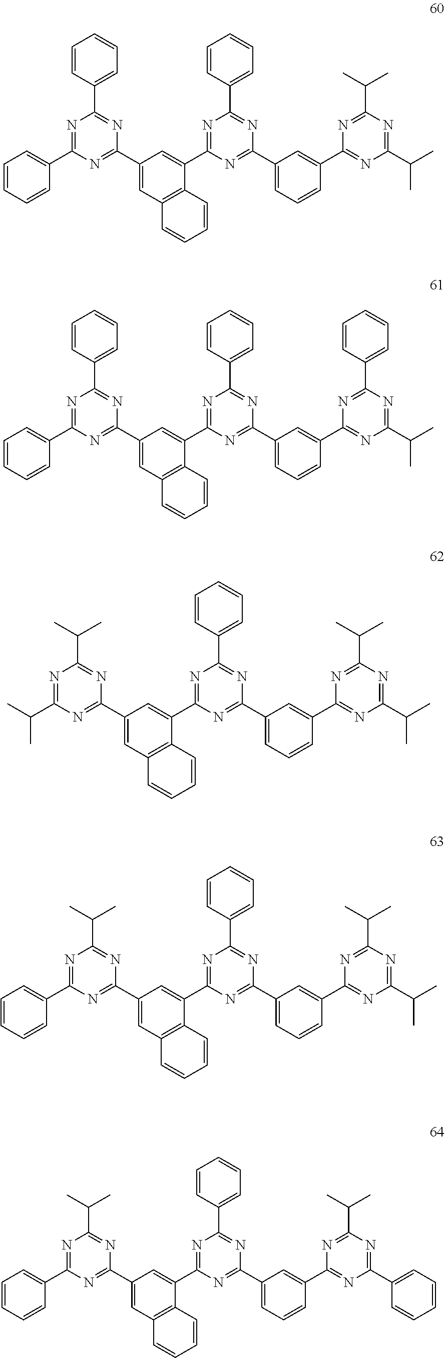

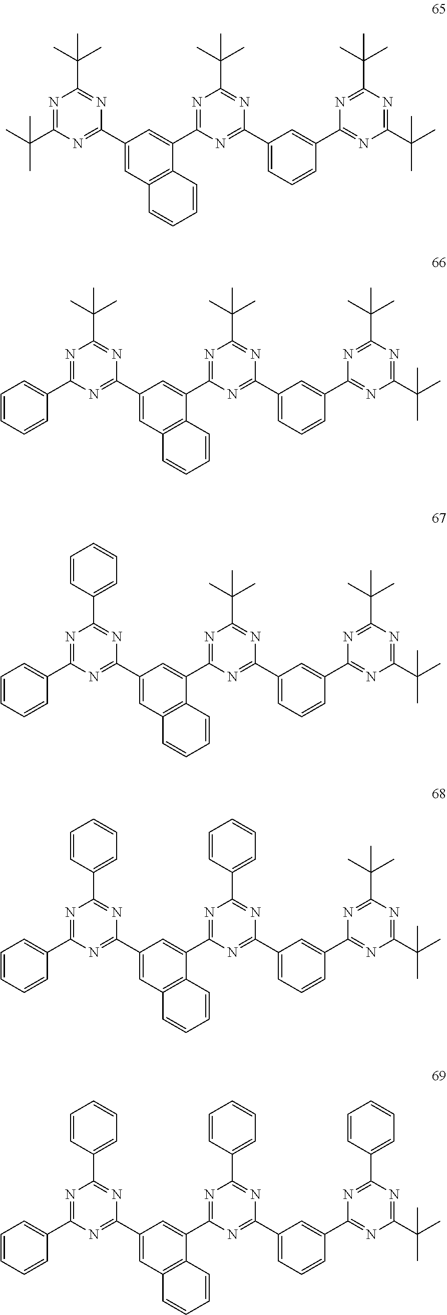

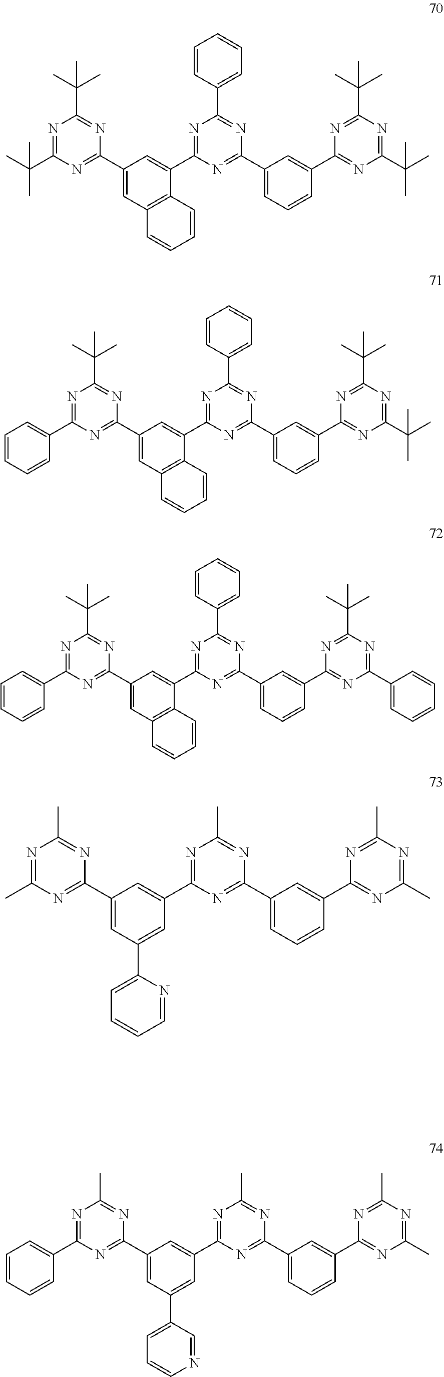

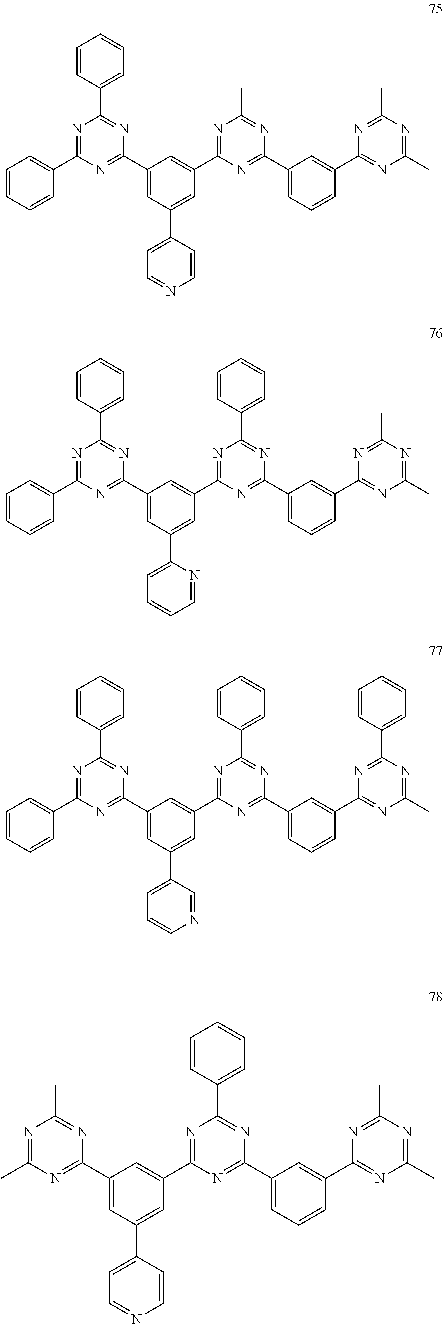

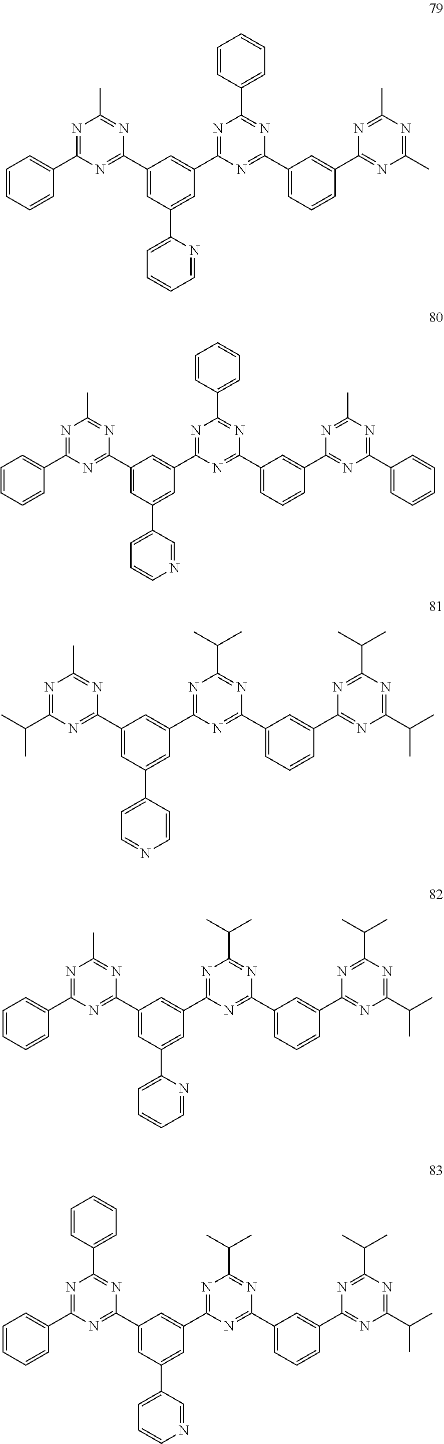

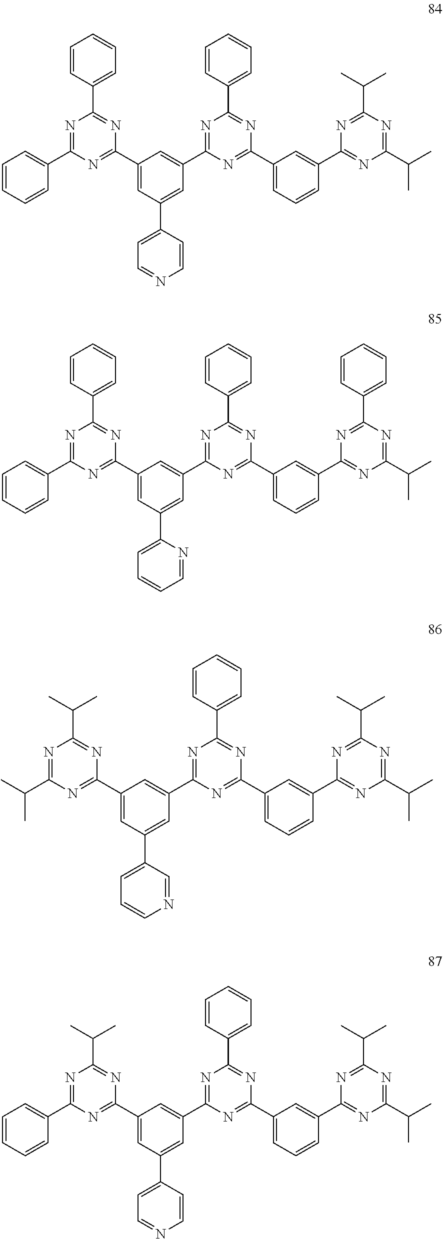

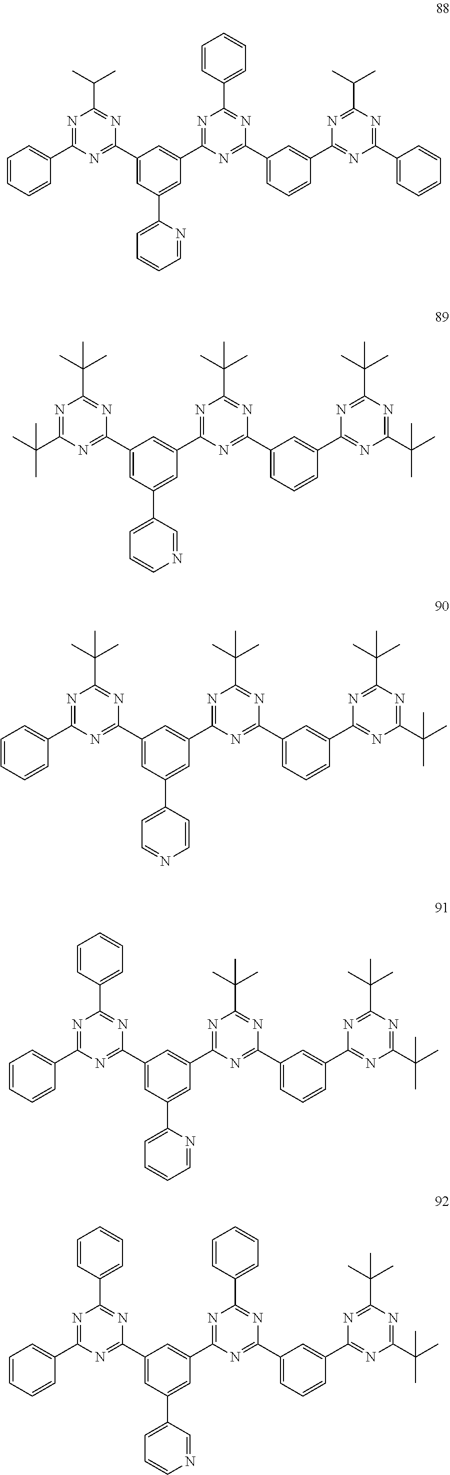

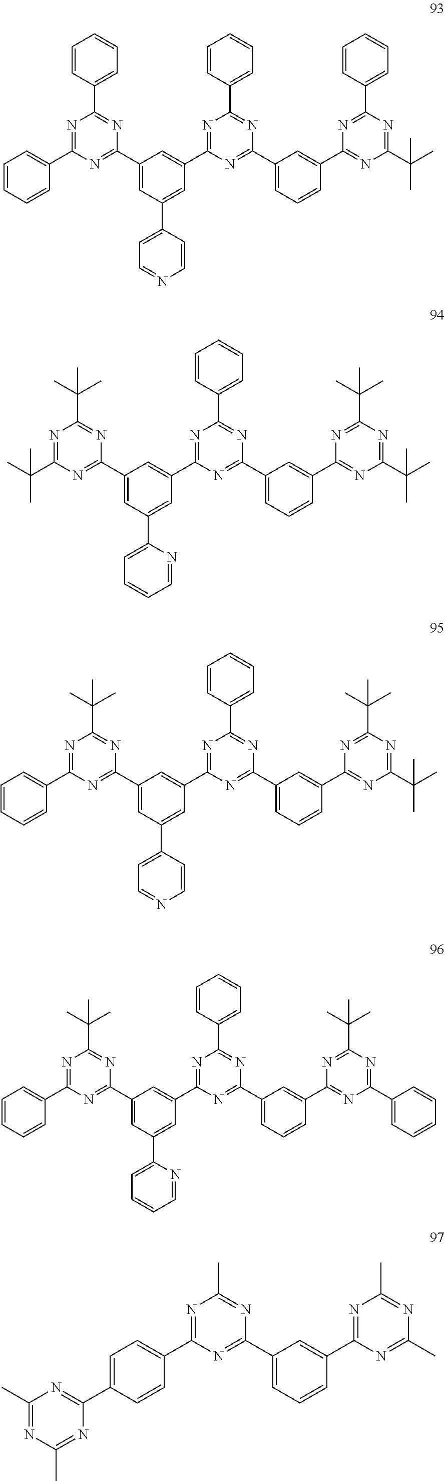

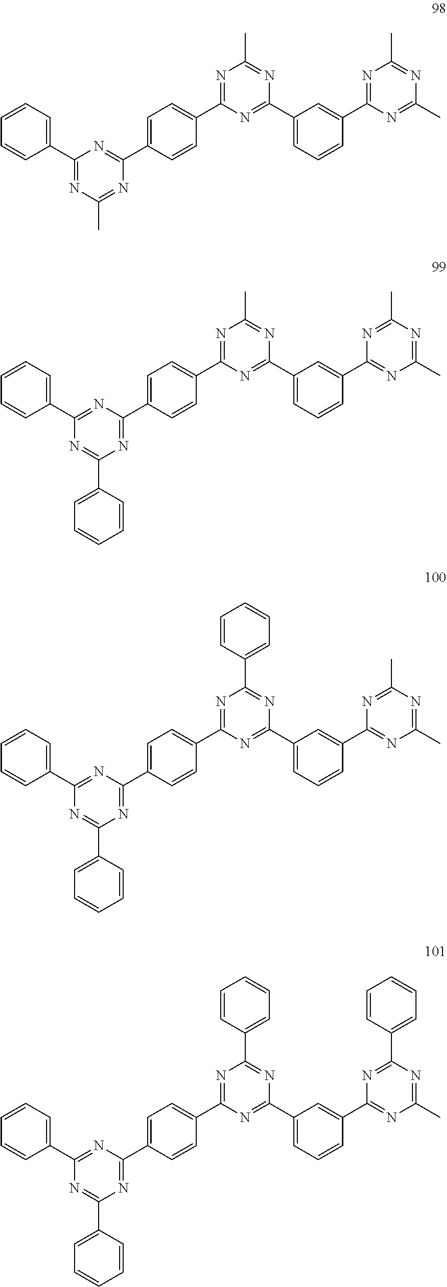 [SMILES: Cc1nc(C)nc(-c2cccc(-c3nc(C)nc(-c4ccc(-c5nc(C)nc(-c6ccccc6)n5)cc4)n3)c2)n1 ; Cc1nc(C)nc(-c2cccc(-c3nc(C)nc(-c4ccc(-c5nc(-c6ccccc6)nc(-c6ccccc6)n5)cc4)n3)c2)n1 ; Cc1nc(C)nc(-c2cccc(-c3nc(-c4ccccc4)nc(-c4ccc(-c5nc(-c6ccccc6)nc(-c6ccccc6)n5)cc4)n3)c2)n1 ; Cc1nc(-c2ccccc2)nc(-c2cccc(-c3nc(-c4ccccc4)nc(-c4ccc(-c5nc(-c6ccccc6)nc(-c6ccccc6)n5)cc4)n3)c2)n1]

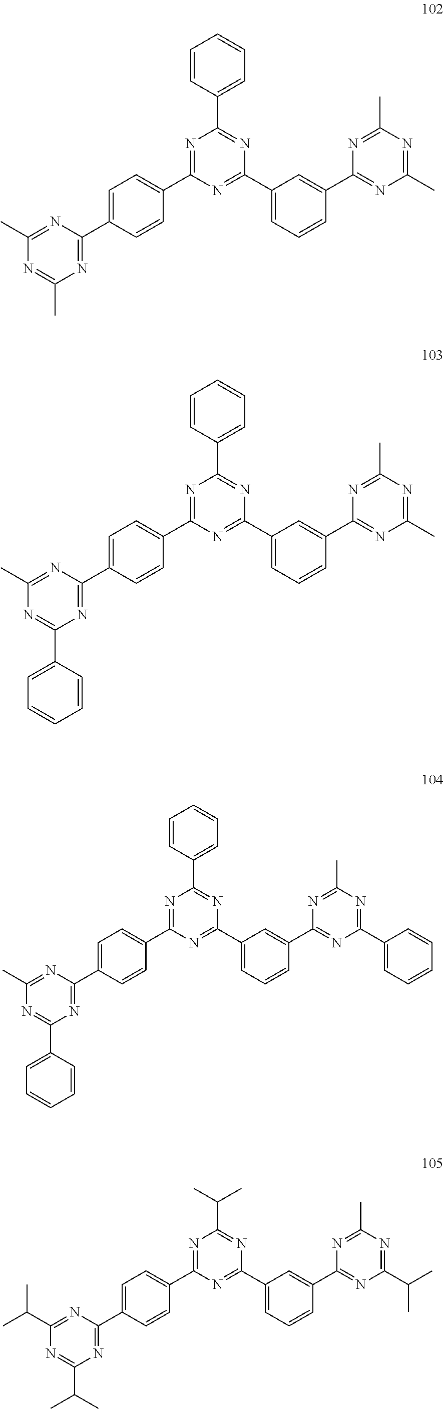

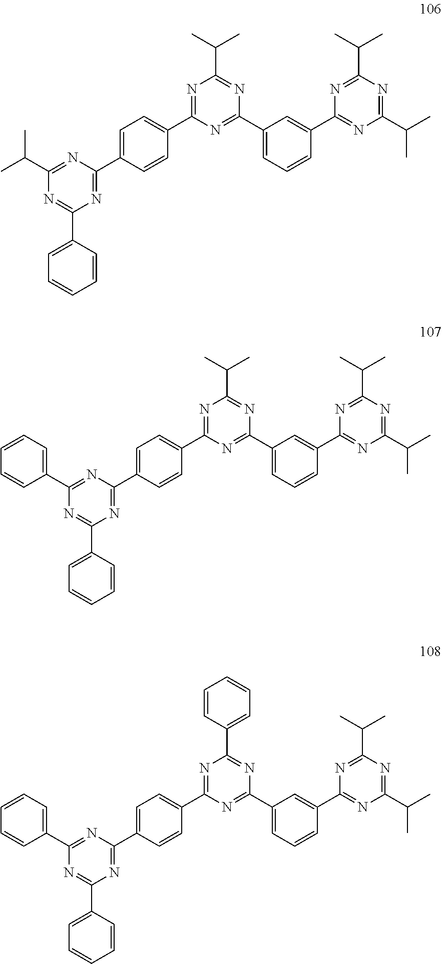 [SMILES: CC(C)c1nc(-c2ccccc2)nc(-c2ccc(-c3nc(-c4cccc(-c5nc(C(C)C)nc(C(C)C)n5)c4)nc(C(C)C)n3)cc2)n1 ; CC(C)c1nc(-c2ccc(-c3nc(-c4ccccc4)nc(-c4ccccc4)n3)cc2)nc(-c2cccc(-c3nc(C(C)C)nc(C(C)C)n3)c2)n1 ; CC(C)c1nc(-c2cccc(-c3nc(-c4ccccc4)nc(-c4ccc(-c5nc(-c6ccccc6)nc(-c6ccccc6)n5)cc4)n3)c2)nc(C(C)C)n1]

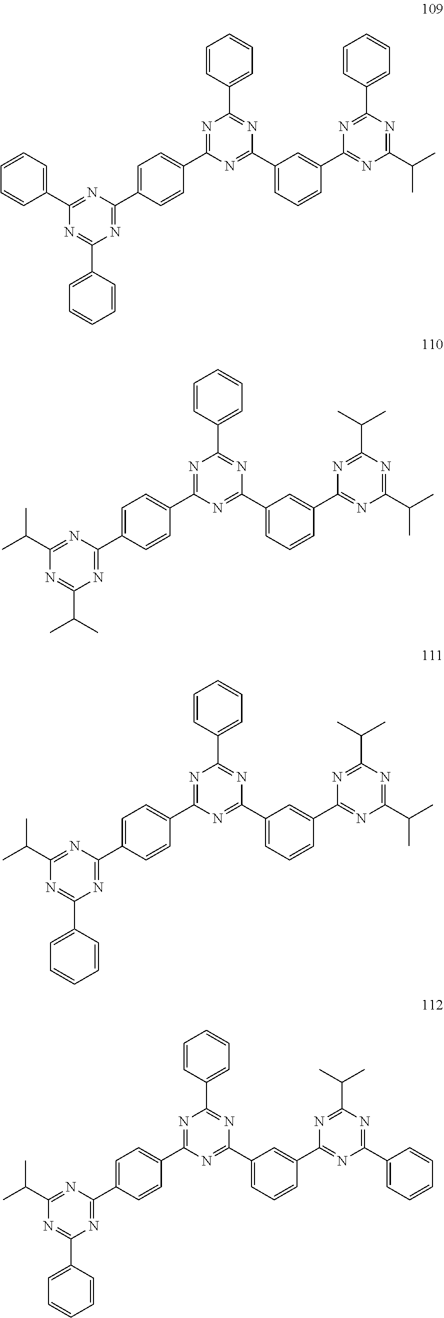

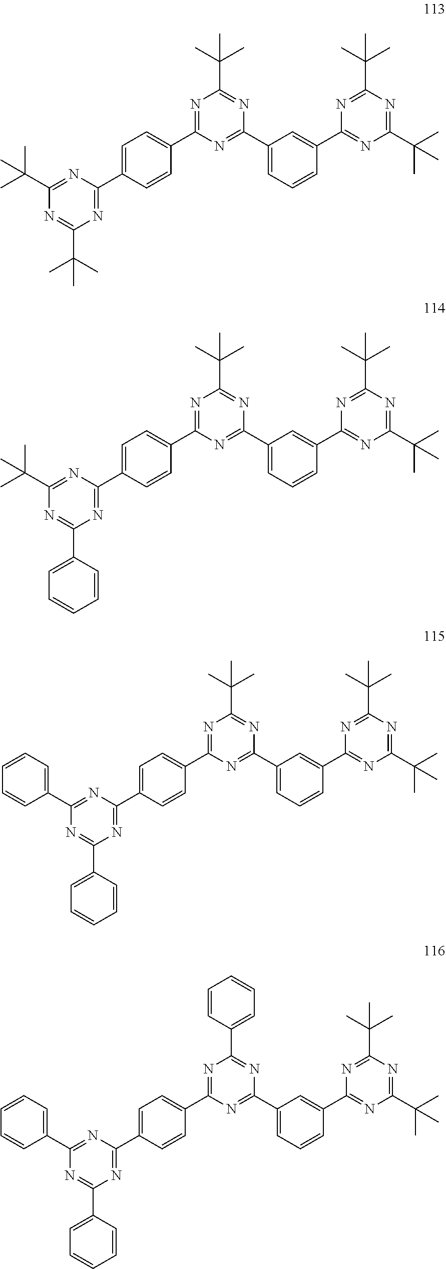

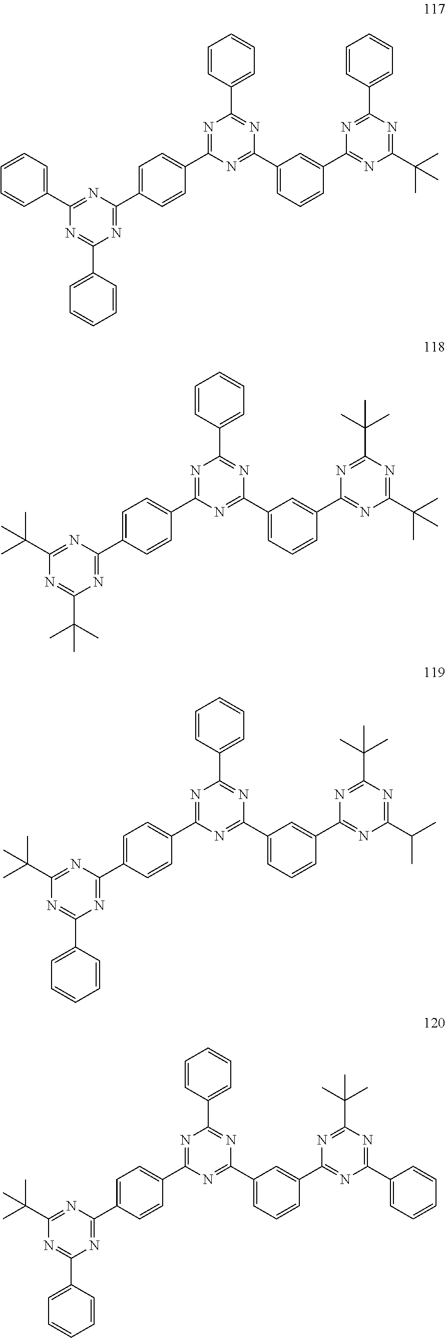

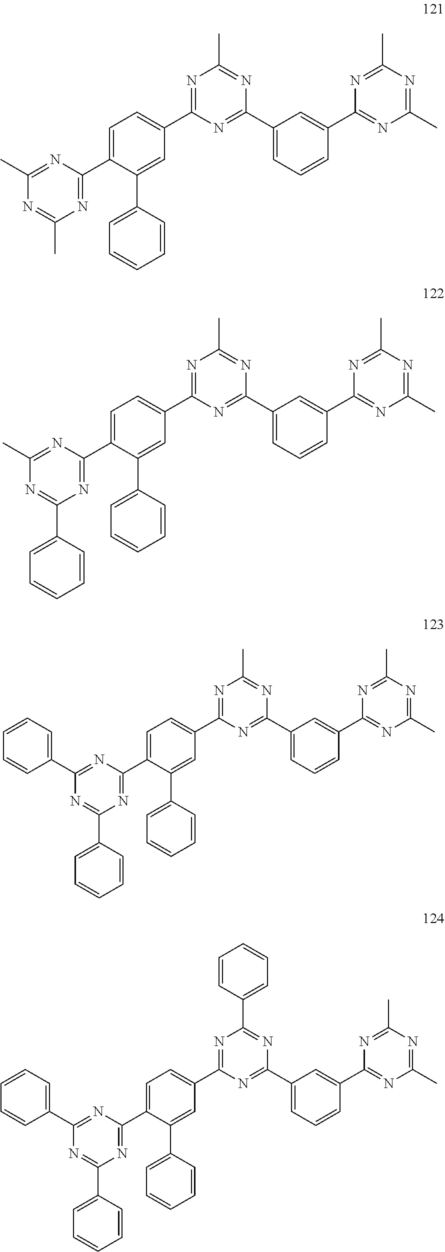

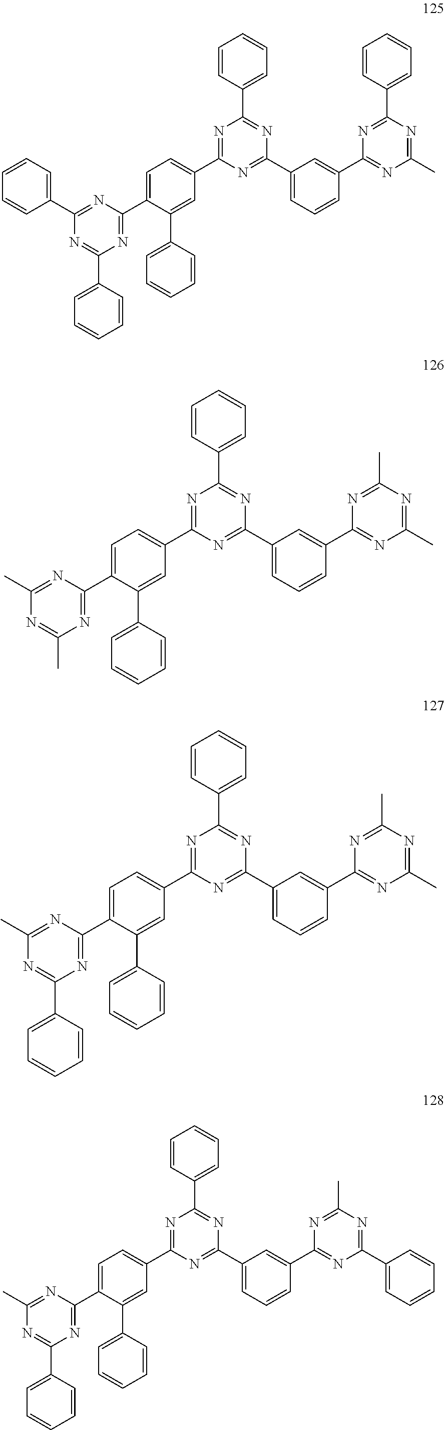

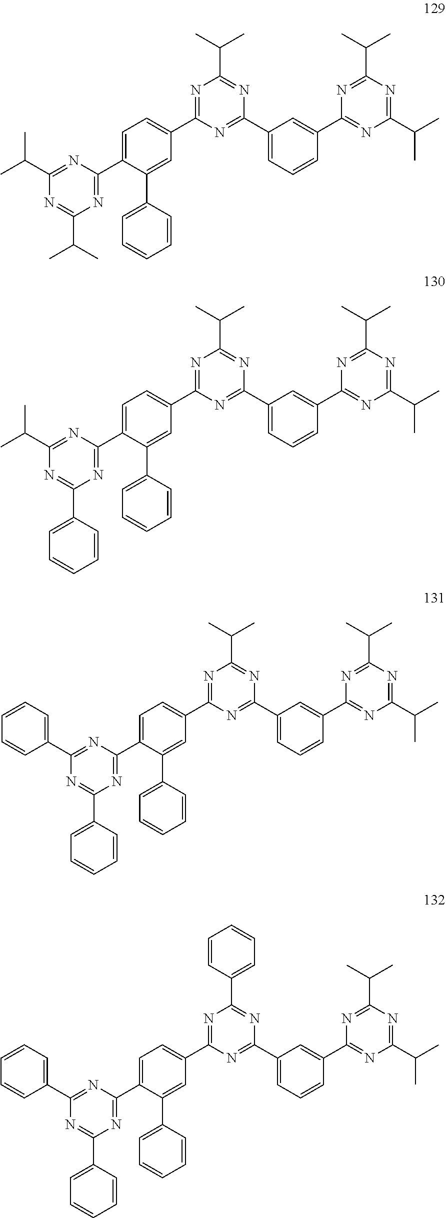

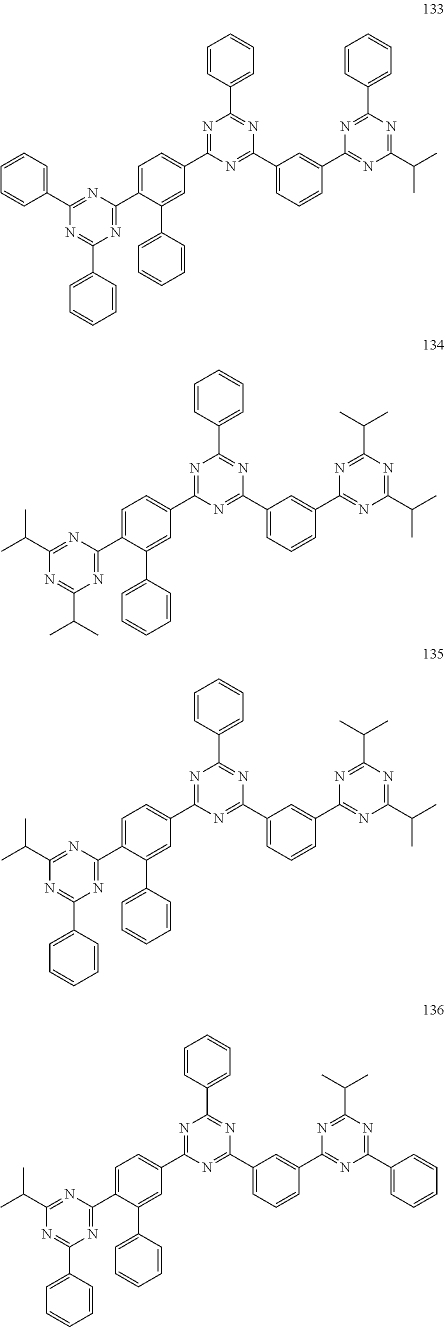

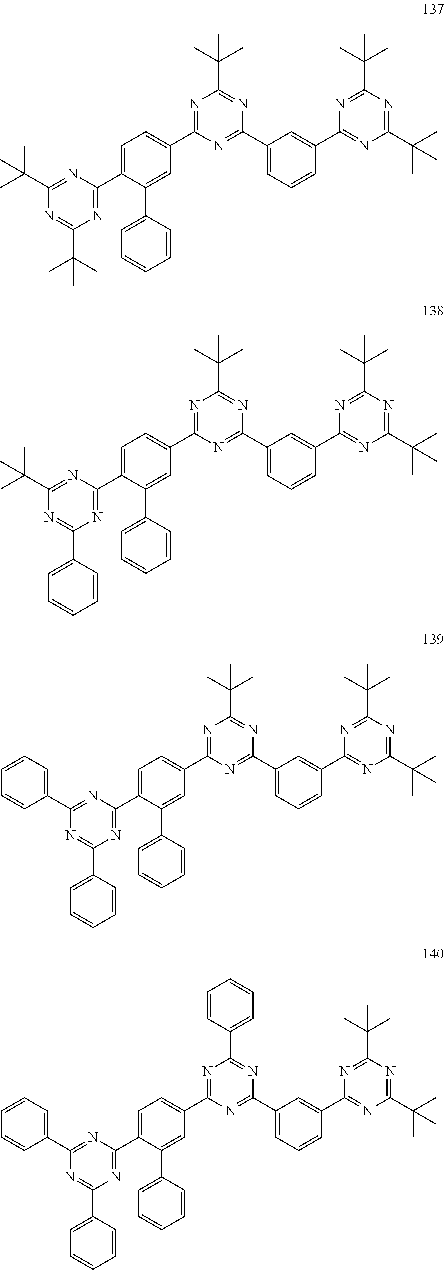

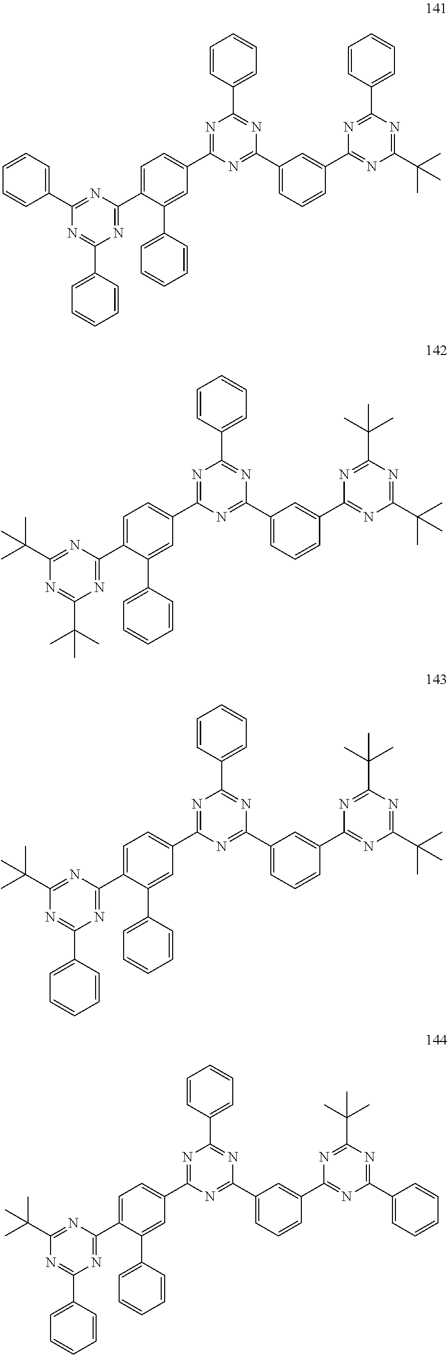

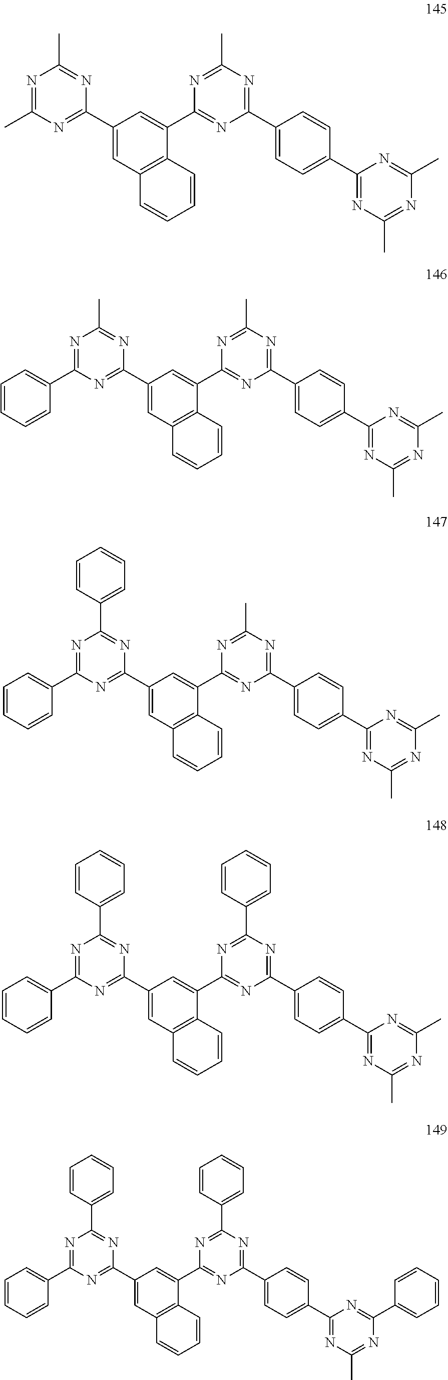

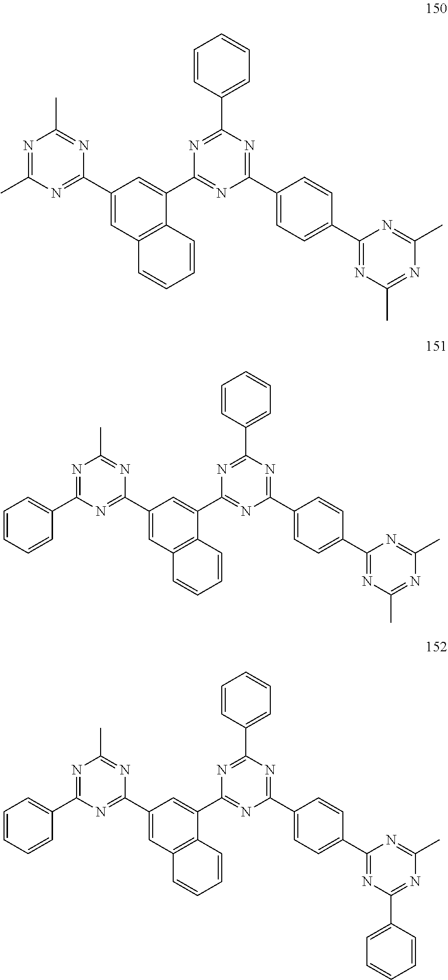

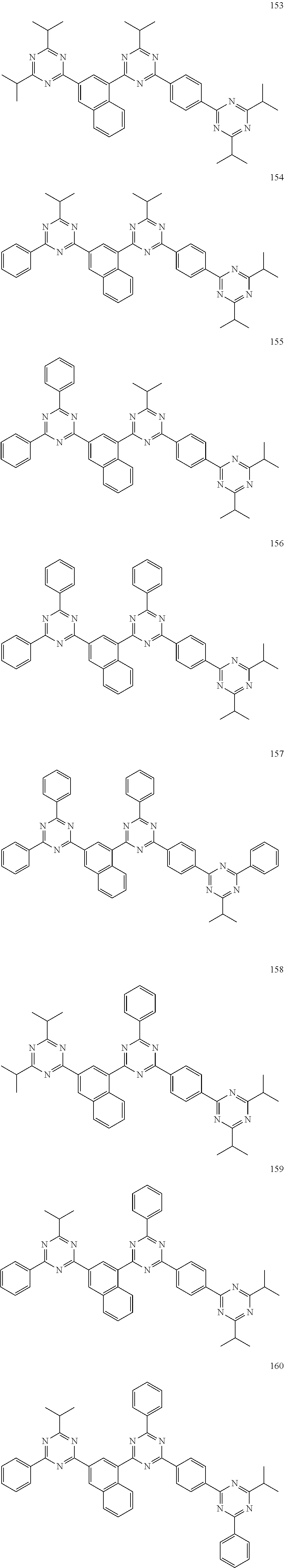

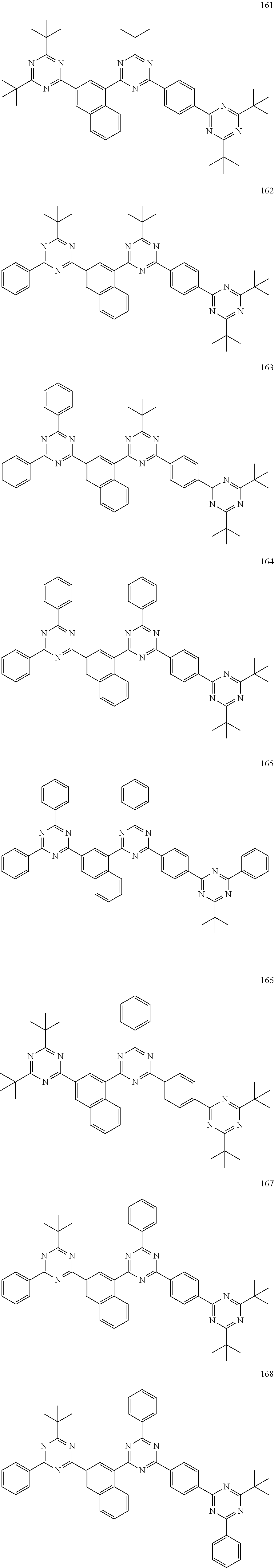

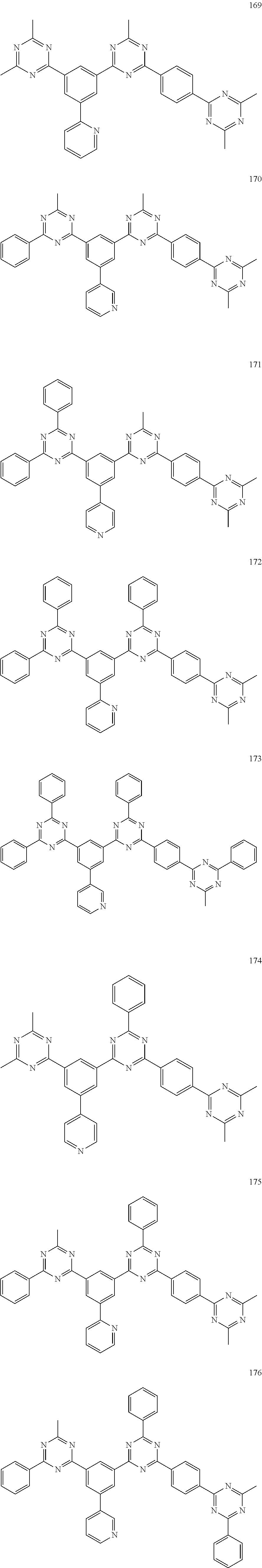

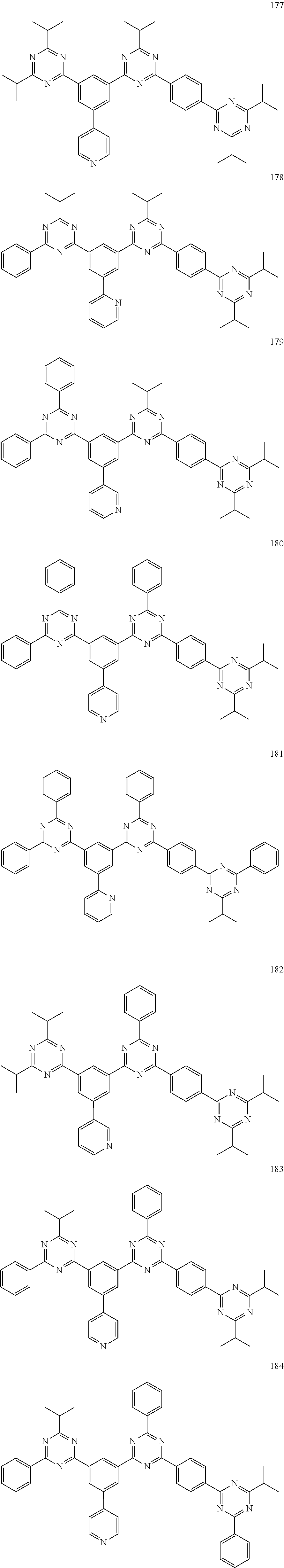

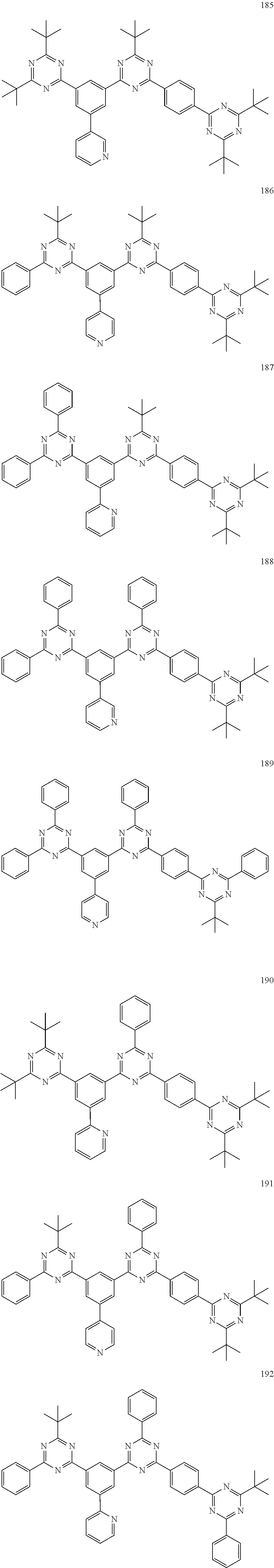

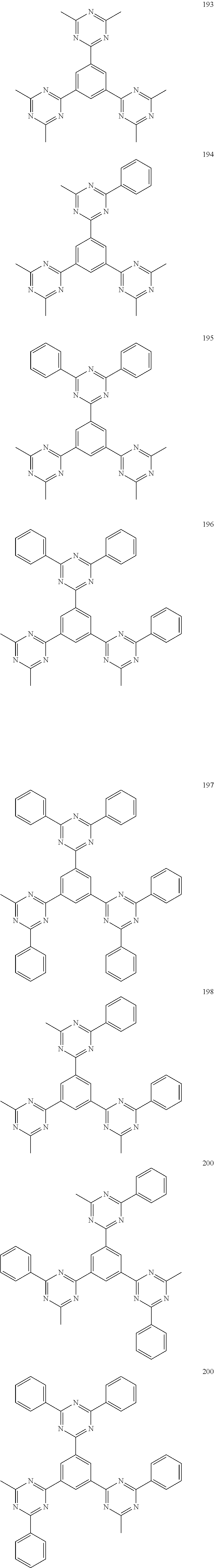

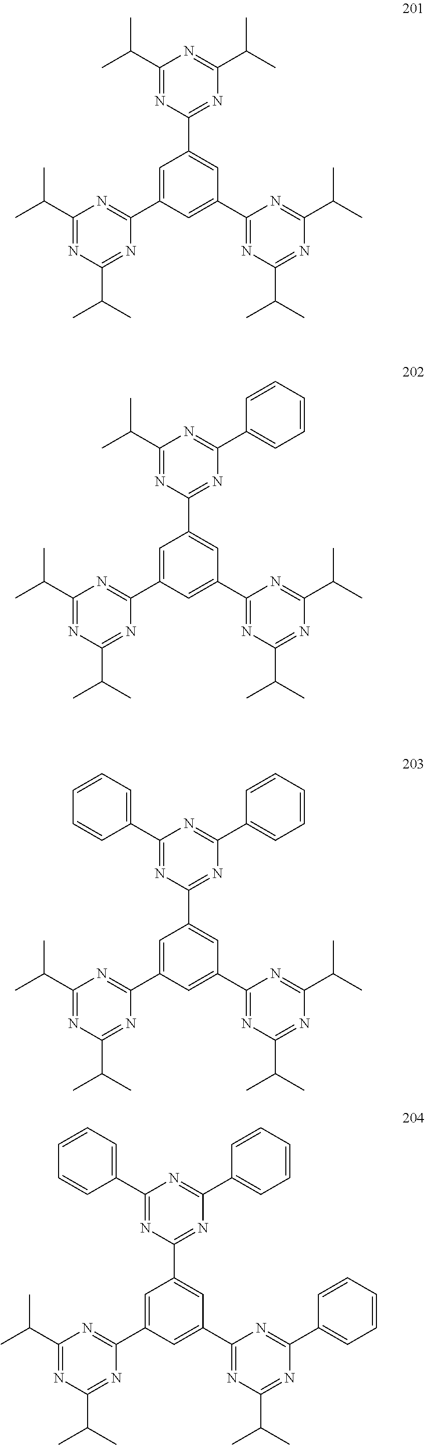

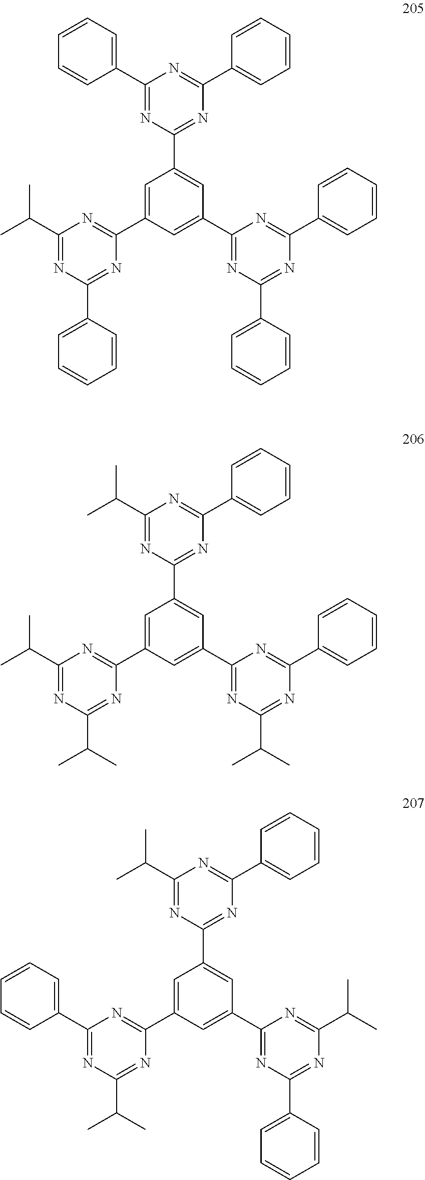 [SMILES: CC(C)c1nc(-c2ccccc2)nc(-c2cc(-c3nc(-c4ccccc4)nc(-c4ccccc4)n3)cc(-c3nc(-c4ccccc4)nc(-c4ccccc4)n3)c2)n1 ; CC(C)c1nc(-c2ccccc2)nc(-c2cc(-c3nc(-c4ccccc4)nc(C(C)C)n3)cc(-c3nc(C(C)C)nc(C(C)C)n3)c2)n1 ; CC(C)c1nc(-c2ccccc2)nc(-c2cc(-c3nc(-c4ccccc4)nc(C(C)C)n3)cc(-c3nc(-c4ccccc4)nc(C(C)C)n3)c2)n1]

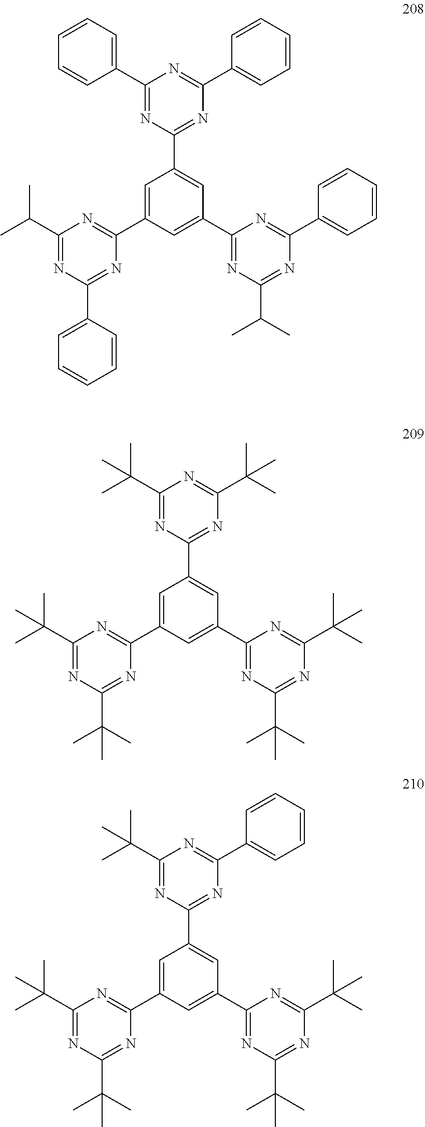

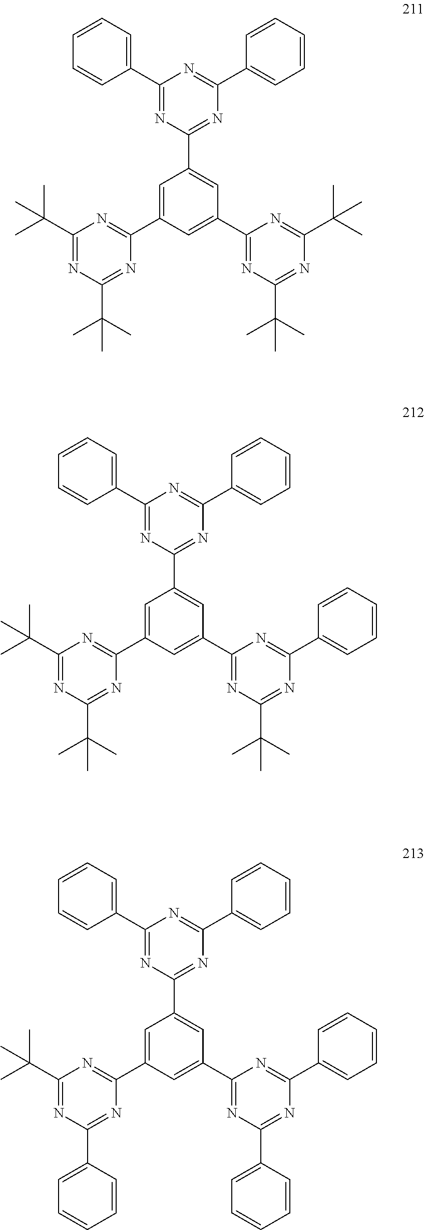

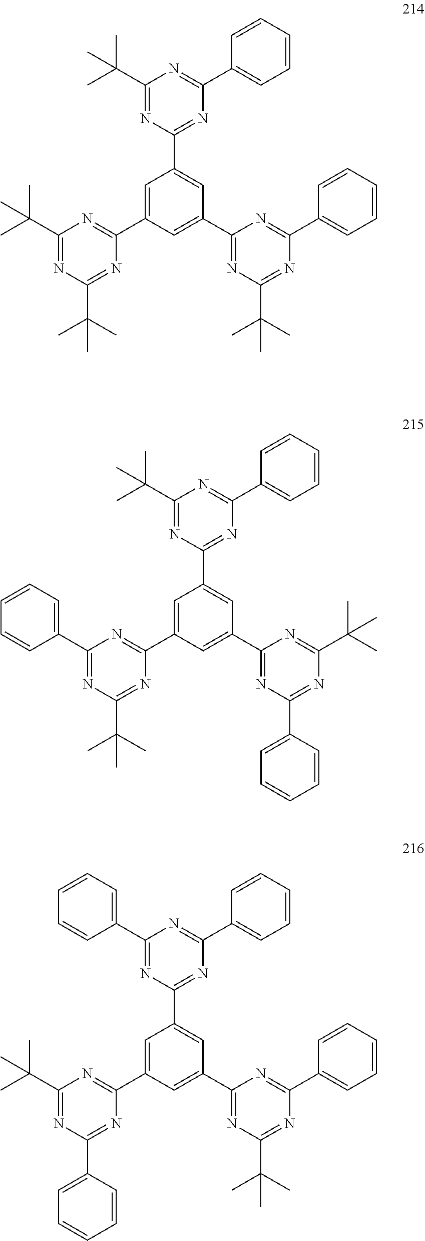

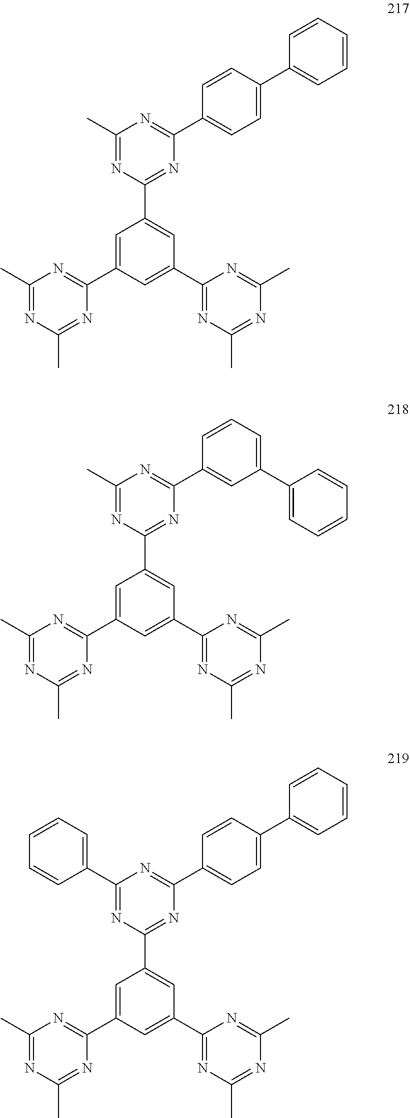 [SMILES: Cc1nc(C)nc(-c2cc(-c3nc(C)nc(C)n3)cc(-c3nc(C)nc(-c4ccc(-c5ccccc5)cc4)n3)c2)n1 ; Cc1nc(C)nc(-c2cc(-c3nc(C)nc(C)n3)cc(-c3nc(C)nc(-c4cccc(-c5ccccc5)c4)n3)c2)n1 ; Cc1nc(C)nc(-c2cc(-c3nc(C)nc(C)n3)cc(-c3nc(-c4ccccc4)nc(-c4ccc(-c5ccccc5)cc4)n3)c2)n1]

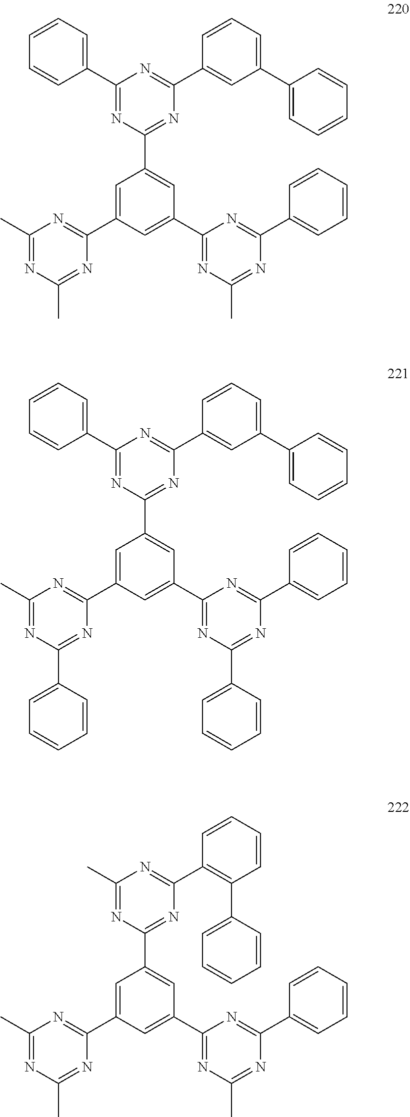 [SMILES: Cc1nc(C)nc(-c2cc(-c3nc(C)nc(-c4ccccc4)n3)cc(-c3nc(-c4ccccc4)nc(-c4cccc(-c5ccccc5)c4)n3)c2)n1 ; Cc1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1 ; Cc1nc(C)nc(-c2cc(-c3nc(C)nc(-c4ccccc4)n3)cc(-c3nc(C)nc(-c4ccccc4-c4ccccc4)n3)c2)n1]

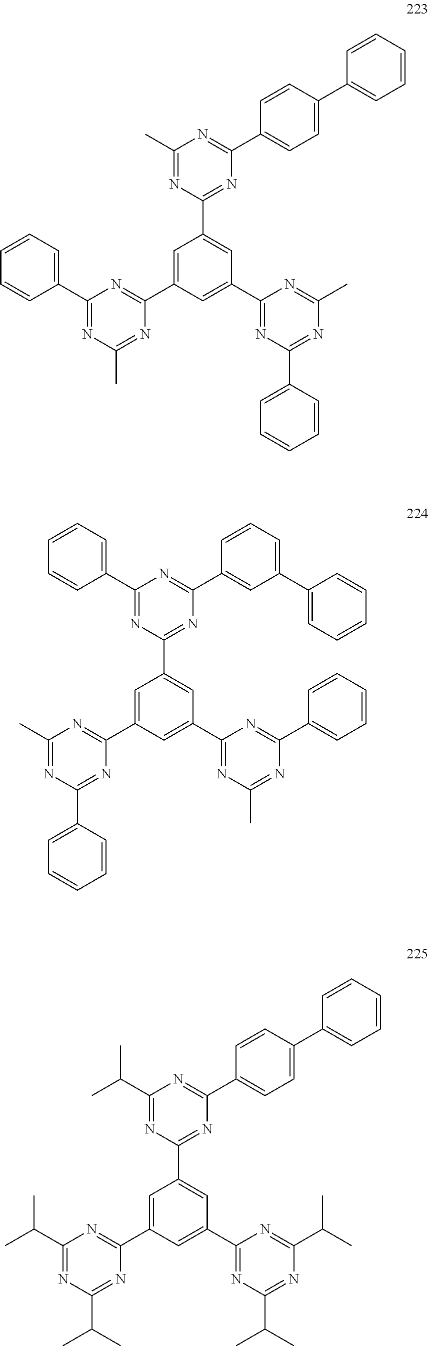 [SMILES: Cc1nc(-c2ccccc2)nc(-c2cc(-c3nc(C)nc(-c4ccccc4)n3)cc(-c3nc(C)nc(-c4ccc(-c5ccccc5)cc4)n3)c2)n1 ; Cc1nc(-c2ccccc2)nc(-c2cc(-c3nc(C)nc(-c4ccccc4)n3)cc(-c3nc(-c4ccccc4)nc(-c4cccc(-c5ccccc5)c4)n3)c2)n1 ; CC(C)c1nc(-c2ccc(-c3ccccc3)cc2)nc(-c2cc(-c3nc(C(C)C)nc(C(C)C)n3)cc(-c3nc(C(C)C)nc(C(C)C)n3)c2)n1]

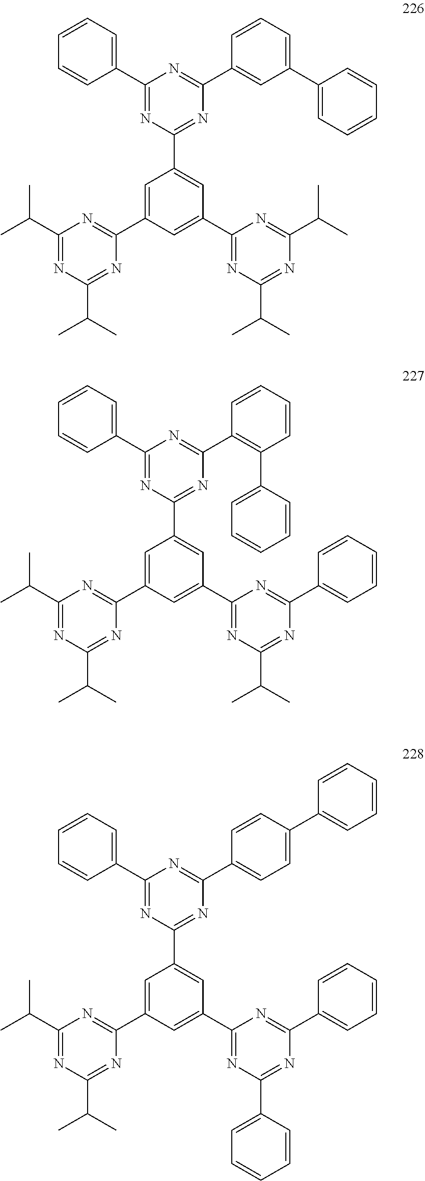 [SMILES: CC(C)c1nc(-c2cc(-c3nc(-c4ccccc4)nc(-c4cccc(-c5ccccc5)c4)n3)cc(-c3nc(C(C)C)nc(C(C)C)n3)c2)nc(C(C)C)n1 ; CC(C)c1nc(-c2ccccc2)nc(-c2cc(-c3nc(-c4ccccc4)nc(-c4ccccc4-c4ccccc4)n3)cc(-c3nc(C(C)C)nc(C(C)C)n3)c2)n1 ; CC(C)c1nc(-c2cc(-c3nc(-c4ccccc4)nc(-c4ccccc4)n3)cc(-c3nc(-c4ccccc4)nc(-c4ccc(-c5ccccc5)cc4)n3)c2)nc(C(C)C)n1]

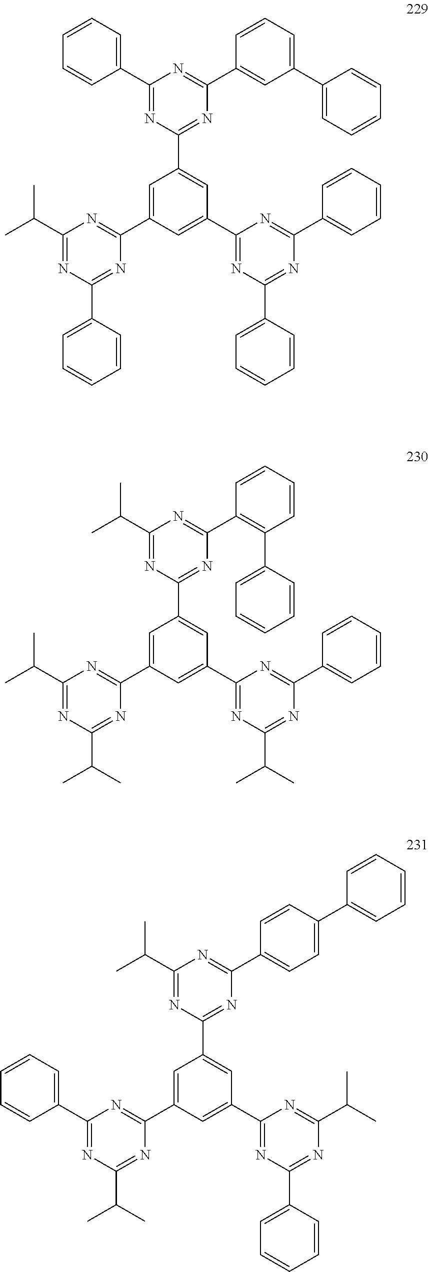 [SMILES: CC(C)c1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1 ; CC(C)c1nc(-c2ccccc2)nc(-c2cc(-c3nc(-c4ccccc4-c4ccccc4)nc(C(C)C)n3)cc(-c3nc(C(C)C)nc(C(C)C)n3)c2)n1 ; CC(C)c1nc(-c2ccccc2)nc(-c2cc(-c3nc(-c4ccccc4)nc(C(C)C)n3)cc(-c3nc(-c4ccc(-c5ccccc5)cc4)nc(C(C)C)n3)c2)n1]

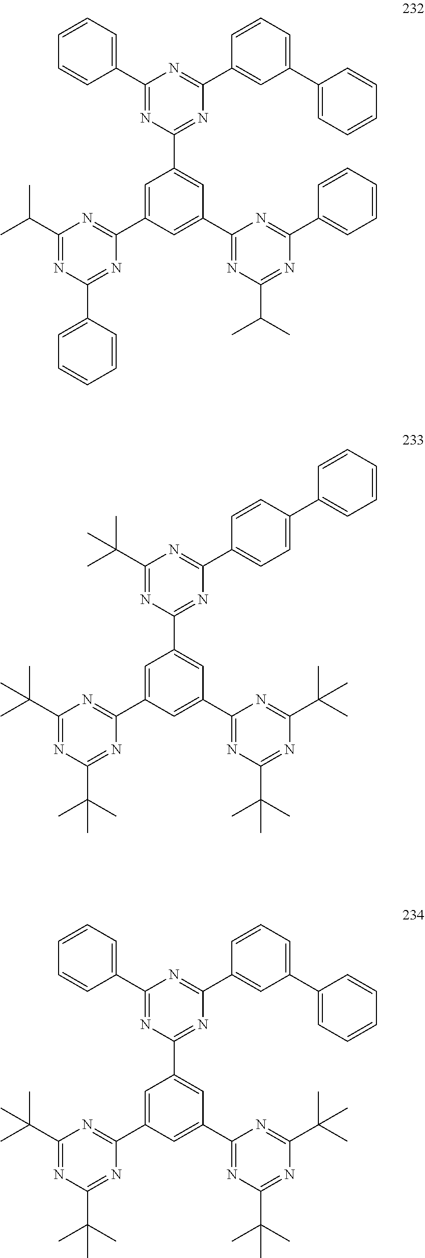 [SMILES: CC(C)c1nc(-c2ccccc2)nc(-c2cc(-c3nc(-c4ccccc4)nc(-c4cccc(-c5ccccc5)c4)n3)cc(-c3nc(-c4ccccc4)nc(C(C)C)n3)c2)n1 ; CC(C)(C)c1nc(-c2ccc(-c3ccccc3)cc2)nc(-c2cc(-c3nc(C(C)(C)C)nc(C(C)(C)C)n3)cc(-c3nc(C(C)(C)C)nc(C(C)(C)C)n3)c2)n1 ; CC(C)(C)c1nc(-c2cc(-c3nc(-c4ccccc4)nc(-c4cccc(-c5ccccc5)c4)n3)cc(-c3nc(C(C)(C)C)nc(C(C)(C)C)n3)c2)nc(C(C)(C)C)n1]

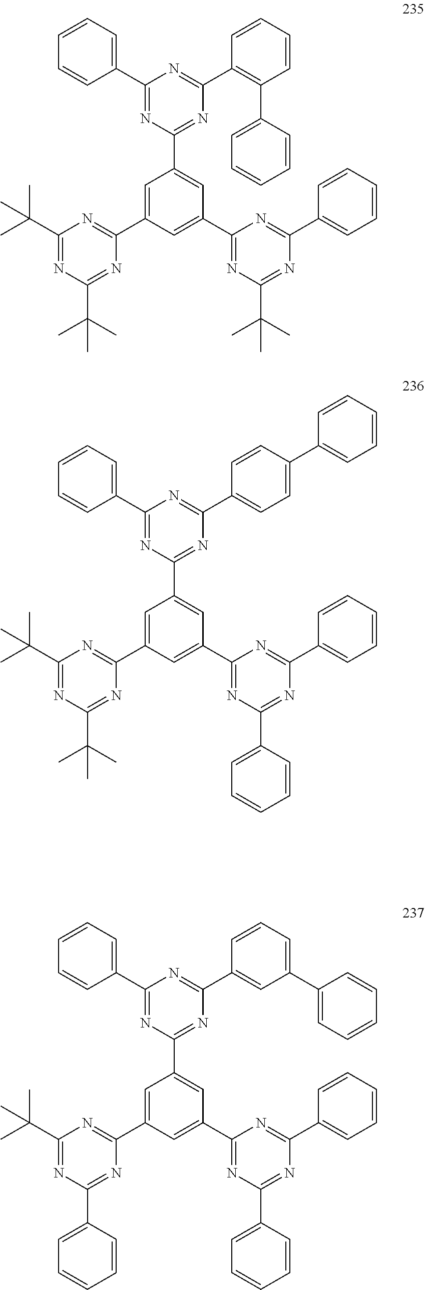 [SMILES: CC(C)(C)c1nc(-c2ccccc2)nc(-c2cc(-c3nc(-c4ccccc4)nc(-c4ccccc4-c4ccccc4)n3)cc(-c3nc(C(C)(C)C)nc(C(C)(C)C)n3)c2)n1 ; CC(C)(C)c1nc(-c2cc(-c3nc(-c4ccccc4)nc(-c4ccccc4)n3)cc(-c3nc(-c4ccccc4)nc(-c4ccc(-c5ccccc5)cc4)n3)c2)nc(C(C)(C)C)n1 ; CC(C)(C)c1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1]

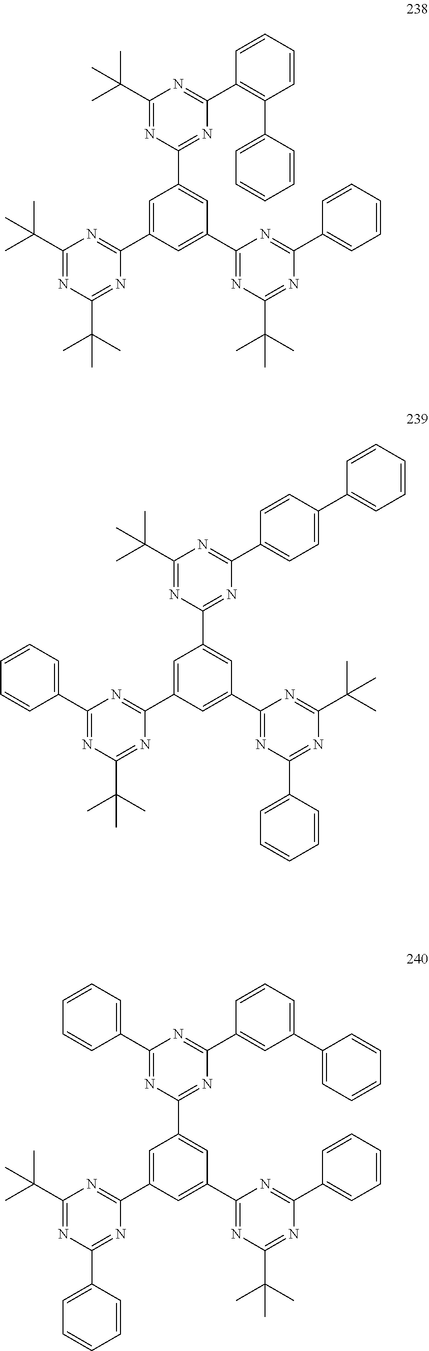 [SMILES: CC(C)(C)c1nc(-c2ccccc2)nc(-c2cc(-c3nc(-c4ccccc4-c4ccccc4)nc(C(C)(C)C)n3)cc(-c3nc(C(C)(C)C)nc(C(C)(C)C)n3)c2)n1 ; CC(C)(C)c1nc(-c2ccccc2)nc(-c2cc(-c3nc(-c4ccccc4)nc(C(C)(C)C)n3)cc(-c3nc(-c4ccc(-c5ccccc5)cc4)nc(C(C)(C)C)n3)c2)n1 ; CC(C)(C)c1nc(-c2ccccc2)nc(-c2cc(-c3nc(-c4ccccc4)nc(-c4cccc(-c5ccccc5)c4)n3)cc(-c3nc(-c4ccccc4)nc(C(C)(C)C)n3)c2)n1]

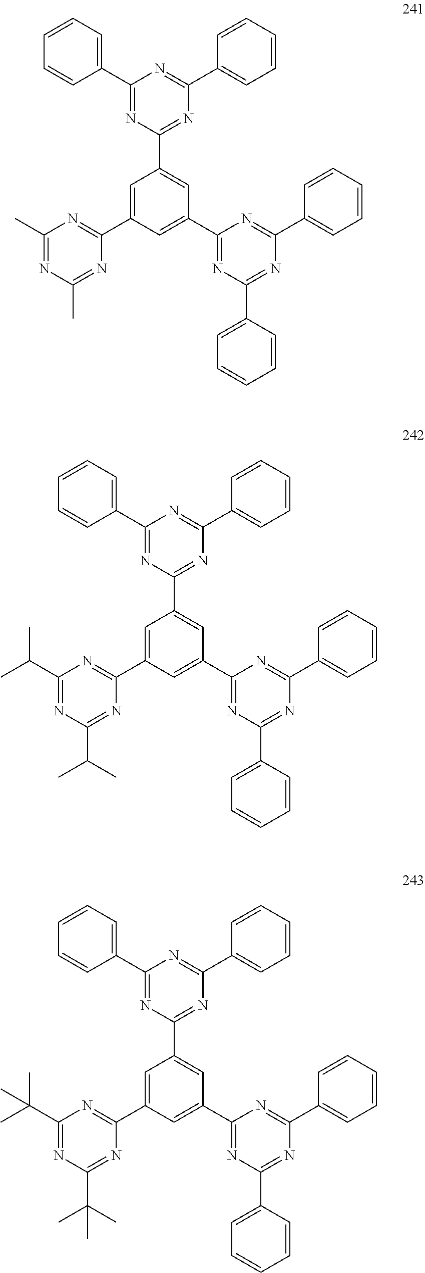 [SMILES: Cc1nc(C)nc(-c2cc(-c3nc(-c4ccccc4)nc(-c4ccccc4)n3)cc(-c3nc(-c4ccccc4)nc(-c4ccccc4)n3)c2)n1 ; CC(C)c1nc(-c2cc(-c3nc(-c4ccccc4)nc(-c4ccccc4)n3)cc(-c3nc(-c4ccccc4)nc(-c4ccccc4)n3)c2)nc(C(C)C)n1 ; CC(C)(C)c1nc(-c2cc(-c3nc(-c4ccccc4)nc(-c4ccccc4)n3)cc(-c3nc(-c4ccccc4)nc(-c4ccccc4)n3)c2)nc(C(C)(C)C)n1]

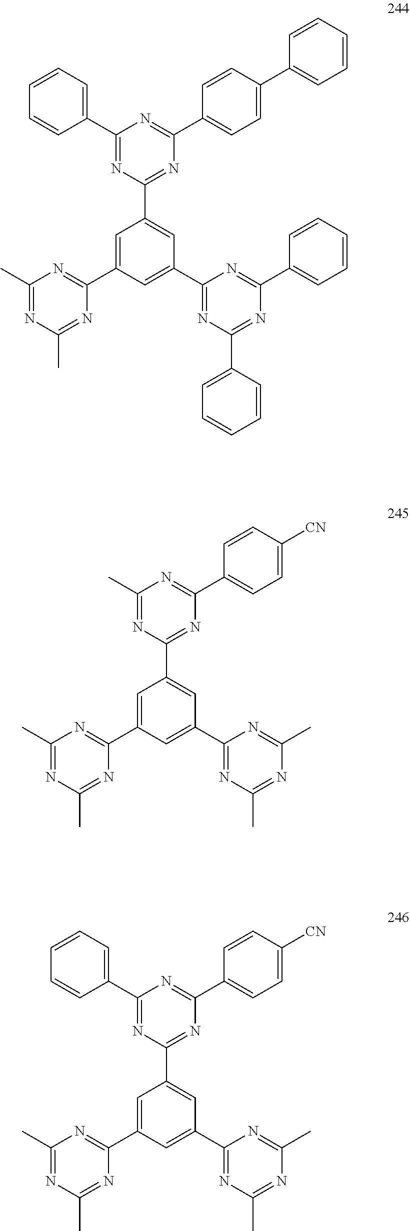 [SMILES: Cc1nc(C)nc(-c2cc(-c3nc(-c4ccccc4)nc(-c4ccccc4)n3)cc(-c3nc(-c4ccccc4)nc(-c4ccc(-c5ccccc5)cc4)n3)c2)n1 ; Cc1nc(C)nc(-c2cc(-c3nc(C)nc(C)n3)cc(-c3nc(C)nc(-c4ccc(C#N)cc4)n3)c2)n1 ; Cc1nc(C)nc(-c2cc(-c3nc(C)nc(C)n3)cc(-c3nc(-c4ccccc4)nc(-c4ccc(C#N)cc4)n3)c2)n1]

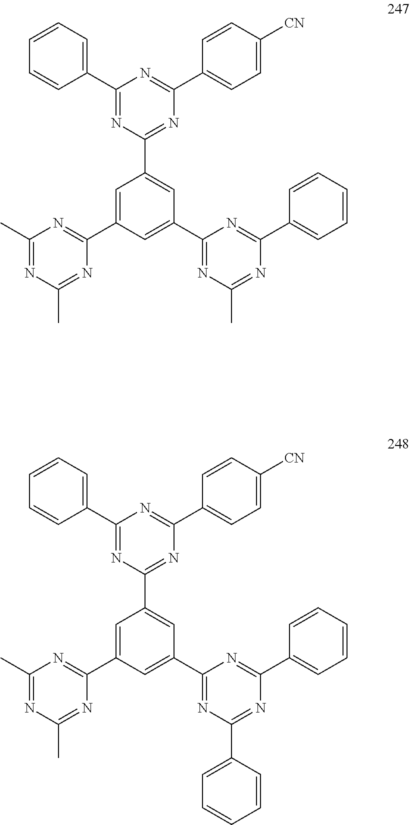 [SMILES: Cc1nc(C)nc(-c2cc(-c3nc(C)nc(-c4ccccc4)n3)cc(-c3nc(-c4ccccc4)nc(-c4ccc(C#N)cc4)n3)c2)n1 ; Cc1nc(C)nc(-c2cc(-c3nc(-c4ccccc4)nc(-c4ccccc4)n3)cc(-c3nc(-c4ccccc4)nc(-c4ccc(C#N)cc4)n3)c2)n1]

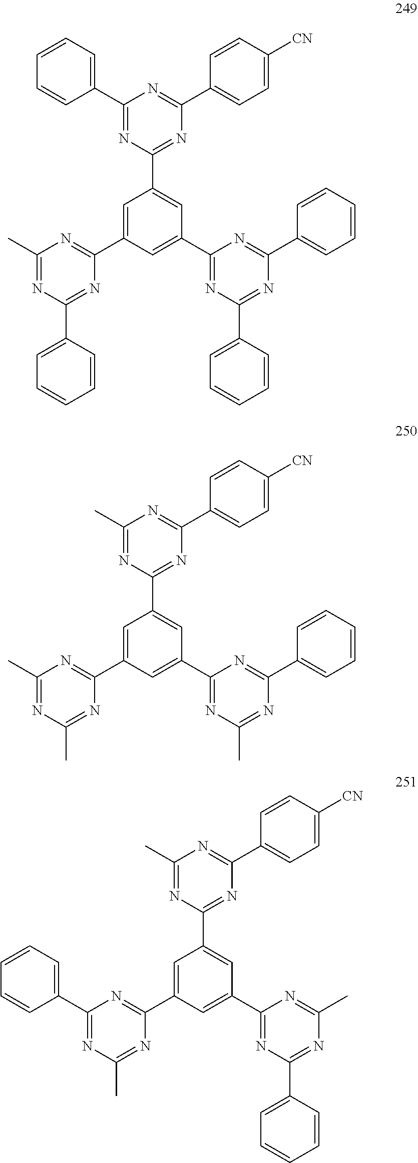 [SMILES: Cc1nc(-c2ccccc2)nc(-c2cc(-c3nc(-c4ccccc4)nc(-c4ccccc4)n3)cc(-c3nc(-c4ccccc4)nc(-c4ccc(C#N)cc4)n3)c2)n1 ; Cc1nc(C)nc(-c2cc(-c3nc(C)nc(-c4ccccc4)n3)cc(-c3nc(C)nc(-c4ccc(C#N)cc4)n3)c2)n1 ; Cc1nc(-c2ccccc2)nc(-c2cc(-c3nc(C)nc(-c4ccccc4)n3)cc(-c3nc(C)nc(-c4ccc(C#N)cc4)n3)c2)n1]

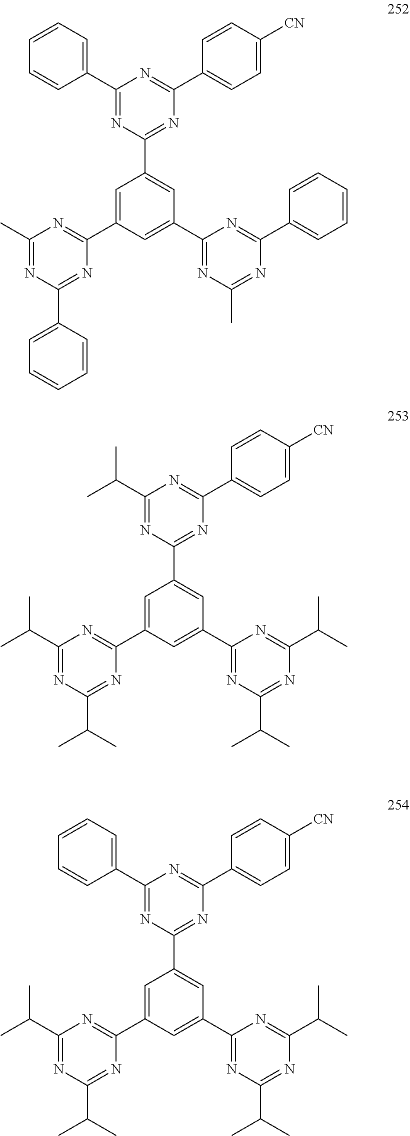

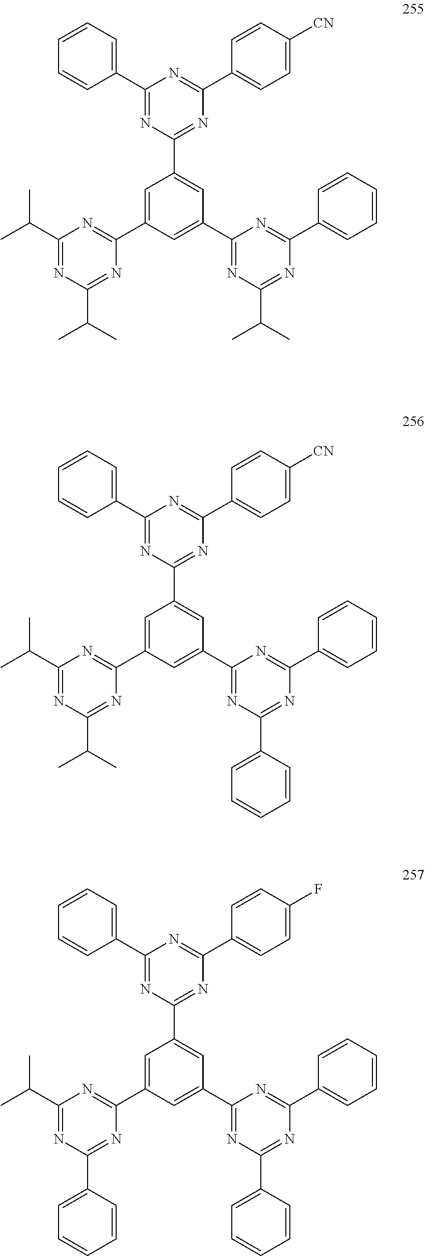 [SMILES: CC(C)c1nc(-c2ccccc2)nc(-c2cc(-c3nc(-c4ccccc4)nc(-c4ccc(C#N)cc4)n3)cc(-c3nc(C(C)C)nc(C(C)C)n3)c2)n1 ; CC(C)c1nc(-c2cc(-c3nc(-c4ccccc4)nc(-c4ccccc4)n3)cc(-c3nc(-c4ccccc4)nc(-c4ccc(C#N)cc4)n3)c2)nc(C(C)C)n1 ; CC(C)c1nc(-c2ccccc2)nc(-c2cc(-c3nc(-c4ccccc4)nc(-c4ccccc4)n3)cc(-c3nc(-c4ccccc4)nc(-c4ccc(F)cc4)n3)c2)n1]

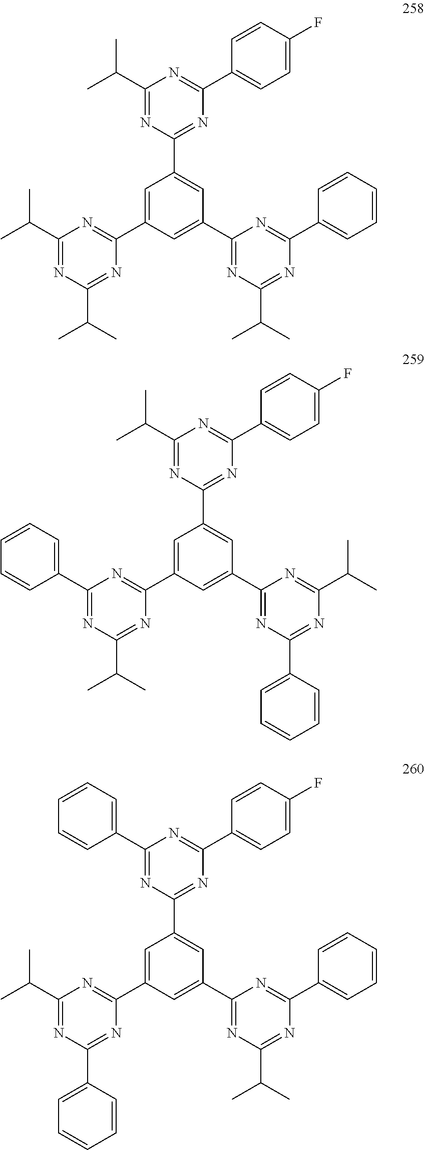

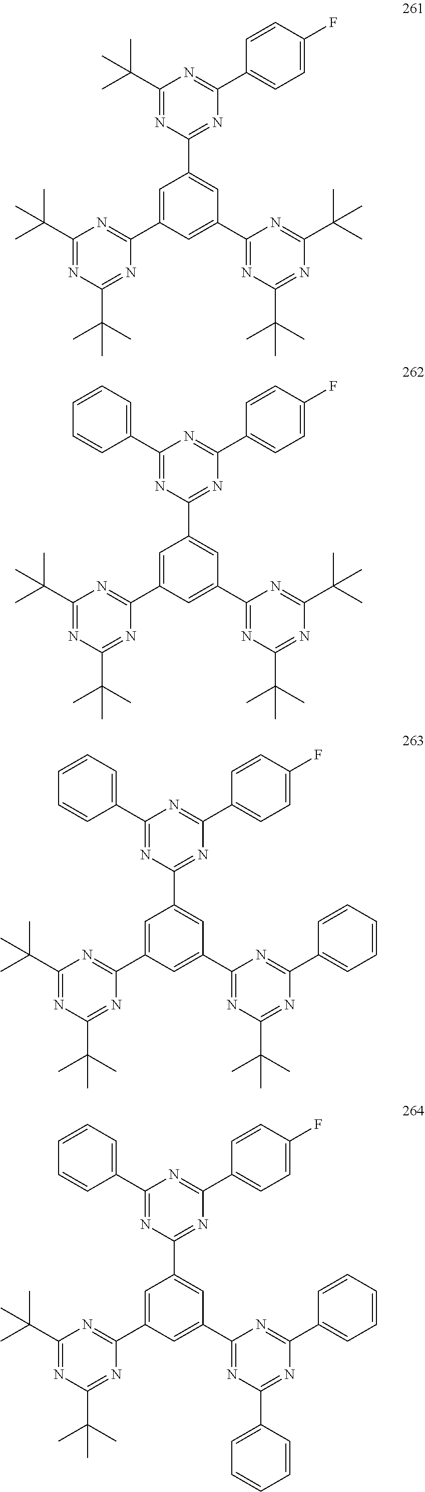

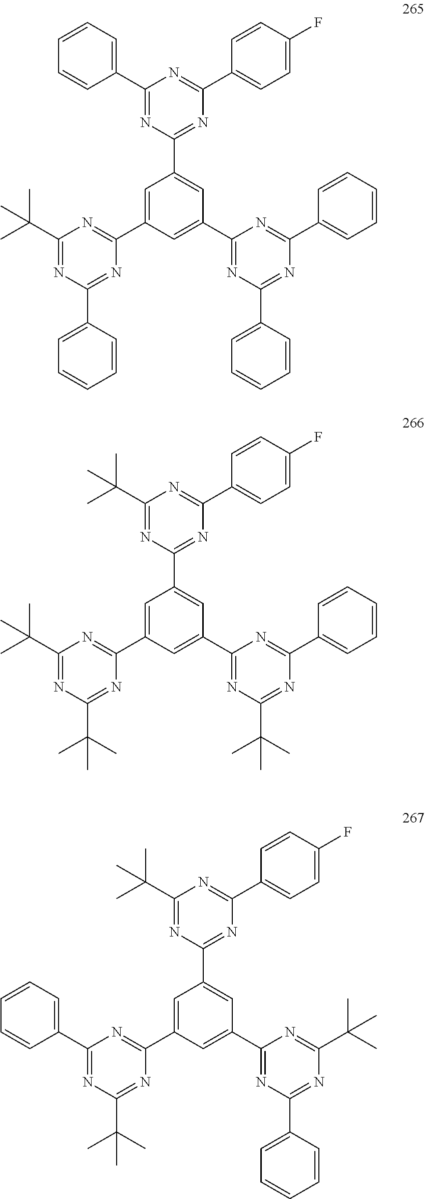 [SMILES: CC(C)(C)c1nc(-c2ccccc2)nc(-c2cc(-c3nc(-c4ccccc4)nc(-c4ccccc4)n3)cc(-c3nc(-c4ccccc4)nc(-c4ccc(F)cc4)n3)c2)n1 ; CC(C)(C)c1nc(-c2ccccc2)nc(-c2cc(-c3nc(-c4ccc(F)cc4)nc(C(C)(C)C)n3)cc(-c3nc(C(C)(C)C)nc(C(C)(C)C)n3)c2)n1 ; CC(C)(C)c1nc(-c2ccccc2)nc(-c2cc(-c3nc(-c4ccccc4)nc(C(C)(C)C)n3)cc(-c3nc(-c4ccc(F)cc4)nc(C(C)(C)C)n3)c2)n1]

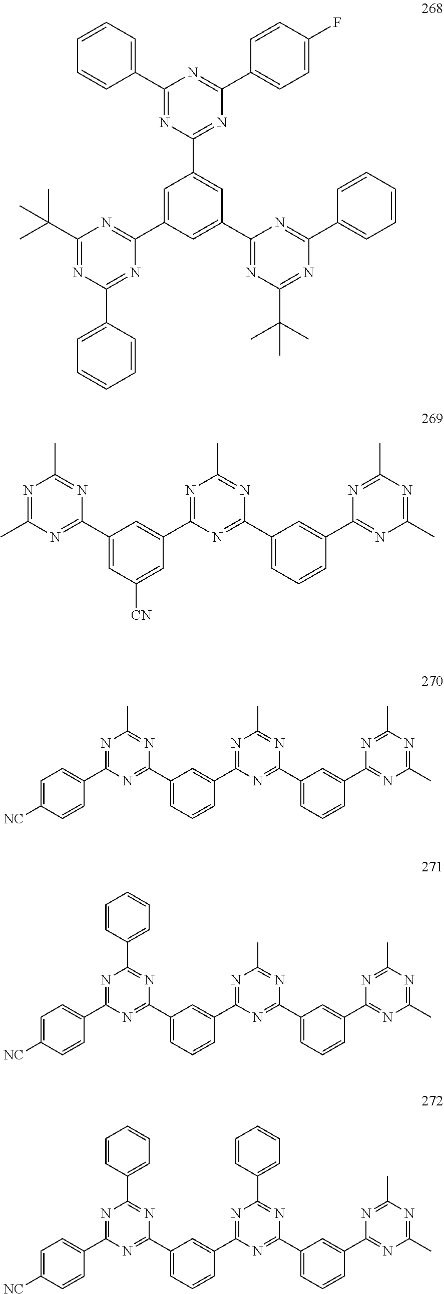

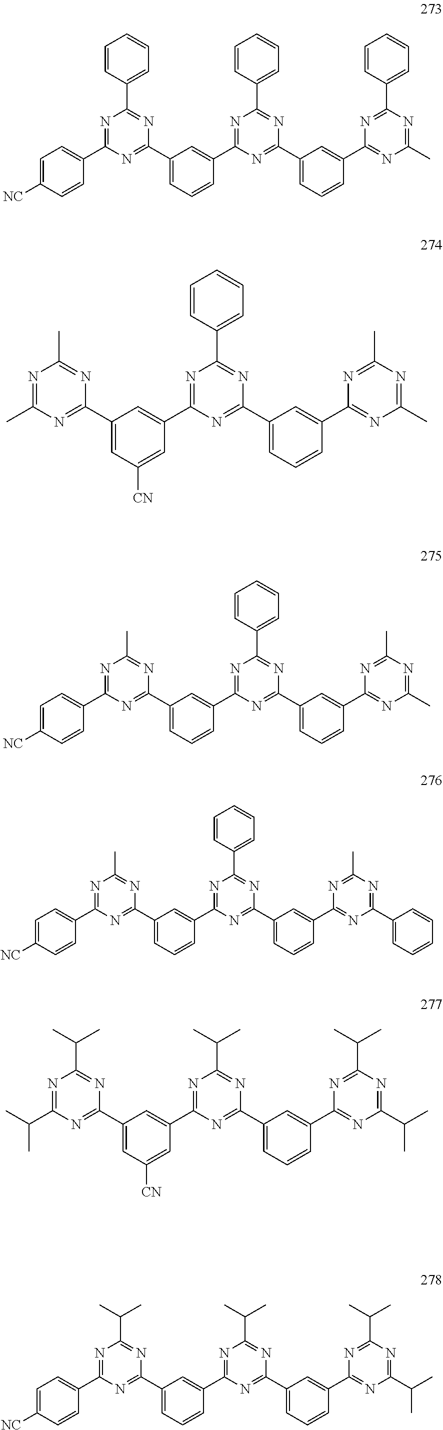

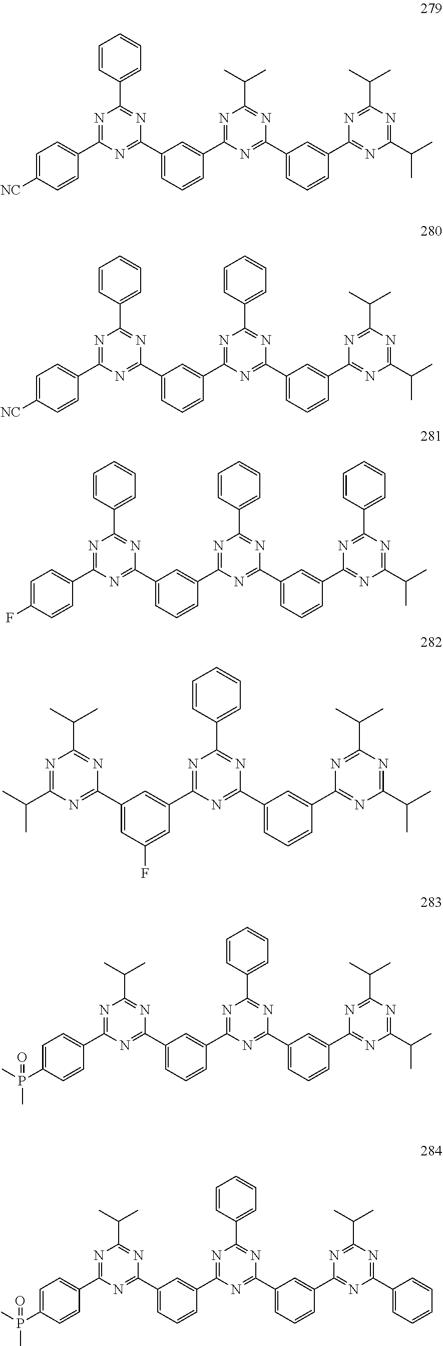

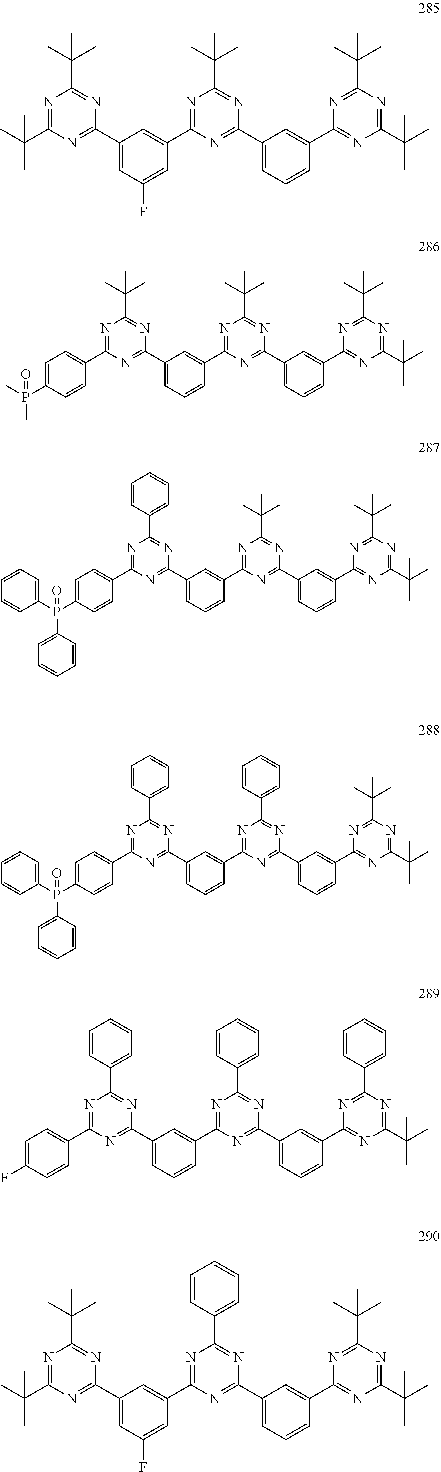

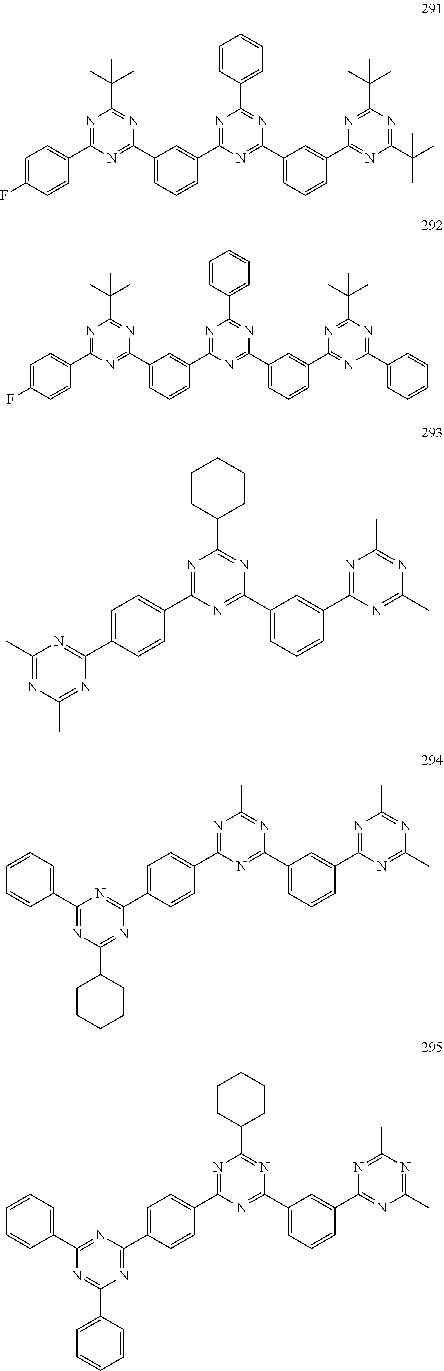

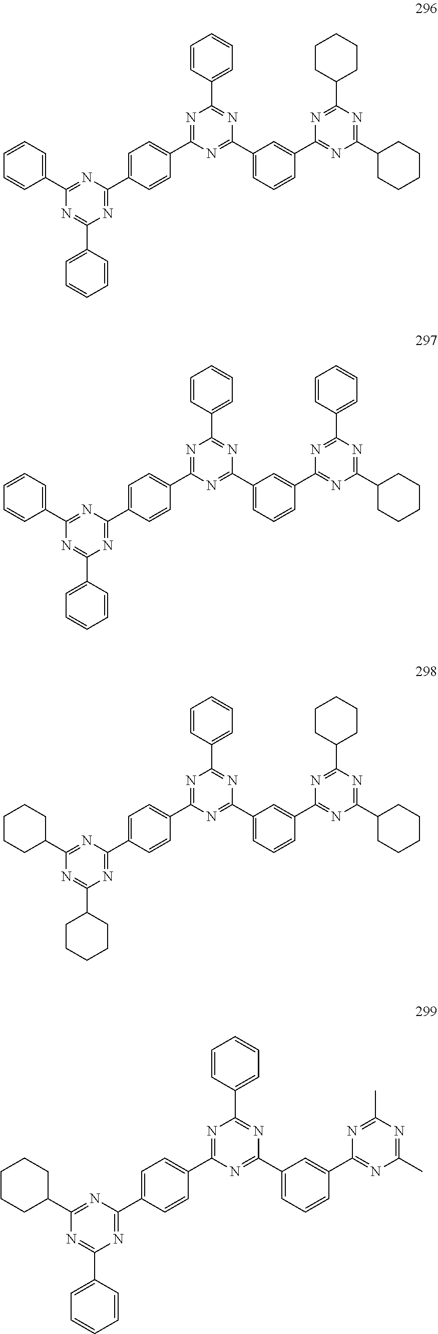 [SMILES: c1ccc(-c2nc(-c3ccccc3)nc(-c3ccc(-c4nc(-c5ccccc5)nc(-c5cccc(-c6nc(C7CCCCC7)nc(C7CCCCC7)n6)c5)n4)cc3)n2)cc1 ; c1ccc(-c2nc(-c3ccccc3)nc(-c3ccc(-c4nc(-c5ccccc5)nc(-c5cccc(-c6nc(-c7ccccc7)nc(C7CCCCC7)n6)c5)n4)cc3)n2)cc1 ; c1ccc(-c2nc(-c3ccc(-c4nc(C5CCCCC5)nc(C5CCCCC5)n4)cc3)nc(-c3cccc(-c4nc(C5CCCCC5)nc(C5CCCCC5)n4)c3)n2)cc1 ; Cc1nc(C)nc(-c2cccc(-c3nc(-c4ccccc4)nc(-c4ccc(-c5nc(-c6ccccc6)nc(C6CCCCC6)n5)cc4)n3)c2)n1]

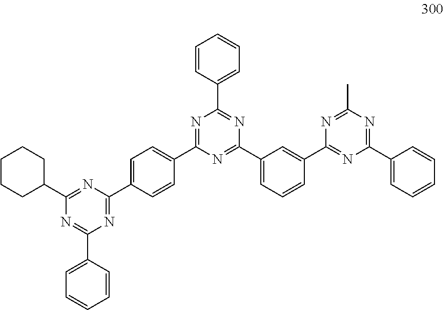 [SMILES: Cc1nc(-c2ccccc2)nc(-c2cccc(-c3nc(-c4ccccc4)nc(-c4ccc(-c5nc(-c6ccccc6)nc(C6CCCCC6)n5)cc4)n3)c2)n1]

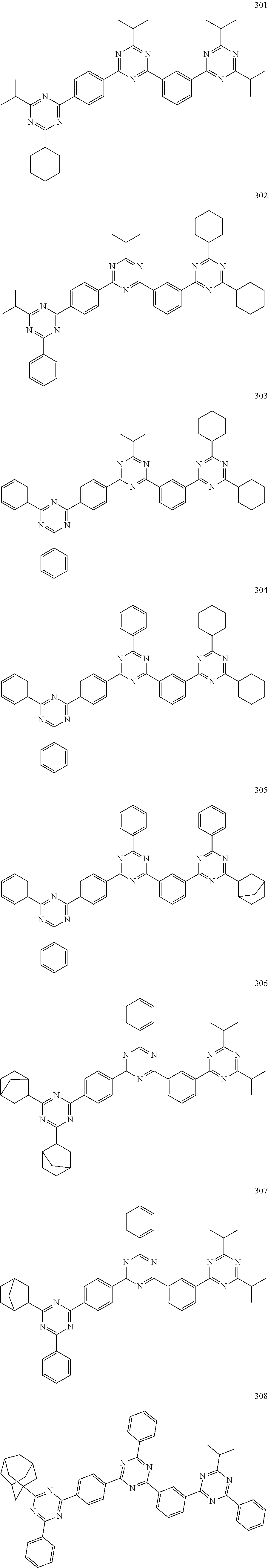

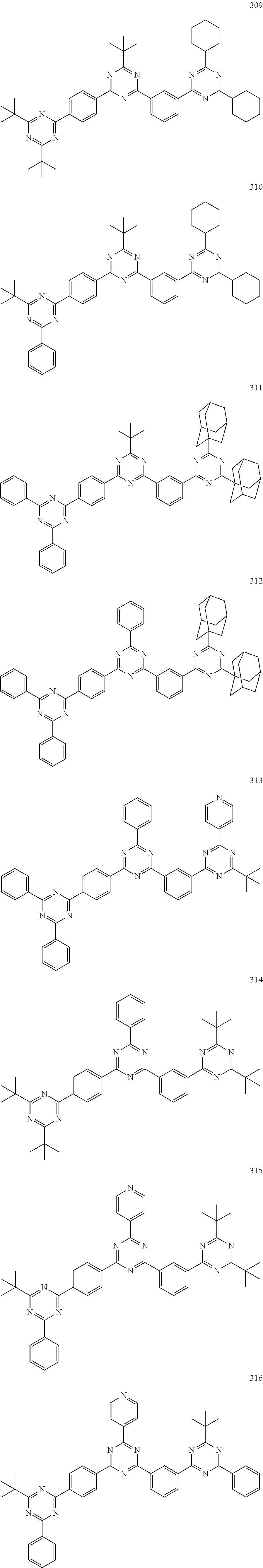

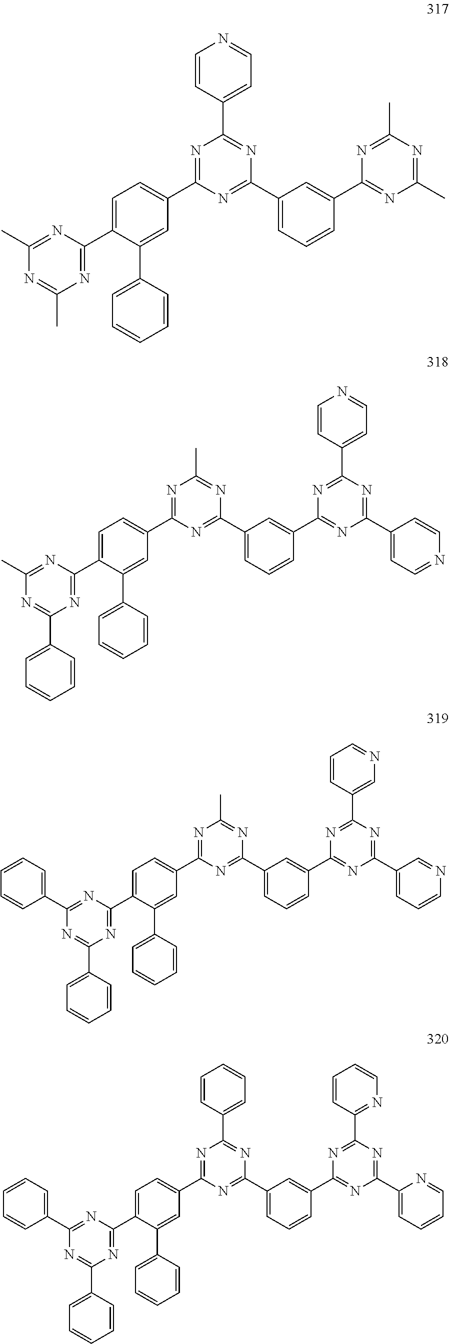

The nitrogen-containing compound according to one or more embodiments includes three triazine moieties, which are connected via a linker, and a first substituent linked to at least one among the three triazine moieties. Because a charge balance is controlled or selected due to a such specific structure, excellent or suitable charge transporting ability may be exhibited, and because high glass transition temperature prevents (or blocks or reduces) crystallization, the nitrogen-containing compound according to one or more embodiments may have excellent or suitable material stability. Because the light-emitting element ED according to one or more embodiments of the disclosure includes the nitrogen-containing compound according to one or more embodiments, electrical and chemical stabilities thereof may be improved, thus the light-emitting element ED according to one or more embodiments of the disclosure may have improved element lifespan characteristics, and a satisfactory level of charge-transporting properties may be obtained without a substantial increase in driving voltage, which may achieve an increase in luminous efficiency.

In one or more embodiments, the electron transport region ETR may further include an electron transport material and an electron injection material which are suitable in the art.

The electron transport region ETR may further include a compound represented by Formula ET-2.

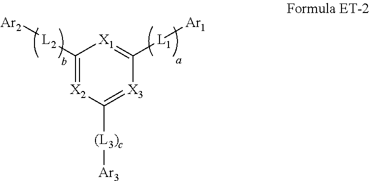

In Formula ET-2, at least one selected from among X₁ to X₃ is N, and the rest (e.g., any remaining selected from among X₁ to X₃) may be CRₐ. Rₐ may be a hydrogen atom, a deuterium atom, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c are each an integer of 2 or greater, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may further include an anthracene-based compound. However, one or more embodiments of the disclosure is not limited thereto. The electron transport region ETR may be, for example, Tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-Tri(1-phenyl-1H-benzo[d]imidazol-2-yl)benzene (TPBi), 2,9-Dimethyl-4,7-diphenyl-1,10-phenanthroline (BCP), 4,7-Diphenyl-1,10-phenanthroline (Bphen), 3-(4-Biphenylyl)-4-phenyl-5-tert-butylphenyl-1,2,4-triazole (TAZ), 4-(Naphthalen-1-yl)-3,5-diphenyl-4H-1,2,4-triazole (NTAZ), 2-(4-Biphenylyl)-5-(4-tert-butylphenyl)-1,3,4-oxadiazole (tBu-PBD), Bis(2-methyl-8-quinolinolato-N1,O8)-(1,1′-Biphenyl-4-olato)aluminum (BAlq), berylliumbis(benzoquinolin-10-olate) (Bebq₂), 9,10-di(naphthalen-2-yl)anthracene (ADN), 1,3-Bis[3,5-di(pyridin-3-yl)phenyl]benzene (BmPyPhB), 4′-(4-(4-(4,6-diphenyl-1,3,5-triazin-2-yl)phenyl)naphthalen-1-yl)-[1,1′-biphenyl]-4-carbonitrile (CNNPTRZ) and/or a (e.g., any suitable) mixture thereof.

In one or more embodiments, electron transport region ETR may further include any one selected from among the compounds in Compound Group 3.

The electron transport region ETR may further include at least one selected from among Compounds ET1 to ET36.

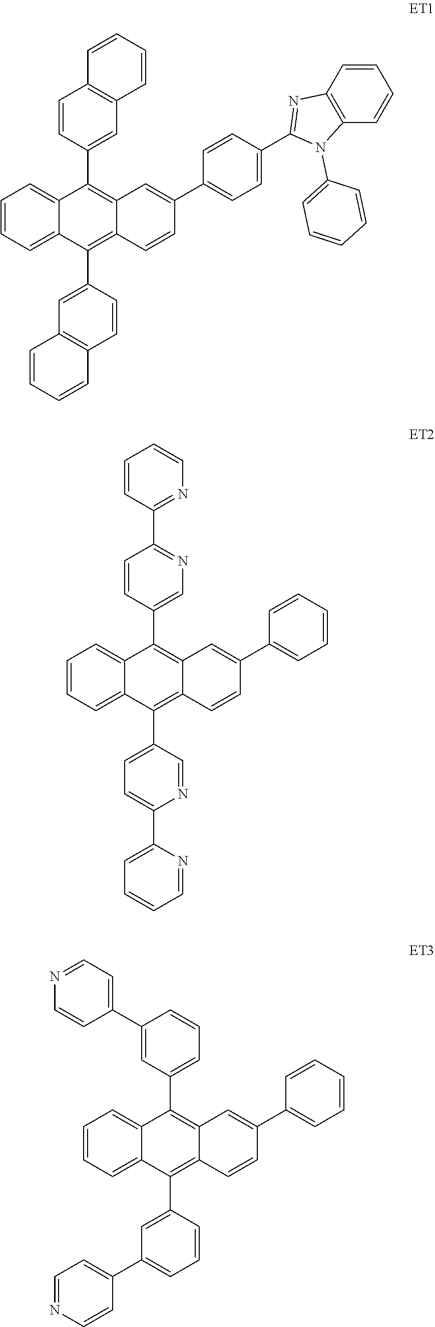 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

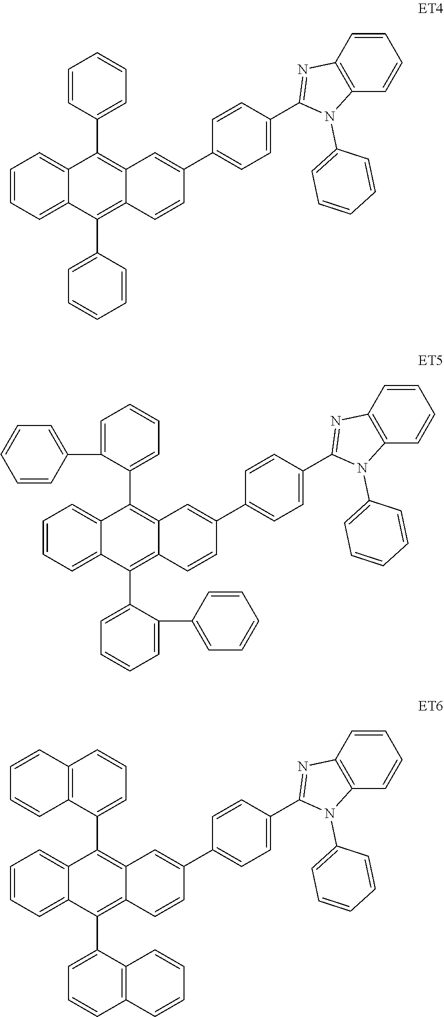 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

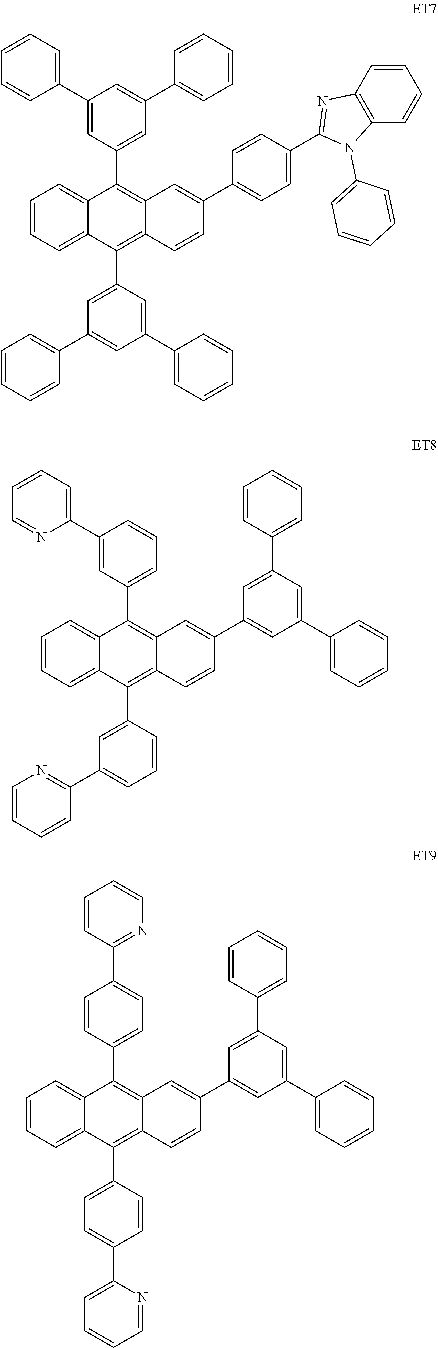 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

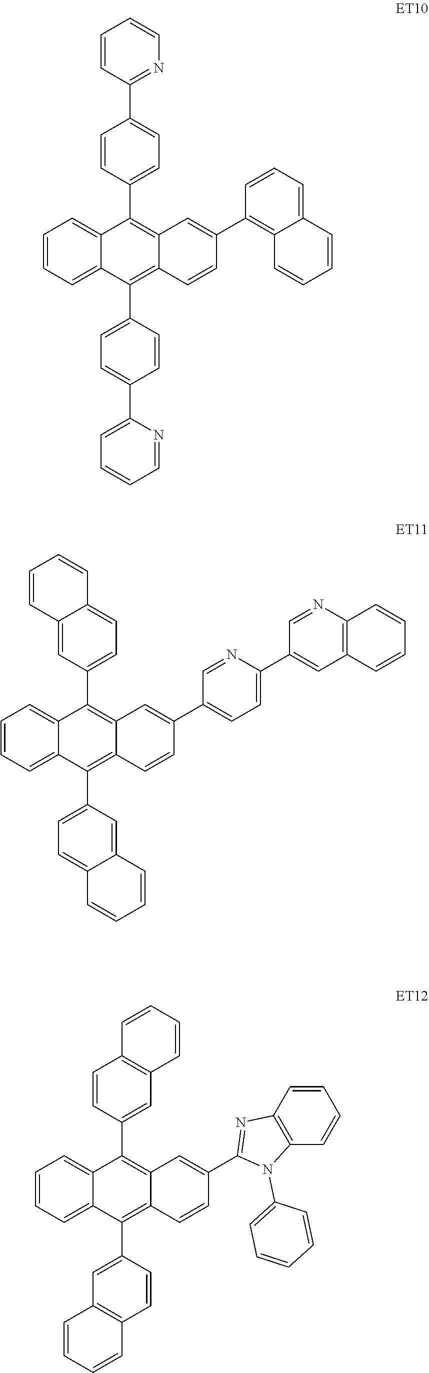 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

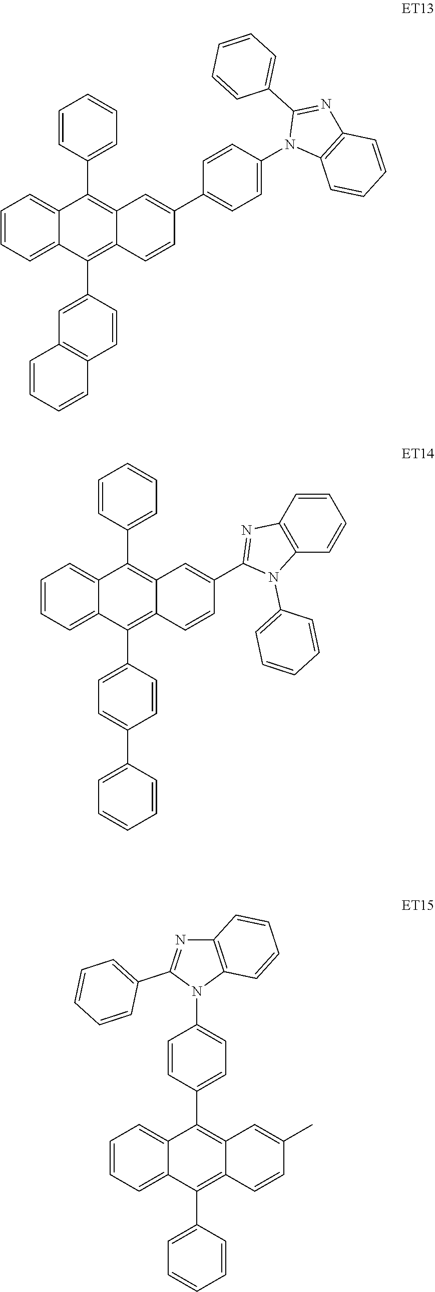 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

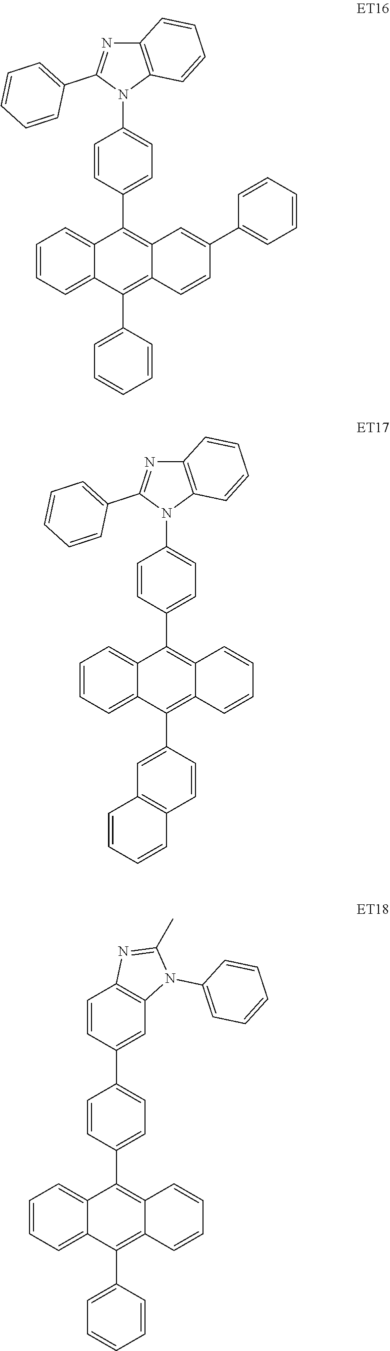 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

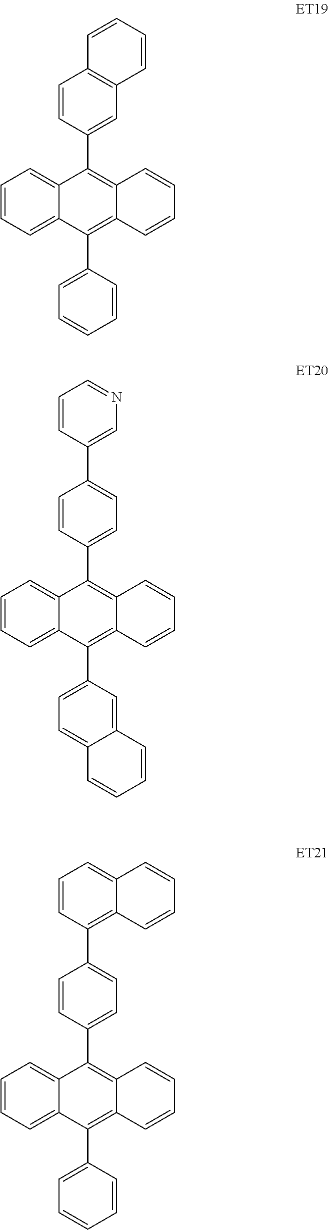

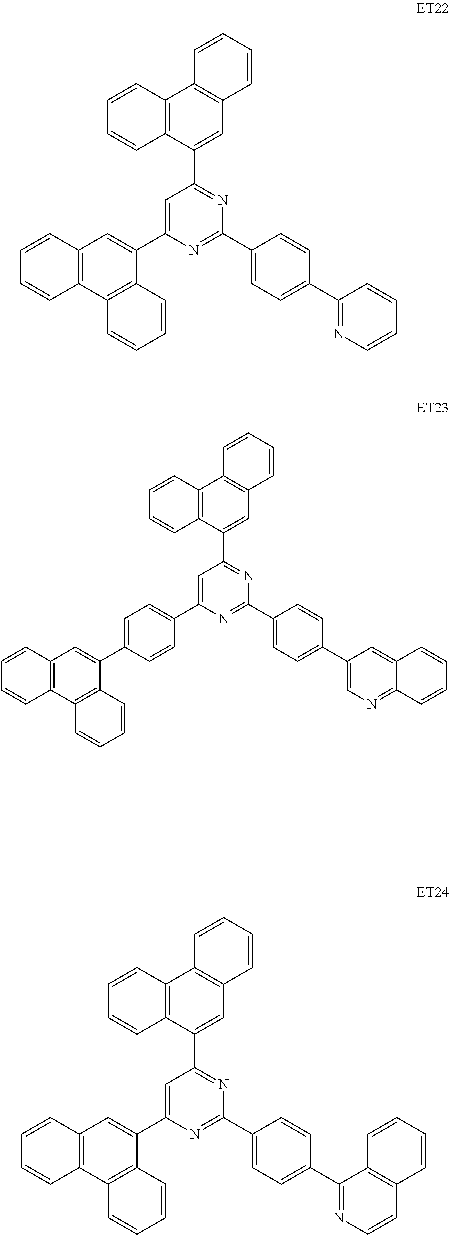 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

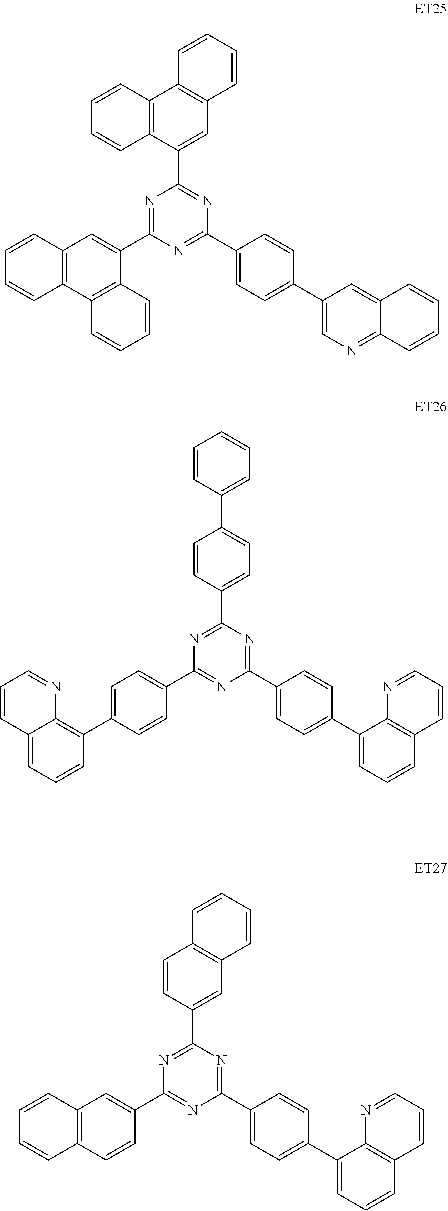 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

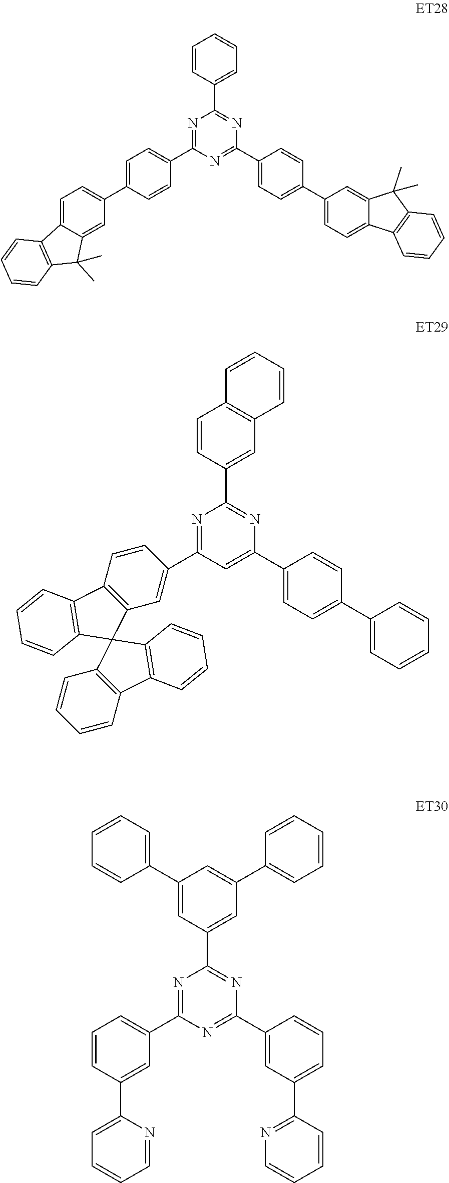 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

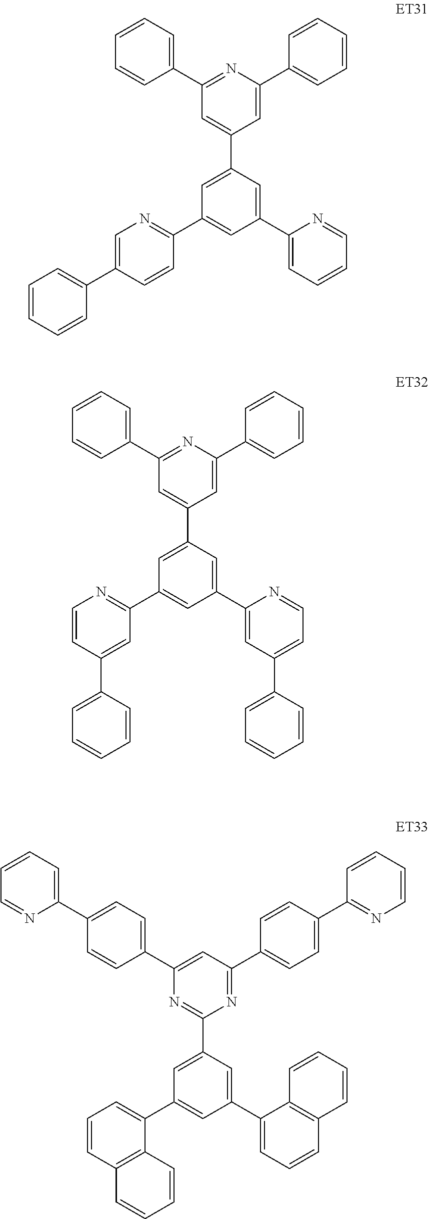 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

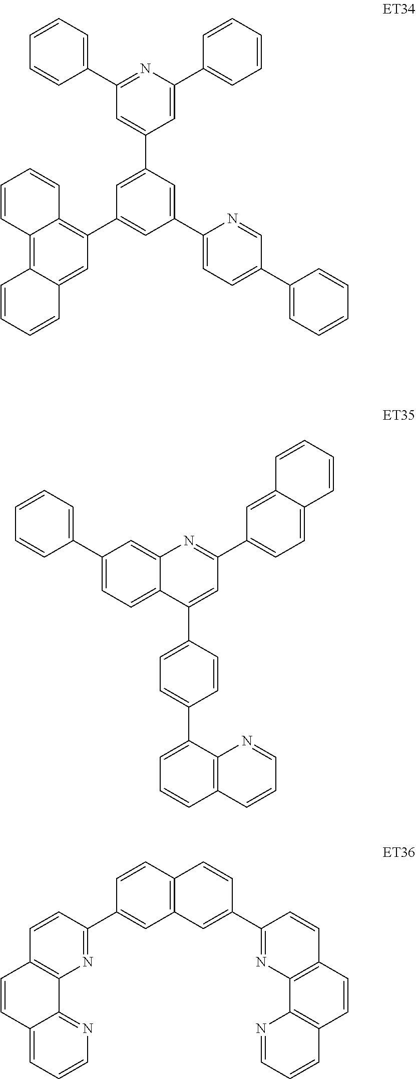 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a halogenated metal such as LiF, NaCl, CsF, RbCl, RbI, CuI, and Kl, a lanthanum group metal such as Yb, or a co-deposition material of the halogenated metal and the lanthanum group metal. For example, the electron transport region ETR may include Kl:Yb, RbI:Yb, LiF:Yb, and/or the like as the co-deposition material. In one or more embodiments, as the electron transport region ETR, a metal oxide such as Li₂O and BaO, or 8-hydroxyl-Lithium quinolate (Liq) and/or the like may be used, but one or more embodiments of the disclosure is not limited thereto. The electron transport region ETR may also be composed of a mixture of an electron transport material and an insulating organo metal salt. The organo metal salt may be a material having an energy band gap of about 4 eV or greater. For example, for example, the organo metal salt may include metal acetate, metal benzoate, metal acetoacetate, metal acetylacetonate, or metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen), but one or more embodiments of the disclosure is not limited thereto.

The electron transport region ETR may include the herein-described compounds of the electron transport region in at least one of the electron injection layer EIL, the electron transport layer ETL, or the hole blocking layer HBL.

When the electron transport region ETR includes the electron transport layer ETL, the thickness of the electron transport layer ETL may be about 100 Å to about 1000 Å, for example, about 150 Å to about 500 Å. When the thickness of the electron transport layer ETL satisfies the herein-described range, satisfactory electron transport properties may be obtained without a substantial increase in driving voltage. When the electron transport region ETR includes the electron injection layer EIL, the thickness of the electron injection layer EIL may be about 1 Å to about 100 Å, or about 3 Å to about 90 Å. When the thickness of the electron injection layer EIL satisfies the herein-described range, satisfactory electron injection properties may be obtained without a substantial increase in a driving voltage.

The second electrode EL2 is provided on the electron transport region ETR. The second electrode EL2 may be a common electrode. The second electrode EL2 may be a cathode or an anode, but one or more embodiments of the disclosure is not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is a transflective electrode or reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound and/or a (e.g., any suitable) mixture thereof (for example, AgMg, AgYb, or MgYb). In one or more embodiments, the second electrode EL2 may have a multi-layered structure including a reflective film or transflective film formed of the herein exemplified materials, and a transparent conductive film formed of indium tin oxide (ITO), indium zinc oxide (IZO), zinc oxide (ZnO), indium tin zinc oxide (ITZO), and/or the like. For example, the second electrode EL2 may include any one of the herein-described metal materials, a combination of two or more selected from among the herein-described metal materials, an oxide of any one of the herein-described metal materials, and/or the like.

In one or more embodiments, the second electrode EL2 may be connected to an auxiliary electrode. When the second electrode EL2 is connected to the auxiliary electrode, the resistance of the second electrode EL2 may be reduced.

In one or more embodiments, on the second electrode EL2 of the light emitting element ED of one or more embodiments, the capping layer CPL may be further arranged. The capping layer CPL may include multilayers, or a single layer.

In one or more embodiments, the capping layer CPL may be an organic layer, or an inorganic layer. For example, if (e.g., when) the capping layer CPL includes an inorganic substance, the inorganic substance may include an alkaline metal compound such as LiF, an alkaline earth metal compound such as MgF₂, SiON, SiN_{X}, SiOy, and/or the like.

For example, if (e.g., when) the capping layer CPL includes an organic substance, the organic substance may include α-NPD, NPB, TPD, m-MTDATA, Alq₃, CuPc, N4,N4,N4′, N4′-tetra (biphenyl-4-yl) biphenyl-4,4′-diamine (TPD15), 4,4′,4″-Tris (carbazol-9-yl)triphenylamine (TCTA), and/or the like, or may include an epoxy resin, or an acrylate such as a methacrylate. However, one or more embodiments of the disclosure is not limited thereto. The capping layer CPL may include at least one of (e.g., selected from among) the following compounds P1 to P5.

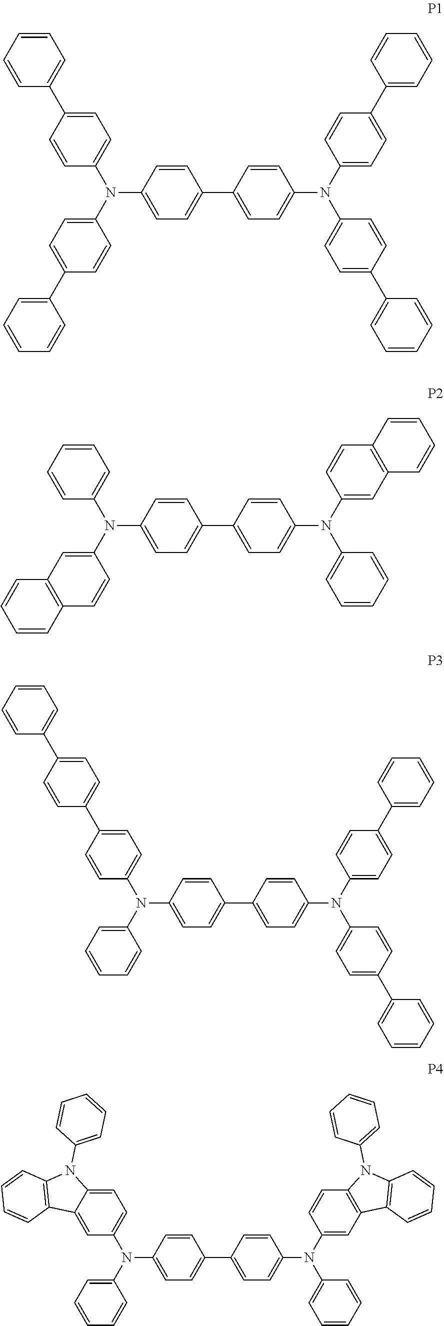 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

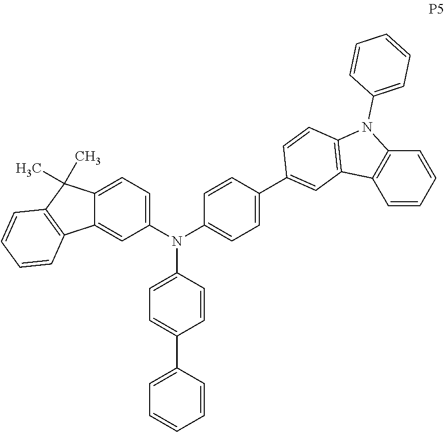 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or greater. For example, for light in a wavelength region of about 550 nm to about 660 nm, the refractive index of the capping layer CPL may be about 1.6 or greater.

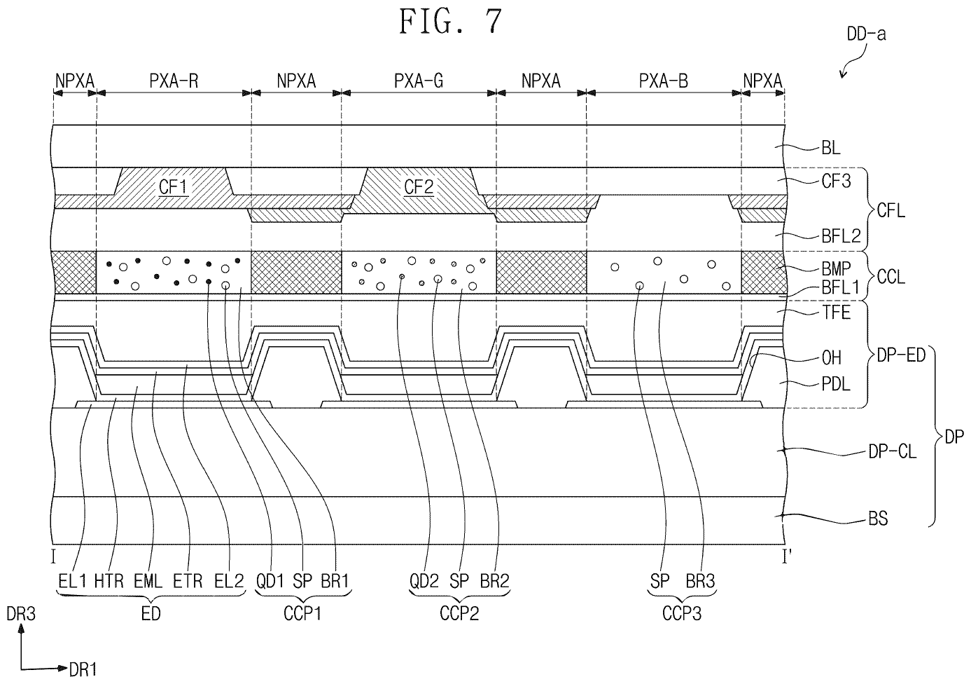

FIG. 7 to FIG. 10 are cross-sectional views on display devices according to one or more embodiments, respectively. In the explanation on the display devices of embodiments referring to FIG. 7 to FIG. 10, the overlapping parts with the explanation on FIG. 1 to FIG. 6 will not be explained again, and the different features will be explained chiefly, e.g., in more detail.

Referring to FIG. 7, the display device DD-a according to one or more embodiments may include a display panel DP including a display device layer DP-ED, a light controlling layer CCL arranged on the display panel DP and a color filter layer CFL. In one or more embodiments shown in FIG. 7, the display panel DP includes a base layer BS, a circuit layer DP-CL provided on the base layer BS and a display device layer DP-ED, and the display device layer DP-ED may include a light-emitting element ED.

The light-emitting element ED may include a first electrode EL1, a hole transport region HTR arranged on the first electrode EL1, an emission layer EML arranged on the hole transport region HTR, an electron transport region ETR arranged on the emission layer EML, and a second electrode EL2 arranged on the electron transport region ETR. In one or more embodiments, the same structures as the light-emitting elements of FIG. 3 to FIG. 6 may be applied to the structure of the light-emitting element ED shown in FIG. 7.

The emission layer EML of the light-emitting element ED included in the display device DD-a according to one or more embodiments may include the fused polycyclic compound of one or more embodiments described herein.

Referring to FIG. 7, the emission layer EML may be arranged in an opening part OH defined in a pixel definition layer PDL. For example, the emission layer EML divided (e.g., defined) by the pixel definition layer PDL, and correspondingly provided to each of luminous areas PXA-R, PXA-G and PXA-B, may be to emit light in substantially the same wavelength region. In the display device DD-a of one or more embodiments, the emission layer EML may be to emit blue light. In one or more embodiments, the emission layer EML may be provided as a common layer for all luminous areas PXA-R, PXA-G and PXA-B.

The light controlling layer CCL may be arranged on the display panel DP. The light controlling layer CCL may include a light converter. The light converter may be a quantum dot or a phosphor. The light converter may transform the wavelength of light provided and then emit. For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be arranged between the separated light controlling parts CCP1, CCP2 and CCP3, but one or more embodiments of the disclosure is not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting a first color light provided from the light-emitting element ED into a second color light, a second light controlling part CCP2 including a second quantum dot QD2 converting the first color light into a third color light, and a third light controlling part CCP3 transmitting the first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light-emitting element ED. For example, the first quantum dot QD1 may be a red quantum dot, and the second quantum dot QD2 may be a green quantum dot. On the quantum dots QD1 and QD2, the same contents as those described herein may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a (e.g., may exclude any) quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3.

The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, and/or the like. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may block the exposure of the light controlling parts CCP1, CCP2 and CCP3 to humidity/oxygen. In one or more embodiments, the barrier layer BFL1 may cover the light controlling parts CCP1, CCP2 and CCP3. In some embodiments, a color filter layer CFL, which will be explained later, may include a barrier layer BFL2 arranged on the light controlling parts CCP1, CCP2 and CCP3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride or a metal thin film securing light transmittance. In one or more embodiments, the barrier layers BFL1 and BFL2 may further include an organic layer. The barrier layers BFL1 and BFL2 may be composed of a single layer of multiple layers.

In the display device DD-a of one or more embodiments, the color filter layer CFL may be arranged on the light controlling layer CCL. For example, the color filter layer CFL may be arranged directly on the light controlling layer CCL. In this case, the barrier layer BFL2 may not be provided.

The color filter layer CFL may include filters CF1, CF2 and CF3. Each of the first to third filters CF1, CF2 and CF3 may be arranged corresponding to a red luminous area PXA-R, a green luminous area PXA-G, and a blue luminous area PXA-B, respectively.

The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment or dye, the second filter CF2 may include a green pigment or dye, and the third filter CF3 may include a blue pigment or dye. However, one or more embodiments of the present disclosure is not limited thereto, and the third filter CF3 may not include (e.g., may exclude any of) the pigment or dye. The third filter CF3 may include a polymer photosensitive resin and not include a pigment or dye. The third filter CF3 may be transparent. The third filter CF3 may be formed using a transparent photosensitive resin.

In some embodiments, in one or more embodiments, the first filter CF1 and the second filter CF2 may each be yellow filters. The first filter CF1 and the second filter CF2 may be provided in one body without distinction.

In some embodiments, the color filter layer CFL may further include a light blocking part. The light blocking part may be a black matrix. The light blocking part may be formed by including an organic light blocking material or an inorganic light blocking material including a black pigment or black dye. The light blocking part may prevent or reduce light leakage phenomenon and divide the boundaries among adjacent filters CF1, CF2 and CF3.

On the color filter layer CFL, a base substrate BL may be arranged. The base substrate BL may be a member providing a base surface on which the color filter layer CFL, the light controlling layer CCL, and/or the like. are arranged. The base substrate BL may be a glass substrate, a metal substrate, a plastic substrate, and/or the like. However, one or more embodiments of the disclosure is not limited thereto, and the base substrate BL may be an inorganic layer, an organic layer or a composite material layer. In some embodiments, different from the drawing, the base substrate BL may not be provided in one or more embodiments.

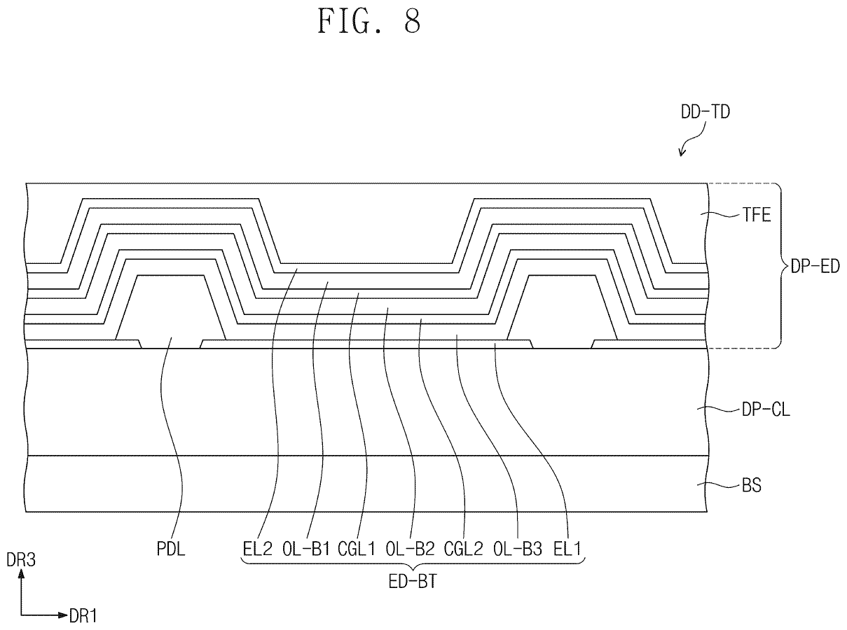

FIG. 8 is a cross-sectional view showing a portion of the display device according to one or more embodiments. In a display device DD-TD of one or more embodiments, the light-emitting element ED-BT may include multiple light emitting structures OL-B1, OL-B2 and OL-B3. The light-emitting element ED-BT may include oppositely arranged first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 7), and a hole transport region HTR and an electron transport region ETR arranged with the emission layer EML (FIG. 7) therebetween.

For example, the light-emitting element ED-BT included in the display device DD-TD of one or more embodiments may be a light-emitting element of a tandem structure including multiple emission layers.

In one or more embodiments shown in FIG. 8, light emitted from the light emitting structures OL-B1, OL-B2 and OL-B3 may be all blue light. However, one or more embodiments of the disclosure is not limited thereto, and the wavelength regions of light emitted from the light emitting structures OL-B1, OL-B2 and OL-B3 may be different from each other. For example, the light-emitting element ED-BT including the multiple light emitting structures OL-B1, OL-B2 and OL-B3 emitting light in different wavelength regions may be to emit white light.

Between neighboring light emitting structures OL-B1, OL-B2 and OL-B3, charge generating layers CGL1 and CGL2 may be arranged. The charge generating layers CGL1 and CGL2 may include a p-type or kind charge generating layer (e.g., p-charge generating layer) and/or an n-type or kind charge generating layer (e.g., n-charge generating layer).

At least one among the emission structures OL-B1, OL-B2, and OL-B3 included the display device DD-TD according to one or more embodiments may contain the herein-described nitrogen-containing compound according to one or more embodiments. For example, at least one among a plurality of electron transport regions included in the light-emitting element ED-BT may contain the nitrogen-containing compound according to one or more embodiments.

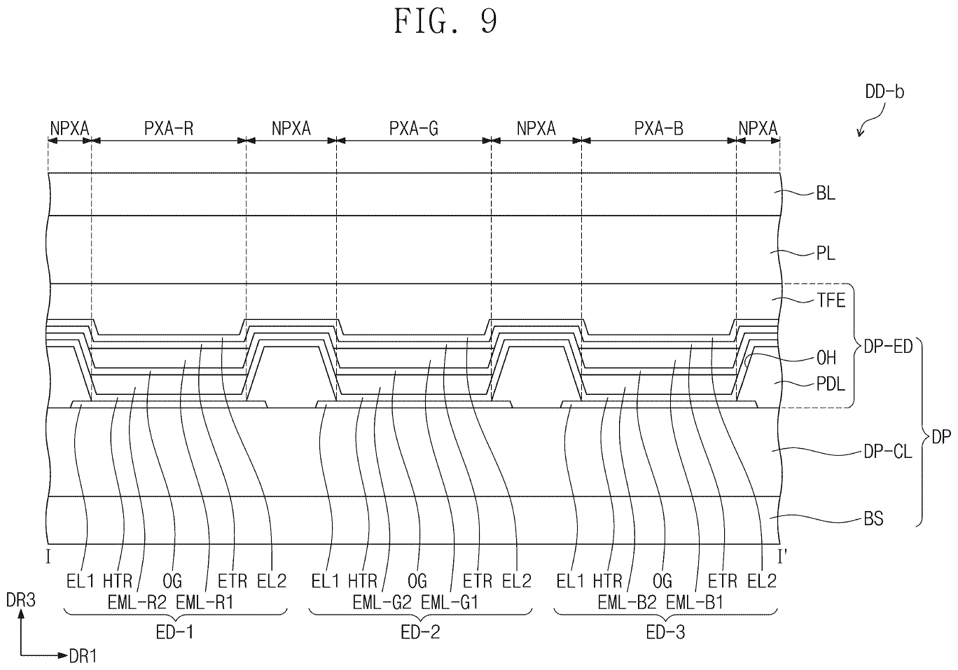

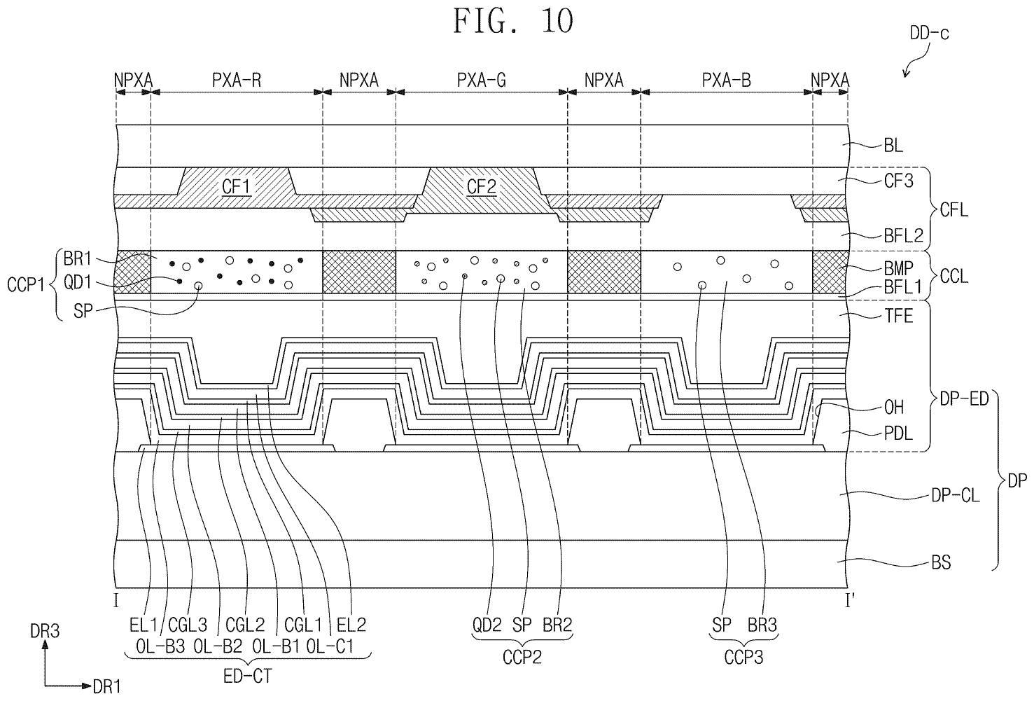

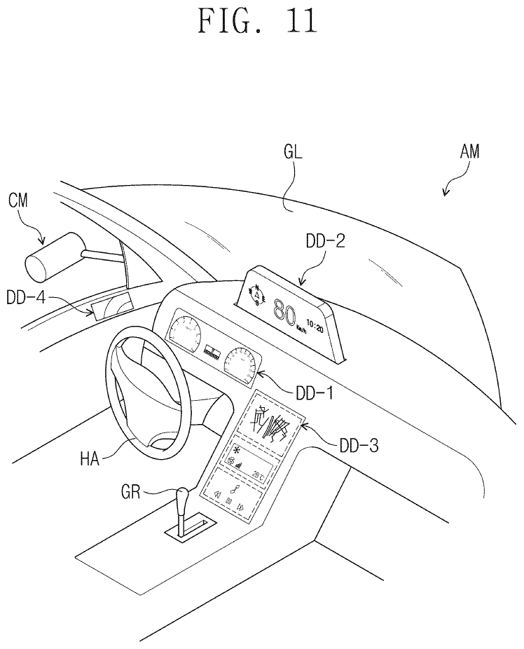

FIG. 9 is a cross-sectional view showing a display device according to one or more embodiments of the disclosure. FIG. 10 is a cross-sectional view showing a display device according to one or more embodiments of the disclosure.

Referring to FIG. 9, a display device DD-b according to one or more embodiments may include light-emitting elements ED-1, ED-2 and ED-3, formed by stacking two emission layers. Compared to the display device DD of one or more embodiments, shown in FIG. 2, one or more embodiments shown in FIG. 9 is different in that first to third light-emitting elements ED-1, ED-2 and ED-3 include two emission layers stacked in a thickness direction, each. In the first to third light-emitting elements ED-1, ED-2 and ED-3, two emission layers may be to emit light in substantially the same wavelength region.

The first light-emitting element ED-1 may include a first red emission layer EML-R1 and a second red emission layer EML-R2. The second light-emitting element ED-2 may include a first green emission layer EML-G1 and a second green emission layer EML-G2. In some embodiments, the third light-emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be arranged.

The emission auxiliary part OG may include a single layer or a multilayer. The emission auxiliary part OG may include a charge generating layer. For example, the emission auxiliary part OG may include an electron transport region, a charge generating layer, and a hole transport region stacked in order. The emission auxiliary part OG may be provided as a common layer in all of the first to third light-emitting elements ED-1, ED-2 and ED-3. However, one or more embodiments of the disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be arranged between the electron transport region ETR and the emission auxiliary part OG. The second red emission layer EML-R2, the second green emission layer EML-G2 and the second blue emission layer EML-B2 may be arranged between the emission auxiliary part OG and the hole transport region HTR.

For example, the first light-emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light-emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light-emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In one or more embodiments, an optical auxiliary layer PL may be arranged on a display device layer DP-ED. The optical auxiliary layer PL may include a polarization layer. The optical auxiliary layer PL may be arranged on a display panel DP and may control reflected light at the display panel DP by external light. Different from the drawings, the optical auxiliary layer PL may not be provided from the display device according to one or more embodiments.

At least one electron transport region included in a display device DD-b according to one or more embodiments illustrated in FIG. 9 may contain the herein-described nitrogen-containing compound according to one or more embodiments. For example, in one or more embodiments, at least one among the electron transport regions ETRs included in the light-emitting elements ED-1, ED-2, and ED-3 may contain the nitrogen-containing Different from FIG. 8 and FIG. 9, a display device DD-c in FIG. 10 is shown to include four light emitting structures OL-B1, OL-B2, OL-B3 and OL-C1. A light-emitting element ED-CT may include oppositely arranged first electrode EL1 and second electrode EL2, and first to fourth light emitting structures OL-B1, OL-B2, OL-B3 and OL-C1 stacked in order in a thickness direction between the first electrode EL1 and the second electrode EL2. The third light emitting structures OL-B3, the second light emitting structures OL-B2, the first light emitting structures OL-B1, and the fourth light emitting structures OL-C1 are stacked in order in a thickness direction. Between the first to fourth light emitting structures OL-B1, OL-B2, OL-B3 and OL-C1, charge generating layers CGL1, CGL2 and CGL3 may be arranged. For example, A first charge generating layer CGL1 is arranged between the first light emitting structures OL-B1 and the fourth light emitting structures OL-C1. A second charge generating layer CGL2 is arranged between the first light emitting structures OL-B1 and the second light emitting structures OL-B2. A third charge generating layer CGL3 is arranged between the second light emitting structures OL-B2 and the third light emitting structures OL-B3.

Among the four light emitting structures, the first to third light emitting structures OL-B1, OL-B2 and OL-B3 may be to emit blue light, and the fourth light emitting structure OL-C1 may be to emit green light. However, one or more embodiments of the disclosure is not limited thereto, and the first to fourth light emitting structures OL-B1, OL-B2, OL-B3 and OL-C1 may be to emit different wavelengths of light.

Between neighboring light emitting structures OL-B1, OL-B2 and OL-B3, charge generating layers CGL1 and CGL2 may be arranged. The charge generating layers CGL1 and CGL2 may include a p-type or kind charge generating layer (e.g., p-charge generating layer) and/or an n-type or kind charge generating layer (e.g., n-charge generating layer).

At least one selected from among emission structures OL-B1, OL-B2, OL-B3, and OL-C1 included in a display device DD-c according to one or more embodiments may contain the herein-described nitrogen-containing compound according to one or more embodiments. For example, in one or more embodiments, at least one among the first to third emission structures OL-B1, OL-B2, and OL-B3 may contain the herein-described nitrogen-containing compound according to one or more embodiments.

The light-emitting element ED according to one or more embodiments of the disclosure may contain the nitrogen-containing compound according to one or more embodiments represented by Formula 1 described herein in at least one functional group arranged between the first electrode EL1 and the second electrode EL2, and thus may exhibit excellent or suitable luminous efficiency and improved lifespan characteristics. For example, the nitrogen-containing compound according to one or more embodiments may be included in the electron transport region ETR of the light-emitting element ED according to one or more embodiments, and the light-emitting element according to one or more embodiments may exhibit long lifespan characteristics.

In one or more embodiments, an electronic apparatus may include a display device including multiple light-emitting elements and a control part controlling the display device. The electronic apparatus of one or more embodiments may be an apparatus activated according to electrical signals. The electronic apparatus may include display devices of one or more suitable embodiments. For example, the electronic apparatus may include televisions, monitors, large-size display devices such as outside billboards, personal computers, laptop computers, personal digital terminals, display devices for automobiles, game consoles, portable electronic devices, medium- and small-size display devices such as cameras.

FIG. 11 is a diagram showing an automobile AM in which first to fourth display devices DD-1, DD-2, DD-3 and DD-4 are arranged. At least one among the first to fourth display devices DD-1, DD-2, DD-3 and DD-4 may include the same configurations of the display devices DD, DD-TD, DD-a, DD-b and DD-c of embodiments, explained referring to FIGS. 1, 2, and 7 to 10.

In FIG. 11, a vehicle is shown as an automobile AM, but this is an illustration, and the first to fourth display devices DD-1, DD-2, DD-3 and DD-4 may be arranged on other transport modes such as bicycles, motorcycles, trains, ships and airplanes. In some embodiments, at least one selected from among the first to fourth display devices DD-1, DD-2, DD-3 and DD-4 including the same configurations of the display devices DD, DD-TD, DD-a, DD-b and DD-c may be introduced in personal computers, laptop computers, personal digital terminals, game consoles, portable electronic devices, televisions, monitors, external billboards, and/or the like. In some embodiments, these are suggested as examples, and the display device may be introduced in other electronic devices as long as not deviated from the disclosure.

At least one selected from among the first to fourth display devices DD-1, DD-2, DD-3, and DD-4 may include the light-emitting element ED of one or more embodiments as described with reference to FIGS. 3 to 6. The light-emitting element ED of one or more embodiments may include a fused polycyclic compound of one or more embodiments. At least one selected from among the first to fourth display devices DD-1, DD-2, DD-3, and DD-4 may include the light-emitting element ED including the fused polycyclic compound of one or more embodiments, thereby improving a display service life.

Referring to FIG. 11, an automobile AM may include a steering wheel HA for the operation of the automobile AM and a gear GR. In some embodiments, the automobile AM may include a front window GL arranged to face a driver.

A first display device DD-1 may be arranged in a first region overlapping with the steering wheel HA. For example, the first display device DD-1 may be a digital cluster displaying the first information of the automobile AM. The first information may include a first graduation showing the running speed of the automobile AM, a second graduation showing the number of revolution of an engine (i.e., revolutions per minute (RPM)), and images showing a fuel state. First graduation and second graduation may be represented by digital images.

A second display device DD-2 may be arranged in a second region opposite to (e.g., facing) a driver's seat and overlapping with the front window GL. The driver's seat may be a seat where the steering wheel HA is arranged. For example, the second display device DD-2 may be a head up display (HUD) showing the second information of the automobile AM. The second display device DD-2 may be optically clear. The second information may include digital numbers showing the running speed of the automobile AM and may further include information including the current time. Different from the drawing, the second information of the second display device DD-2 may be projected and displayed on the front window GL.

A third display device DD-3 may be arranged in a third region adjacent to the gear GR. For example, the third display device DD-3 may be a center information display (CID) for an automobile, arranged between a driver's seat and a passenger seat and showing third information. The passenger seat may be a seat separated from the driver's seat with the gear GR therebetween. The third information may include information on road conditions (for example, navigation information), on playing music or radio, on playing a dynamic image (or image), on the temperature in the automobile AM, and/or the like.

A fourth display device DD-4 may be arranged in a fourth region separated from the steering wheel HA and the gear GR and adjacent to the side of the automobile AM. For example, the fourth display device DD-4 may be a digital wing mirror displaying fourth information. The fourth display device DD-4 may display the external image of the automobile AM, taken by a camera module CM arranged at the outside of the automobile AM. The fourth information may include the external image of the automobile AM.

The herein-described first to fourth information is for illustration, and the first to fourth display devices DD-1, DD-2, DD-3 and DD-4 may further display information on the inside and outside of the automobile. The first to fourth information may include different information from each other. However, one or more embodiments of the disclosure is not limited thereto, and a portion of the first to fourth information may include the same information.

Hereinafter, with reference to examples and comparative examples, a nitrogen-containing compound according to one or more embodiments of the present disclosure and a light-emitting element according to one or more embodiments will be specifically described. In some embodiments, examples shown are only for the understanding of the present disclosure, and the scope of the present disclosure is not limited thereto.

### Examples

### 1. Synthesis of Nitrogen-containing Compounds

A synthetic method of a nitrogen-containing compound according to this embodiment will be described in more detail by exemplifying synthetic methods of Compounds 1, 4, 8, 44, 76, 102, 188, 193, 215, and 272. In some embodiments, in the following descriptions, the synthetic method of the nitrogen-containing compound is provided as an example, but the synthetic method of the nitrogen-containing compound according to one or more embodiments of the disclosure is not limited to the examples disclosed herein.

### (1) Synthesis of Compound 1

Nitrogen-containing Compound 1 according to an example may be synthesized by, for example, a reaction.

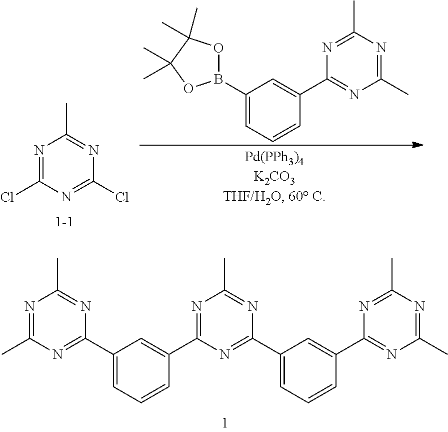

### Synthesis of Compound 1

Intermediate 1-1 (1.63 g), Pd(PPh₃)₄ (0.56 g), K₂CO₃ (3.45 g), and 2,4-dimethyl-6-(3-(4,4,5,5-tetramethyl-1,3,2-dioxaborolan-2-yl)phenyl)-1,3,5-triazine (6.22 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 1 (3.23 g, yield of 70%).

### (2) Synthesis of Compound 4

Nitrogen-containing Compound 4 according to an example may be synthesized by, for example, a reaction.

### Synthesis of Intermediate 4-2

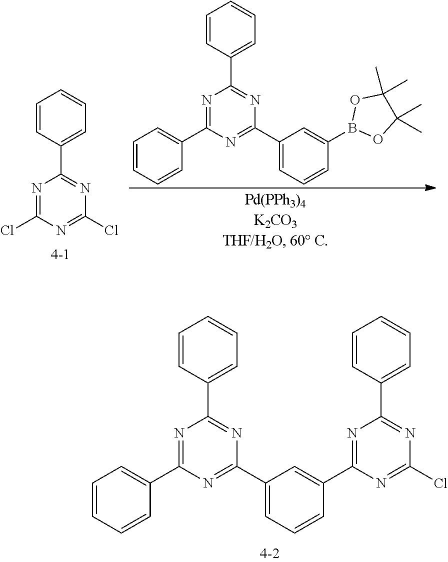 [SMILES: Clc1nc(Cl)nc(-c2ccccc2)n1 ; CC1(C)OB(c2cccc(-c3nc(-c4ccccc4)nc(-c4ccccc4)n3)c2)OC1(C)C ; Clc1nc(-c2ccccc2)nc(-c2cccc(-c3nc(-c4ccccc4)nc(-c4ccccc4)n3)c2)n1]

Intermediate 4-1 (2.26 g), Pd(PPh₃)₄ (0.56 g), K₂CO₃ (3.45 g), and 2,4-diphenyl-6-(3-(4,4,5,5-tetramethyl-1,3,2-dioxaborolan-2-yl)phenyl)-1,3,5-triazine (4.35 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Intermediate 4-2 (3.18 g, yield of 64%).

### Synthesis of Compound 4

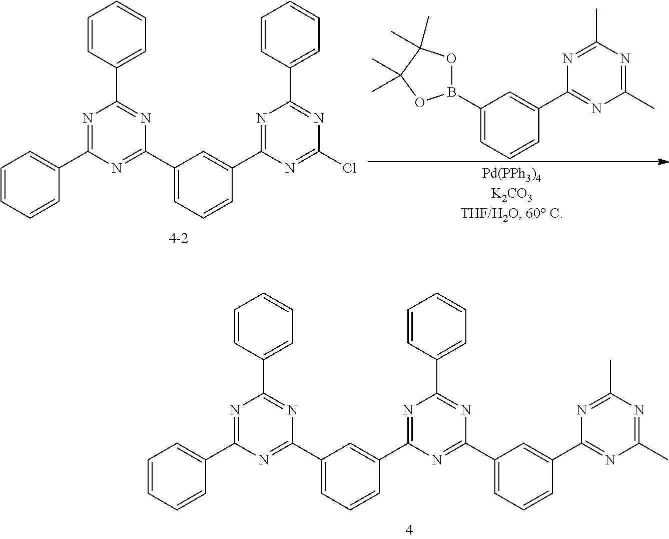

Intermediate 4-2 (4.98 g), Pd(PPh₃)₄ (0.56 g), K₂CO₃ (3.45 g), 2,4-dimethyl-6-(3-(4,4,5,5-tetramethyl-1,3,2-dioxaborolan-2-yl)phenyl)-1,3,5-triazine (3.11 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 4 (3.88 g, yield of 60%).

### (3) Synthesis of Compound 8

Nitrogen-containing Compound 8 according to an example may be synthesized by, for example, a reaction.

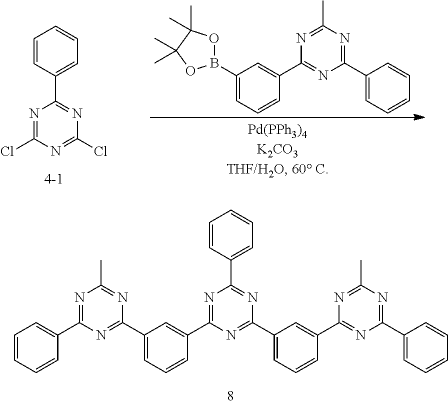 [SMILES: Clc1nc(Cl)nc(-c2ccccc2)n1 ; Cc1nc(-c2ccccc2)nc(-c2cccc(B3OC(C)(C)C(C)(C)O3)c2)n1 ; Cc1nc(-c2ccccc2)nc(-c2cccc(-c3nc(-c4ccccc4)nc(-c4cccc(-c5nc(C)nc(-c6ccccc6)n5)c4)n3)c2)n1]

Intermediate 4-1 (2.26 g), Pd(PPh₃)₄ (0.56 g), K₂CO₃ (3.45 g), and 2-methyl-4-phenyl-6-(3-(4,4,5,5-tetramethyl-1,3,2-dioxaborolan-2-yl)phenyl)-1,3,5-triazine (3.73 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 8 (3.75 g, yield of 58%).

### (4) Synthesis of Compound 44

Nitrogen-containing Compound 44 according to an example may be synthesized by, for example, a reaction.

### Synthesis of Intermediate 44-1

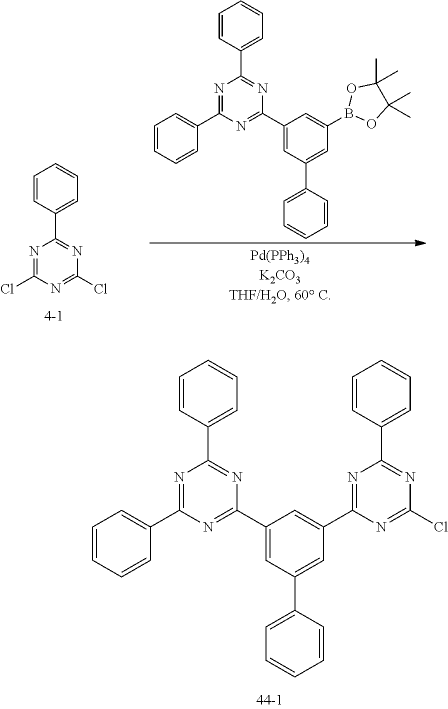 [SMILES: Clc1nc(Cl)nc(-c2ccccc2)n1 ; CC1(C)OB(c2cc(-c3ccccc3)cc(-c3nc(-c4ccccc4)nc(-c4ccccc4)n3)c2)OC1(C)C ; Clc1nc(-c2ccccc2)nc(-c2cc(-c3ccccc3)cc(-c3nc(-c4ccccc4)nc(-c4ccccc4)n3)c2)n1]

Intermediate 4-1 (2.26 g), Pd(PPh₃)₄ (0.56 g), K₂CO₃ (3.45 g), and 2,4-diphenyl-6-(5-(4,4,5,5-tetramethyl-1,3,2-dioxaborolan-2-yl)-[1,1′-biphenyl]-3-yl)-1,3,5-triazine (5.11 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Intermediate 44-1 (3.79 g, yield of 66%).

### Synthesis of Compound 44

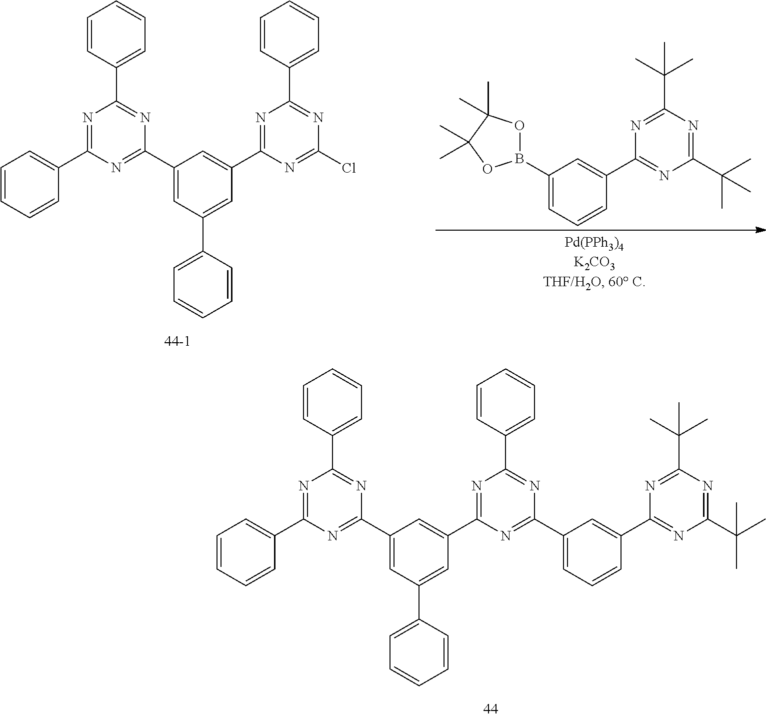 [SMILES: Clc1nc(-c2ccccc2)nc(-c2cc(-c3ccccc3)cc(-c3nc(-c4ccccc4)nc(-c4ccccc4)n3)c2)n1 ; CC(C)(C)c1nc(-c2cccc(B3OC(C)(C)C(C)(C)O3)c2)nc(C(C)(C)C)n1 ; CC(C)(C)c1nc(-c2cccc(-c3nc(-c4ccccc4)nc(-c4cc(-c5ccccc5)cc(-c5nc(-c6ccccc6)nc(-c6ccccc6)n5)c4)n3)c2)nc(C(C)(C)C)n1]

Intermediate 44-1 (5.75 g), Pd(PPh₃)₄ (0.56 g), K₂CO₃ (3.45 g), 2,4-di-tert-butyl-6-(3-(4,4,5,5-tetramethyl-1,3,2-dioxaborolan-2-yl)phenyl)-1,3,5-triazine (3.95 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 44 (4.44 g, yield of 55%).

### (5) Synthesis of Compound 76

Nitrogen-containing Compound 76 according to an example may be synthesized by, for example, a reaction.

### Synthesis of Intermediate 76-1

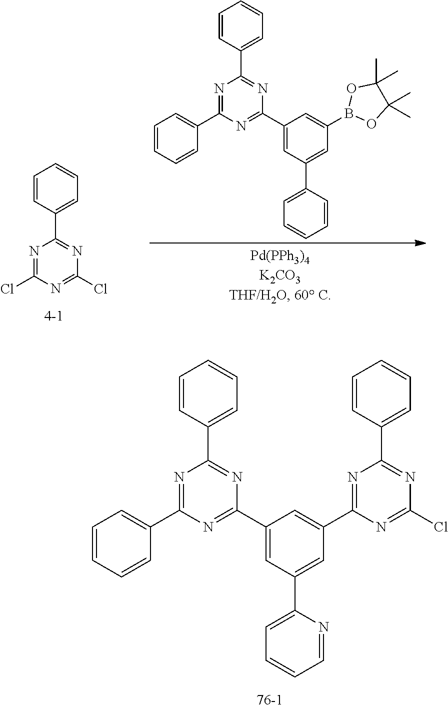 [SMILES: Clc1nc(Cl)nc(-c2ccccc2)n1 ; CC1(C)OB(c2cc(-c3ccccc3)cc(-c3nc(-c4ccccc4)nc(-c4ccccc4)n3)c2)OC1(C)C ; Clc1nc(-c2ccccc2)nc(-c2cc(-c3ccccn3)cc(-c3nc(-c4ccccc4)nc(-c4ccccc4)n3)c2)n1]

1 Intermediate 4-1 (2.26 g), Pd(PPh₃)₄ (0.56 g), K₂CO₃ (3.45 g), and 2,4-diphenyl-6-(3-(pyridin-2-yl)-5-(4,4,5,5-tetramethyl-1,3,2-dioxaborolan-2-yl)phenyl)-1,3,5-triazine (5.12 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Intermediate 76-1 (3.62 g, yield of 63%).

### Synthesis of Compound 76

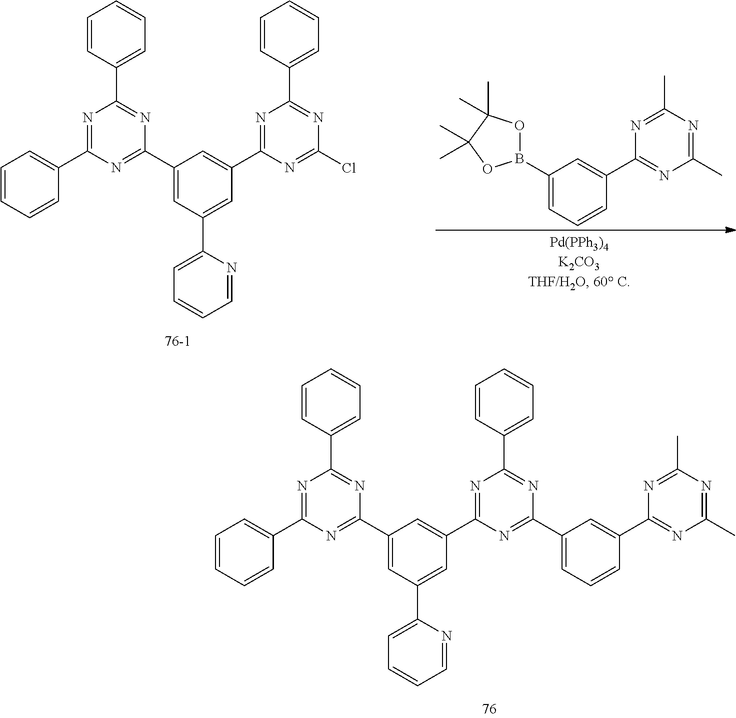

Intermediate 76-1 (5.76 g), Pd(PPh₃)₄ (0.56 g), K₂CO₃ (3.45 g), and 2,4-dimethyl-6-(3-(4,4,5,5-tetramethyl-1,3,2-dioxaborolan-2-yl)phenyl)-1,3,5-triazine (3.11 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 76 (4.34 g, yield of 60%).

### (6) Synthesis of Compound 102

Nitrogen-containing Compound 102 according to an example may be synthesized by, for example, a reaction.

### Synthesis of Intermediate 102-1

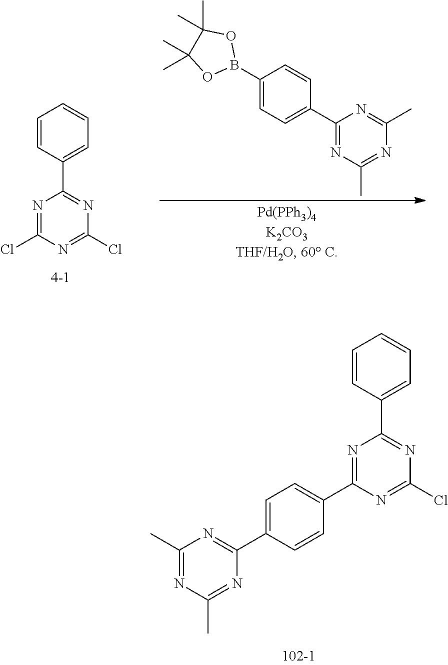

Intermediate 4-1 (2.26 g), Pd(PPh₃)₄ (0.56 g), K₂CO₃ (3.45 g), and 2,4-dimethyl-6-(4-(4,4,5,5-tetramethyl-1,3,2-dioxaborolan-2-yl)phenyl)-1,3,5-triazine (3.11 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Intermediate 102-1 (2.50 g, yield of 67%).

### Synthesis of Compound 102

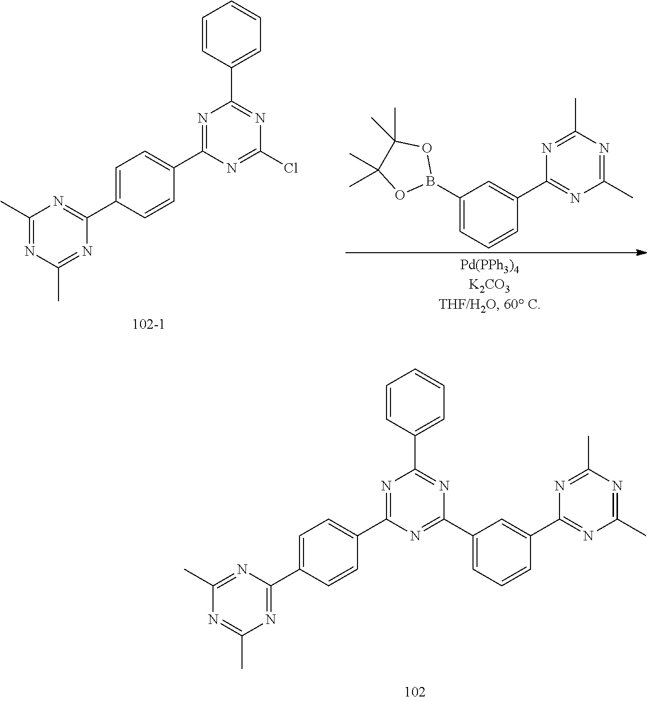

Intermediate 102-1 (3.74 g), Pd(PPh₃)₄ (0.56 g), K₂CO₃ (3.45 g), and 2,4-dimethyl-6-(3-(4,4,5,5-tetramethyl-1,3,2-dioxaborolan-2-yl)phenyl)-1,3,5-triazine (3.11 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 102 (3.50 g, yield of 67%).

### (7) Synthesis of Compound 188

Nitrogen-containing Compound 188 according to an example may be synthesized by, for example, a reaction.

### Synthesis of Intermediate 188-1

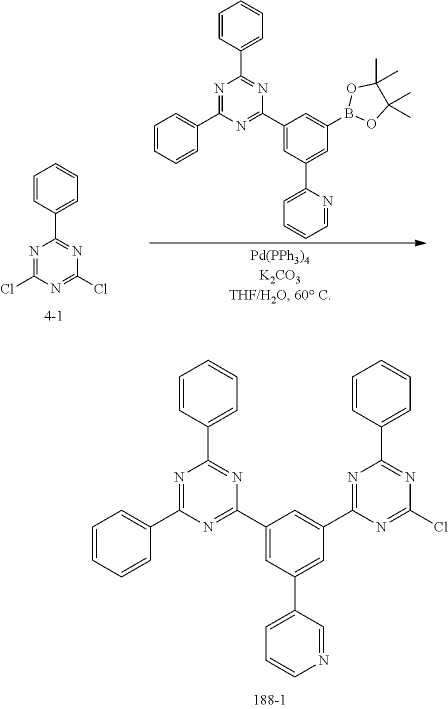 [SMILES: Clc1nc(Cl)nc(-c2ccccc2)n1 ; CC1(C)OB(c2cc(-c3ccccn3)cc(-c3nc(-c4ccccc4)nc(-c4ccccc4)n3)c2)OC1(C)C ; Clc1nc(-c2ccccc2)nc(-c2cc(-c3cccnc3)cc(-c3nc(-c4ccccc4)nc(-c4ccccc4)n3)c2)n1]

Intermediate 4-1 (2.26 g), Pd(PPh₃)₄ (0.56 g), K₂CO₃ (3.45 g), and 2,4-diphenyl-6-(3-(pyridin-2-yl)-5-(4,4,5,5-tetramethyl-1,3,2-dioxaborolan-2-yl)phenyl)-1,3,5-triazine (5.12 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Intermediate 188-1 (4.03 g, yield of 70%).

### Synthesis of Compound 188

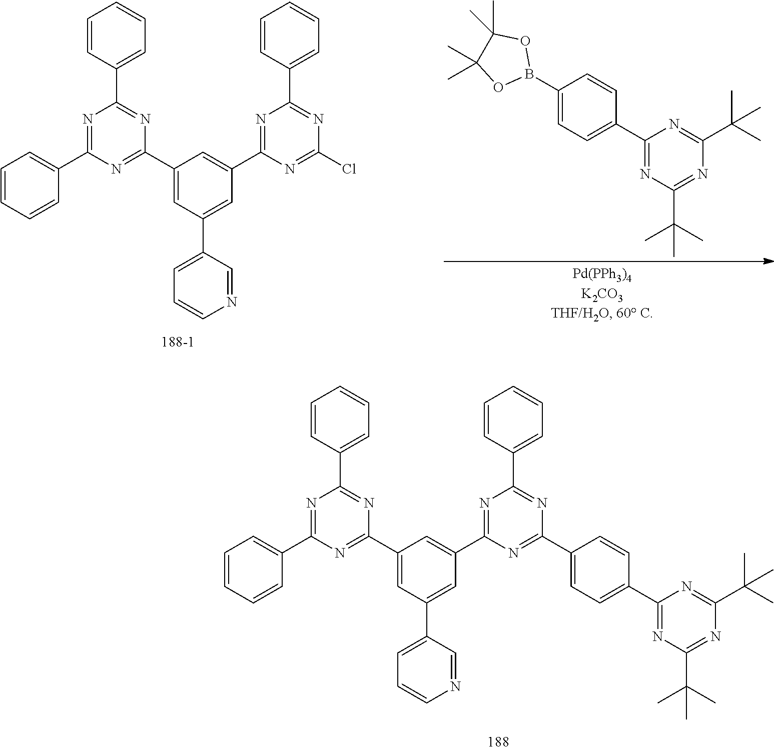 [SMILES: Clc1nc(-c2ccccc2)nc(-c2cc(-c3cccnc3)cc(-c3nc(-c4ccccc4)nc(-c4ccccc4)n3)c2)n1 ; CC(C)(C)c1nc(-c2ccc(B3OC(C)(C)C(C)(C)O3)cc2)nc(C(C)(C)C)n1 ; CC(C)(C)c1nc(-c2ccc(-c3nc(-c4ccccc4)nc(-c4cc(-c5cccnc5)cc(-c5nc(-c6ccccc6)nc(-c6ccccc6)n5)c4)n3)cc2)nc(C(C)(C)C)n1]

Intermediate 188-1 (3.74 g), Pd(PPh₃)₄ (0.56 g), K₂CO₃ (3.45 g), and 2,4-di-tert-butyl-6-(4-(4,4,5,5-tetramethyl-1,3,2-dioxaborolan-2-yl)phenyl)-1,3,5-triazine (3.95 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 188 (5.66 g, yield of 70%).

### (8) Synthesis of Compound 193

Nitrogen-containing Compound 193 according to an example may be synthesized by, for example, a reaction.

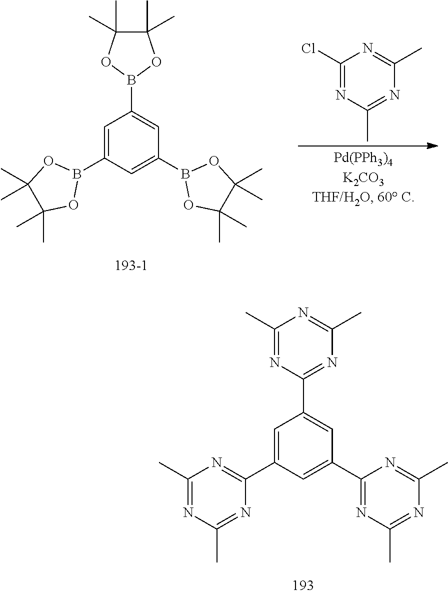

Intermediate 193-1 (4.56 g), Pd(PPh₃)₄ (1.68 g), K₂CO₃ (10.35 g), and 2-chloro-4,6-dimethyl-1,3,5-triazine (4.29 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 193 (2.55 g, yield of 64%).

### (9) Synthesis of Compound 215

Nitrogen-containing Compound 215 according to an example may be synthesized by, for example, a reaction.

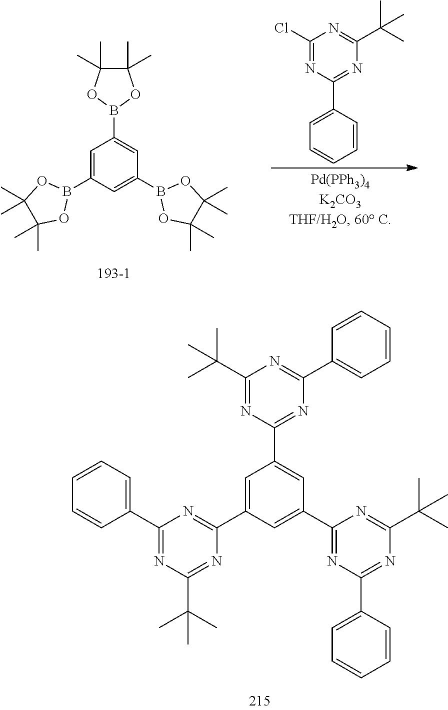

Intermediate 193-1 (4.56 g), Pd(PPh₃)₄ (1.68 g), K₂CO₃ (10.35 g), and 2-(tert-butyl)-4-chloro-6-phenyl-1,3,5-triazine (2.47 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 215 (2.55 g, yield of 64%).

### (10) Synthesis of Compound 272

Nitrogen-containing Compound 272 according to an example may be synthesized by, for example, a reaction.

### Synthesis of Intermediate 272-1

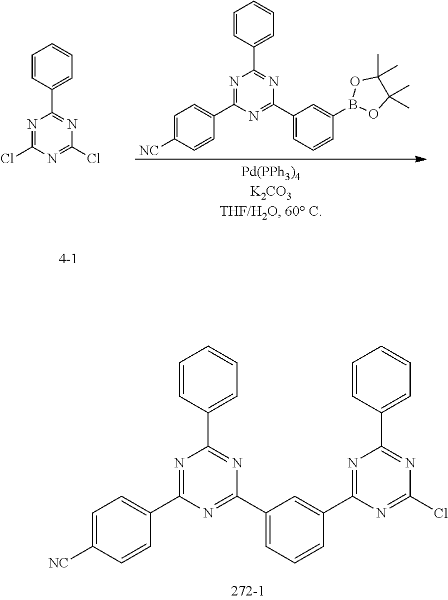 [SMILES: Clc1nc(Cl)nc(-c2ccccc2)n1 ; CC1(C)OB(c2cccc(-c3nc(-c4ccccc4)nc(-c4ccc(C#N)cc4)n3)c2)OC1(C)C ; N#Cc1ccc(-c2nc(-c3ccccc3)nc(-c3cccc(-c4nc(Cl)nc(-c5ccccc5)n4)c3)n2)cc1]

Intermediate 4-1 (2.26 g), Pd(PPh₃)₄ (0.56 g), K₂CO₃ (3.45 g), and 4-(4-phenyl-6-(3-(4,4,5,5-tetramethyl-1,3,2-dioxaborolan-2-yl)phenyl)-1,3,5-triazin-2-yl)benzonitrile (4.60 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under reduced pressure, and the obtained residue was separated and purified using column chromatography to obtain Intermediate 272-1 (3.39 g, yield of 65%).

### Synthesis of Compound 272

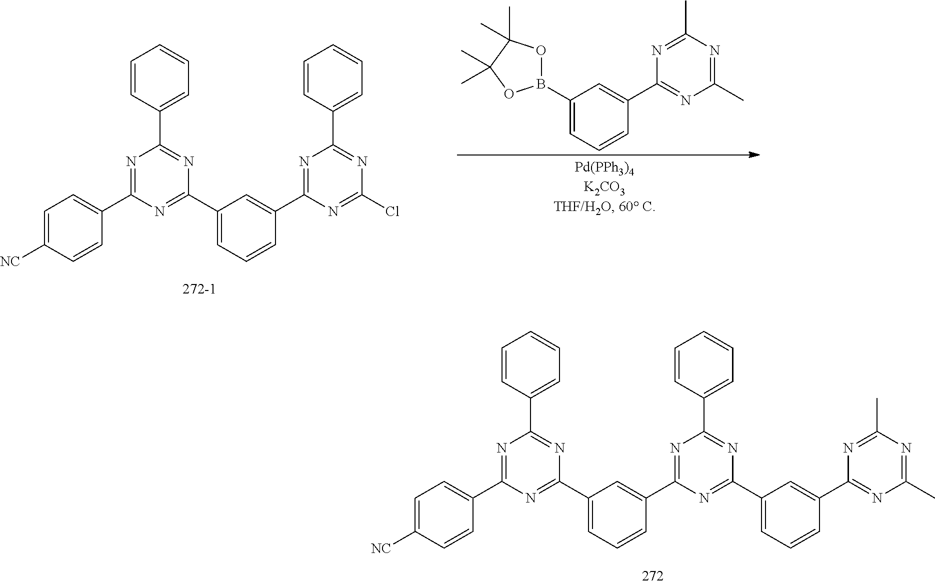

Intermediate 272-1 (5.23 g), Pd(PPh₃)₄ (0.56 g), K₂CO₃ (3.45 g), 2,4-dimethyl-6-(3-(4,4,5,5-tetramethyl-1,3,2-dioxaborolan-2-yl)phenyl)-1,3,5-triazine (3.11 g) were dissolved in THF/H₂O (100 mL/25 mL), and then the mixture was stirred at 60° C. for 12 hours. The reaction solution was cooled to room temperature, then the reaction was terminated with water, and an organic layer was extracted three times with ethyl ether. The separated organic layer was dried over anhydrous magnesium sulfate and distilled under a reduced pressure, and the obtained residue was separated and purified using column chromatography to obtain Compound 272 (4.36 g, yield of 65%).

### 2. Manufacture and Evaluation of Light-Emitting Element

The light-emitting elements according to one or more embodiments including the nitrogen-containing compound according to one or more embodiments in the electron transport layer were manufactured by the following method. The light-emitting elements according to Example 1 to Example 10 were manufactured using Nitrogen-containing Compounds 1, 4, 8, 44, 76, 102, 188, 193, 215, and 272, which are the herein-described example compounds, as an electron transport layer material. The light-emitting elements according to Comparative Example 1 to Comparative Example 6 correspond to light-emitting elements manufactured by using Comparative Example Compound C1 to Comparative Example Compound C6, respectively, as an electron transport layer material.

### Example Compounds

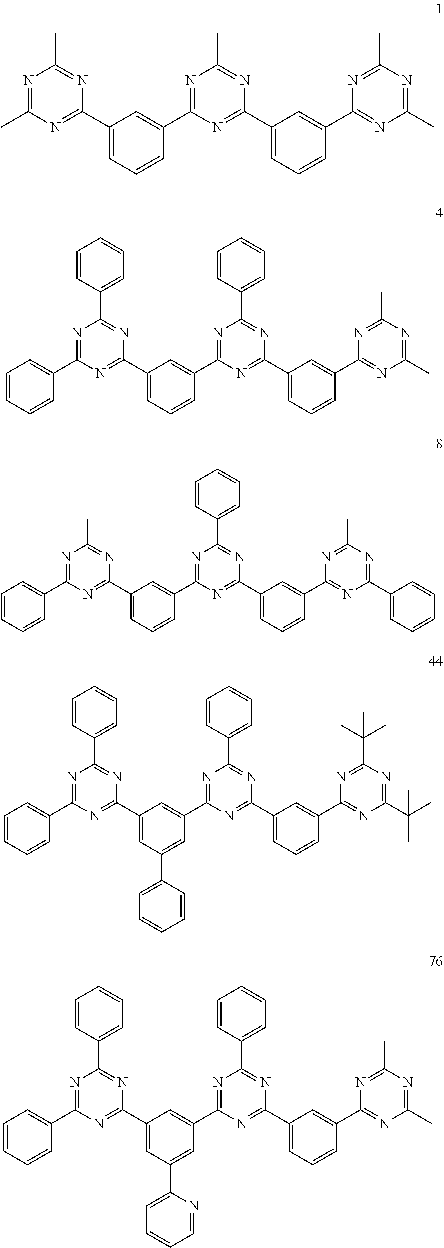

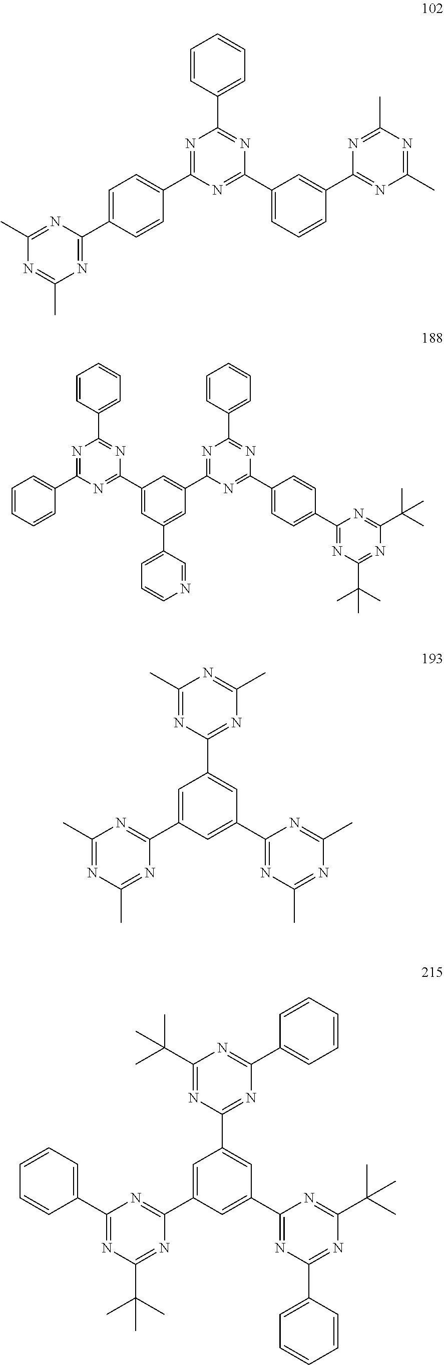

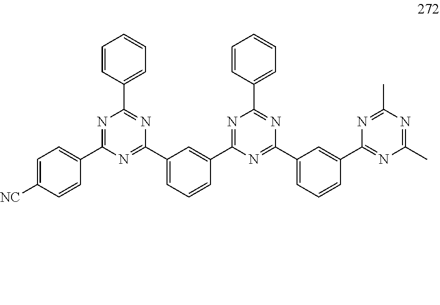

### Comparative Example Compounds

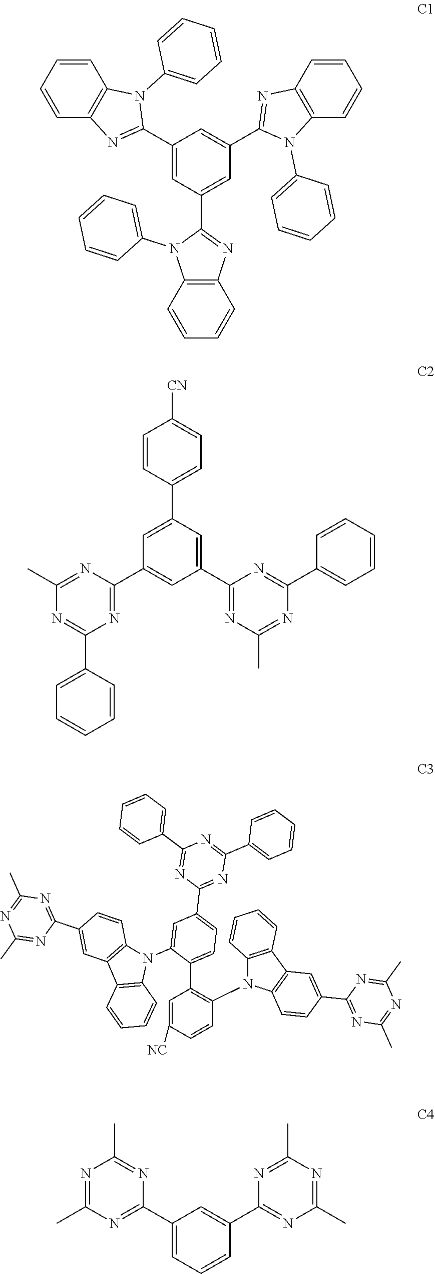

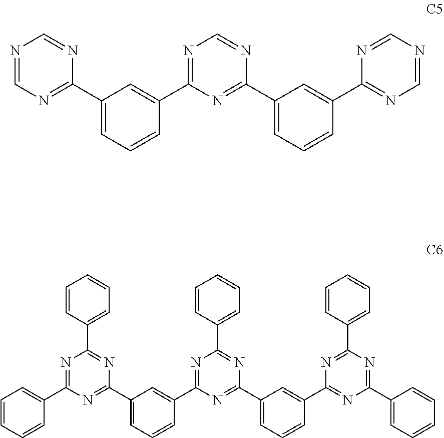 [SMILES: c1cc(-c2ncncn2)cc(-c2ncnc(-c3cccc(-c4ncncn4)c3)n2)c1 ; c1ccc(-c2nc(-c3ccccc3)nc(-c3cccc(-c4nc(-c5ccccc5)nc(-c5cccc(-c6nc(-c7ccccc7)nc(-c7ccccc7)n6)c5)n4)c3)n2)cc1]

### Manufacture of Light-Emitting Element

In the light-emitting elements according to examples and comparative examples, a glass substrate (made by Corning Co.), on which an ITO electrode of about 15 ohm per square centimeter (Ω/cm²) (a thickness of 1,200 angstrom (Å)) was formed as a first electrode, was cut to a size of about 50 millimeter (mm)×50 mm×0.7 mm, cleansed by ultrasonic waves using isopropyl alcohol and pure water for about five minutes each, then irradiated with ultraviolet rays for about 30 minutes and exposed to ozone and cleansed. Then, the glass substrate was mounted on a vacuum deposition apparatus.

NPD was deposited on the first electrode to form a hole injection layer having a thickness of about 300 Å, then H-1-1 was deposited on the hole injection layer to form a hole transport layer having a thickness of about 200 Å, and then CzSi was deposited on the hole transport layer to form an emission auxiliary layer having a thickness of about 100 Å.

A mixed host material, in which a second compound and a third compound were mixed at a weight ratio of 1:1, a fourth compound which is a sensitizer, and a first compound which is a dopant material were co-deposited on the emission auxiliary layer at a weight ratio of 84:15:1 to form an emission layer having a thickness of about 200 Å. Subsequently, TSPO1 was deposited on the emission layer to form a hole-blocking layer having a thickness of about 200 Å, then an example compound or a comparative example compound was deposited on the hole-blocking layer to form an electron transport layer having a thickness of about 300 Å, then LiF was deposited on the electron transport layer to form an electron injection layer having a thickness of about 10 Å, and Al was deposited on the electron injection layer to form a second electrode having a thickness of about 3000 Å, thereby manufacturing a light-emitting element. Each layer was formed by a vacuum deposition method.

In one or more embodiments, for use as the second compound, HT2 or HT3 was selected from among the compounds in Compound Group 2 described herein, for use as the third compound ETH66 or ETH86 was selected from among the compounds in Compound Group 3 described herein, for use as the fourth compound, AD-37 or AD-38 was selected from among the compounds in Compound Group 4 described herein, and t-DABNA was used as the first compound.

Compounds used for the manufacture of the light-emitting elements according to examples and comparative examples are disclosed. The following materials were used in the manufacture of elements by purifying commercial products by sublimation.

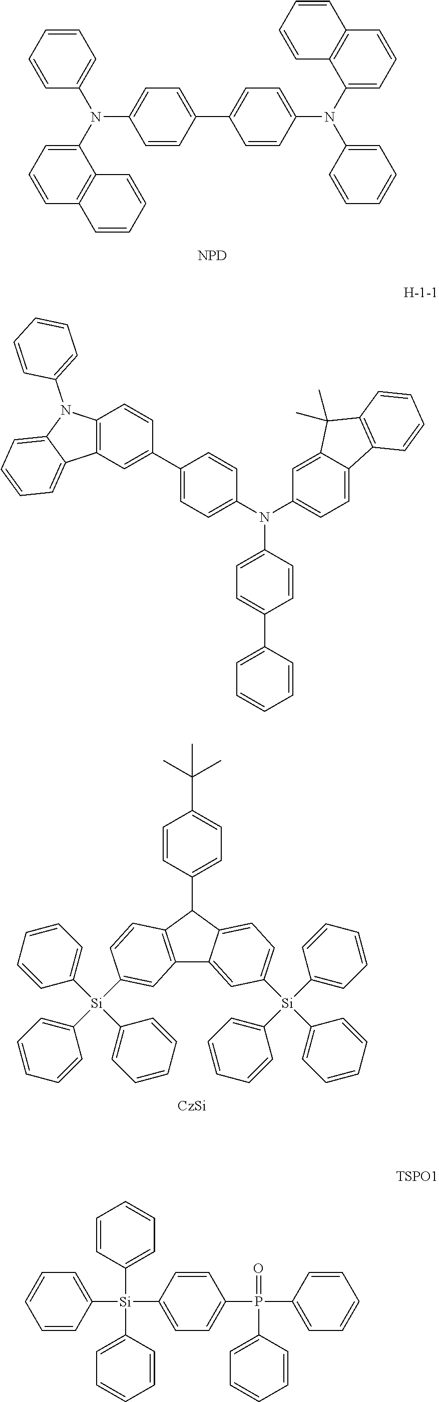 [SMILES: c1ccc(N(c2ccc(-c3ccc(N(c4ccccc4)c4cccc5ccccc45)cc3)cc2)c2cccc3ccccc23)cc1 ; "NPD" ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC(C)(C)c1ccc(C2c3ccc([Si](c4ccccc4)(c4ccccc4)c4ccccc4)cc3-c3cc([Si](c4ccccc4)(c4ccccc4)c4ccccc4)ccc32)cc1 ; O=P(c1ccccc1)(c1ccccc1)c1ccc([Si](c2ccccc2)(c2ccccc2)c2ccccc2)cc1]

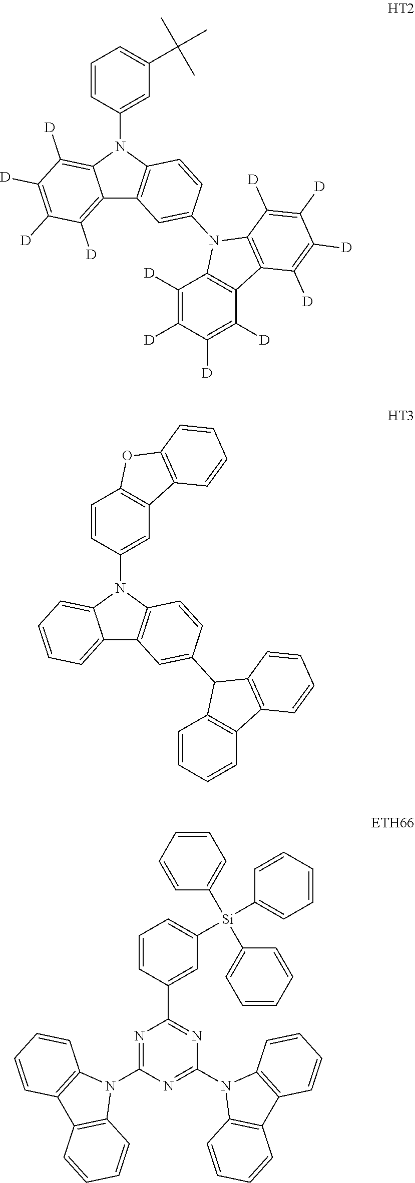 [SMILES: [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)-c1ccccc1C2c1ccc2c(c1)c1ccccc1n2-c1ccc2oc3ccccc3c2c1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

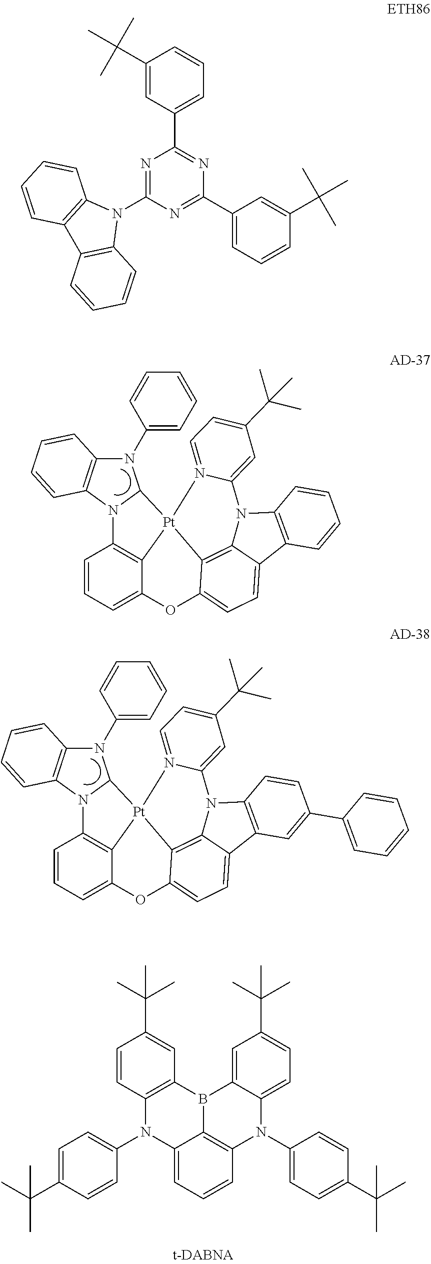

### Evaluation of Characteristics of Light-emitting Element

Element efficiency and element lifespan of the light-emitting elements manufactured using Example Compounds 1, 4, 8, 44, 76, 102, 188, 193, 215, and 272, and Comparative Example Compounds C1 to C6 were evaluated. The evaluated results of the light-emitting elements according to Examples 1 to 10, and Comparative Examples 1 to 6 are listed in Table 1. In order to evaluate the characteristics of the light-emitting elements manufactured according to Example 1 to Example 10, and Comparative Example 1 to Comparative Example 6, driving voltage (V) and luminous efficiency (candela per ampere (cd/A)) at a current density of about 1000 candela per square meter (cd/m²) were each measured using Keithley MU 236 and luminance meter PR650. Time taken for initial luminance to decrease from 100% to 95% was measured as a lifespan (T95), a relative lifespan was calculated with respect to the lifespan of Comparative Example 1, and the calculated value was listed in Table 1. In Table 1, the Example Compounds represented with “EC” preceding the compound number, the Comparative Example Compounds C1 to C6 are represented as “CEC 1 to CEC 6”, and t-DABNA was used as the first compound in each light-emitting element.

TABLE 1

| | Host | | | | | |
|---|---|---|---|---|---|---|
| | (Second | | | | | |
| | compound: | | Electron | | | Life- |
| | third | Fourth | transport | Driving | Effici- | span |
| | compound = | com- | layer | voltage | ency | ratio |
| | 5:5) | pound | material | (V) | (cd/A) | (T95) |
| Example 1 | HT3/ETH66 | AD-38 | EC 1 | 4.31 | 27.1 | 5.0 |
| Example 2 | HT3/ETH66 | AD-38 | EC 4 | 3.94 | 26.8 | 5.7 |
| Example 3 | HT3/ETH66 | AD-38 | EC 8 | 3.99 | 24.3 | 4.8 |
| Example 4 | HT3/ETH66 | AD-38 | EC 44 | 3.96 | 27.3 | 5.3 |
| Example 5 | HT3/ETH66 | AD-38 | EC 76 | 4.10 | 26.2 | 5.4 |
| Example 6 | HT3/ETH66 | AD-38 | EC 102 | 4.05 | 24.9 | 5.5 |
| Example 7 | HT3/ETH66 | AD-38 | EC 188 | 4.13 | 25.7 | 5.5 |
| Example 8 | HT3/ETH66 | AD-38 | EC 193 | 4.30 | 26.9 | 4.7 |
| Example 9 | HT2/ETH86 | AD-37 | EC 215 | 4.21 | 26.4 | 4.9 |
| Example 10 | HT2/ETH86 | AD-37 | EC 272 | 4.07 | 25.0 | 4.9 |
| Comparative | HT3/ETH66 | AD-38 | CEC 1 | 5.6 | 18.8 | 1 |
| Example 1 | | | | | | |
| Comparative | HT3/ETH66 | AD-38 | CEC 2 | 4.35 | 22.7 | 4.3 |
| Example 2 | | | | | | |
| Comparative | HT3/ETH66 | AD-38 | CEC 3 | 5.31 | 19.3 | 3.3 |
| Example 3 | | | | | | |
| Comparative | HT3/ETH66 | AD-38 | CEC 4 | 4.73 | 20.5 | 2.7 |
| Example 4 | | | | | | |
| Comparative | HT3/ETH66 | AD-38 | CEC 5 | 4.57 | 21.3 | 3.0 |
| Example 5 | | | | | | |
| Comparative | HT3/ETH66 | AD-38 | CEC 6 | 4.63 | 20.5 | 3.9 |
| Example 6 | | | | | | |

Referring to Table 1, the light-emitting elements according to Example 1 to Example 10 exhibited characteristics of high efficiency and long lifespan, compared to the light-emitting elements according to Comparative Examples 1 to 6.

Example Compounds 1, 4, 8, 44, 76, 102, 188, 193, 215, and 272 each include three triazine moieties linked via at least one linker, and an alkyl substituent that is connected (linked) to at least one selected from among the three triazine moieties, and thus may exhibit excellent or suitable charge-transporting properties and material stability due to such structural properties, e.g., when compared to other Comparative Example Compounds C1 to C6. For example, it may be confirmed that the Example Compounds have excellent or suitable charge-transporting properties and charge balance due to the molecular structural characteristics of the Example Compounds, which distinguish the Example Compounds from the Comparative Example Compounds, and thus the light-emitting elements according to the Examples, which include the nitrogen-containing compounds in the electron transport layer, may exhibit characteristics of high efficiency and long lifespan.

Compared to Comparative Example C1, Example Compounds include three triazine moieties linked to each other via at least one linker, and include at least one alkyl substituent in the triazine moieties, thereby exhibiting excellent or suitable charge-transporting property and material stability. Therefore, the light-emitting elements according to Example 1 to Example 10 including the Example Compounds have improved luminous efficiency and lifespan, compared to the light-emitting element according to Comparative Example 1 including Comparative Example Compound C1.

Comparative Example Compound C2 and Comparative Example Compound C4, which are compounds in Comparative Example 2 and Comparative Example 4, respectively, each include only two triazine moieties, which is different from the Example Compounds, and thus, it may be seen that decreases in luminous efficiency and lifespan are exhibited or caused.

Comparative Example Compound C3 according to Comparative Example 3 includes three triazine moieties connected via at least one linker and includes a structure in which an alkyl substituent is connected to the triazine moiety, but includes carbazole as a linker, and thus luminous efficiency and lifespan were both (e.g., simultaneously) reduced compared to Examples. In a case where carbazole is used or contained as a linker, such as Comparative Example Compound C3, electron transport properties are insufficient, and thus may be disadvantageous in luminous efficiency and lifespan characteristics. Compared to this, because the Example Compounds include no carbazole linker as a linker, it may be confirmed that charge-transporting property was improved, and thus luminous efficiency and lifespan characteristics were all improved. The three triazine moieties in Comparative Example Compound C3 are separated from each other at greater distances when compared to the Example Compounds.

Comparative Example Compound C5 and Comparative Example Compound C6 according to Comparative Example 5 and Comparative Example 6, respectively, each include no alkyl substituent, which is different from the Example Compounds and thus, (e.g., due to this), a decrease in luminous efficiency and lifespan may be confirmed. The light-emitting elements according to Example 1 to Example 10 exhibit improved luminous efficiency and lifespan concurrently (e.g., simultaneously), compared to the light-emitting elements according to Comparative Example 5 and Comparative Example 6. For example, the light-emitting element according to one or more embodiments may have improved element efficiency and lifespan at the same time by using the nitrogen-containing compound according to one or more embodiments, which includes an alkyl substituent introduced in at least one three triazine moieties in an electron transport region.

The light-emitting element according to one or more embodiments may exhibit improved element characteristics.

The nitrogen-containing compound according to one or more embodiments is included in the electron transport region of the light-emitting element, which may contribute to improvements in high efficiency and long lifespan of the light-emitting element.

The display device according to one or more embodiments may exhibit excellent or suitable display quality.

The display device, and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

Hitherto, although one or more embodiments of the present disclosure have been described, it is understood that the present disclosure should not be limited to these embodiments but one or more suitable changes and modifications can be made by one ordinary skilled in the art within the spirit and scope of the present disclosure as hereinafter claimed. Also, Descriptions of features or aspects within each embodiment should typically be considered as available for other similar features or aspects in other embodiments.

Therefore, the technical scope of the present disclosure is not intended to be limited to the contents set forth in the detailed description of the specification, but is intended to be defined by the appended claims and equivalents thereof.

What is claimed is:

1. A light-emitting element comprising:
a first electrode;
a second electrode on the first electrode; and
at least one functional layer between the first electrode and the second electrode, and comprising a nitrogen-containing compound represented by Formula 1: 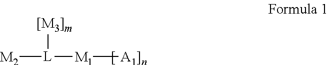
wherein, in Formula 1,
A₁ is -(L₁)ₐ₁M₄,
L₁ is a substituted or unsubstituted arylene group having 6 to 60 ring-forming carbons, or a substituted or unsubstituted heteroarylene group having 2 to 60 ring-forming carbons,
M₂ to M₄ are each independently a substituted or unsubstituted triazine group,
a1 is an integer of 1 to 3,
m and n are each independently 0 or 1,
a sum of m and n is 1,
when n is 0, then M₁ is a substituted or unsubstituted triazine group,
when n is 1, then M₁ is a substituted or unsubstituted divalent triazine group,
when m is 0, then L is represented by -(L₂)ₐ₂-,
L₂ is a substituted or unsubstituted arylene group having 6 to 60 ring-forming carbons, or a substituted or unsubstituted heteroarylene group having 2 to 60 ring-forming carbons,
a2 is an integer of 1 to 3,
when m is 1, then L is a substituted or unsubstituted trivalent aryl group having 6 to 60 ring-forming carbons, or a substituted or unsubstituted trivalent heteroaryl group having 2 to 60 ring-forming carbons,
at least one selected from among M₁ to M₄ comprises, as a substituent, an alkyl group having 1 to 30 carbons, or a substituted or unsubstituted cycloalkyl group having 3 to 30 ring-forming carbons, and
each of L, L₁, and L₂ do not comprise a substituted or unsubstituted carbazole linker.

2. The light-emitting element of claim 1,
wherein the at least one functional layer comprises:
an emission layer;
a hole transport region between the first electrode and the emission layer; and
an electron transport region between the emission layer and the second electrode; and
the electron transport region comprises the nitrogen-containing compound represented by Formula 1.

3. The light-emitting element of claim 2, wherein the electron transport region comprises:
an electron transport layer on the emission layer; and
an electron injection layer on the electron transport layer, and
the electron transport layer comprises the nitrogen-containing compound represented by Formula 1.

4. The light-emitting element of claim 2, wherein an adjacent layer to the emission layer selected from among a plurality of layers in the electron transport region comprises the nitrogen-containing compound represented by Formula 1.

5. The light-emitting element of claim 1, wherein at least one selected from among M₁ to M₄ is substituted with a substituted or unsubstituted methyl group, a substituted or unsubstituted isopropyl group, a substituted or unsubstituted t-butyl group, a substituted or unsubstituted cyclohexyl group, a substituted or unsubstituted bicycloheptanyl group, or a substituted or unsubstituted adamantyl group.

6. The light-emitting element of claim 1, wherein:
L₁ and L₂ are each independently a substituted or unsubstituted divalent phenyl group, a substituted or unsubstituted divalent biphenyl group, or a substituted or unsubstituted divalent naphthyl group; and
L is a substituted or unsubstituted trivalent phenyl group, a substituted or unsubstituted trivalent biphenyl group, or a substituted or unsubstituted trivalent naphthyl group.

7. The light-emitting element of claim 1, wherein the nitrogen-containing compound is represented by Formula 2-1 or Formula 2-2: 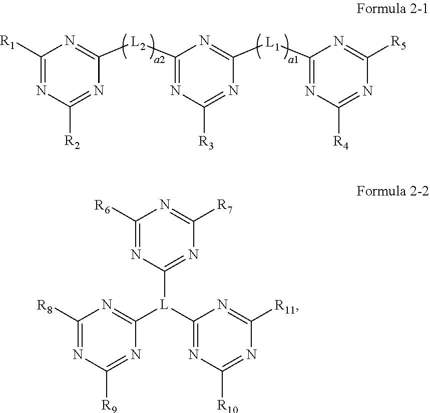
wherein, in Formula 2-1 and Formula 2-2,
R₁ to R₁₁ are each independently a hydrogen atom, a deuterium atom, a halogen atom, a nitro group, a cyano group, a substituted or unsubstituted alkyl group having 1 to 30 carbons, a substituted or unsubstituted cycloalkyl group having 3 to 30 ring-forming carbons, a substituted or unsubstituted aryl group having 6 to 60 ring-forming carbons, or a substituted or unsubstituted heteroaryl group having 2 to 60 ring-forming carbons,
in Formula 2-1, at least one selected from among R₁ to R₅ is a substituted or unsubstituted alkyl group having 1 to 30 carbons, or a substituted or unsubstituted cycloalkyl group having 3 to 30 ring-forming carbons,
in Formula 2-2, at least one selected from among R₆ to R₁₁ is a substituted or unsubstituted alkyl group having 1 to 30 carbons, or a substituted or unsubstituted cycloalkyl group having 3 to 30 ring-forming carbons, and
L, L₁, L₂, a1, and a2 being as defined in Formula 1.

8. The light-emitting element of claim 7, wherein the nitrogen-containing compound is represented by any one selected from among Formula 3-1 to Formula 3-3: 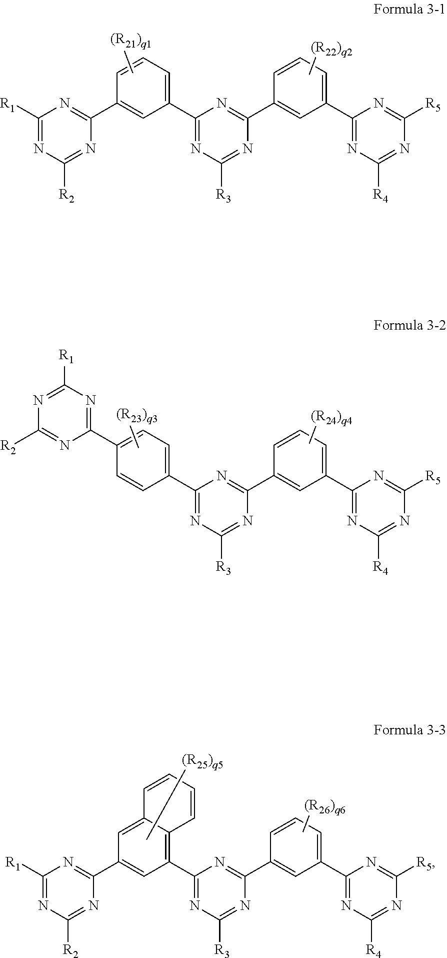
wherein, in Formula 3-1 to Formula 3-3,
R₂₁ to R₂₆ are each independently a hydrogen atom, a deuterium atom, a halogen atom, a cyano group, a nitro group, a substituted or unsubstituted alkyl group having 1 to 30 carbons, a substituted or unsubstituted aryl group having 6 to 60 ring-forming carbons, or a substituted or unsubstituted heteroaryl group having 2 to 60 ring-forming carbons,
q1 to q4, and q6 are each independently an integer of 0 to 4,
q5 is an integer of 0 to 6, and
R₁ to R₅ are as defined in Formula 2-1.

9. The light-emitting element of claim 7, wherein the nitrogen-containing compound is represented by Formula 4: 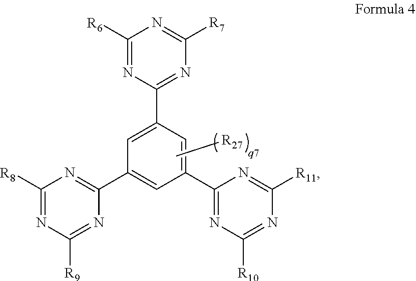
wherein, in Formula 4,
R₂₇ is a hydrogen atom, a deuterium atom, a halogen atom, a cyano group, a nitro group, a substituted or unsubstituted alkyl group having 1 to 20 carbons, a substituted or unsubstituted aryl group having 6 to 60 ring-forming carbons, or a substituted or unsubstituted heteroaryl group having 2 to 60 ring-forming carbons,
q7 is an integer of 0 to 3, and
R₆ to R₁₁ are as defined in Formula 2-2.

10. The light-emitting element of claim 7, wherein:
in Formula 2-1, at least one selected from among R₁ to R₅ is a substituted or unsubstituted alkyl group having 1 to 10 carbons, and any remaining selected from among R₁ to R₅ are each independently a substituted or unsubstituted phenyl group, or a substituted or unsubstituted biphenyl group; and
in Formula 2-2, at least one selected from among R₆ to R₁₁ is a substituted or unsubstituted alkyl group having 1 to 10 carbons, and any remaining selected from among R₆ to R₁₁ are each independently a substituted or unsubstituted phenyl group, or a substituted or unsubstituted biphenyl group.

11. The light-emitting element of claim 1, wherein L₁ and L₂ are each independently represented by any one selected from among Formula L-1 to L-9, and
L is represented by Formula L-10: 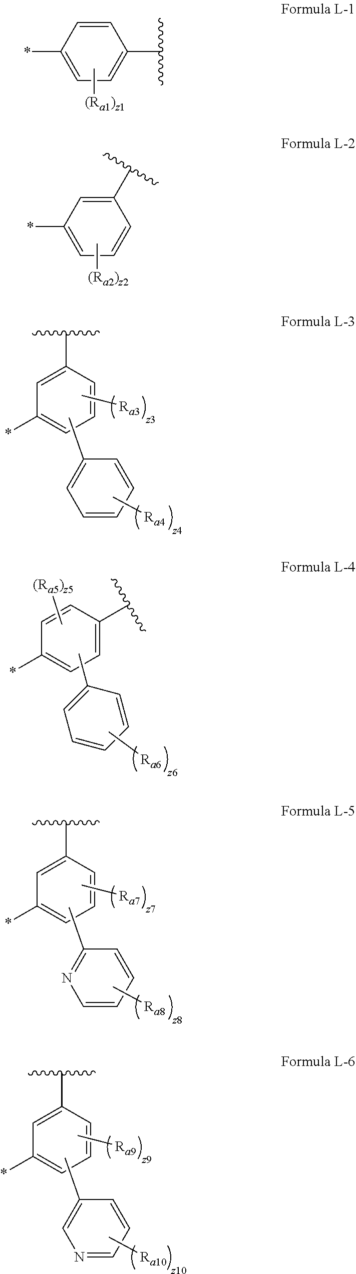 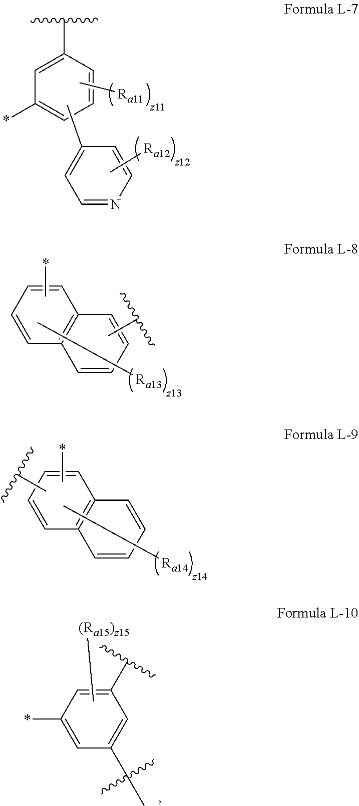
wherein, in Formula L-1 to L-10,
Rₐ₁ to Rₐ₁₅ are each independently a hydrogen atom, a deuterium atom, a halogen atom, a cyano group, a substituted or unsubstituted phosphine oxide group, a substituted or unsubstituted alkyl group having 1 to 20 carbons, a substituted or unsubstituted aryl group having 6 to 30 ring-forming carbons, or a substituted or unsubstituted heteroaryl group having 2 to 30 ring-forming carbons,
z1, z2, z8, z10, and z12 are each independently an integer of 0 to 4,
z3, z5, z7, z9, z11, and z15 are each independently an integer of 0 to 3,
z4 and z6 are each independently an integer of 0 to 5, and
z13 and z14 are each independently an integer of 0 to 6.

12. The light-emitting element of claim 1, wherein the nitrogen-containing compound is represented by any one selected from among compounds in Compound Group 1:

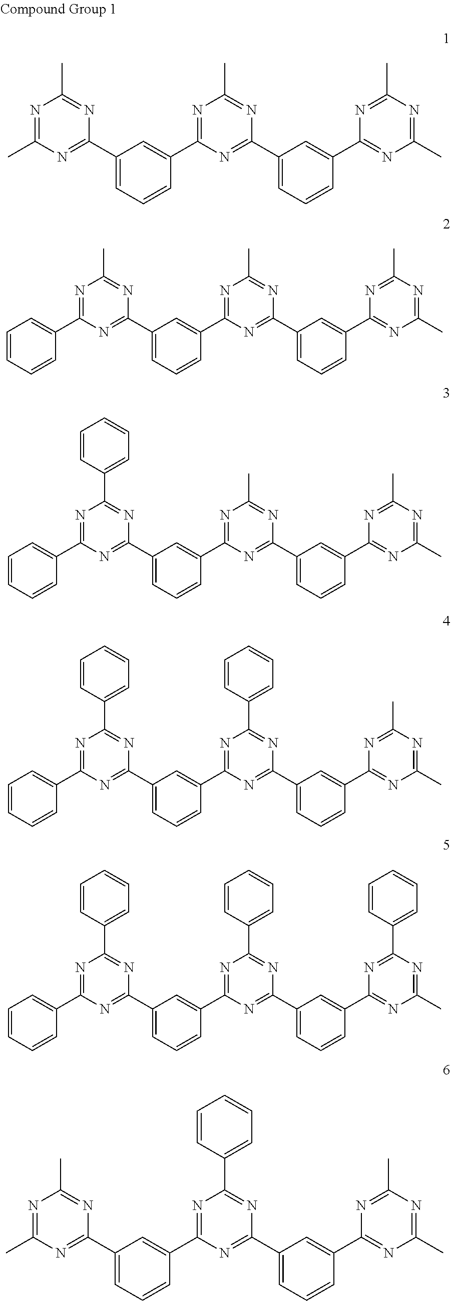

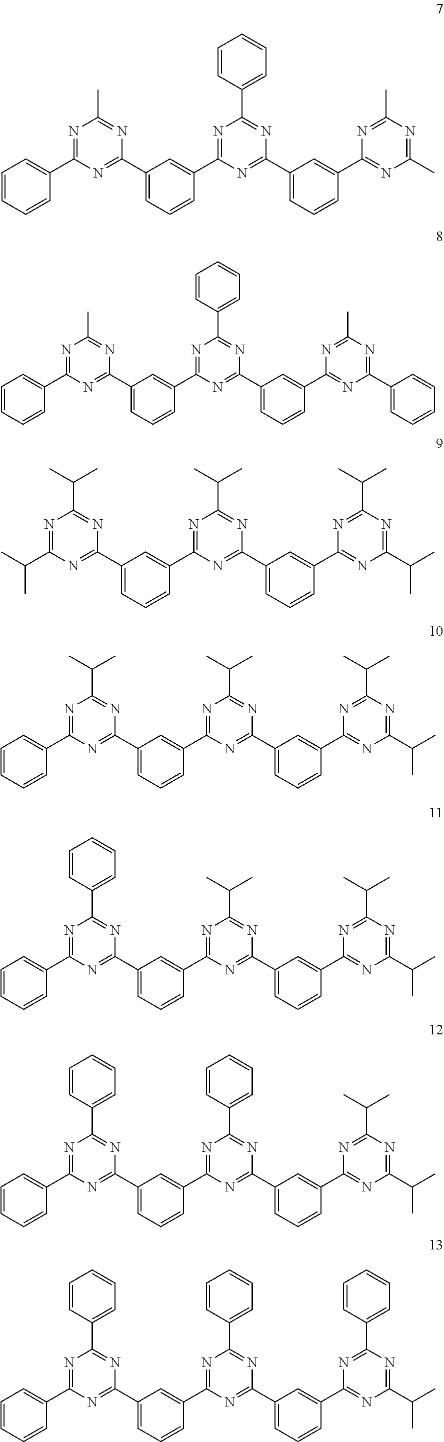

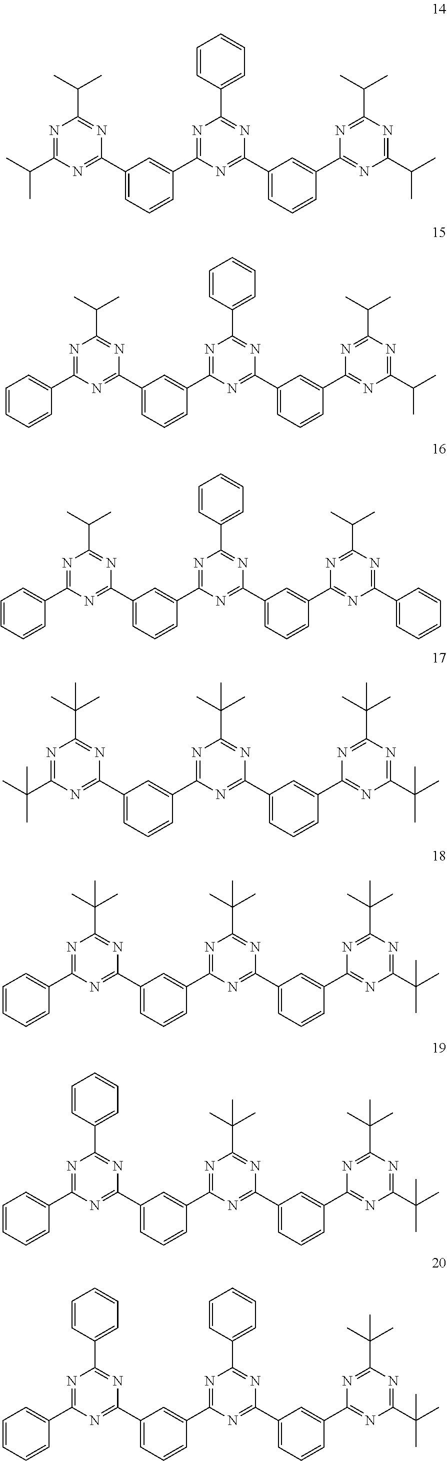

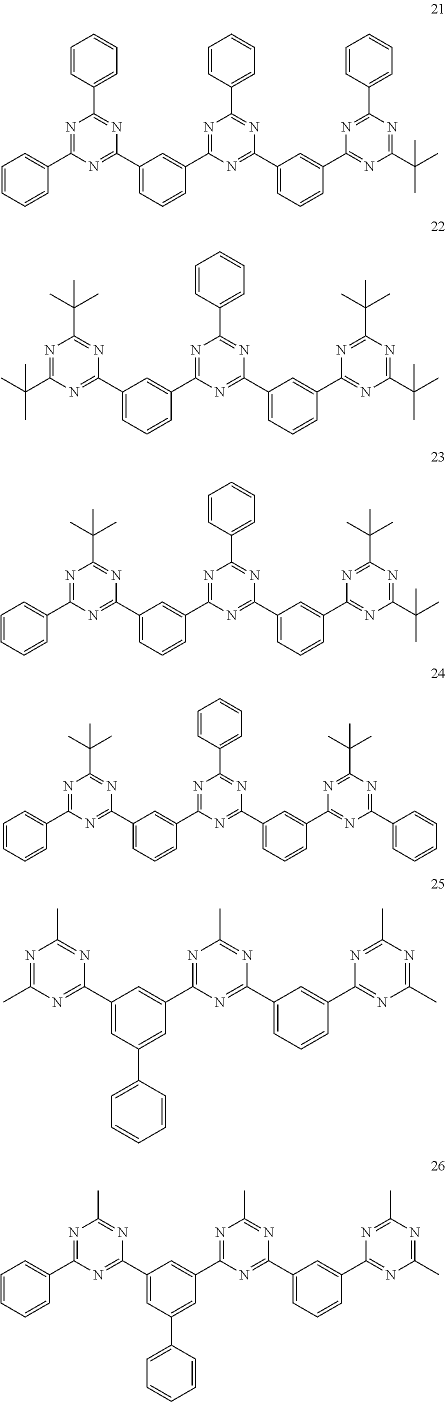

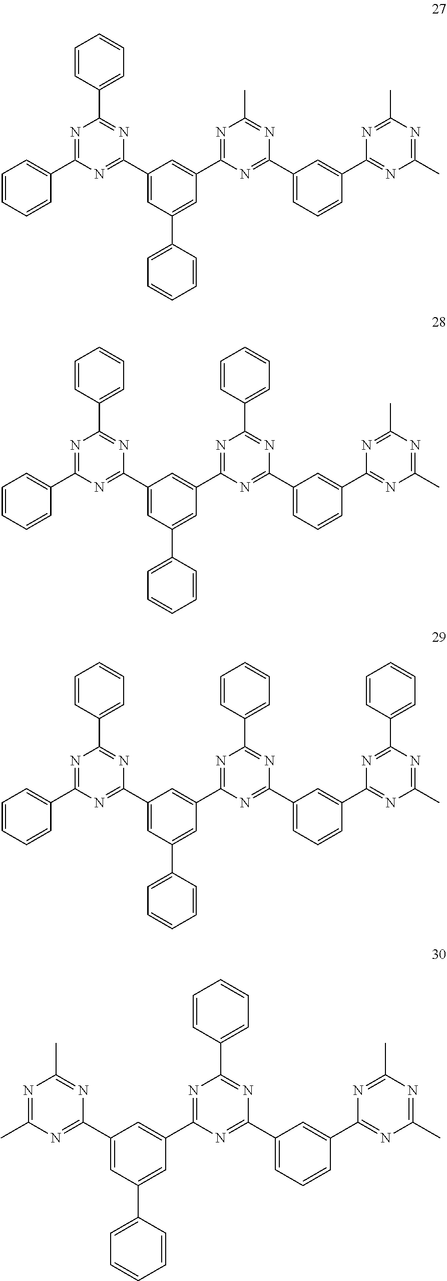

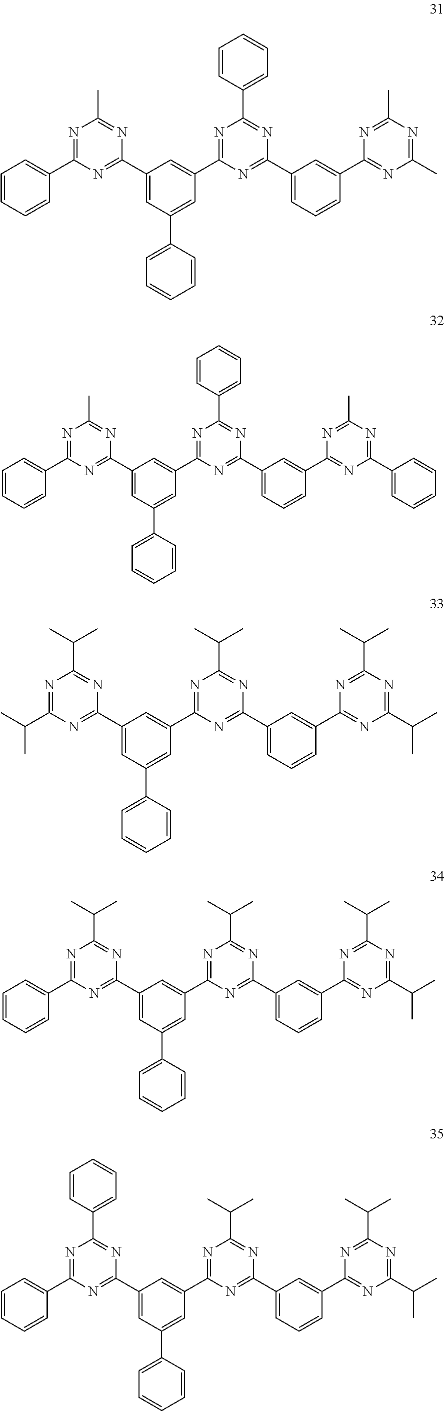

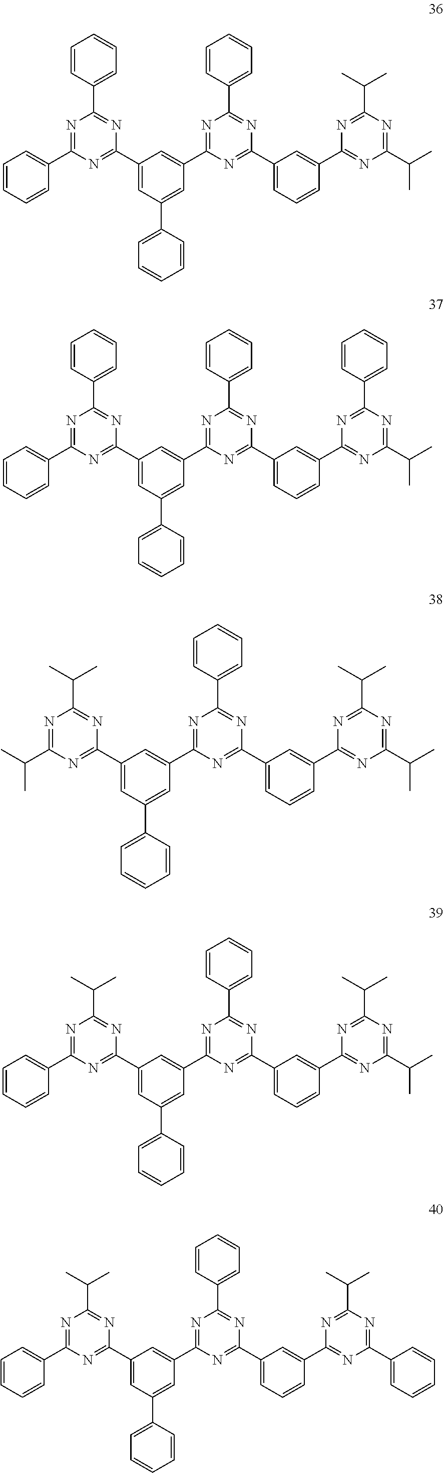

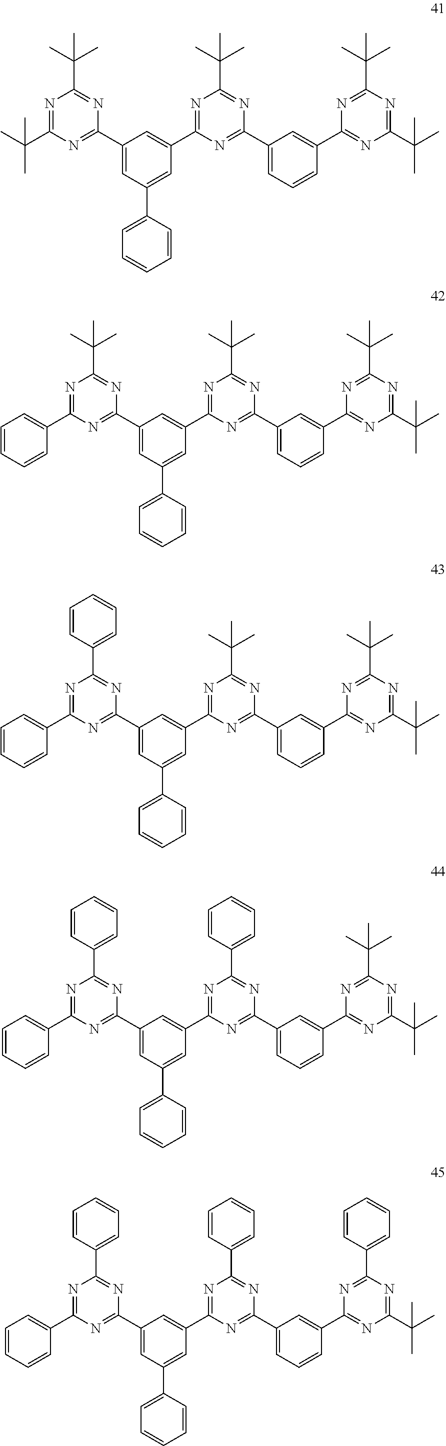

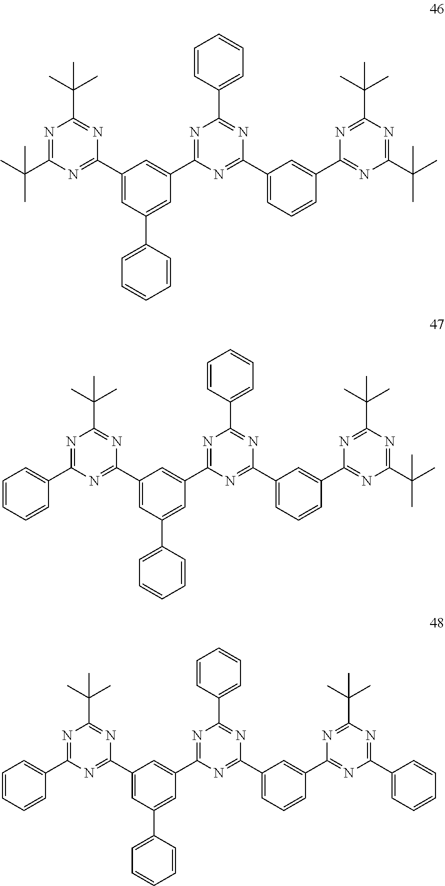 [SMILES: CC(C)(C)c1nc(-c2cccc(-c3nc(-c4ccccc4)nc(-c4cc(-c5ccccc5)cc(-c5nc(C(C)(C)C)nc(C(C)(C)C)n5)c4)n3)c2)nc(C(C)(C)C)n1 ; CC(C)(C)c1nc(-c2ccccc2)nc(-c2cc(-c3ccccc3)cc(-c3nc(-c4ccccc4)nc(-c4cccc(-c5nc(C(C)(C)C)nc(C(C)(C)C)n5)c4)n3)c2)n1 ; CC(C)(C)c1nc(-c2ccccc2)nc(-c2cccc(-c3nc(-c4ccccc4)nc(-c4cc(-c5ccccc5)cc(-c5nc(-c6ccccc6)nc(C(C)(C)C)n5)c4)n3)c2)n1]

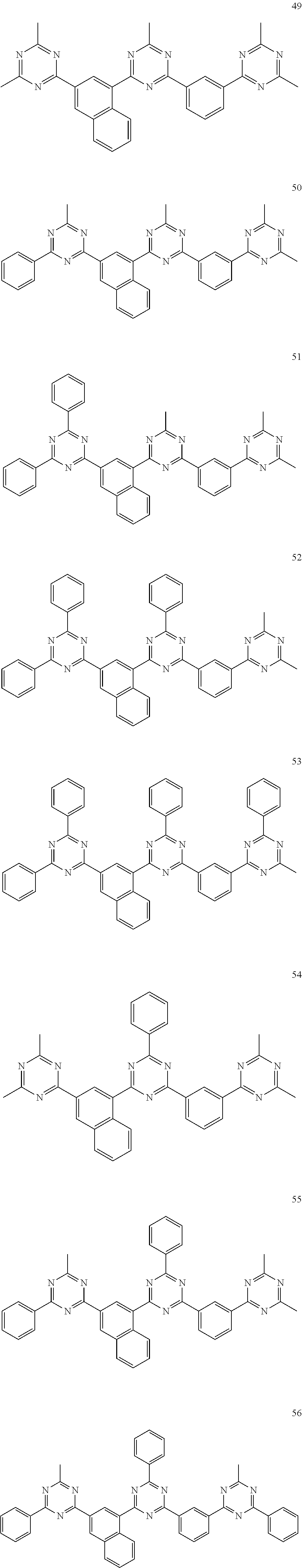

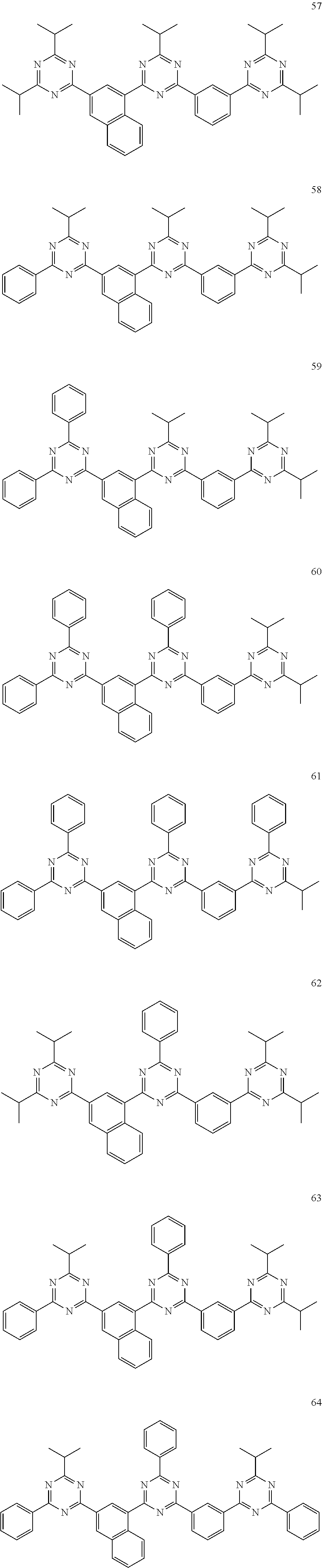

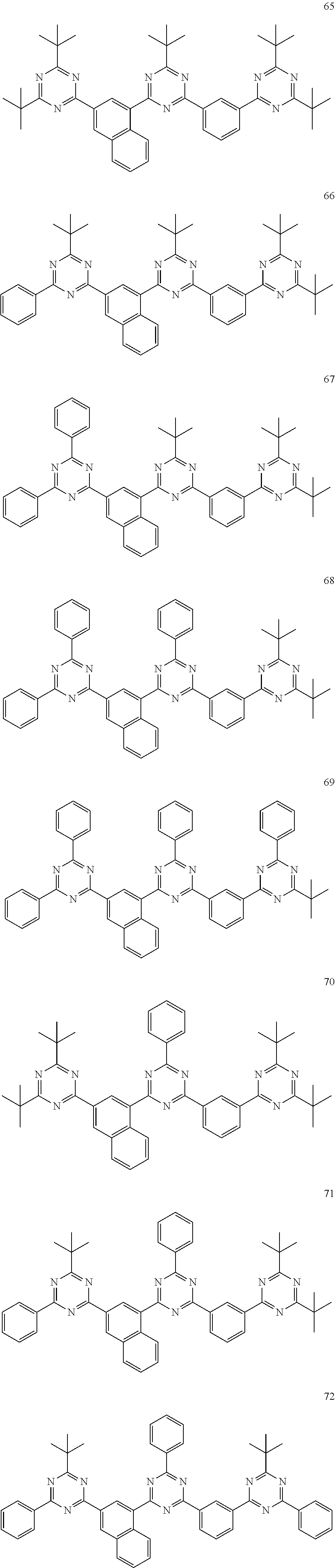

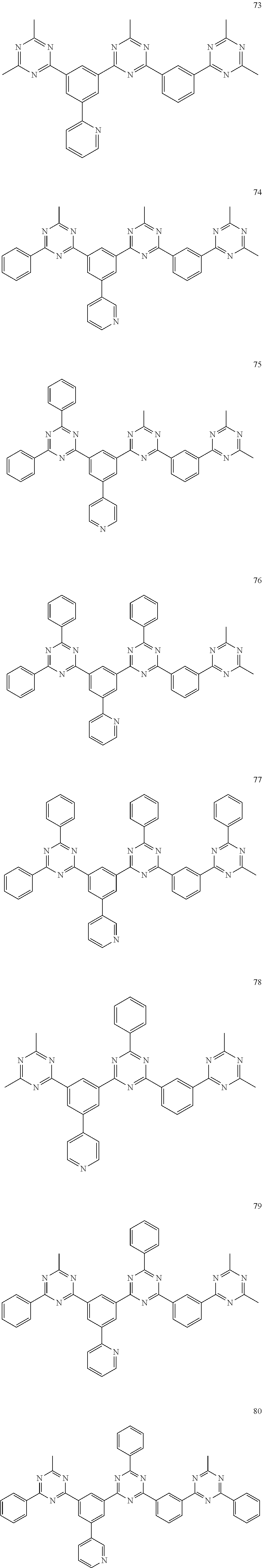

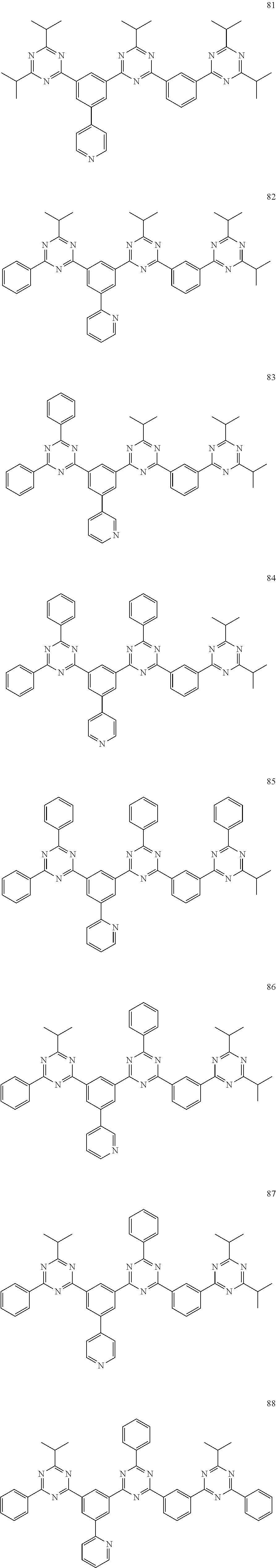

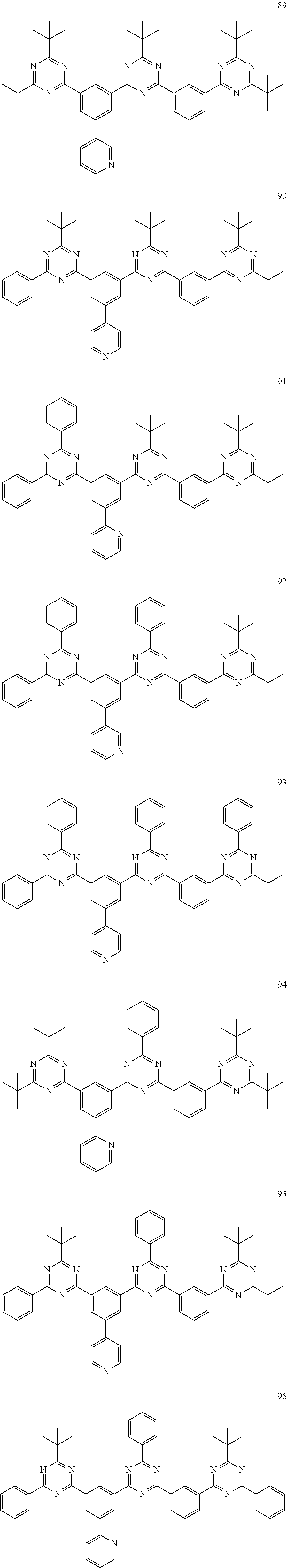

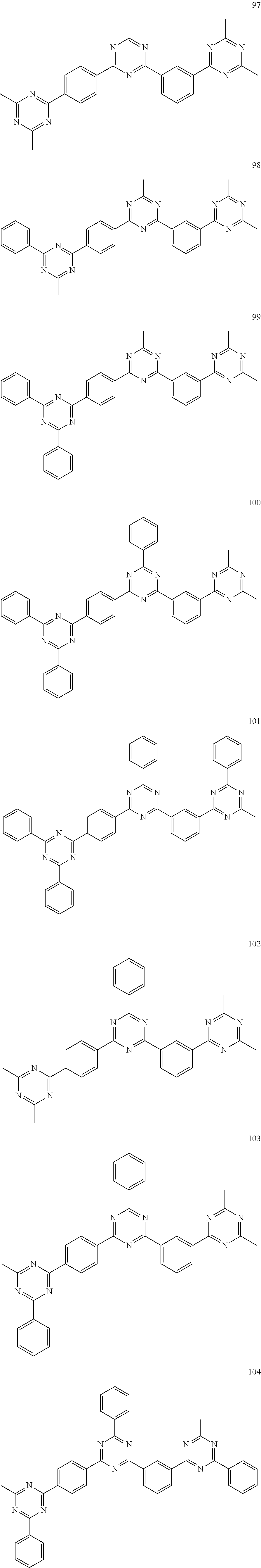

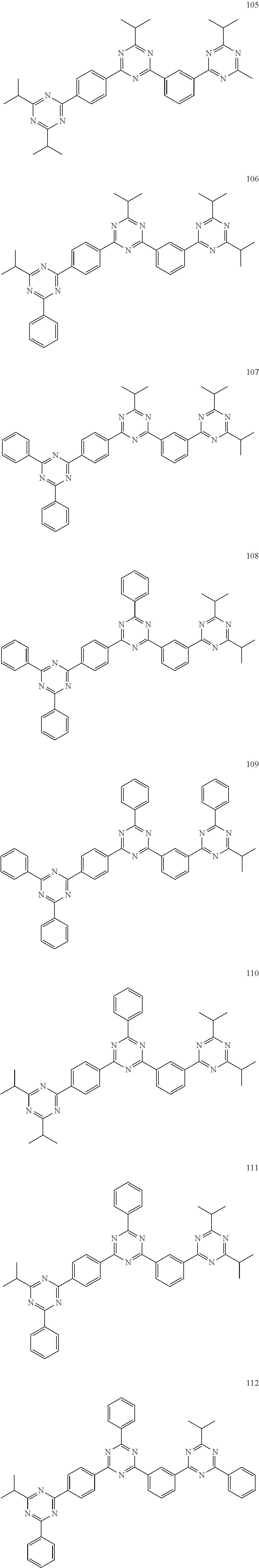

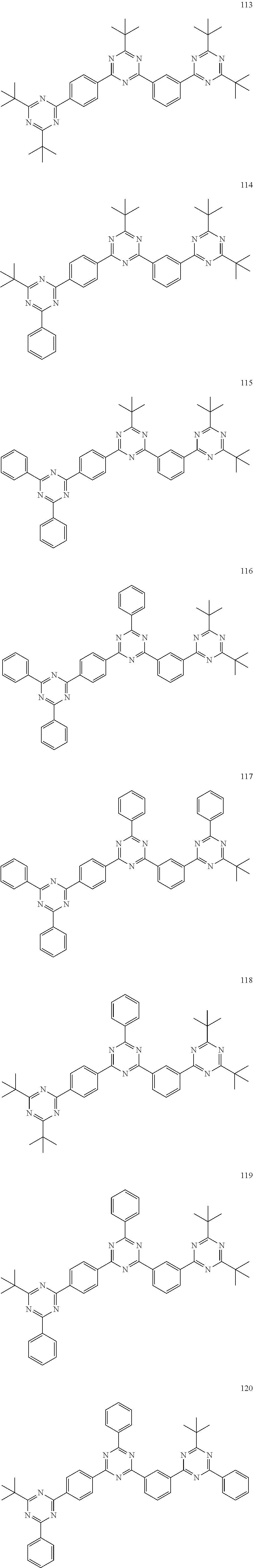

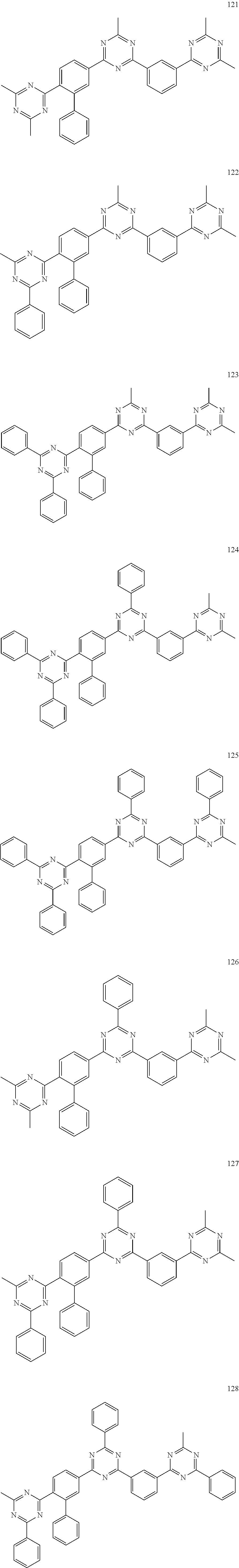

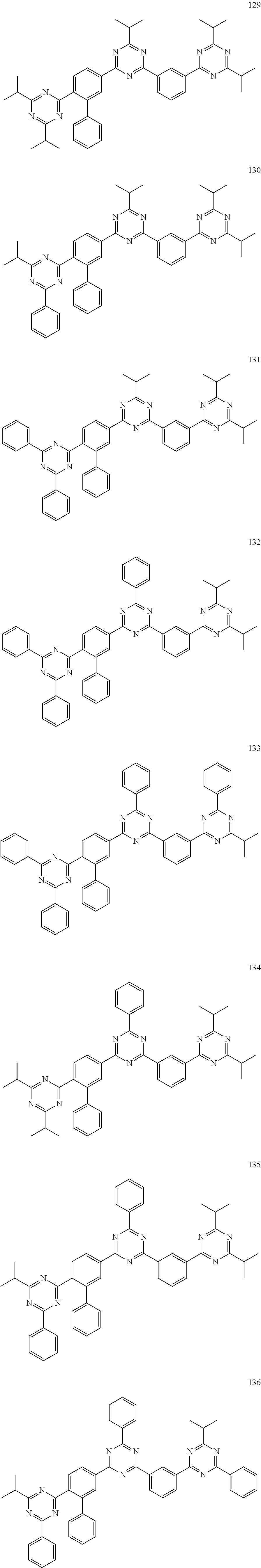

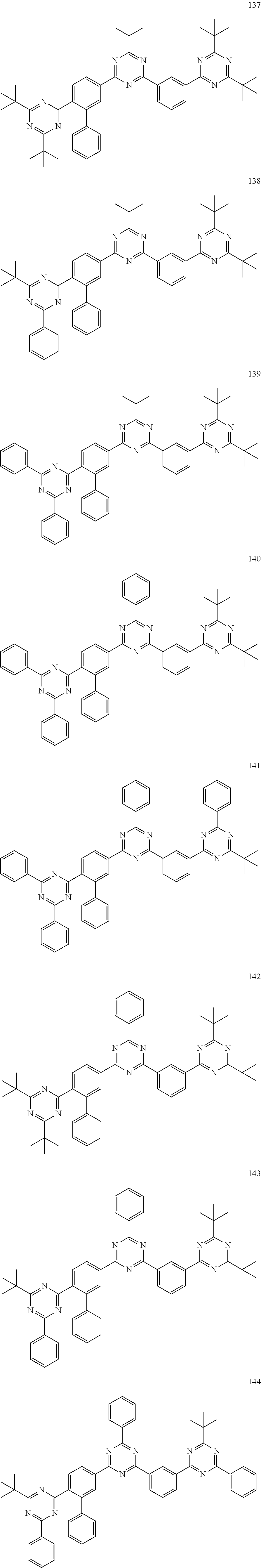

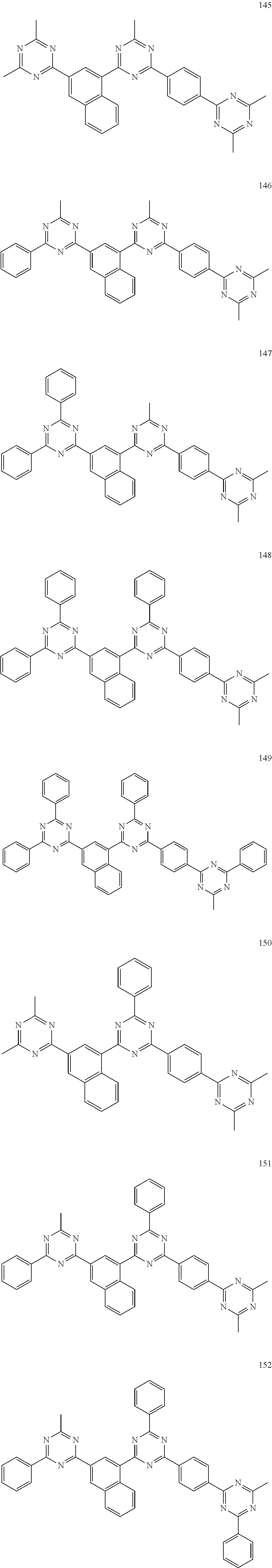

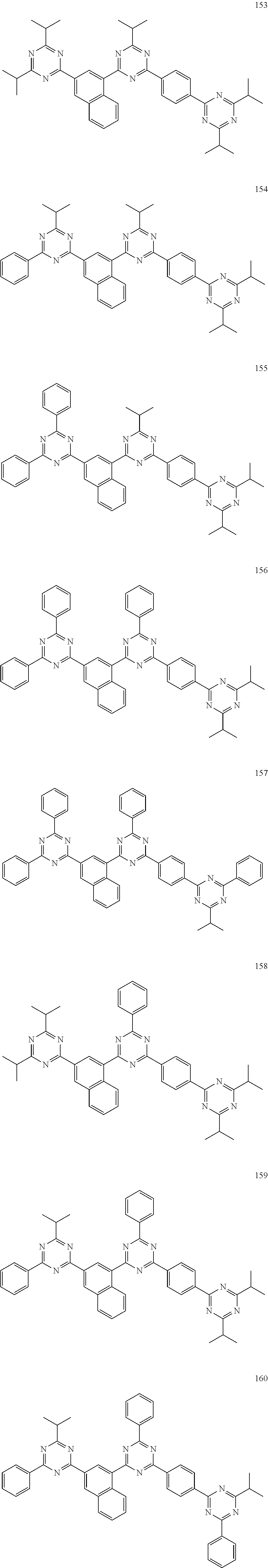

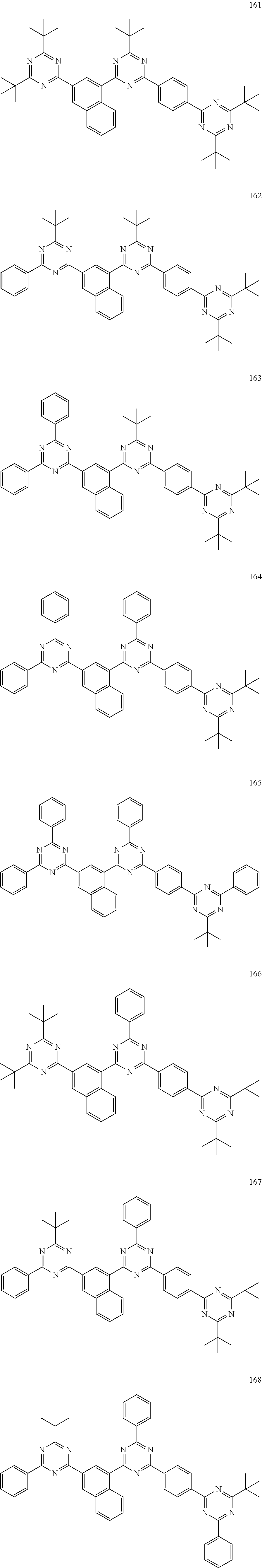

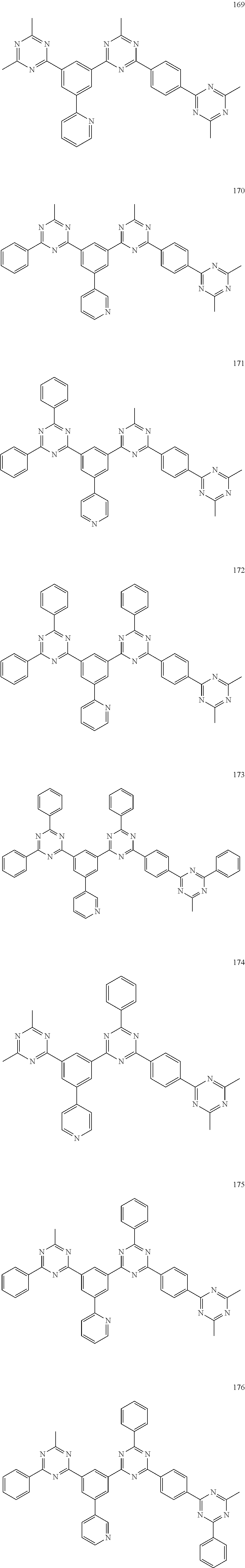

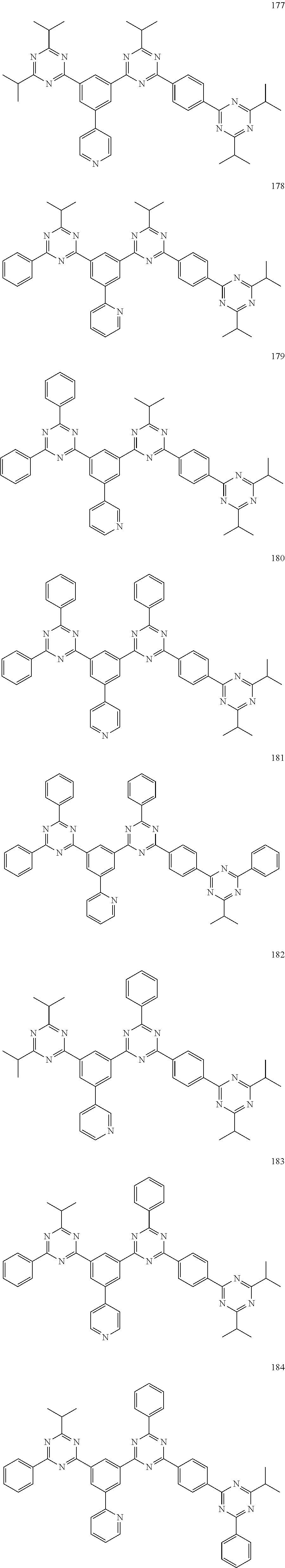

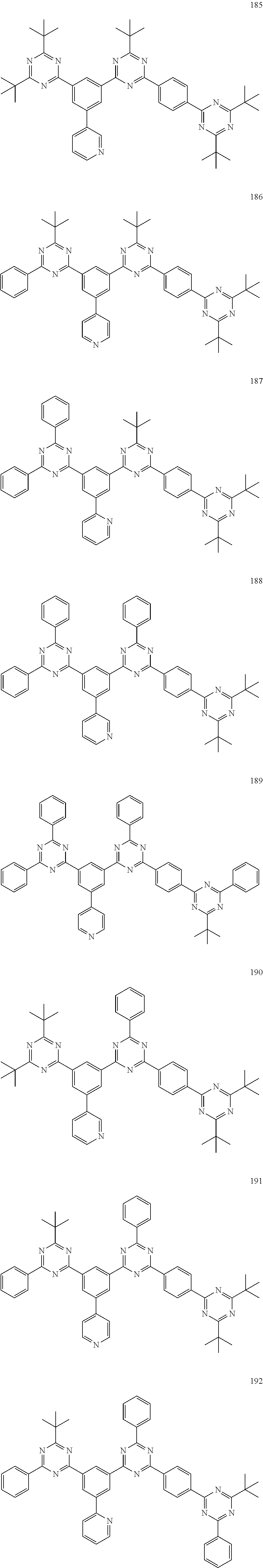

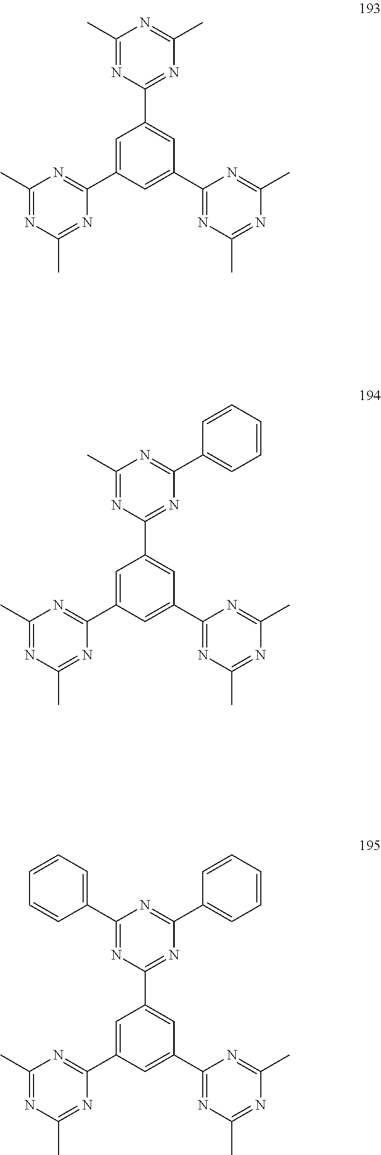

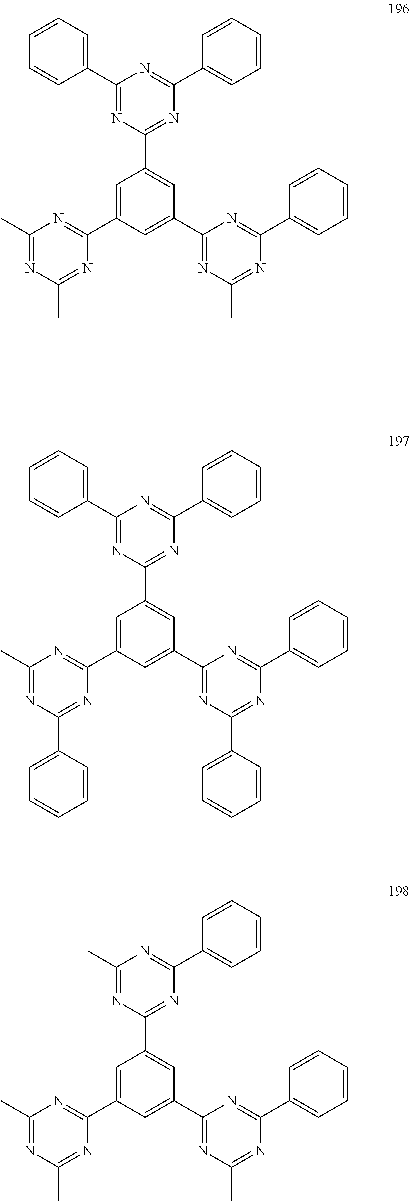 [SMILES: Cc1nc(C)nc(-c2cc(-c3nc(C)nc(-c4ccccc4)n3)cc(-c3nc(-c4ccccc4)nc(-c4ccccc4)n3)c2)n1 ; Cc1nc(-c2ccccc2)nc(-c2cc(-c3nc(-c4ccccc4)nc(-c4ccccc4)n3)cc(-c3nc(-c4ccccc4)nc(-c4ccccc4)n3)c2)n1 ; Cc1nc(C)nc(-c2cc(-c3nc(C)nc(-c4ccccc4)n3)cc(-c3nc(C)nc(-c4ccccc4)n3)c2)n1]

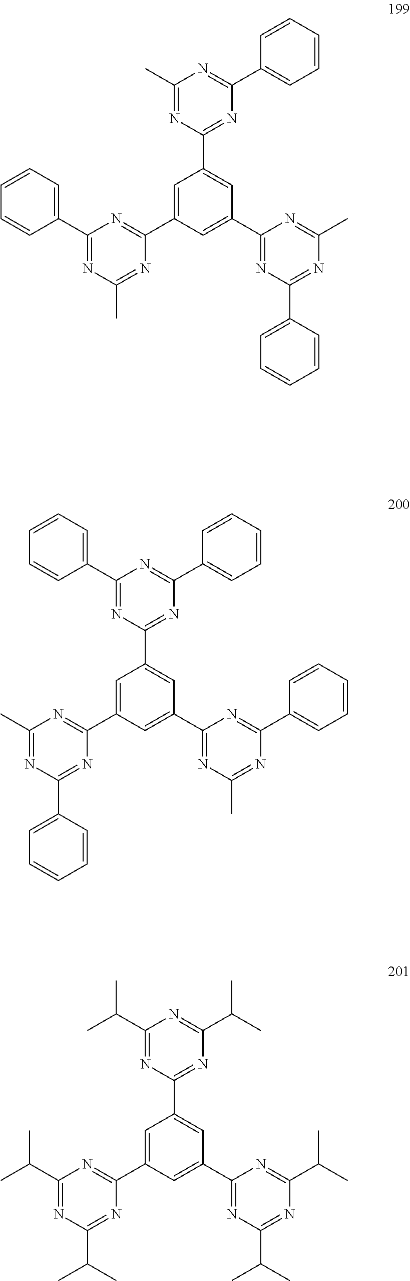

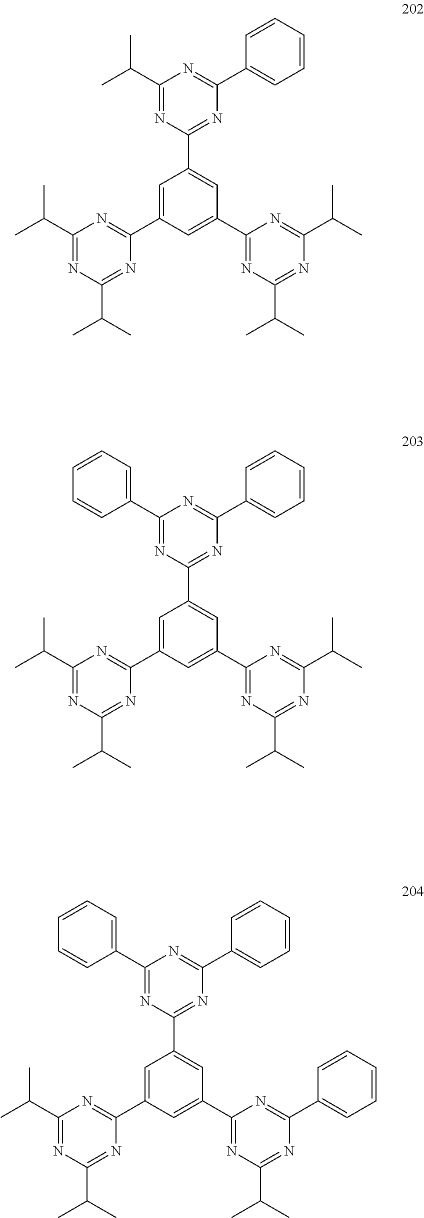

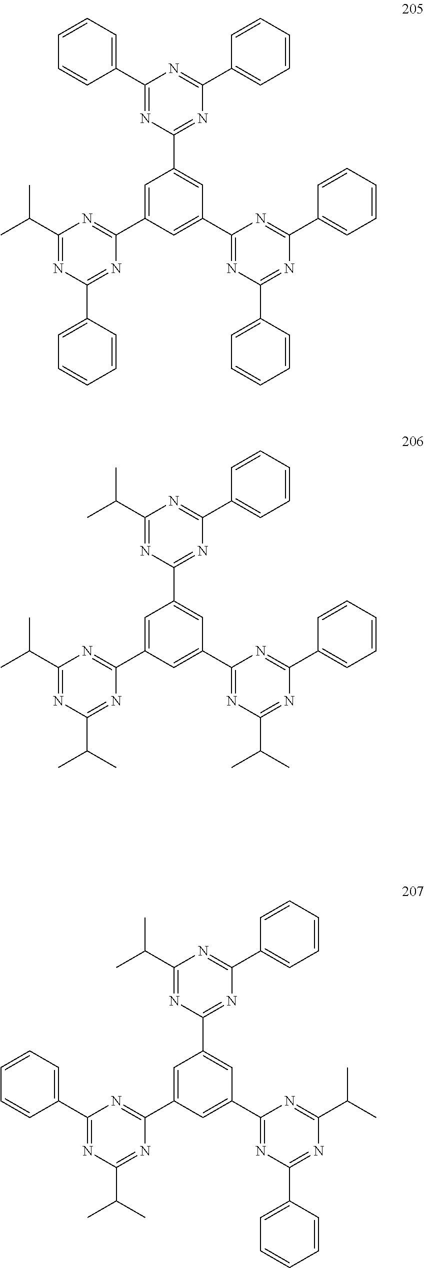 [SMILES: CC(C)c1nc(-c2ccccc2)nc(-c2cc(-c3nc(-c4ccccc4)nc(-c4ccccc4)n3)cc(-c3nc(-c4ccccc4)nc(-c4ccccc4)n3)c2)n1 ; CC(C)c1nc(-c2ccccc2)nc(-c2cc(-c3nc(-c4ccccc4)nc(C(C)C)n3)cc(-c3nc(C(C)C)nc(C(C)C)n3)c2)n1 ; CC(C)c1nc(-c2ccccc2)nc(-c2cc(-c3nc(-c4ccccc4)nc(C(C)C)n3)cc(-c3nc(-c4ccccc4)nc(C(C)C)n3)c2)n1]

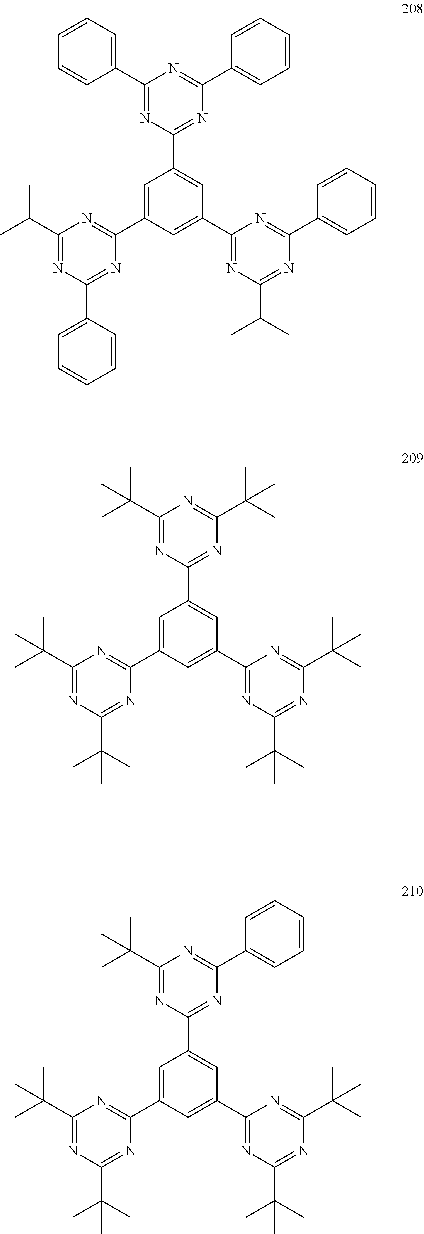

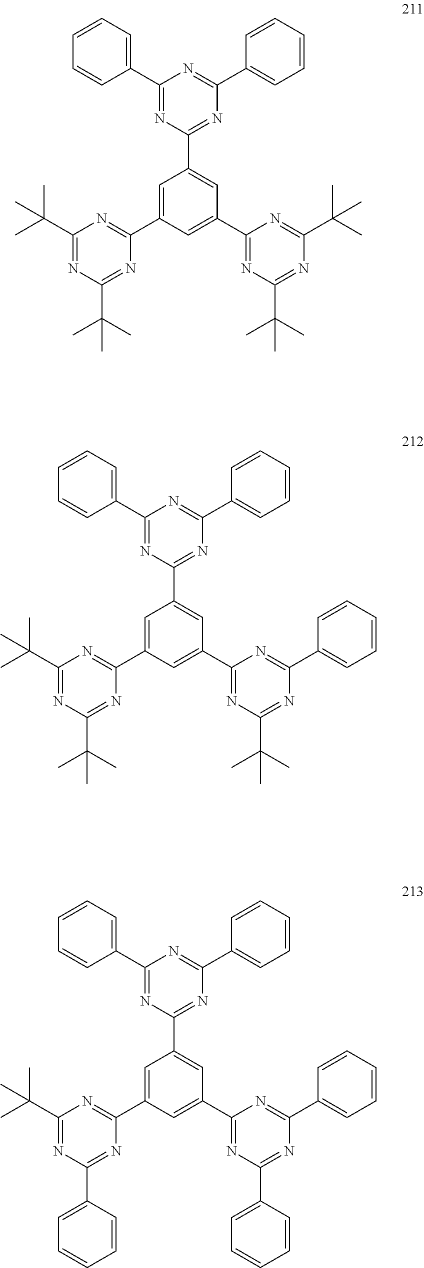

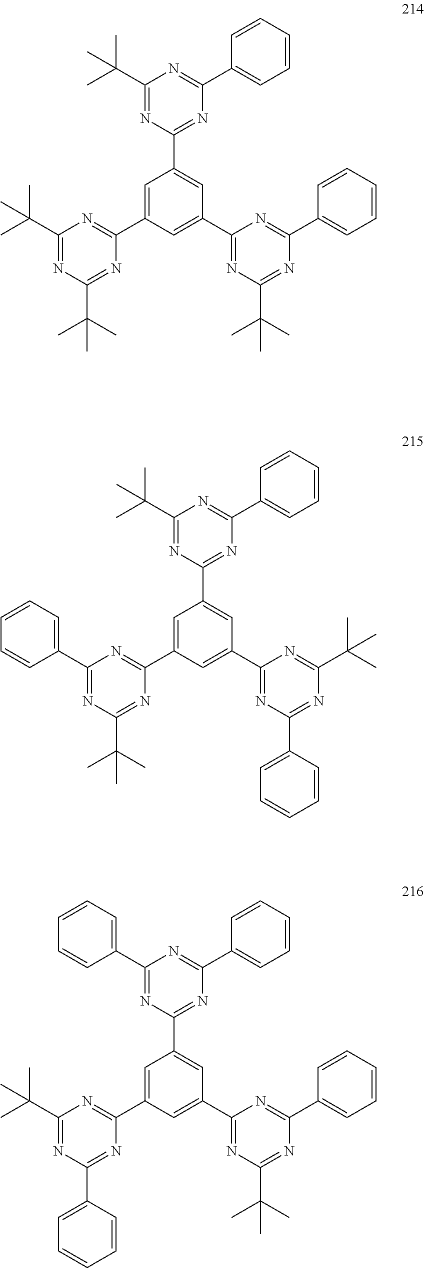

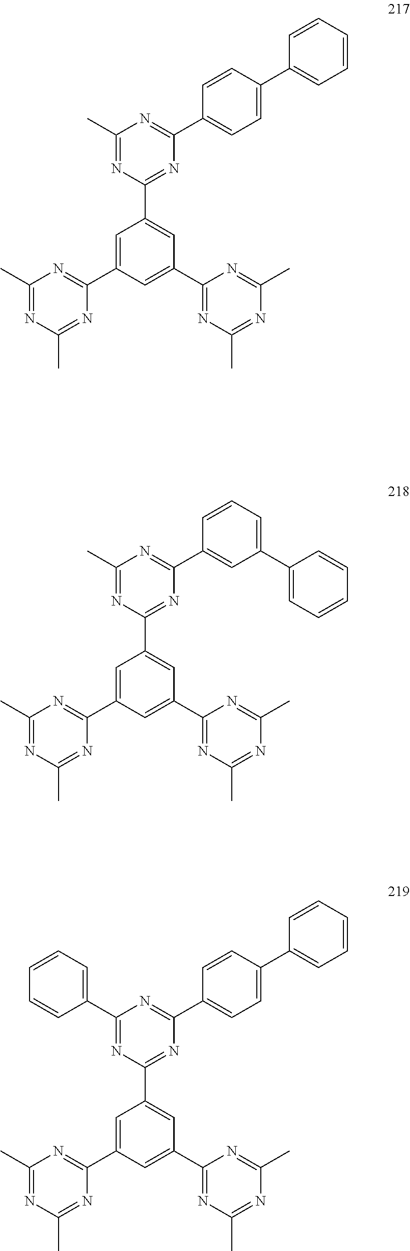 [SMILES: Cc1nc(C)nc(-c2cc(-c3nc(C)nc(C)n3)cc(-c3nc(C)nc(-c4ccc(-c5ccccc5)cc4)n3)c2)n1 ; Cc1nc(C)nc(-c2cc(-c3nc(C)nc(C)n3)cc(-c3nc(C)nc(-c4cccc(-c5ccccc5)c4)n3)c2)n1 ; Cc1nc(C)nc(-c2cc(-c3nc(C)nc(C)n3)cc(-c3nc(-c4ccccc4)nc(-c4ccc(-c5ccccc5)cc4)n3)c2)n1]

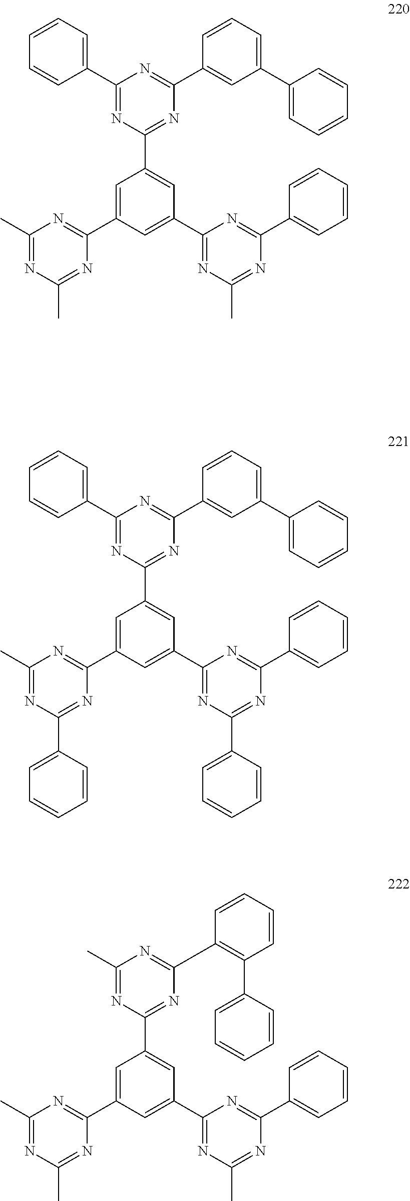 [SMILES: Cc1nc(C)nc(-c2cc(-c3nc(C)nc(-c4ccccc4)n3)cc(-c3nc(-c4ccccc4)nc(-c4cccc(-c5ccccc5)c4)n3)c2)n1 ; Cc1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1 ; Cc1nc(C)nc(-c2cc(-c3nc(C)nc(-c4ccccc4)n3)cc(-c3nc(C)nc(-c4ccccc4-c4ccccc4)n3)c2)n1]

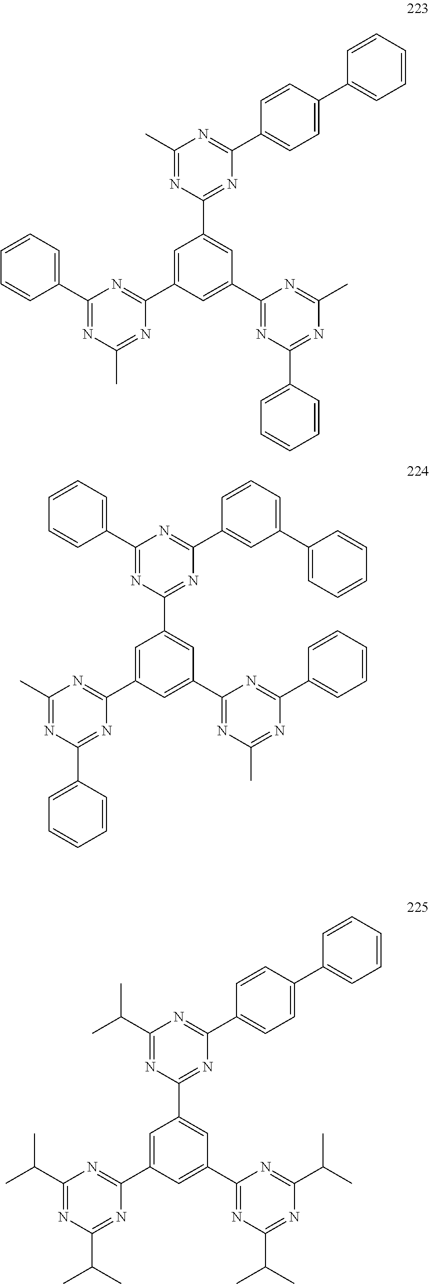 [SMILES: Cc1nc(-c2ccccc2)nc(-c2cc(-c3nc(C)nc(-c4ccccc4)n3)cc(-c3nc(C)nc(-c4ccc(-c5ccccc5)cc4)n3)c2)n1 ; Cc1nc(-c2ccccc2)nc(-c2cc(-c3nc(C)nc(-c4ccccc4)n3)cc(-c3nc(-c4ccccc4)nc(-c4cccc(-c5ccccc5)c4)n3)c2)n1 ; CC(C)c1nc(-c2ccc(-c3ccccc3)cc2)nc(-c2cc(-c3nc(C(C)C)nc(C(C)C)n3)cc(-c3nc(C(C)C)nc(C(C)C)n3)c2)n1]

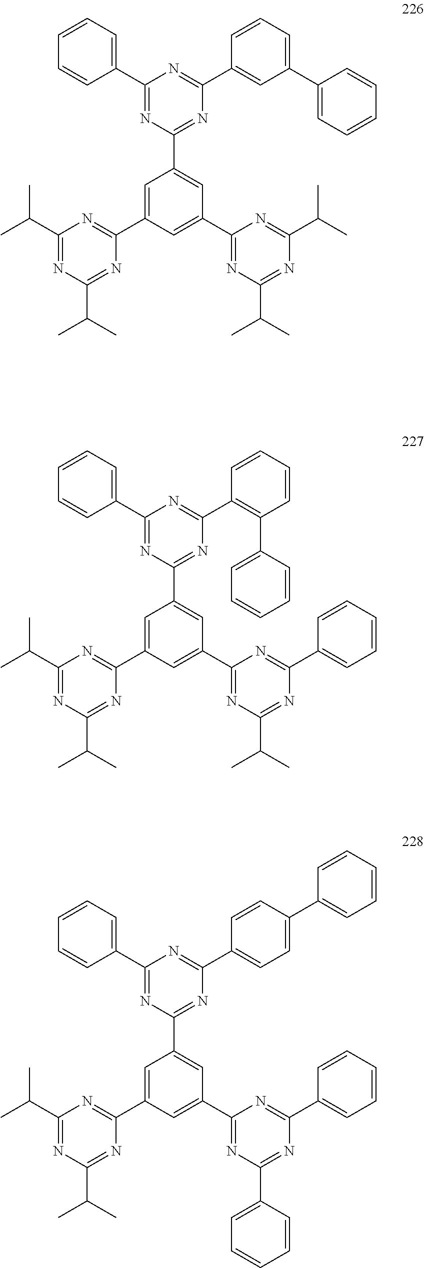 [SMILES: CC(C)c1nc(-c2cc(-c3nc(-c4ccccc4)nc(-c4cccc(-c5ccccc5)c4)n3)cc(-c3nc(C(C)C)nc(C(C)C)n3)c2)nc(C(C)C)n1 ; CC(C)c1nc(-c2ccccc2)nc(-c2cc(-c3nc(-c4ccccc4)nc(-c4ccccc4-c4ccccc4)n3)cc(-c3nc(C(C)C)nc(C(C)C)n3)c2)n1 ; CC(C)c1nc(-c2cc(-c3nc(-c4ccccc4)nc(-c4ccccc4)n3)cc(-c3nc(-c4ccccc4)nc(-c4ccc(-c5ccccc5)cc4)n3)c2)nc(C(C)C)n1]

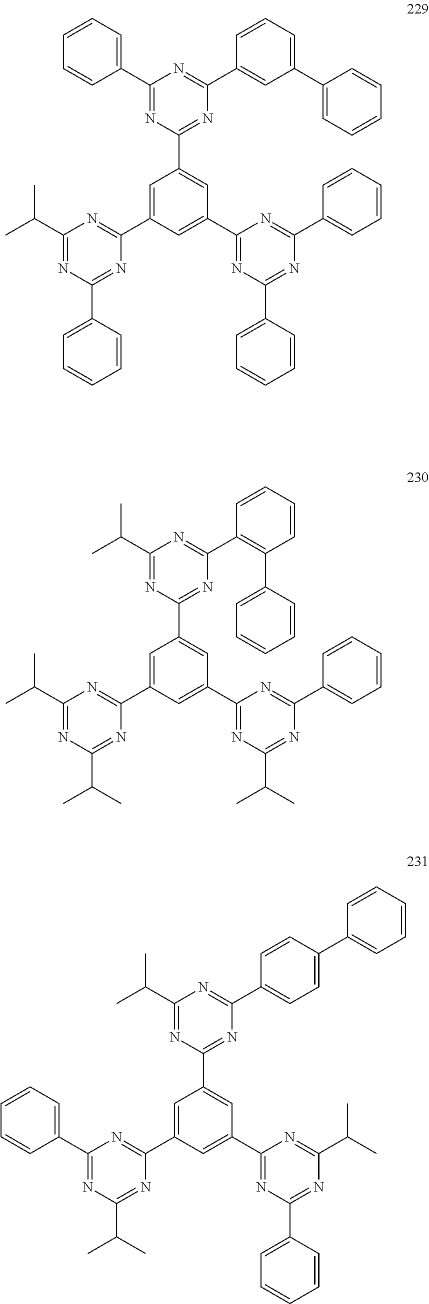 [SMILES: CC(C)c1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1 ; CC(C)c1nc(-c2ccccc2)nc(-c2cc(-c3nc(-c4ccccc4-c4ccccc4)nc(C(C)C)n3)cc(-c3nc(C(C)C)nc(C(C)C)n3)c2)n1 ; CC(C)c1nc(-c2ccccc2)nc(-c2cc(-c3nc(-c4ccccc4)nc(C(C)C)n3)cc(-c3nc(-c4ccc(-c5ccccc5)cc4)nc(C(C)C)n3)c2)n1]

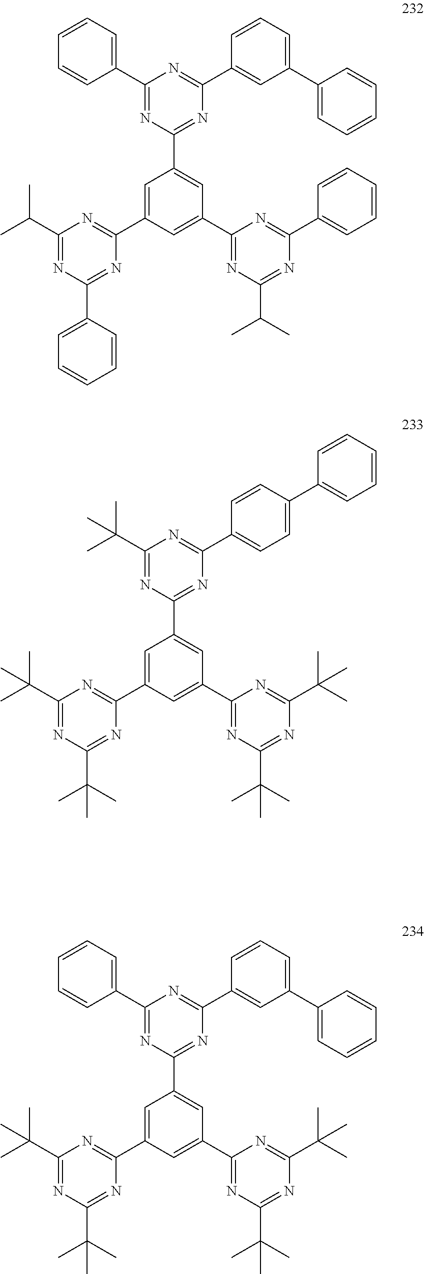 [SMILES: CC(C)c1nc(-c2ccccc2)nc(-c2cc(-c3nc(-c4ccccc4)nc(-c4cccc(-c5ccccc5)c4)n3)cc(-c3nc(-c4ccccc4)nc(C(C)C)n3)c2)n1 ; CC(C)(C)c1nc(-c2ccc(-c3ccccc3)cc2)nc(-c2cc(-c3nc(C(C)(C)C)nc(C(C)(C)C)n3)cc(-c3nc(C(C)(C)C)nc(C(C)(C)C)n3)c2)n1 ; CC(C)(C)c1nc(-c2cc(-c3nc(-c4ccccc4)nc(-c4cccc(-c5ccccc5)c4)n3)cc(-c3nc(C(C)(C)C)nc(C(C)(C)C)n3)c2)nc(C(C)(C)C)n1]

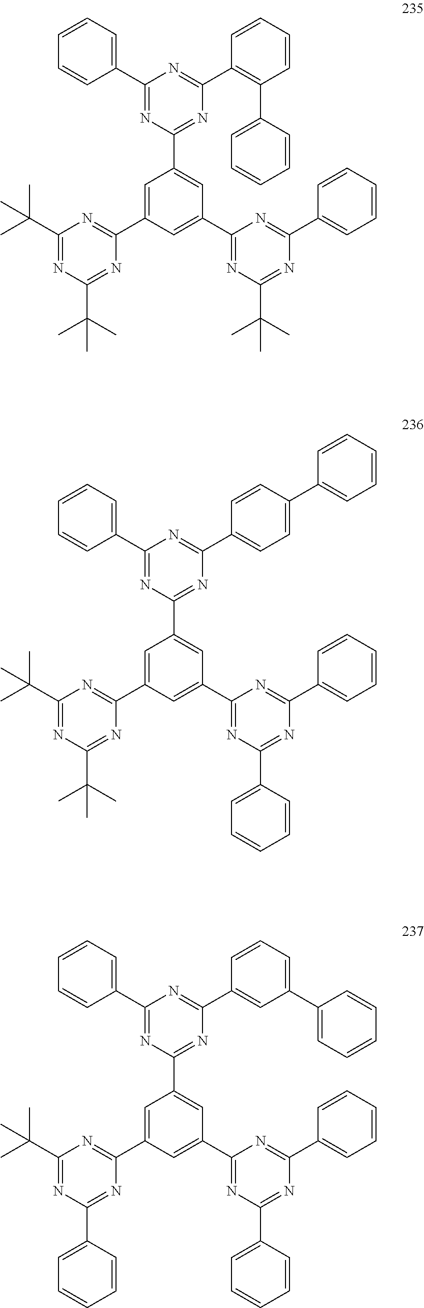 [SMILES: CC(C)(C)c1nc(-c2ccccc2)nc(-c2cc(-c3nc(-c4ccccc4)nc(-c4ccccc4-c4ccccc4)n3)cc(-c3nc(C(C)(C)C)nc(C(C)(C)C)n3)c2)n1 ; CC(C)(C)c1nc(-c2cc(-c3nc(-c4ccccc4)nc(-c4ccccc4)n3)cc(-c3nc(-c4ccccc4)nc(-c4ccc(-c5ccccc5)cc4)n3)c2)nc(C(C)(C)C)n1 ; CC(C)(C)c1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1]

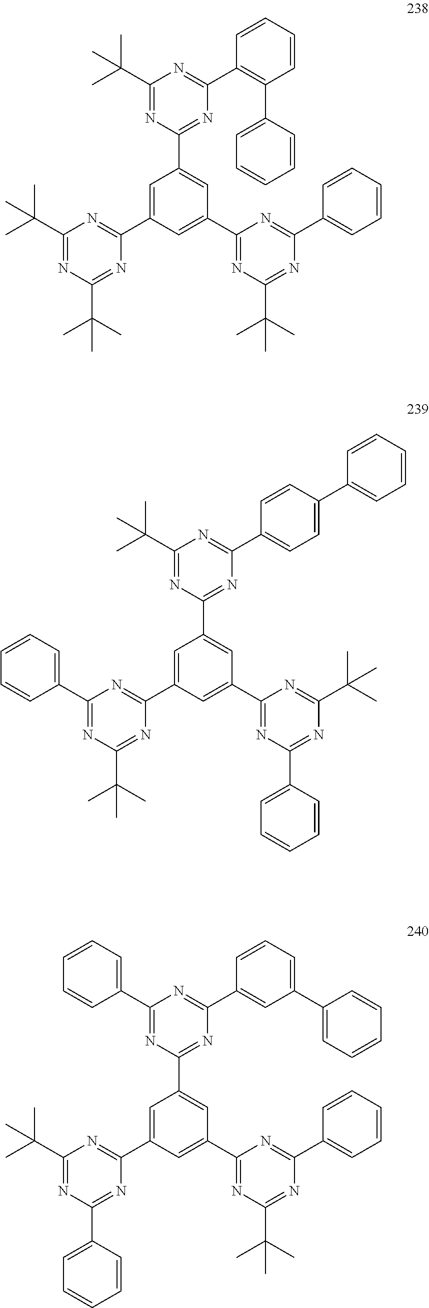 [SMILES: CC(C)(C)c1nc(-c2ccccc2)nc(-c2cc(-c3nc(-c4ccccc4-c4ccccc4)nc(C(C)(C)C)n3)cc(-c3nc(C(C)(C)C)nc(C(C)(C)C)n3)c2)n1 ; CC(C)(C)c1nc(-c2ccccc2)nc(-c2cc(-c3nc(-c4ccccc4)nc(C(C)(C)C)n3)cc(-c3nc(-c4ccc(-c5ccccc5)cc4)nc(C(C)(C)C)n3)c2)n1 ; CC(C)(C)c1nc(-c2ccccc2)nc(-c2cc(-c3nc(-c4ccccc4)nc(-c4cccc(-c5ccccc5)c4)n3)cc(-c3nc(-c4ccccc4)nc(C(C)(C)C)n3)c2)n1]

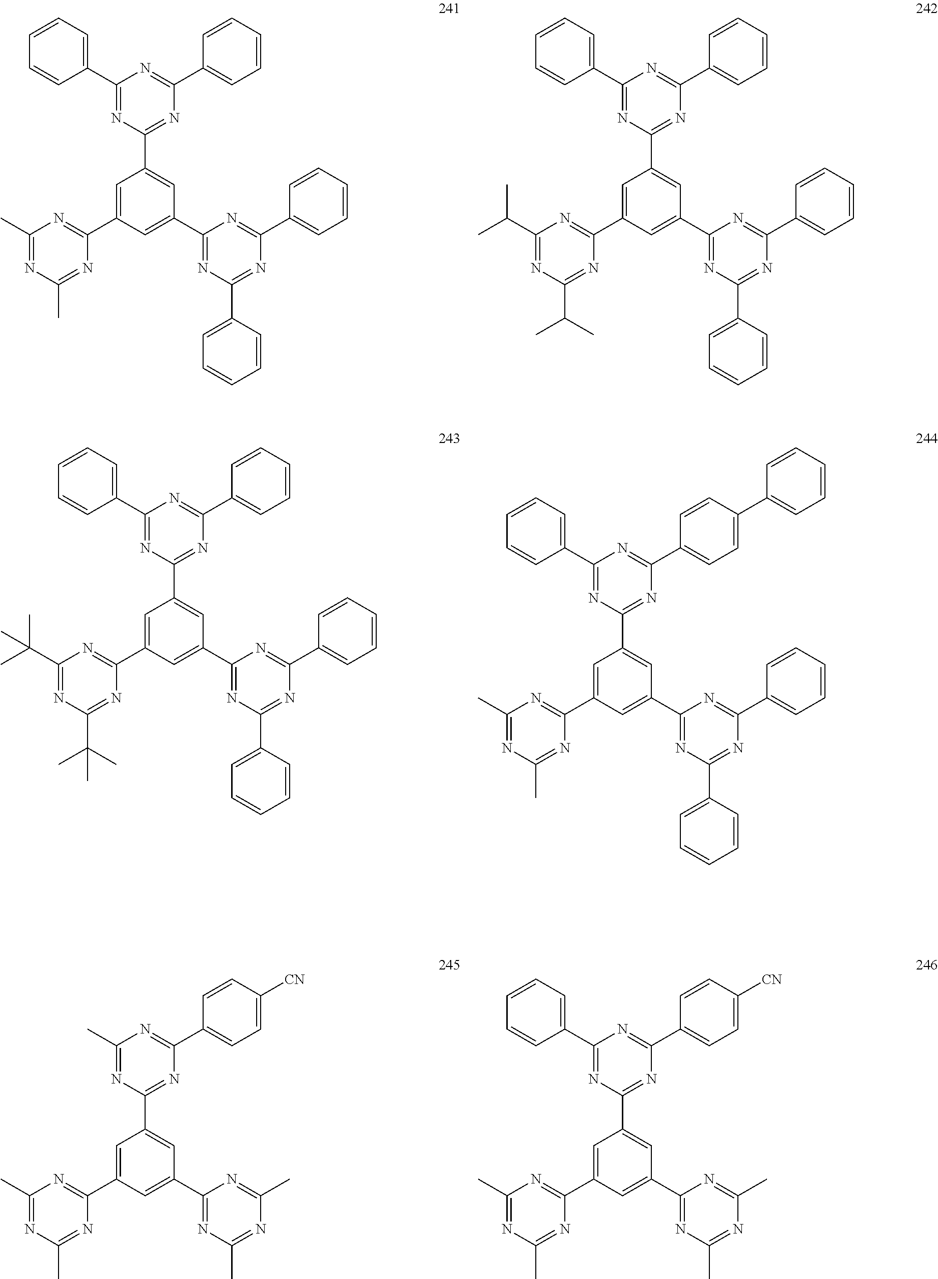

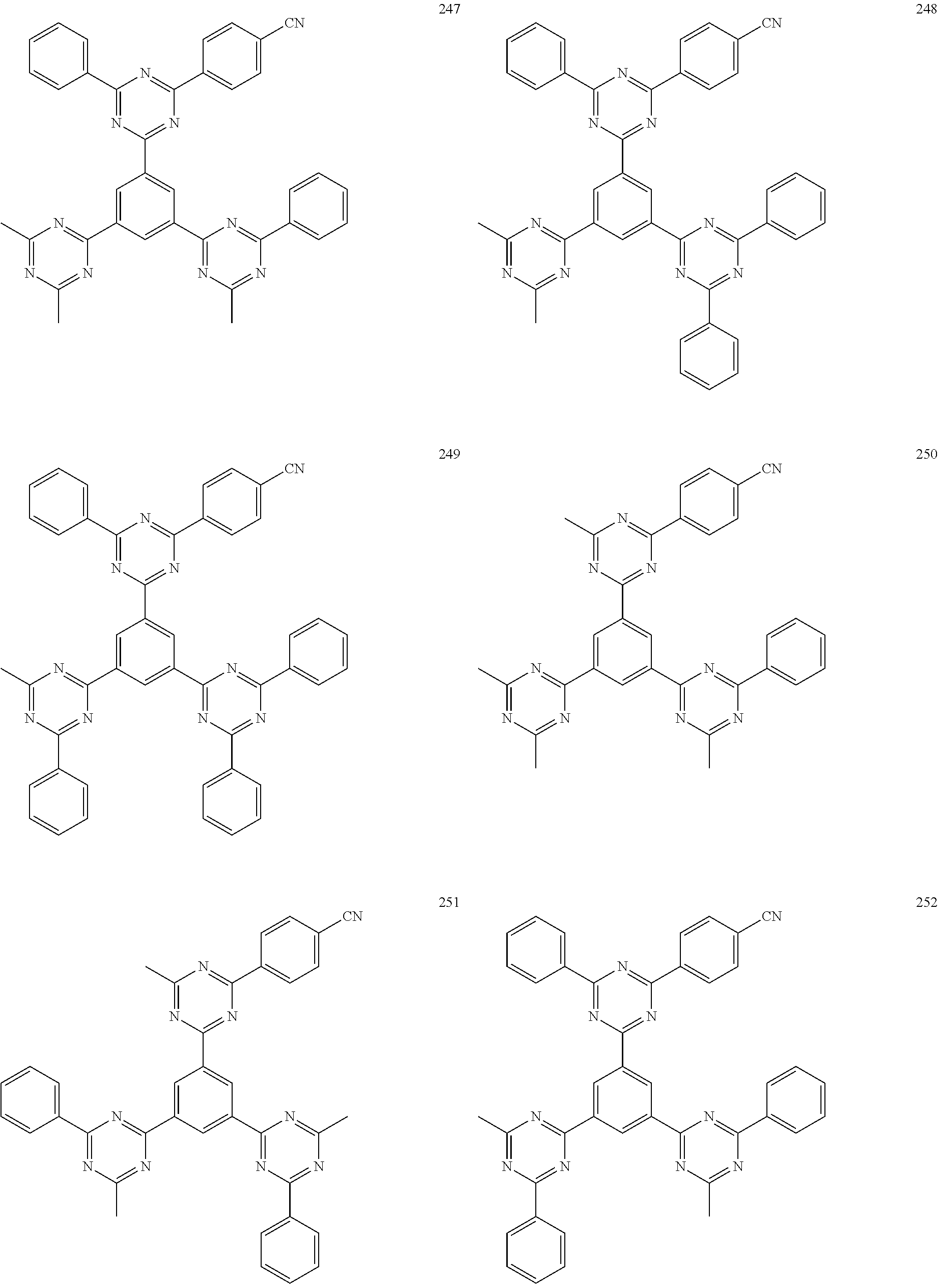

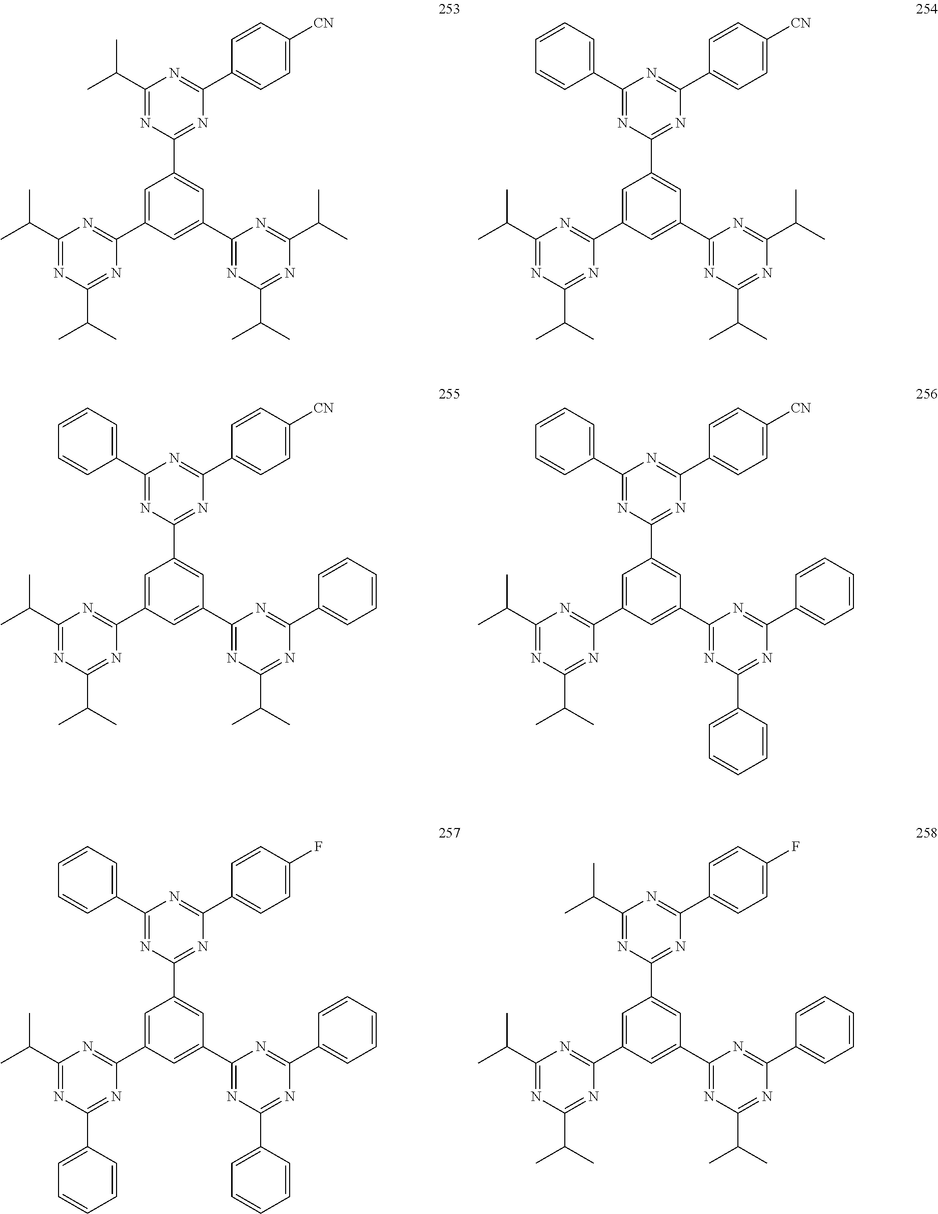

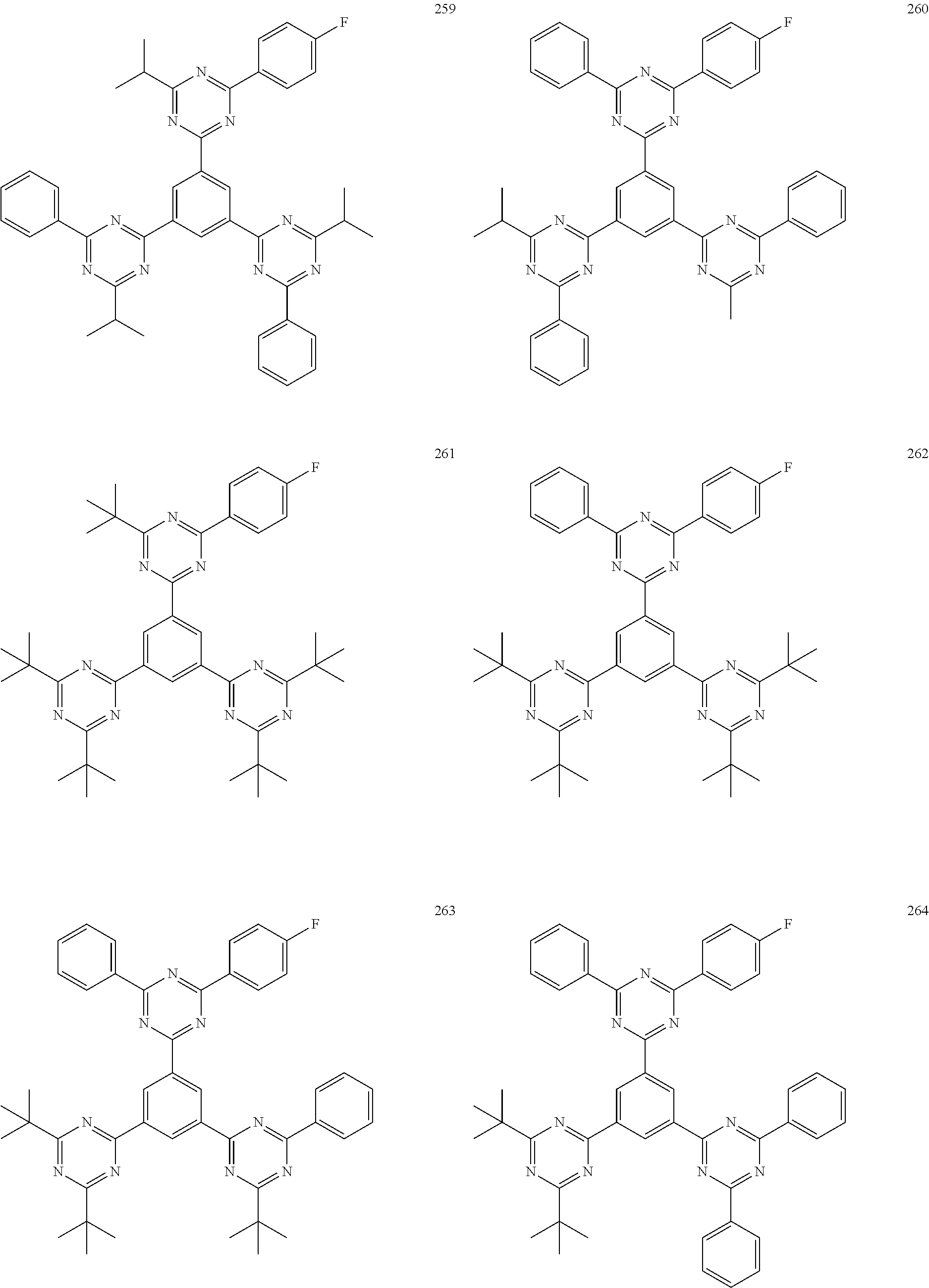

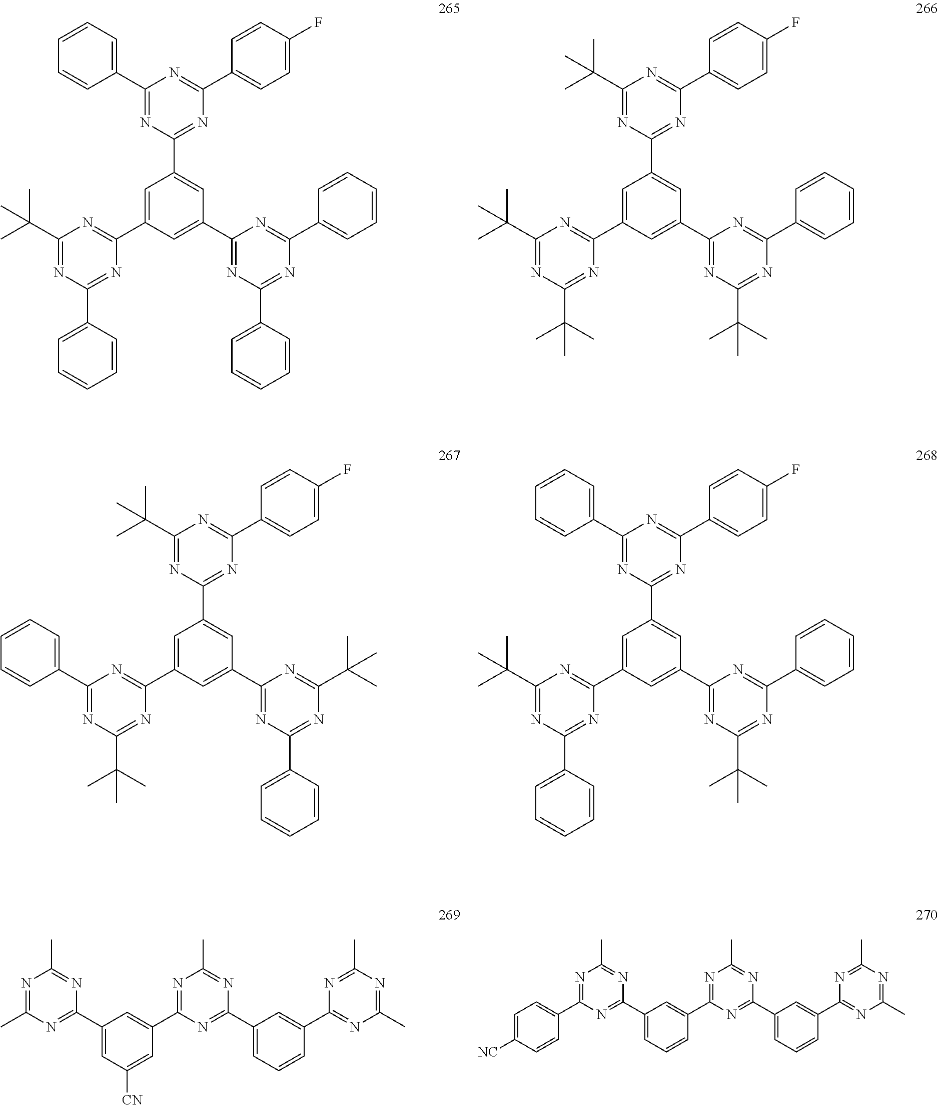

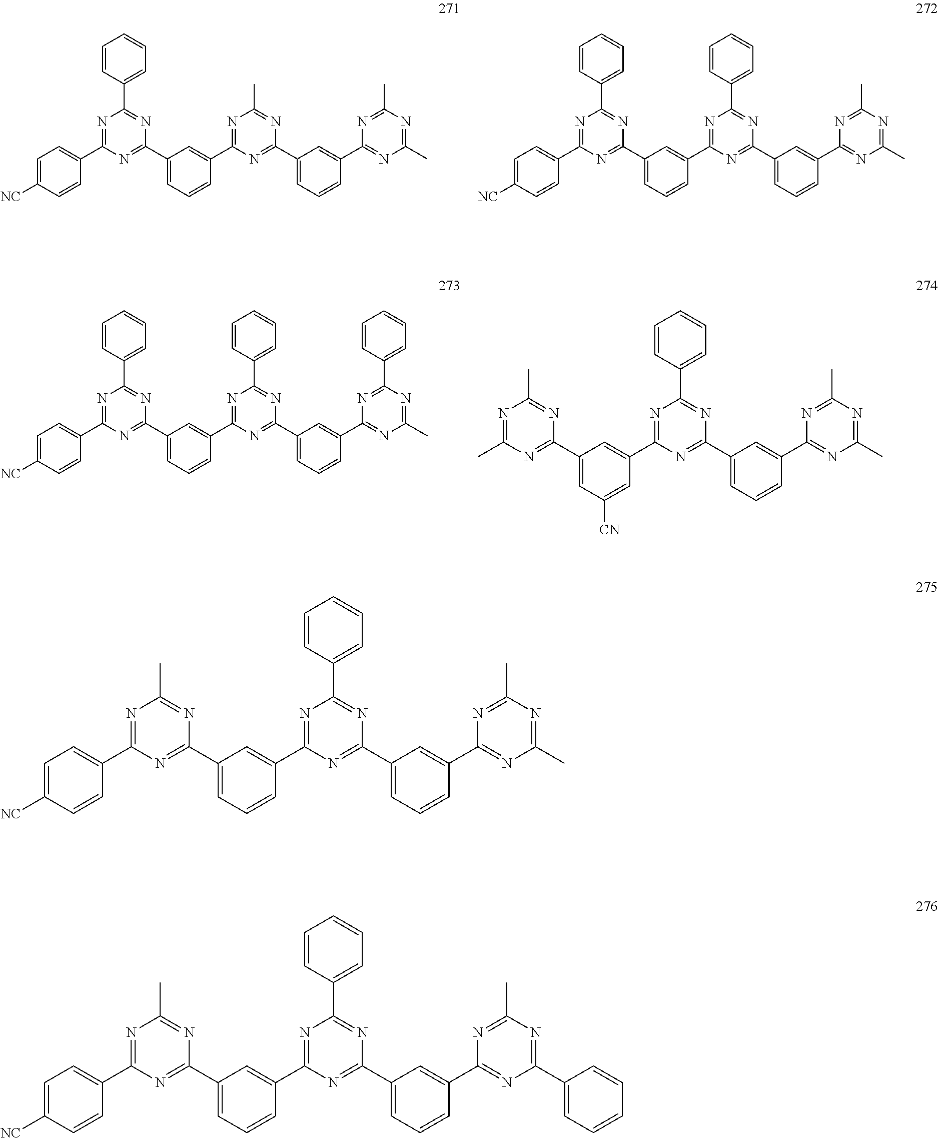

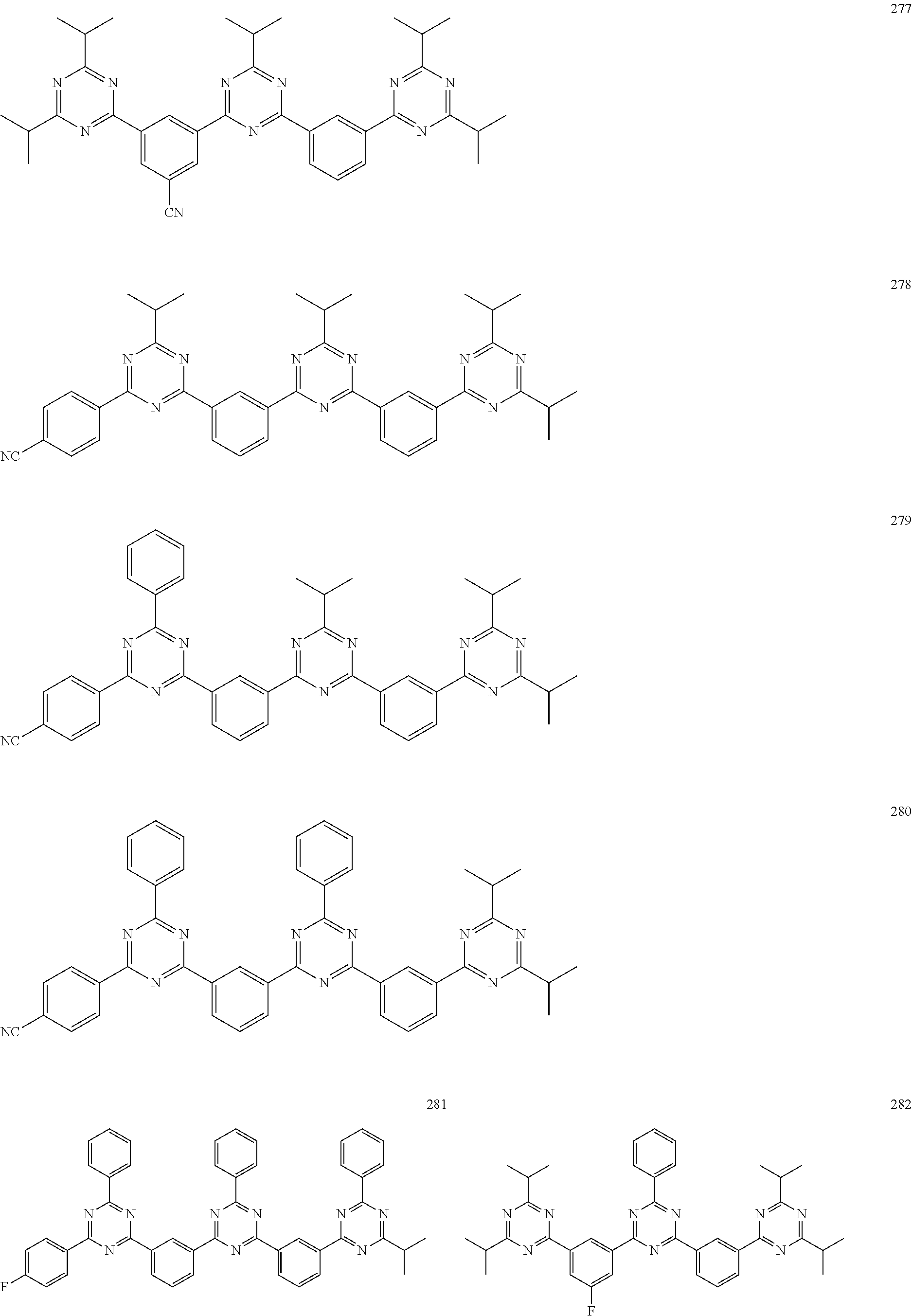

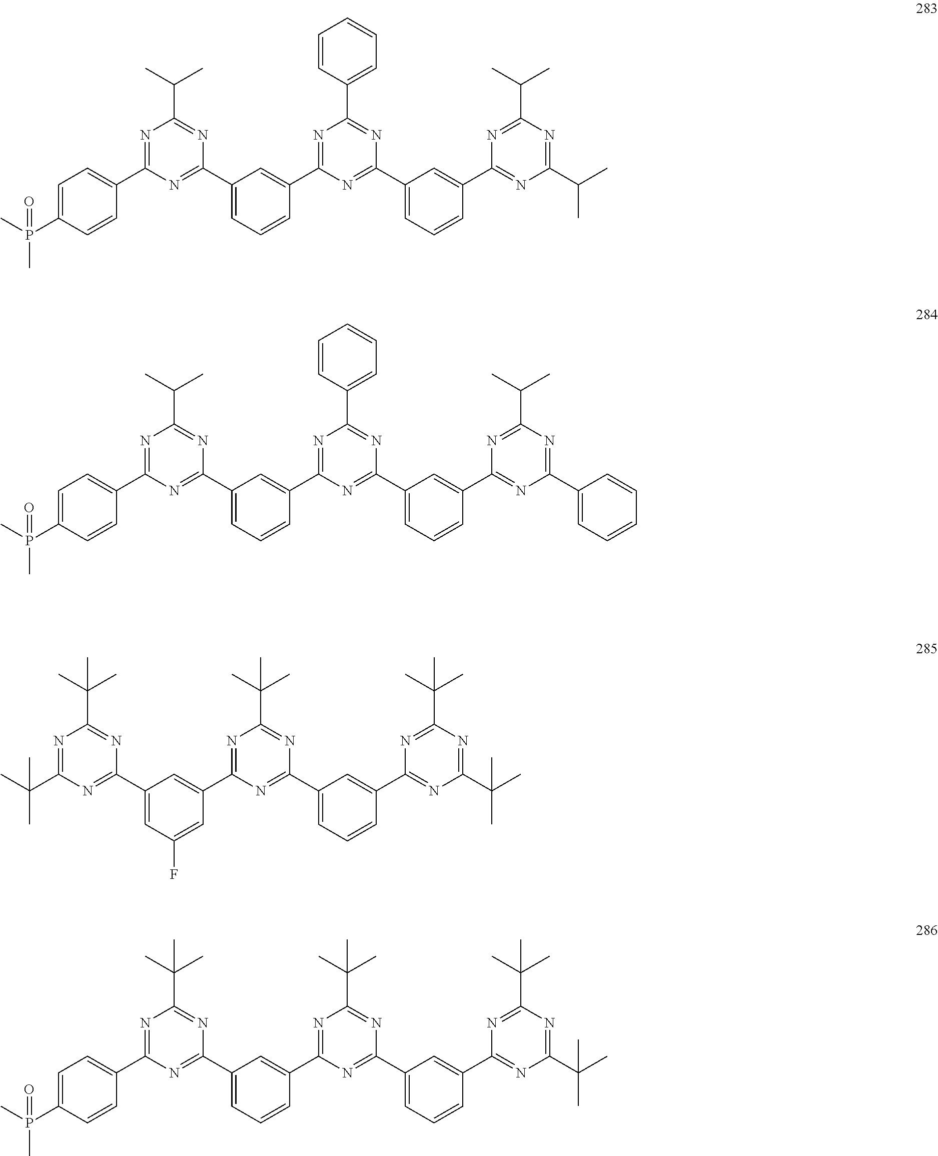

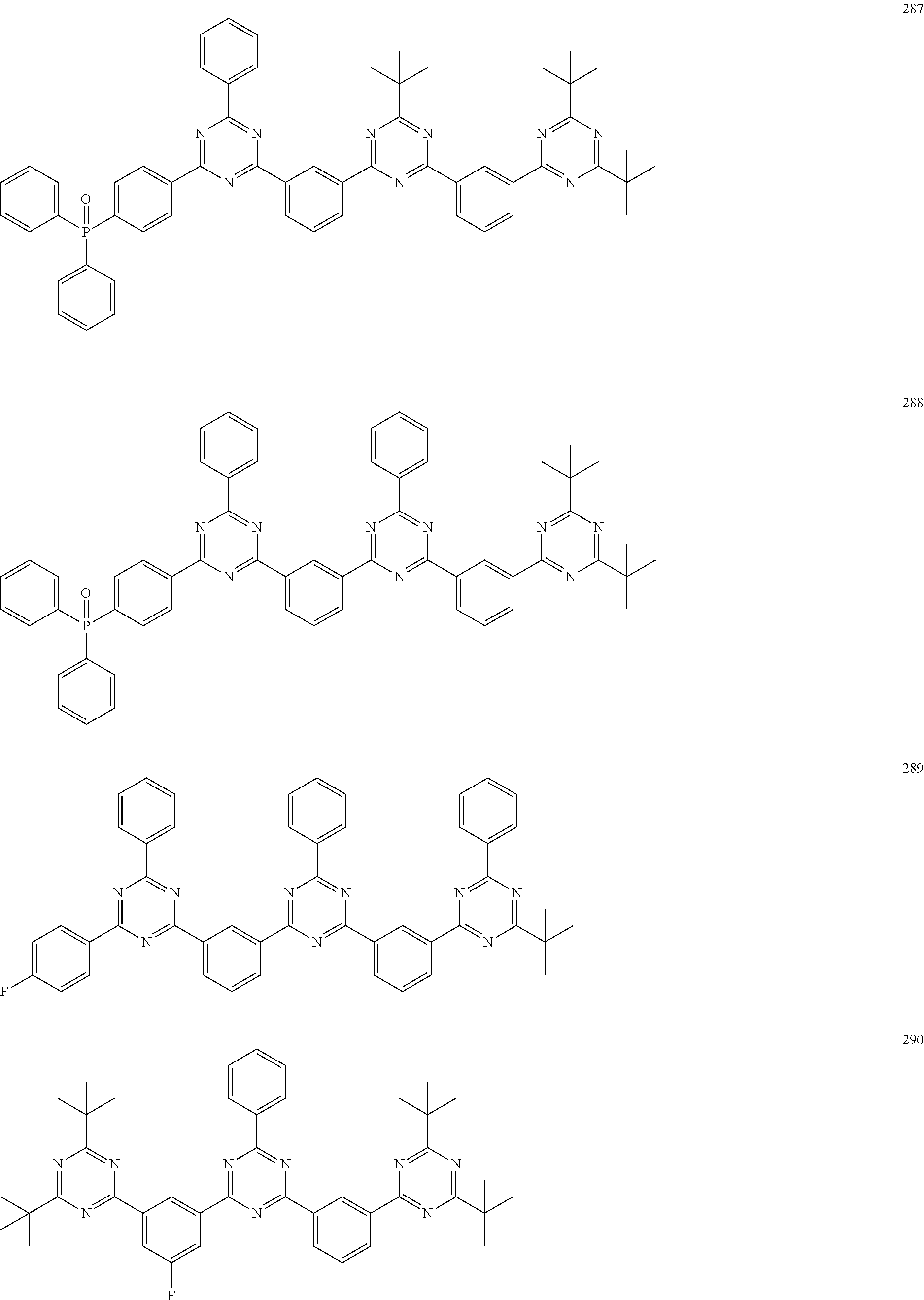

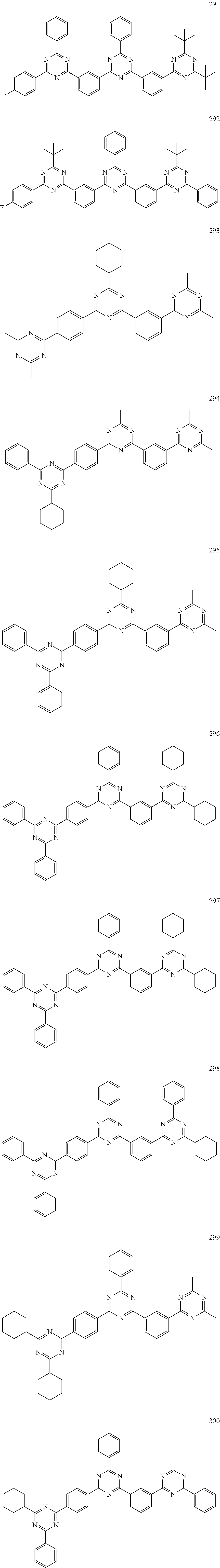

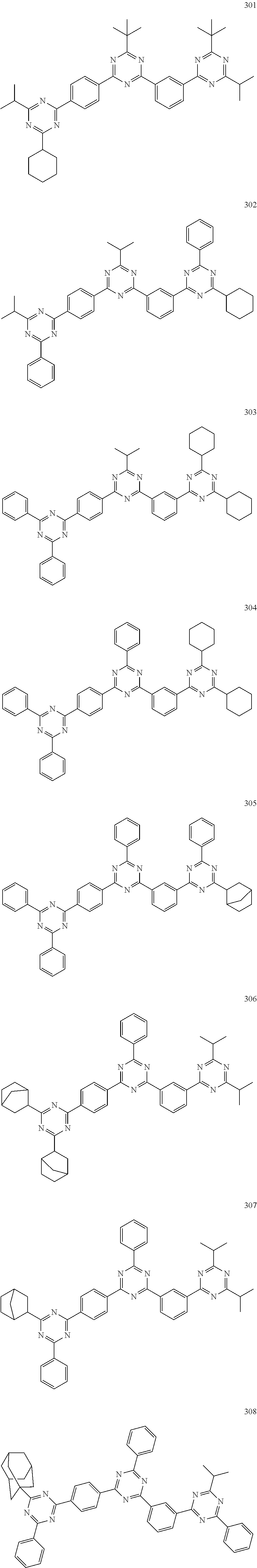

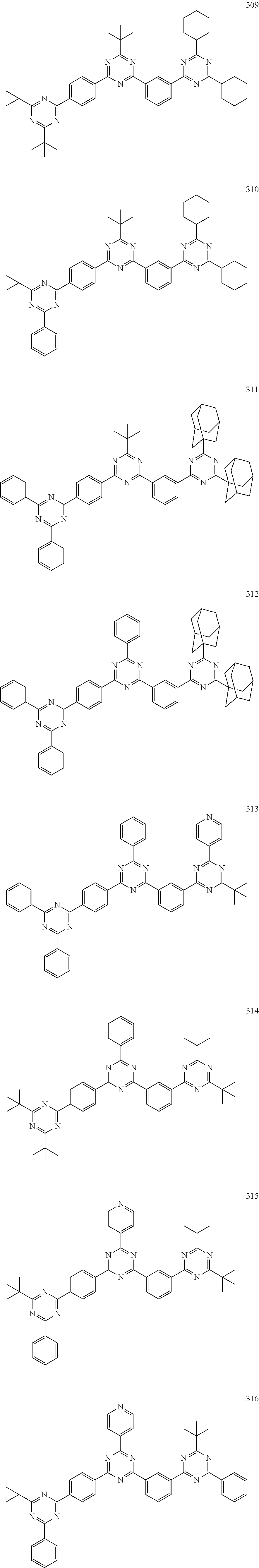

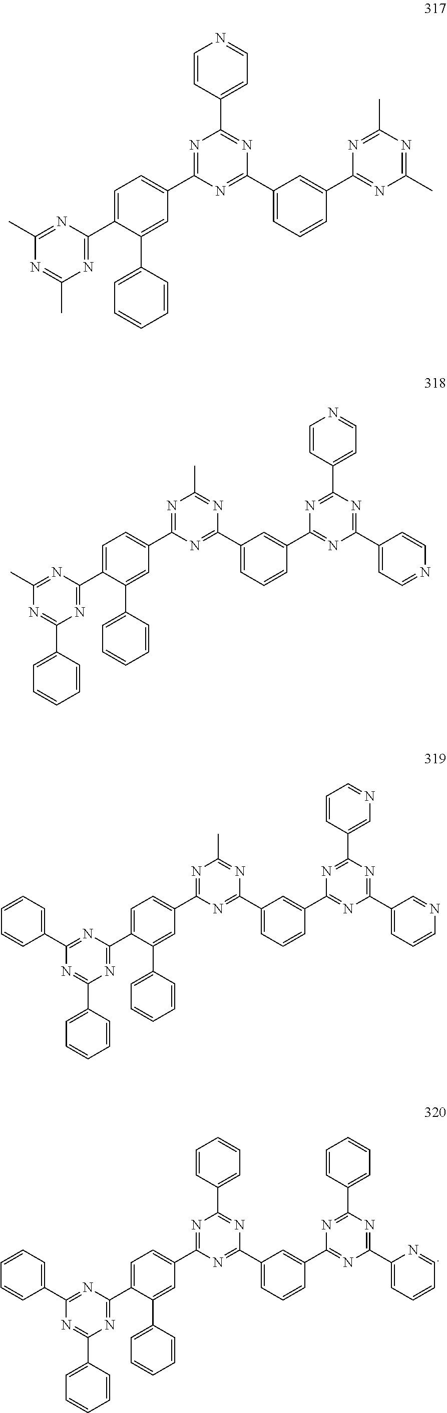

13. A nitrogen-containing compound represented by Formula 1: 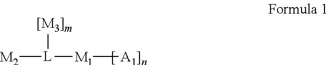
wherein, in Formula 1,
A₁ is -(L₁)ₐ₁M₄,
L₁ is a substituted or unsubstituted arylene group having 6 to 60 ring-forming carbons, or a substituted or unsubstituted heteroarylene group having 2 to 60 ring-forming carbons,
M₂ to M₄ are each independently a substituted or unsubstituted triazine group,
a1 is an integer of 1 to 3,
m and n are each independently 0 or 1,
a sum of m and n is 1,
when n is 0, then M₁ is a substituted or unsubstituted triazine group,
when n is 1, then M₁ is a substituted or unsubstituted divalent triazine group,
when m is 0, then L is represented by -(L₂)ₐ₂-,
L₂ is a substituted or unsubstituted arylene group having 6 to 60 ring-forming carbons, or a substituted or unsubstituted heteroarylene group having 2 to 60 ring-forming carbons,
a2 is an integer of 1 to 3,
when m is 1, then L is a substituted or unsubstituted aryl group having 6 to 60 ring-forming carbons, or a substituted or unsubstituted trivalent heteroaryl group having 2 to 60 ring-forming carbons,
at least one selected from among M₁ to M₄ comprises, as a substituent, a substituted or unsubstituted alkyl group having 1 to 30 carbons, or a substituted or unsubstituted cycloalkyl group having 3 to 30 ring-forming carbon, and
each of L, L₁, and L₂ do not comprise a substituted or unsubstituted divalent carbazole group.

14. The nitrogen-containing compound of claim 13, wherein the nitrogen-containing compound is represented by Formula 2-1 or Formula 2-2: 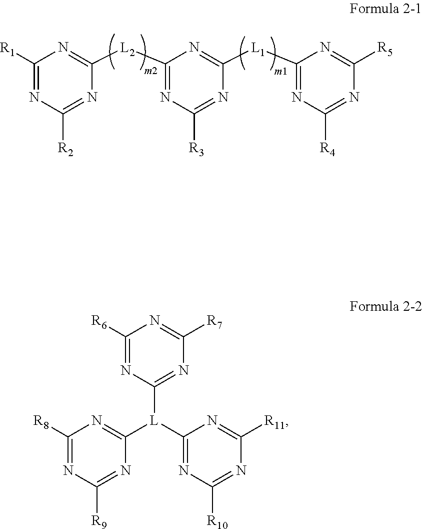
wherein, in Formula 2-1 and Formula 2-2,
R₁ to R₁₁ are each independently a hydrogen atom, a deuterium atom, a halogen atom, a nitro group, a cyano group, a substituted or unsubstituted alkyl group having 1 to 30 carbons, a substituted or unsubstituted cyclcoalkyl group having 3 to 30 ring-forming carbons, a substituted or unsubstituted aryl group having 6 to 60 ring-forming carbons, or a substituted or unsubstituted heteroaryl group having 2 to 60 ring-forming carbons,
in Formula 2-1, at least one selected from among R₁ to R₅ is a substituted or unsubstituted alkyl group having 1 to 30 carbons, or a substituted or unsubstituted cycloalkyl group having 3 to 30 ring-forming carbons,
in Formula 2-2, at least one selected from among R₆ to R₁₁ is a substituted or unsubstituted alkyl group having 1 to 30 carbons, or a substituted or unsubstituted cycloalkyl group having 3 to 30 ring-forming carbons, and
L, L₁, L₂, m1, and m2 are as defined in Formula 1.

15. The nitrogen-containing compound of claim 14, wherein the nitrogen-containing compound is represented by any one selected from among Formula 3-1 to Formula 3-3: 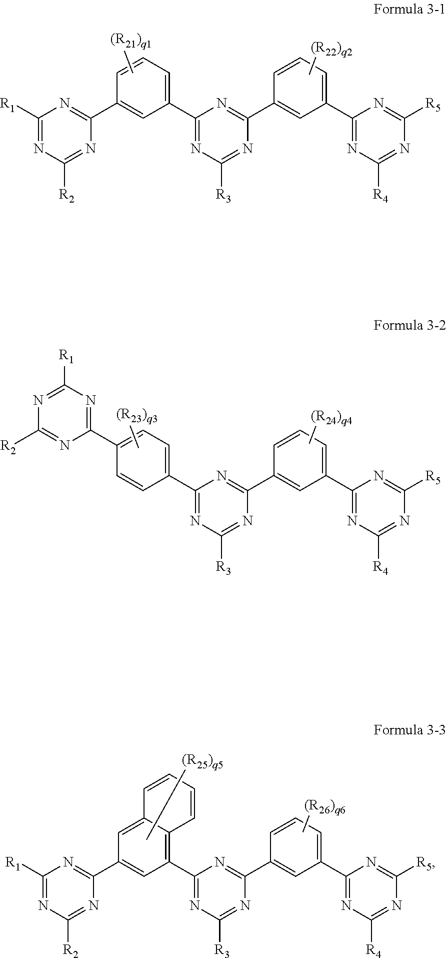
wherein, in Formula 3-1 to Formula 3-3,
R₂₁ to R₂₆ are each independently a hydrogen atom, a deuterium atom, a halogen atom, a cyano group, a nitro group, a substituted or unsubstituted alkyl group having 1 to 30 carbons, a substituted or unsubstituted aryl group having 6 to 60 ring-forming carbons, or a substituted or unsubstituted heteroaryl group having 2 to 60 ring-forming carbons,
q1 to q4, and q6 are each independently an integer of 0 to 4,
q5 is an integer of 0 to 6, and
R₁ to R₅ are as defined in Formula 2-1.

16. The nitrogen-containing compound of claim 14, wherein the nitrogen-containing compound is represented by Formula 4: 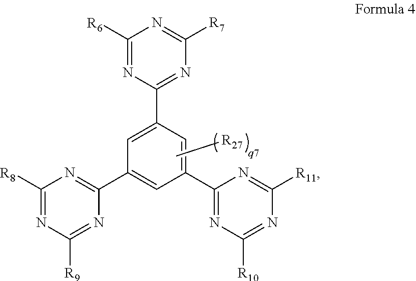
wherein, in Formula 4,
R₂₇ is a hydrogen atom, a deuterium atom, a halogen atom, a cyano group, a nitro group, a substituted or unsubstituted alkyl group having 1 to 20 carbons, a substituted or unsubstituted aryl group having 6 to 60 ring-forming carbons, or a substituted or unsubstituted heteroaryl group having 2 to 60 ring-forming carbons,
q7 is an integer of 0 to 3, and
R₆ to R₁₁ are as defined in Formula 2-2.

17. The nitrogen-containing compound of claim 13, wherein the nitrogen-containing compound is represented by any one selected from among compounds in Compound Group 1:

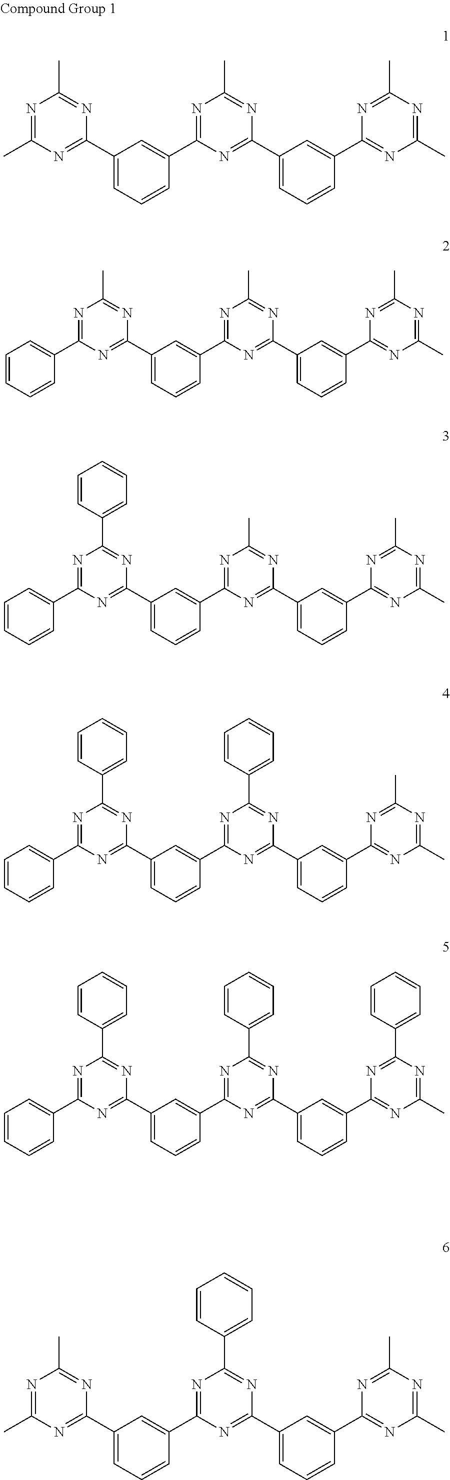

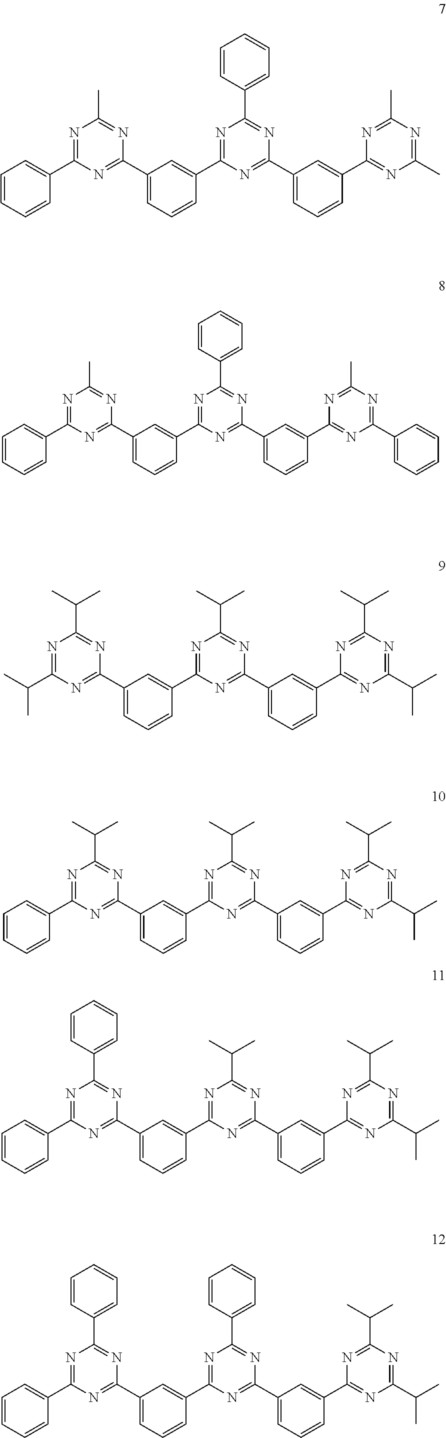

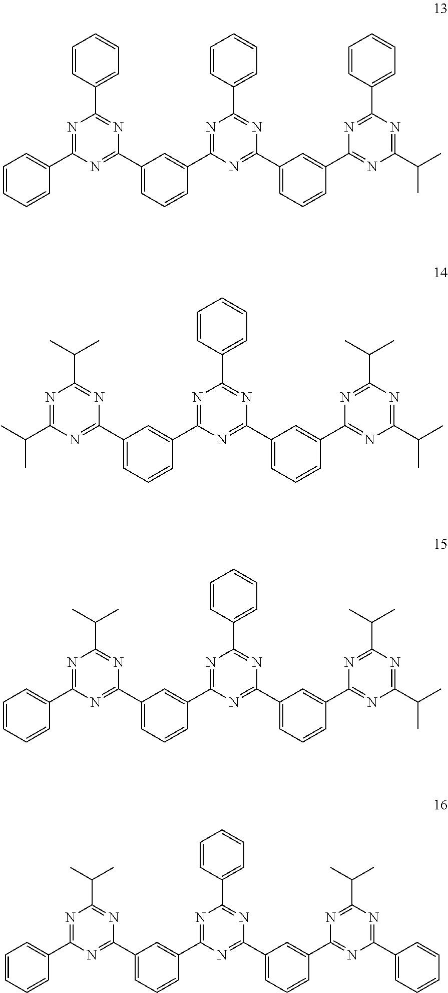

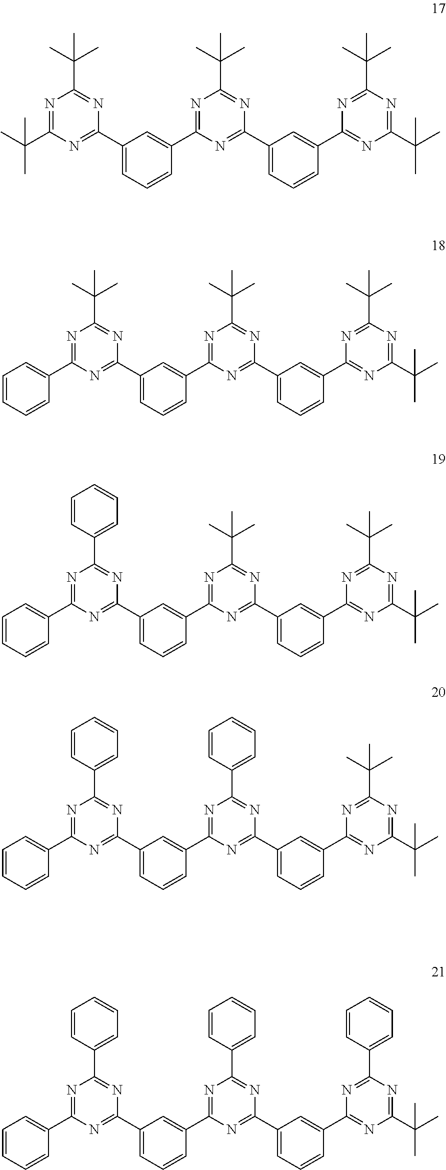

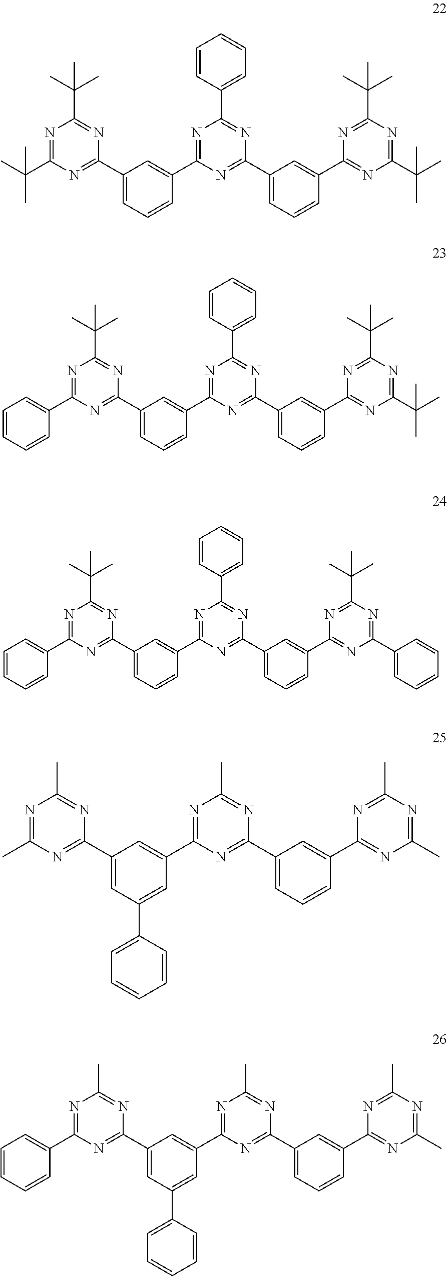

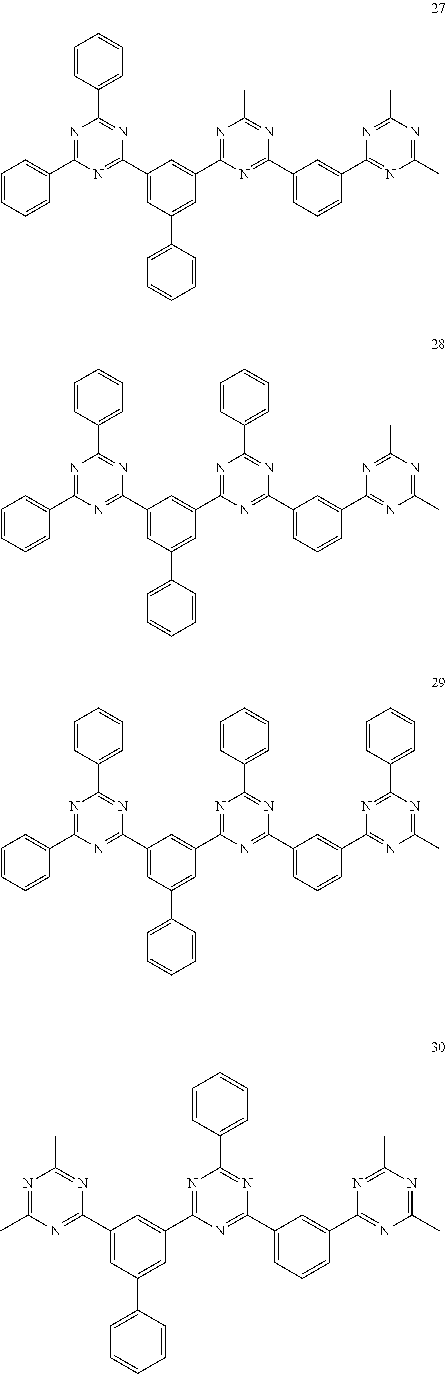

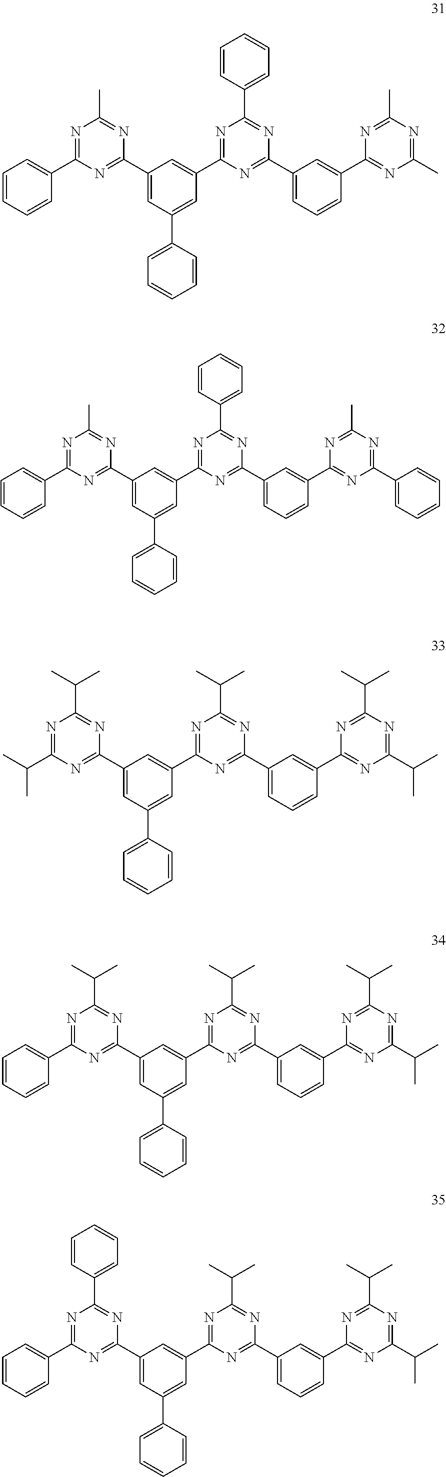

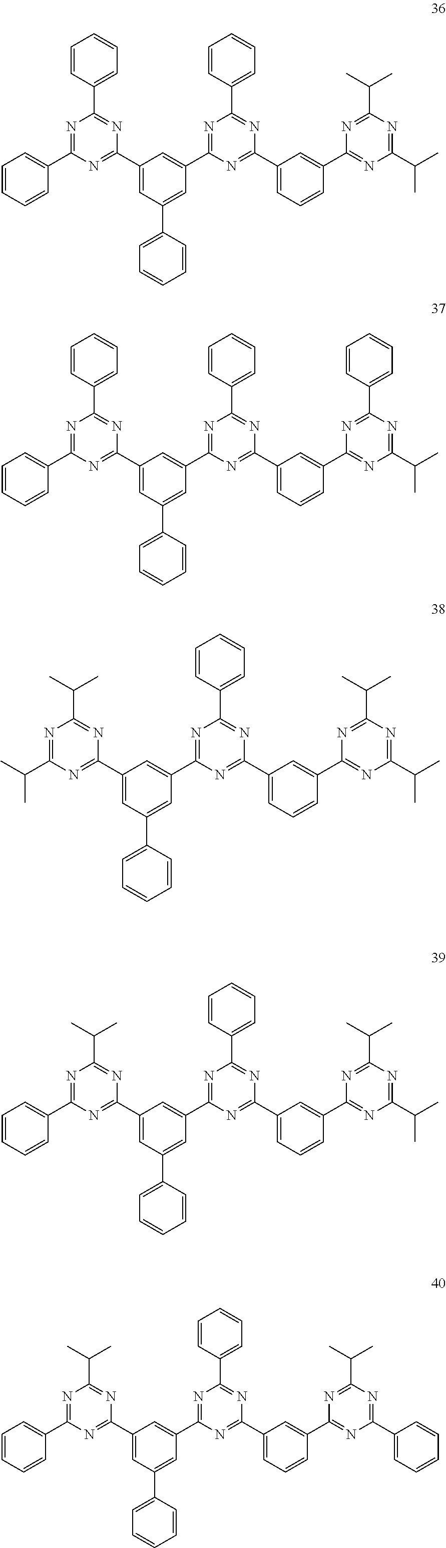

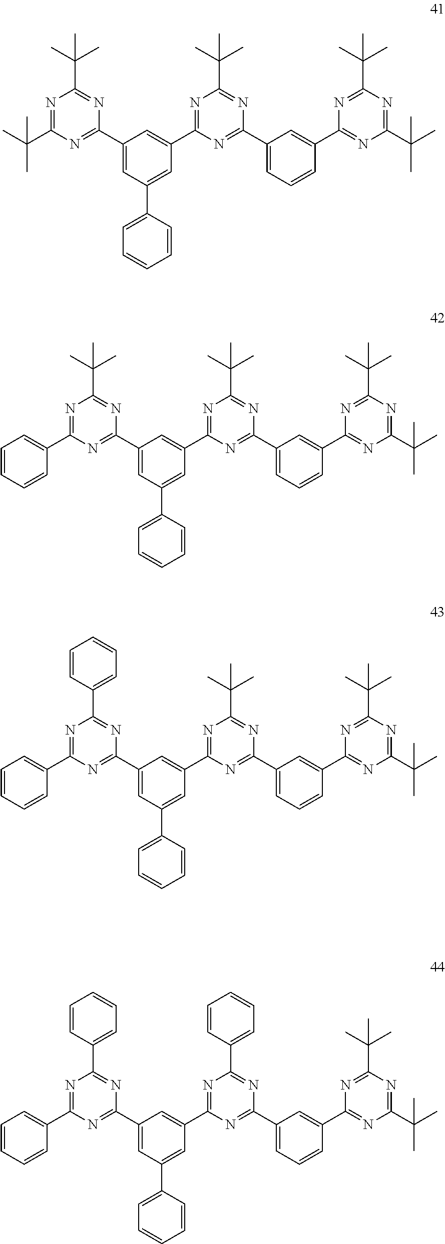 [SMILES: CC(C)(C)c1nc(-c2cccc(-c3nc(C(C)(C)C)nc(C(C)(C)C)n3)c2)nc(-c2cc(-c3ccccc3)cc(-c3nc(C(C)(C)C)nc(C(C)(C)C)n3)c2)n1 ; CC(C)(C)c1nc(-c2ccccc2)nc(-c2cc(-c3ccccc3)cc(-c3nc(-c4cccc(-c5nc(C(C)(C)C)nc(C(C)(C)C)n5)c4)nc(C(C)(C)C)n3)c2)n1 ; CC(C)(C)c1nc(-c2cc(-c3ccccc3)cc(-c3nc(-c4ccccc4)nc(-c4ccccc4)n3)c2)nc(-c2cccc(-c3nc(C(C)(C)C)nc(C(C)(C)C)n3)c2)n1 ; CC(C)(C)c1nc(-c2cccc(-c3nc(-c4ccccc4)nc(-c4cc(-c5ccccc5)cc(-c5nc(-c6ccccc6)nc(-c6ccccc6)n5)c4)n3)c2)nc(C(C)(C)C)n1]

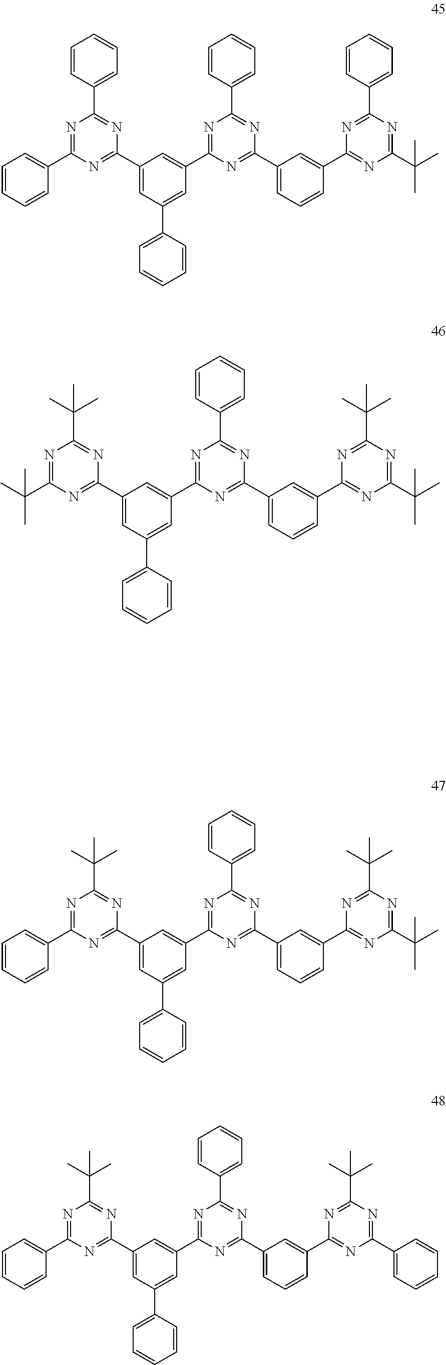

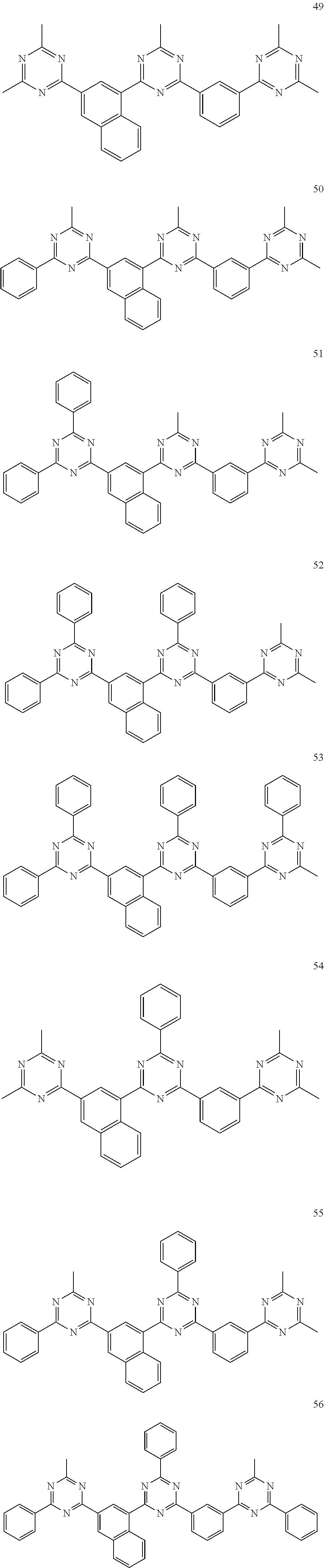

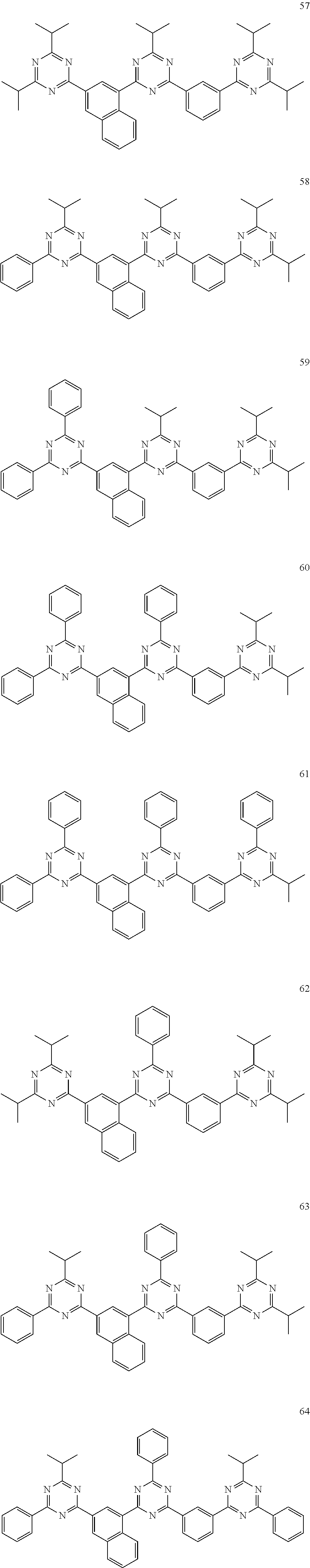

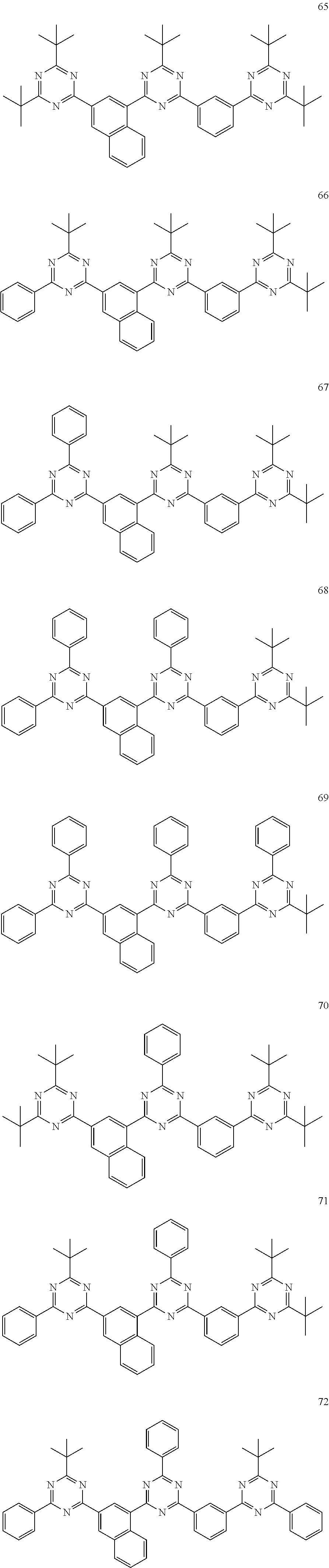

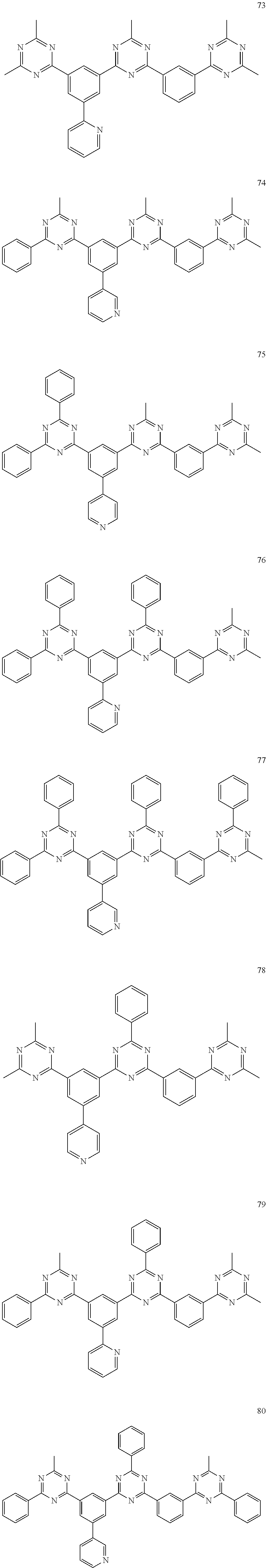

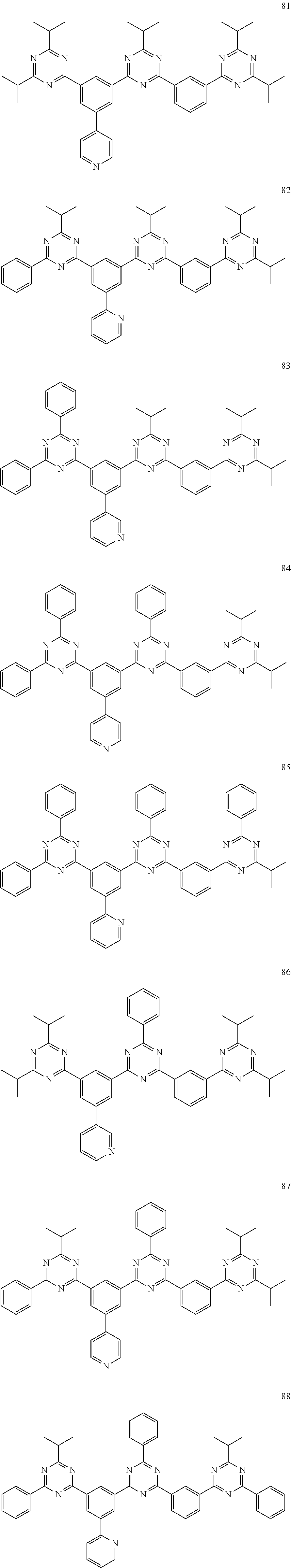

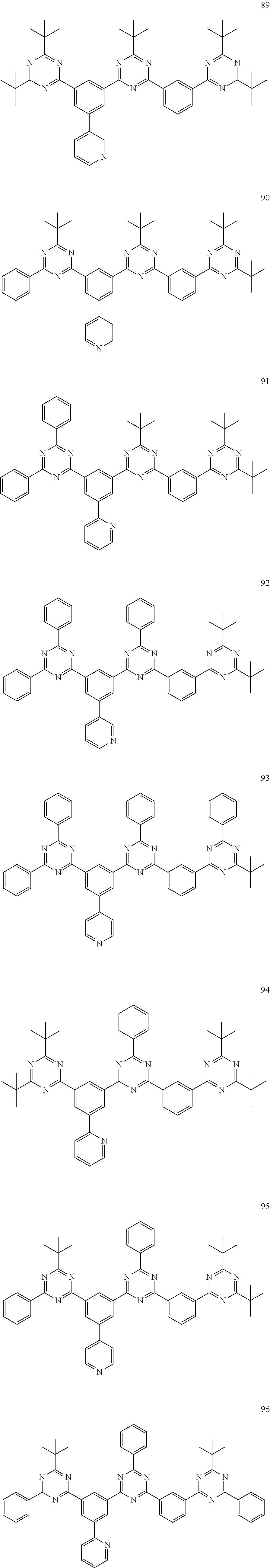

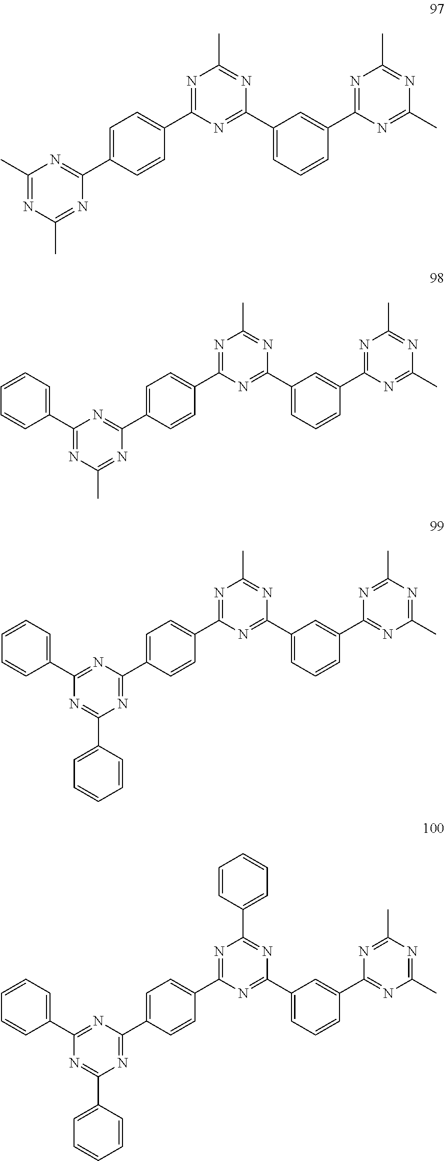

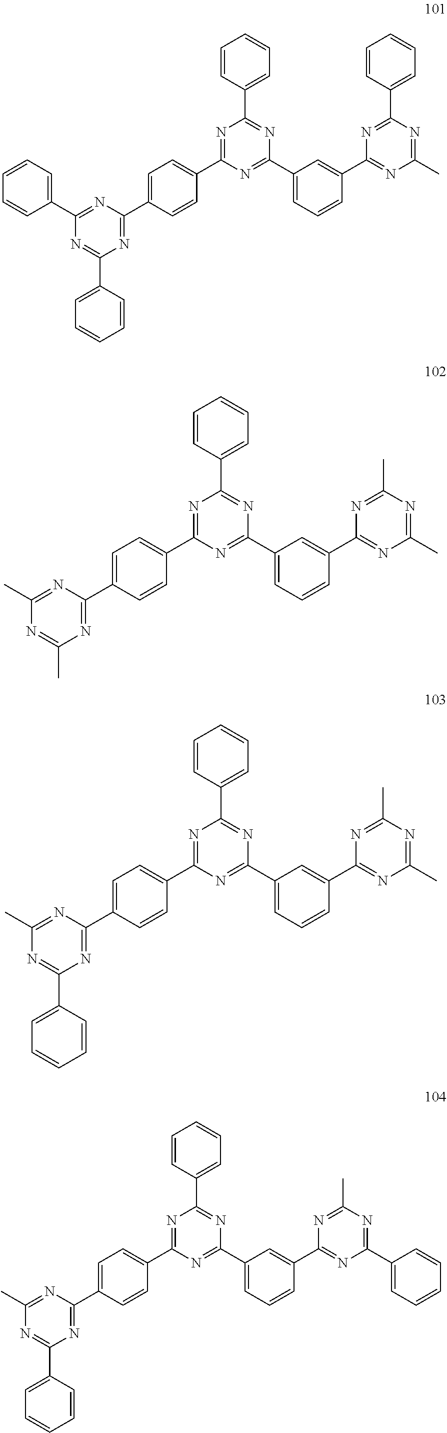

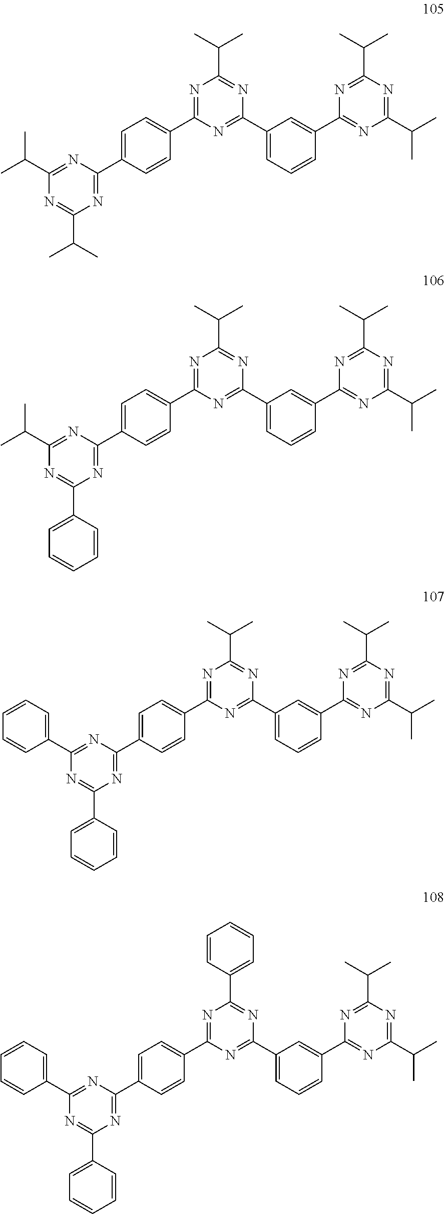

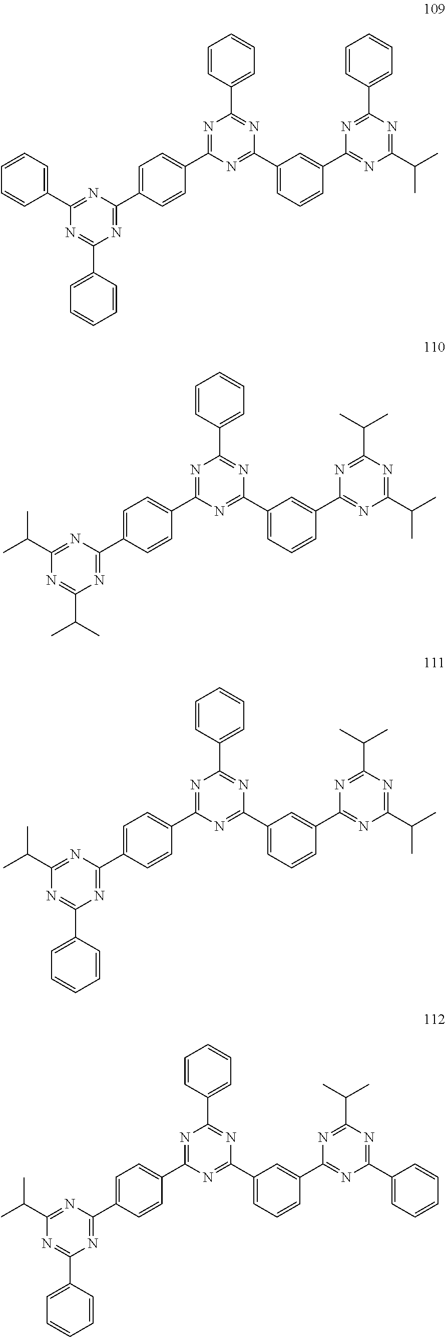

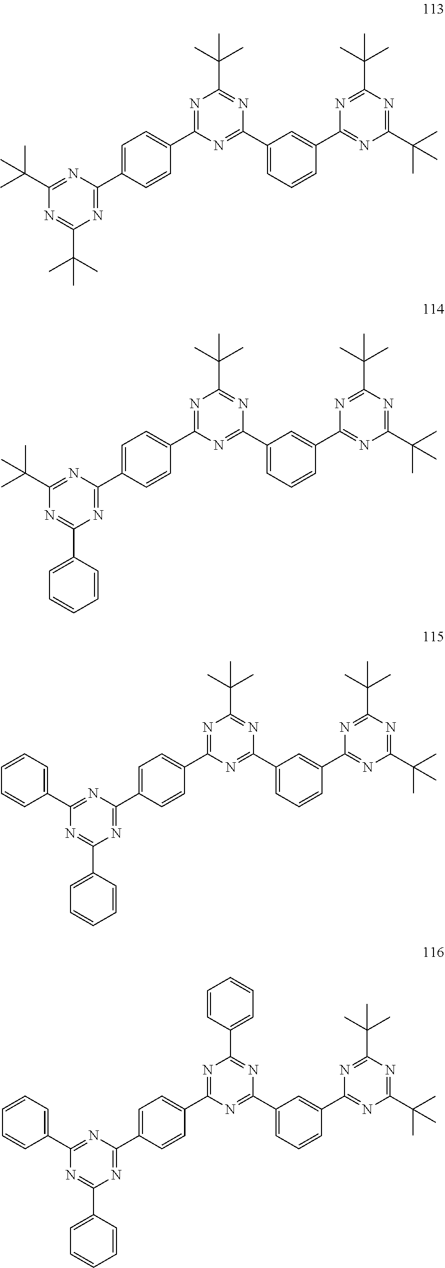

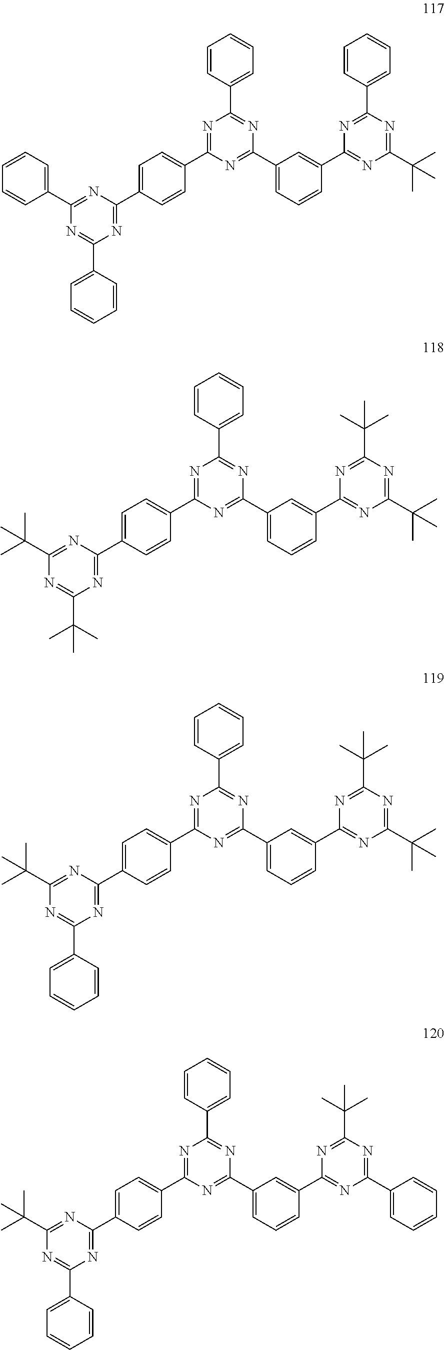

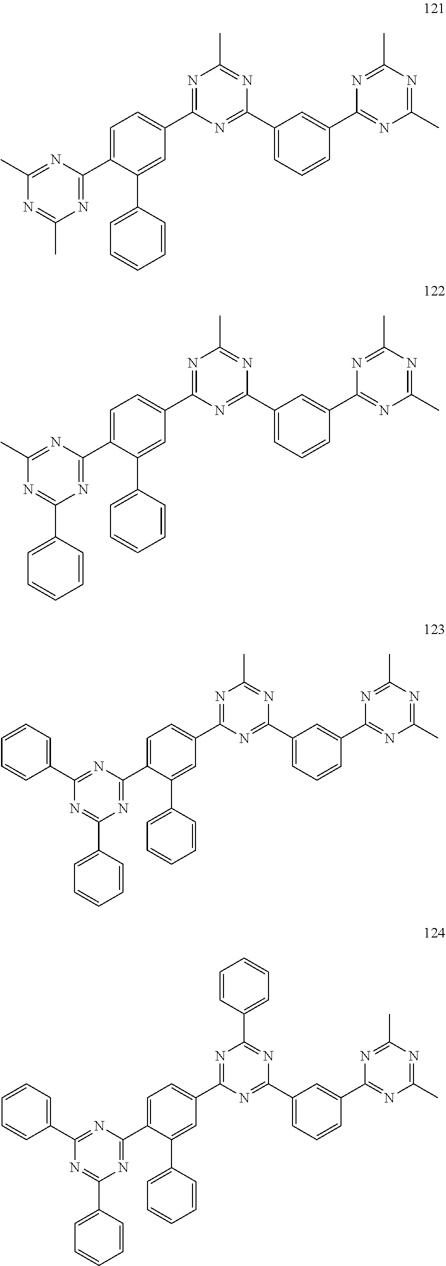

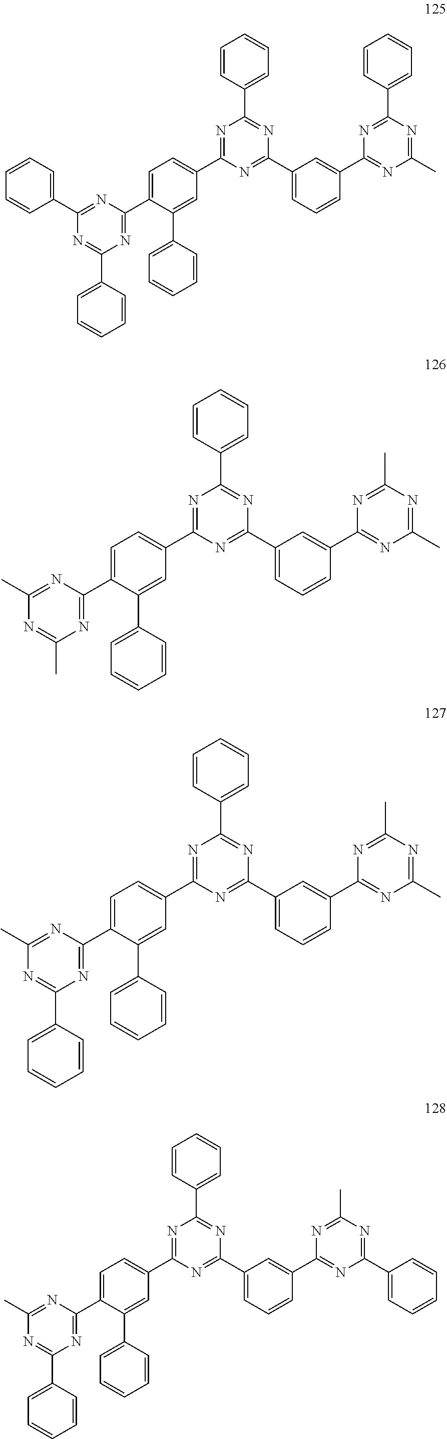

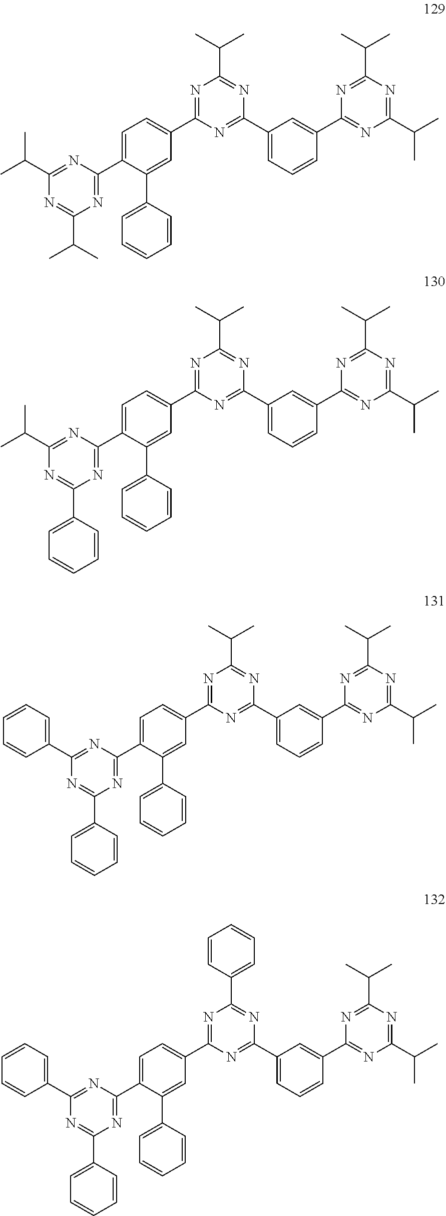

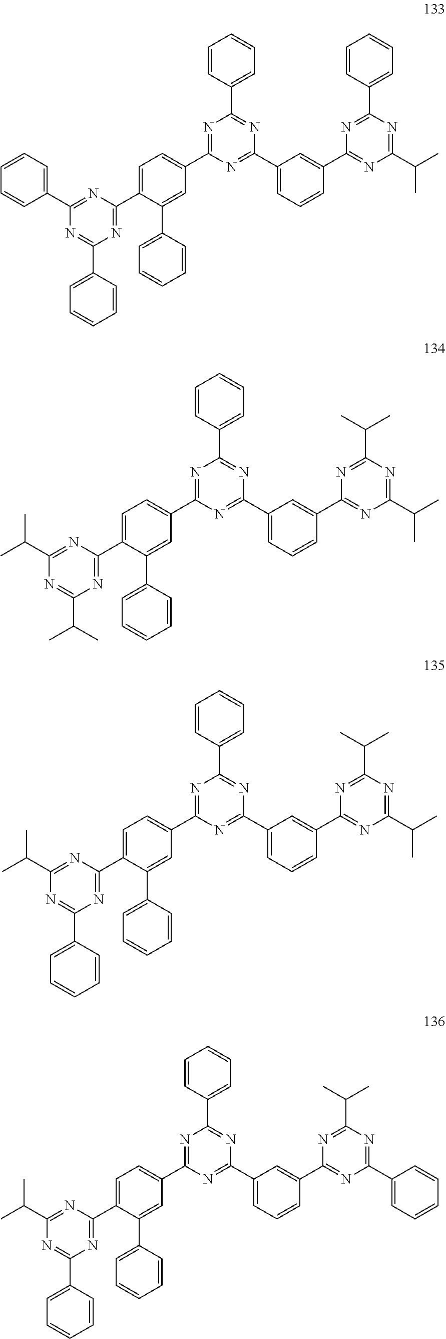

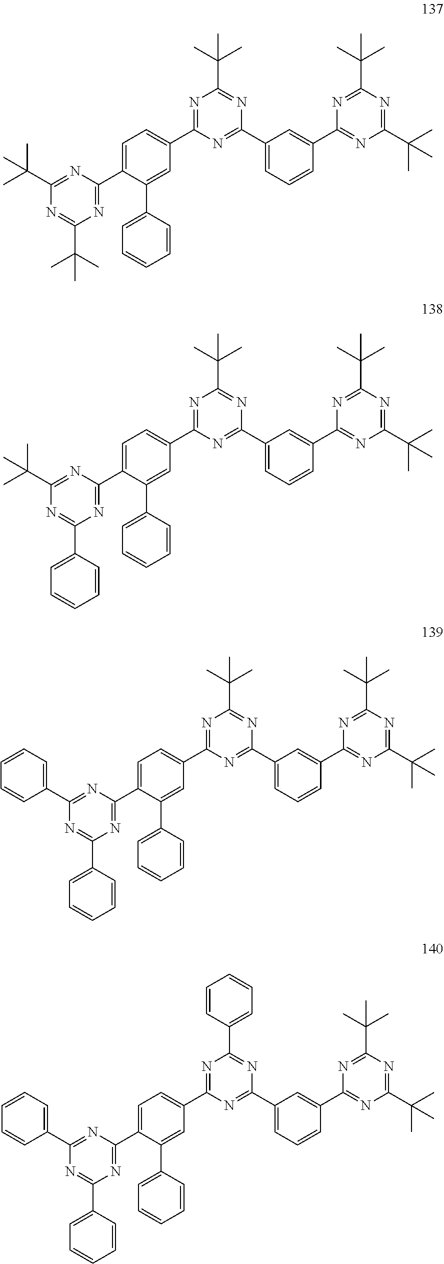

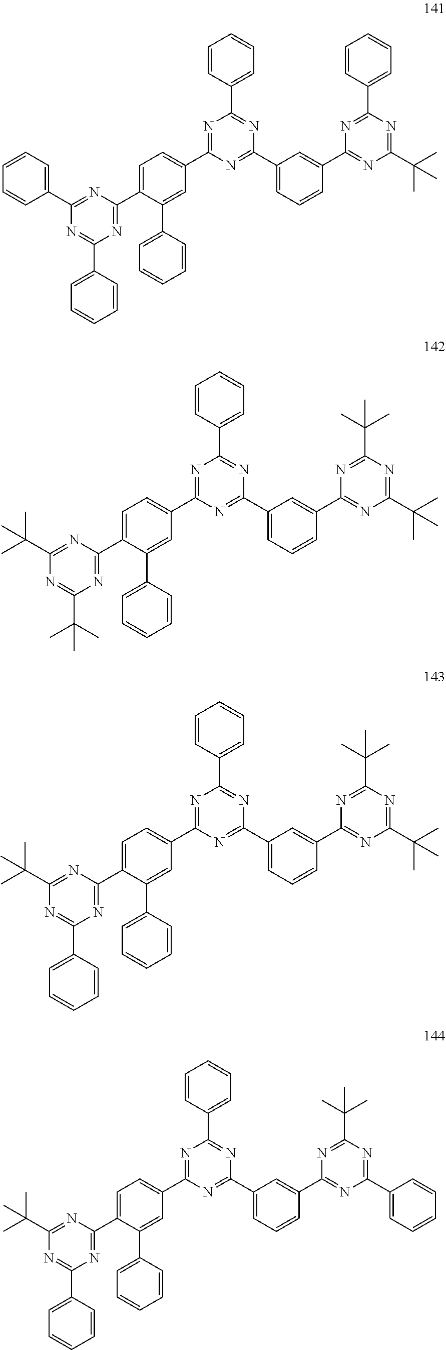

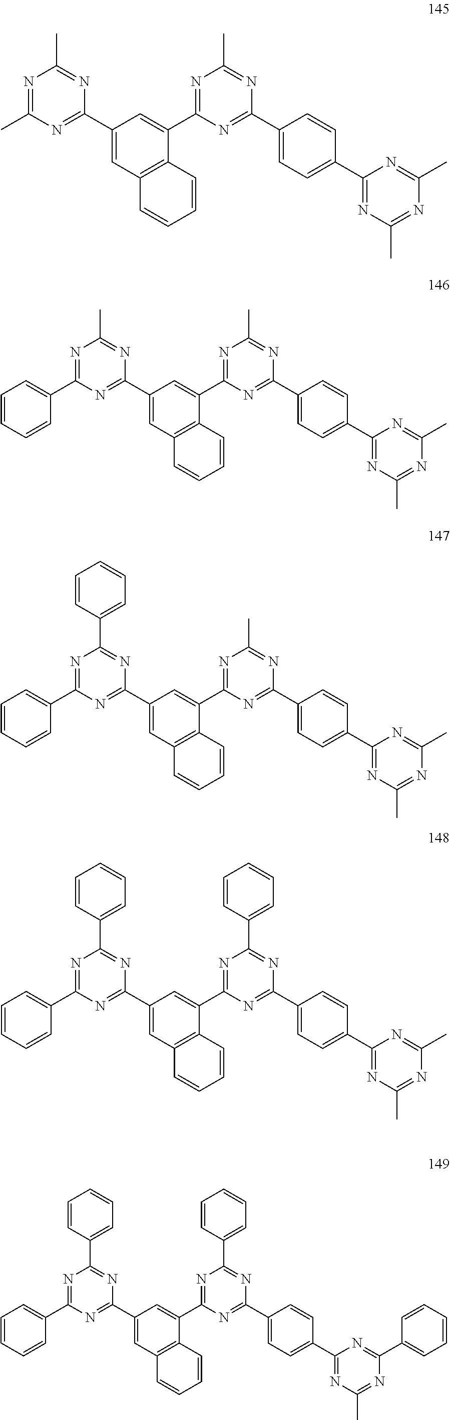

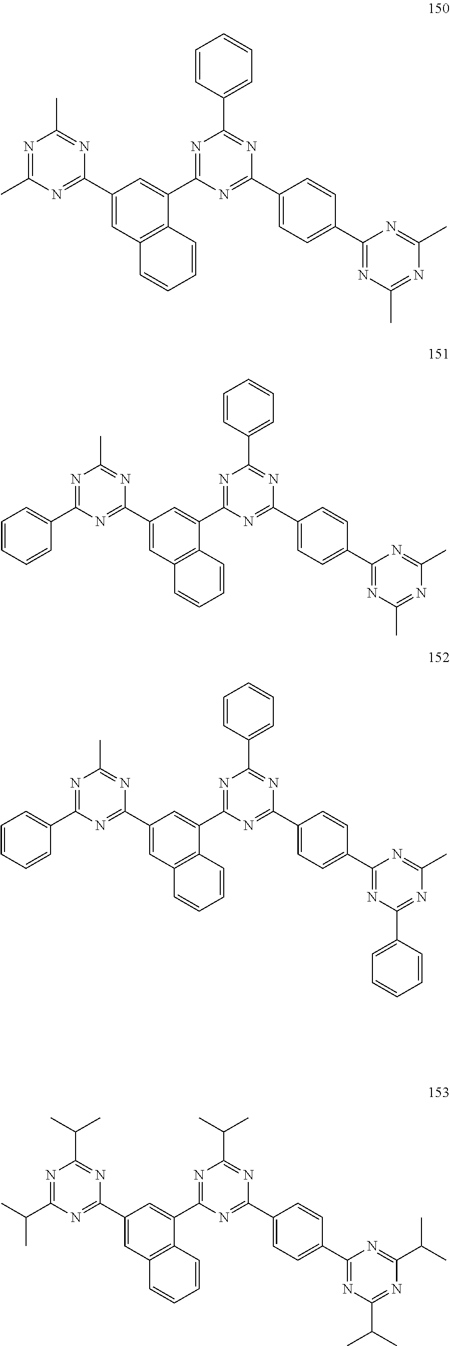

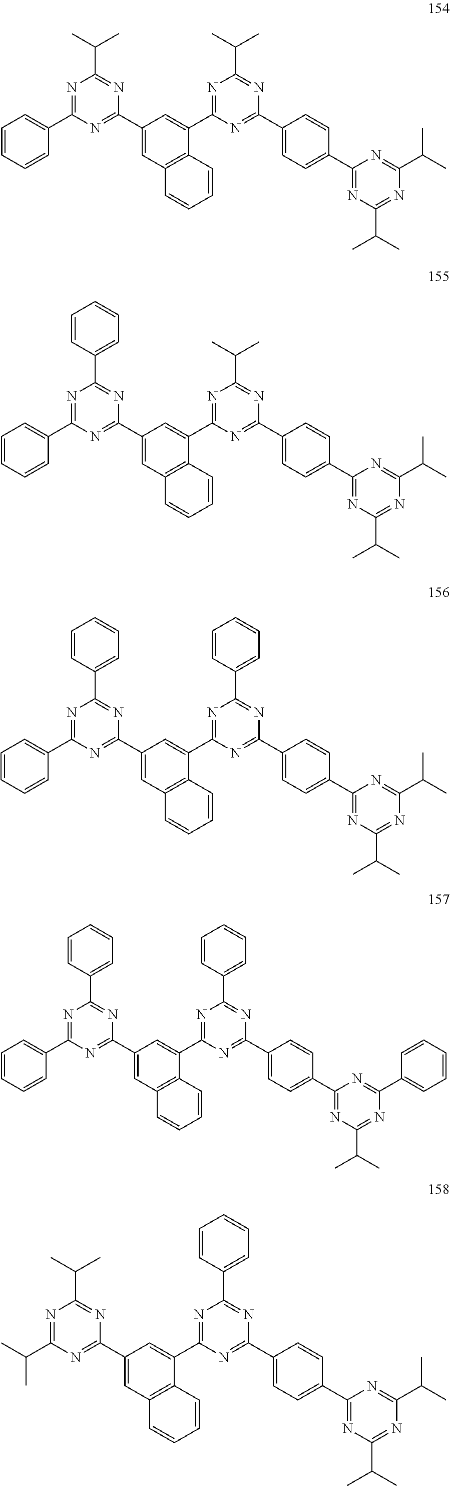

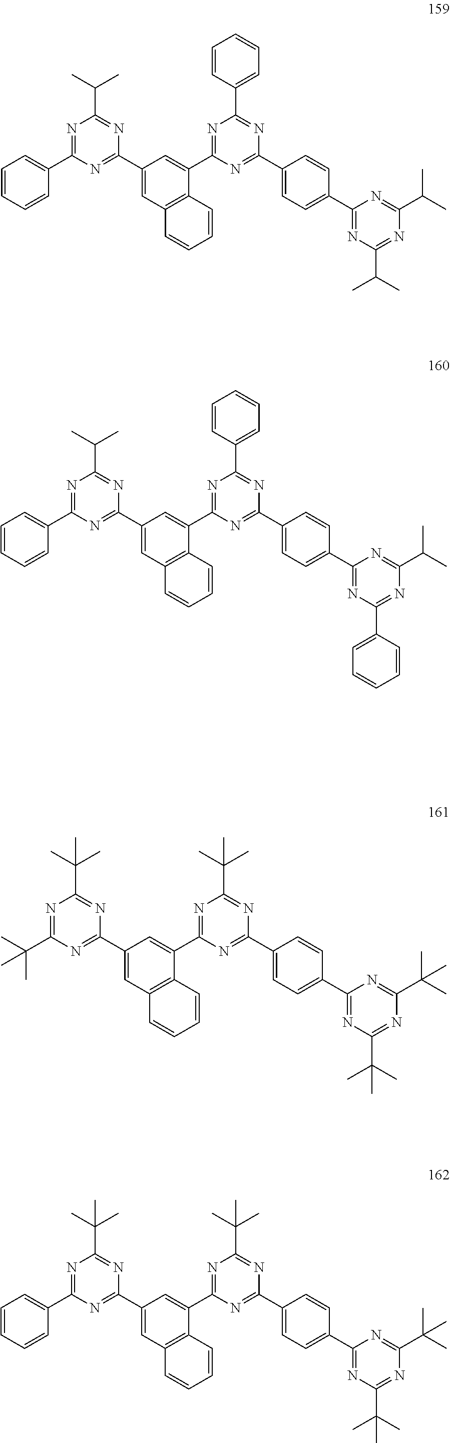

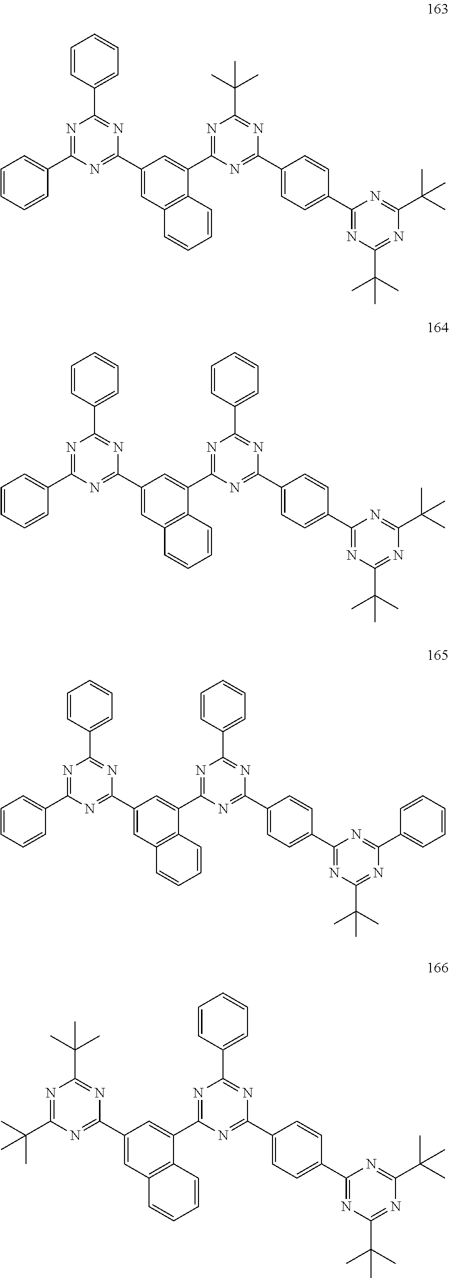

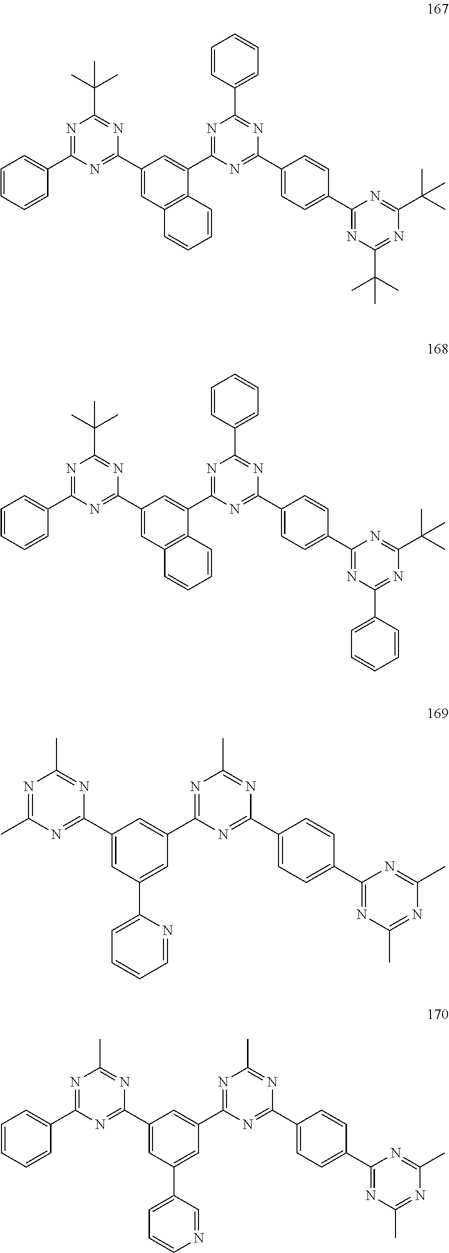

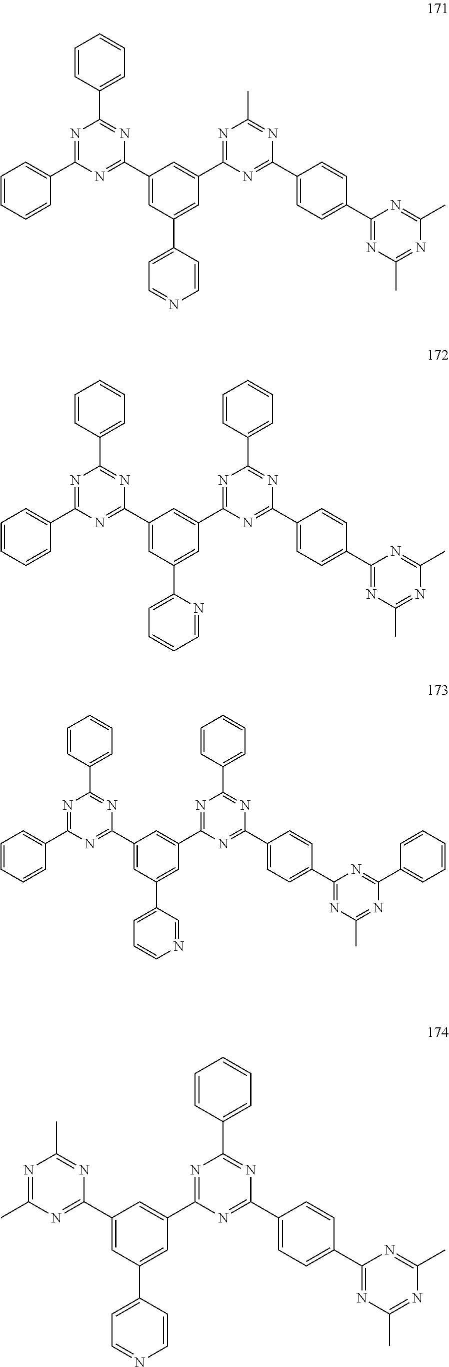

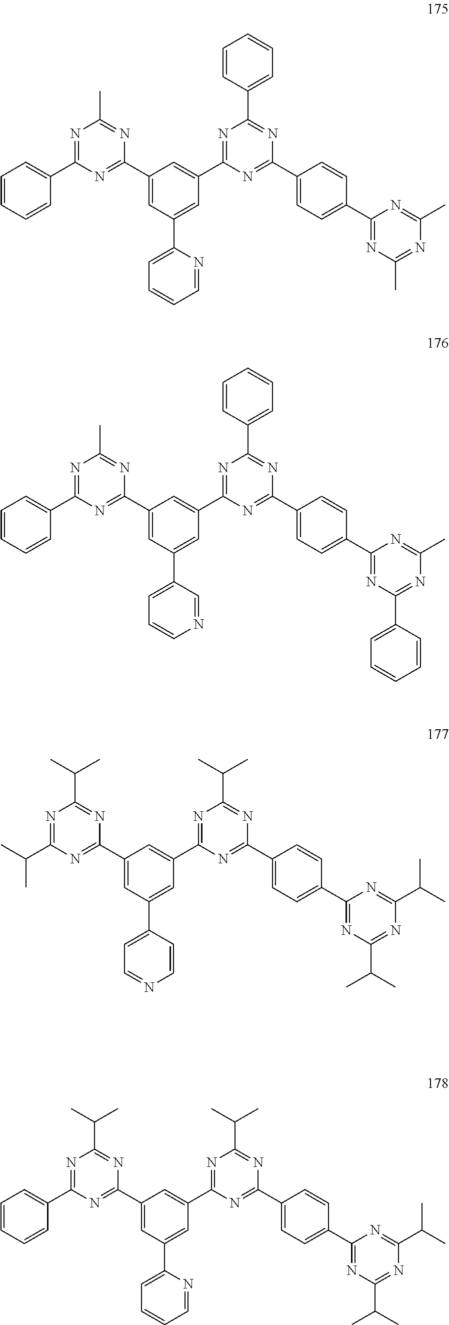

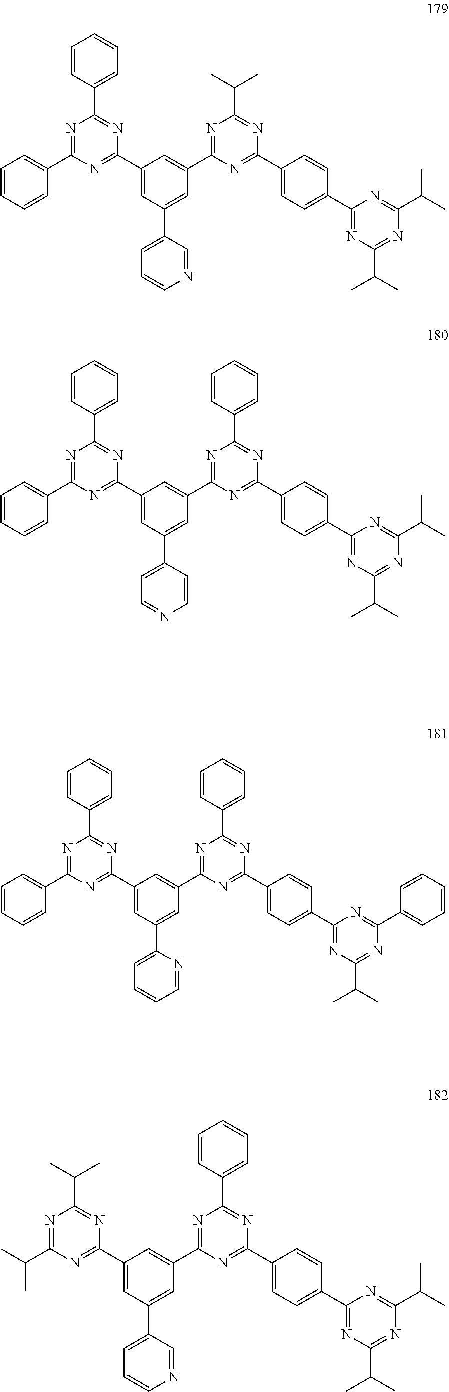

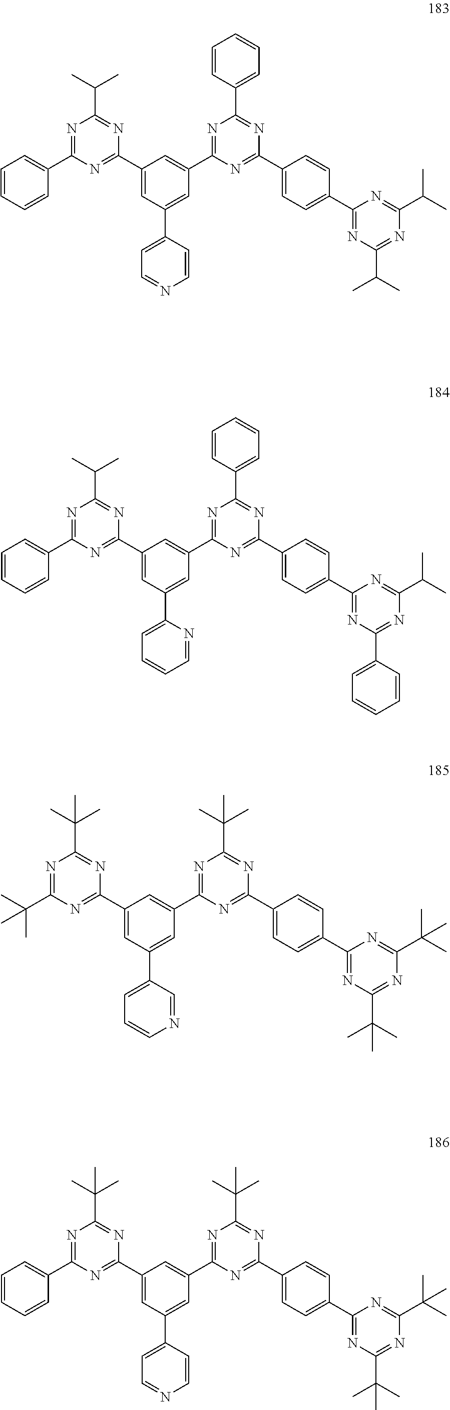

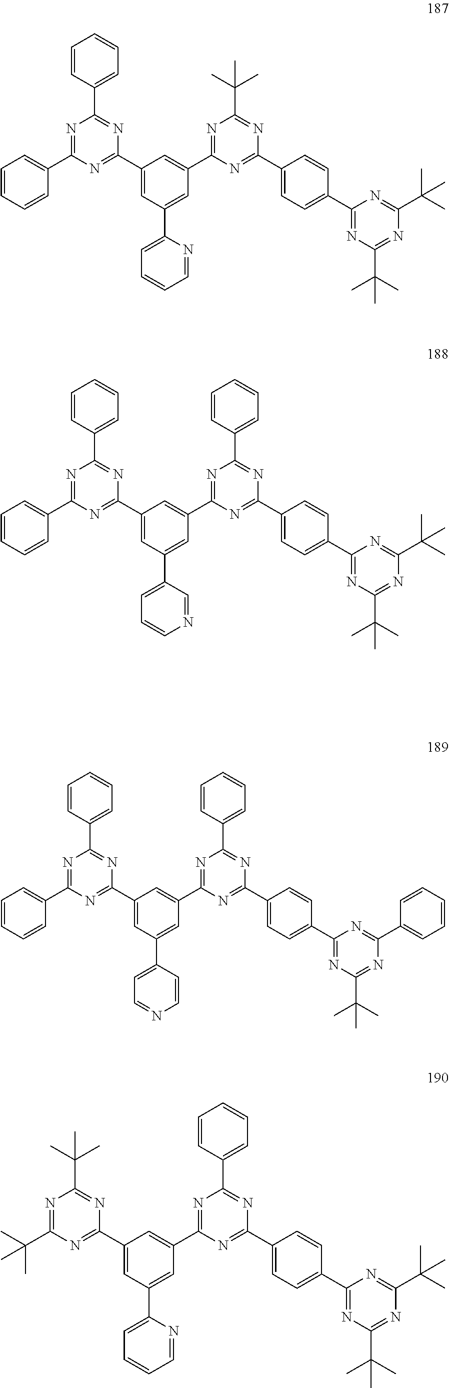

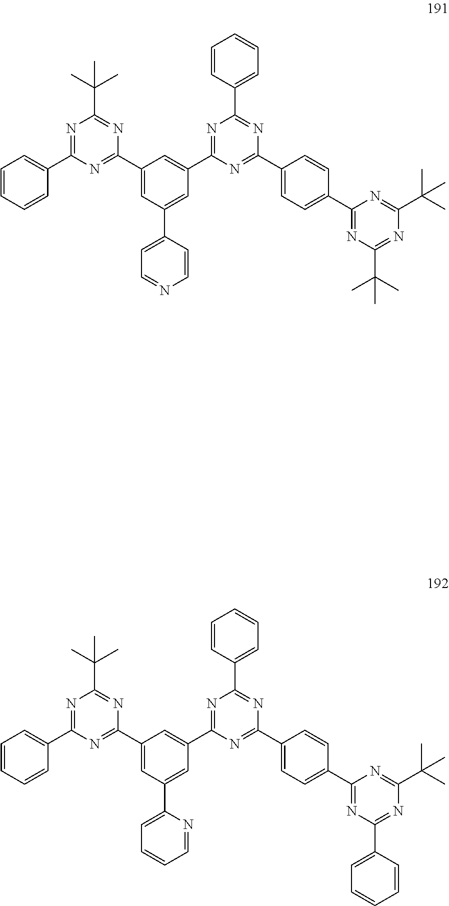 [SMILES: CC(C)(C)c1nc(-c2ccccc2)nc(-c2cc(-c3ccncc3)cc(-c3nc(-c4ccccc4)nc(-c4ccc(-c5nc(C(C)(C)C)nc(C(C)(C)C)n5)cc4)n3)c2)n1 ; CC(C)(C)c1nc(-c2ccccc2)nc(-c2ccc(-c3nc(-c4ccccc4)nc(-c4cc(-c5ccccn5)cc(-c5nc(-c6ccccc6)nc(C(C)(C)C)n5)c4)n3)cc2)n1]

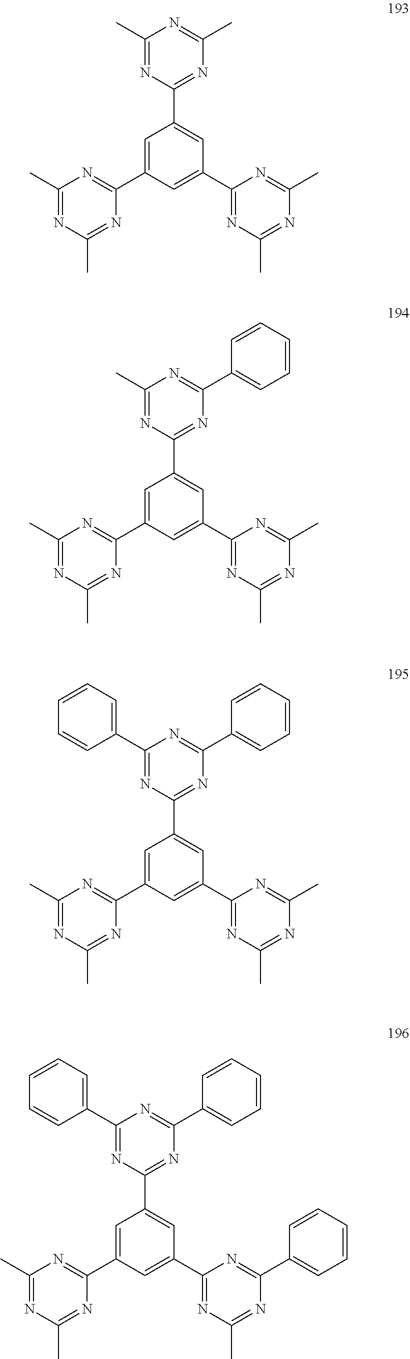

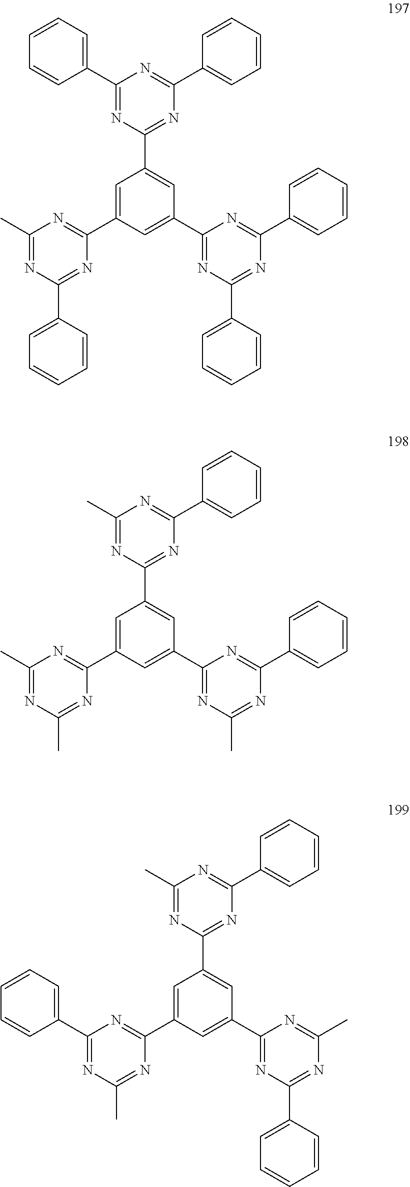 [SMILES: Cc1nc(-c2ccccc2)nc(-c2cc(-c3nc(-c4ccccc4)nc(-c4ccccc4)n3)cc(-c3nc(-c4ccccc4)nc(-c4ccccc4)n3)c2)n1 ; Cc1nc(C)nc(-c2cc(-c3nc(C)nc(-c4ccccc4)n3)cc(-c3nc(C)nc(-c4ccccc4)n3)c2)n1 ; Cc1nc(-c2ccccc2)nc(-c2cc(-c3nc(C)nc(-c4ccccc4)n3)cc(-c3nc(C)nc(-c4ccccc4)n3)c2)n1]

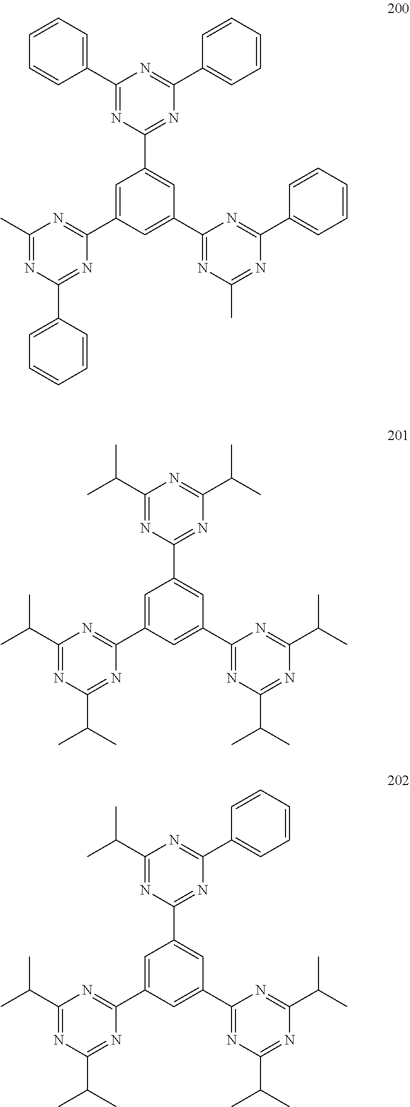

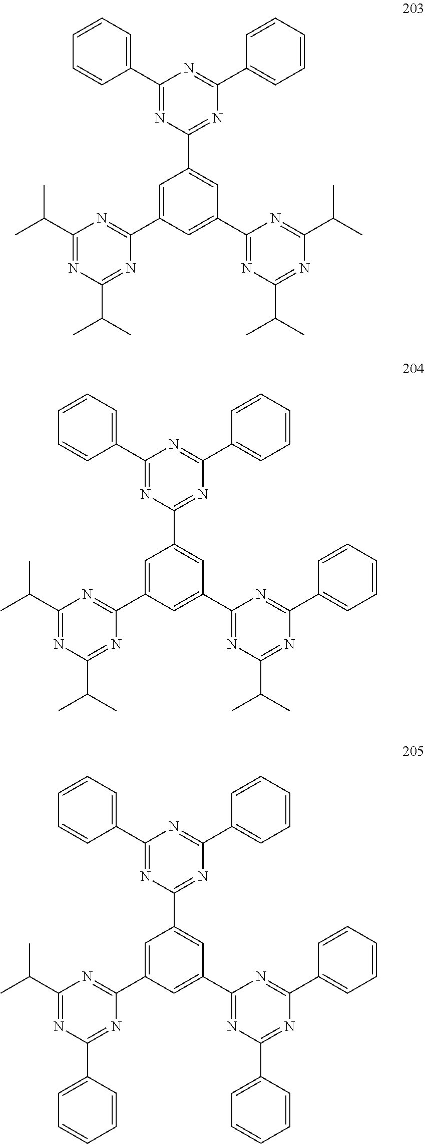

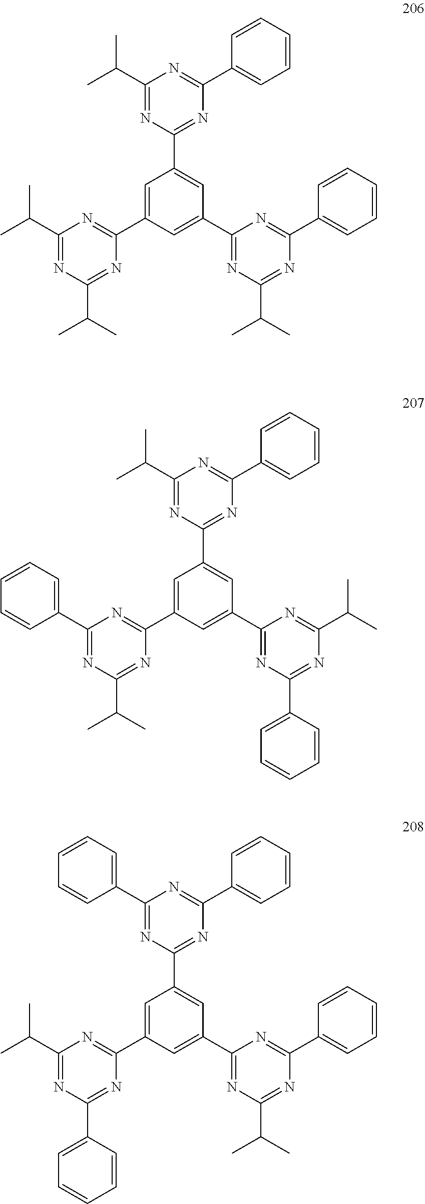

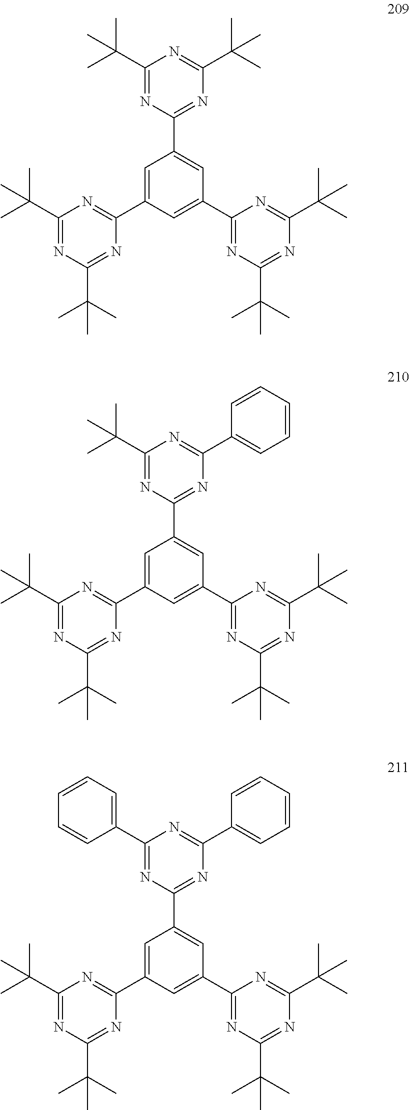

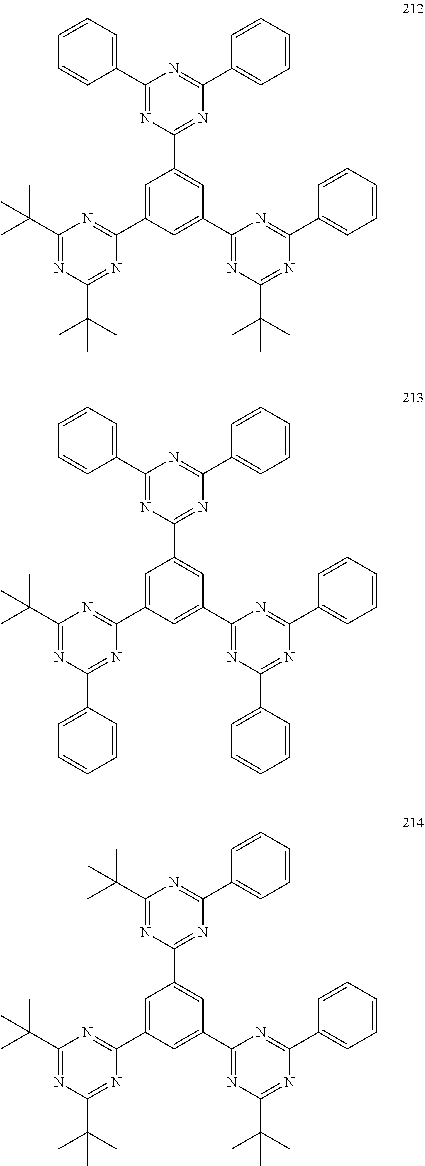 [SMILES: CC(C)(C)c1nc(-c2ccccc2)nc(-c2cc(-c3nc(-c4ccccc4)nc(-c4ccccc4)n3)cc(-c3nc(C(C)(C)C)nc(C(C)(C)C)n3)c2)n1 ; CC(C)(C)c1nc(-c2ccccc2)nc(-c2cc(-c3nc(-c4ccccc4)nc(-c4ccccc4)n3)cc(-c3nc(-c4ccccc4)nc(-c4ccccc4)n3)c2)n1 ; CC(C)(C)c1nc(-c2ccccc2)nc(-c2cc(-c3nc(-c4ccccc4)nc(C(C)(C)C)n3)cc(-c3nc(C(C)(C)C)nc(C(C)(C)C)n3)c2)n1]

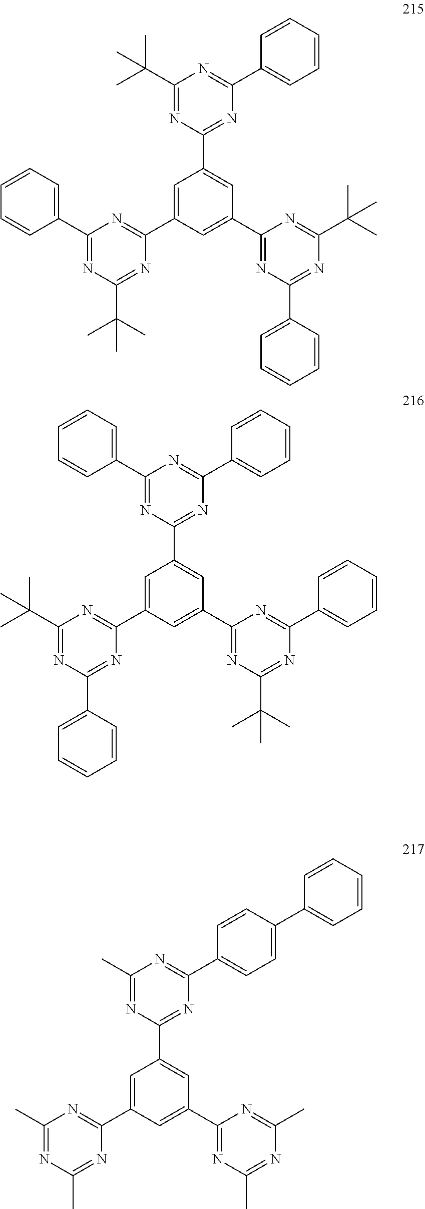

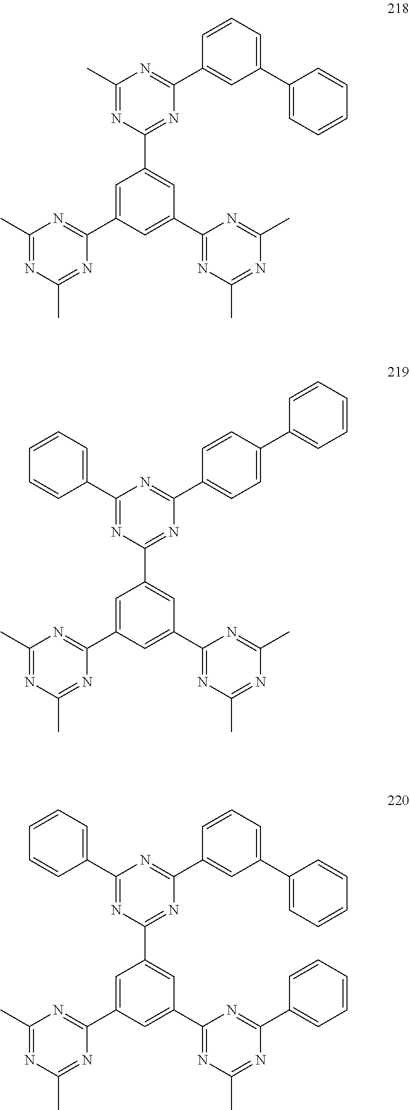 [SMILES: Cc1nc(C)nc(-c2cc(-c3nc(C)nc(C)n3)cc(-c3nc(C)nc(-c4cccc(-c5ccccc5)c4)n3)c2)n1 ; Cc1nc(C)nc(-c2cc(-c3nc(C)nc(C)n3)cc(-c3nc(-c4ccccc4)nc(-c4ccc(-c5ccccc5)cc4)n3)c2)n1 ; Cc1nc(C)nc(-c2cc(-c3nc(C)nc(-c4ccccc4)n3)cc(-c3nc(-c4ccccc4)nc(-c4cccc(-c5ccccc5)c4)n3)c2)n1]

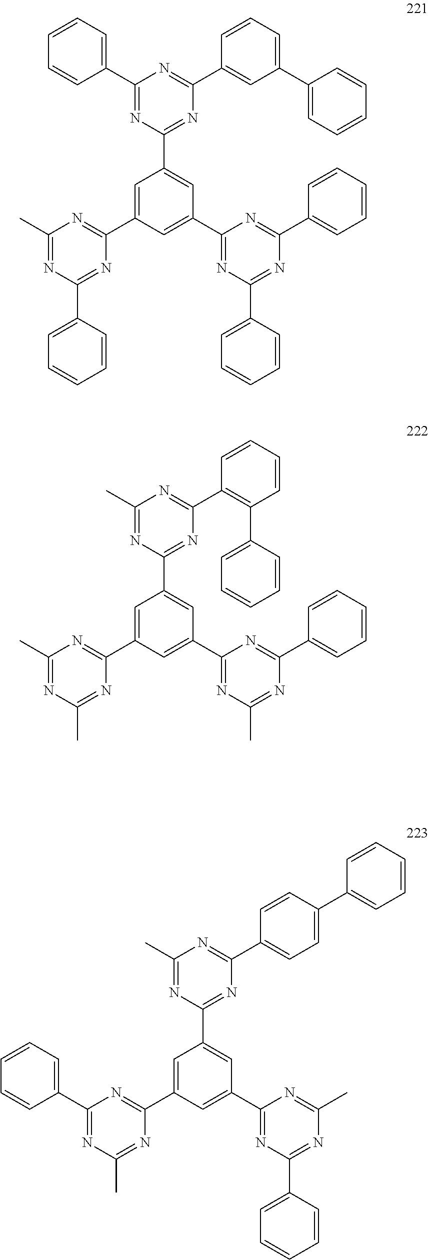 [SMILES: Cc1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1 ; Cc1nc(C)nc(-c2cc(-c3nc(C)nc(-c4ccccc4)n3)cc(-c3nc(C)nc(-c4ccccc4-c4ccccc4)n3)c2)n1 ; Cc1nc(-c2ccccc2)nc(-c2cc(-c3nc(C)nc(-c4ccccc4)n3)cc(-c3nc(C)nc(-c4ccc(-c5ccccc5)cc4)n3)c2)n1]

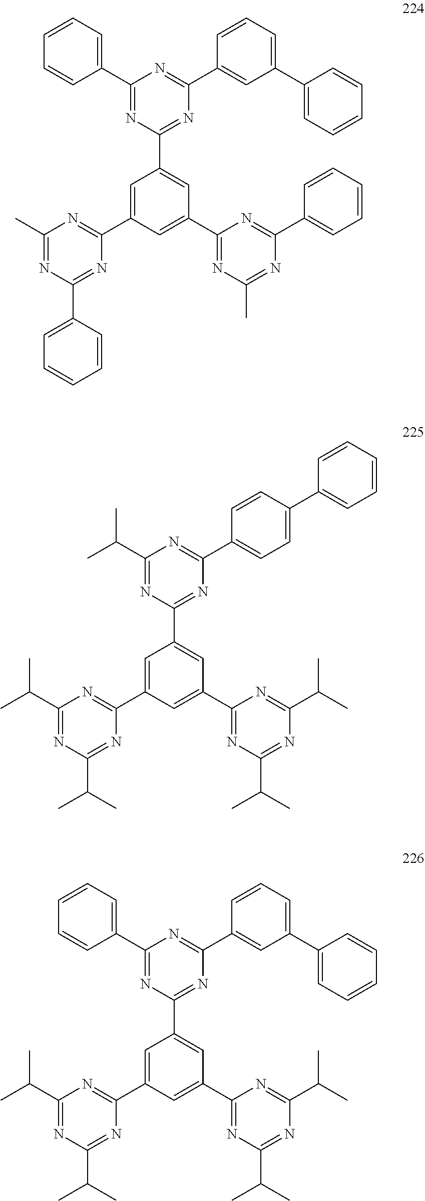 [SMILES: Cc1nc(-c2ccccc2)nc(-c2cc(-c3nc(C)nc(-c4ccccc4)n3)cc(-c3nc(-c4ccccc4)nc(-c4cccc(-c5ccccc5)c4)n3)c2)n1 ; CC(C)c1nc(-c2ccc(-c3ccccc3)cc2)nc(-c2cc(-c3nc(C(C)C)nc(C(C)C)n3)cc(-c3nc(C(C)C)nc(C(C)C)n3)c2)n1 ; CC(C)c1nc(-c2cc(-c3nc(-c4ccccc4)nc(-c4cccc(-c5ccccc5)c4)n3)cc(-c3nc(C(C)C)nc(C(C)C)n3)c2)nc(C(C)C)n1]

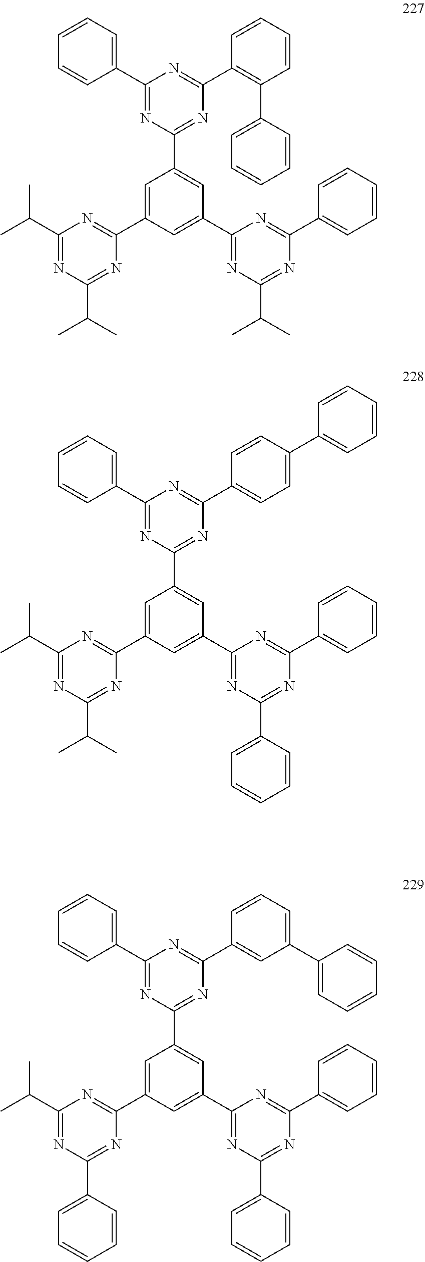 [SMILES: CC(C)c1nc(-c2ccccc2)nc(-c2cc(-c3nc(-c4ccccc4)nc(-c4ccccc4-c4ccccc4)n3)cc(-c3nc(C(C)C)nc(C(C)C)n3)c2)n1 ; CC(C)c1nc(-c2cc(-c3nc(-c4ccccc4)nc(-c4ccccc4)n3)cc(-c3nc(-c4ccccc4)nc(-c4ccc(-c5ccccc5)cc4)n3)c2)nc(C(C)C)n1 ; CC(C)c1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1]

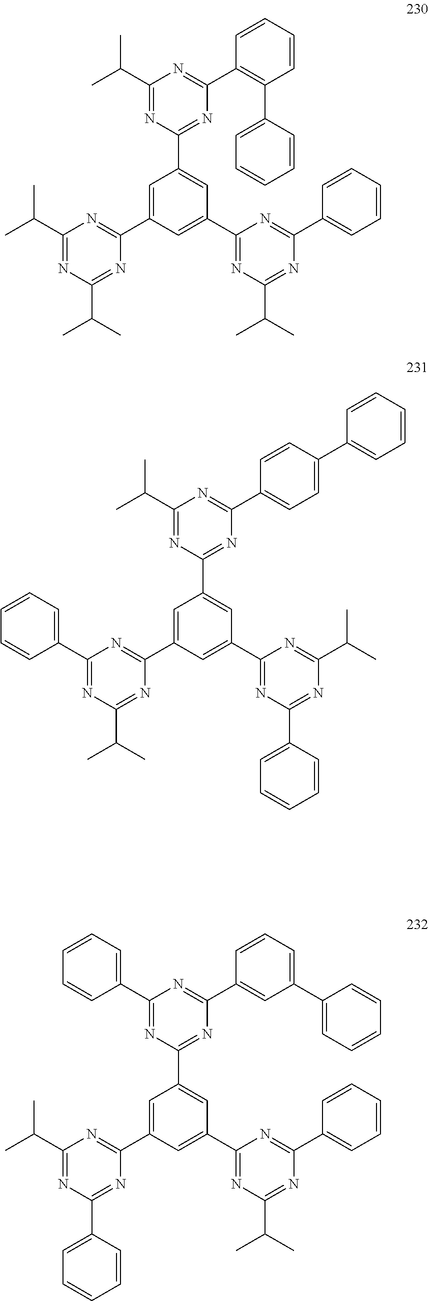 [SMILES: CC(C)c1nc(-c2ccccc2)nc(-c2cc(-c3nc(-c4ccccc4-c4ccccc4)nc(C(C)C)n3)cc(-c3nc(C(C)C)nc(C(C)C)n3)c2)n1 ; CC(C)c1nc(-c2ccccc2)nc(-c2cc(-c3nc(-c4ccccc4)nc(C(C)C)n3)cc(-c3nc(-c4ccc(-c5ccccc5)cc4)nc(C(C)C)n3)c2)n1 ; CC(C)c1nc(-c2ccccc2)nc(-c2cc(-c3nc(-c4ccccc4)nc(-c4cccc(-c5ccccc5)c4)n3)cc(-c3nc(-c4ccccc4)nc(C(C)C)n3)c2)n1]

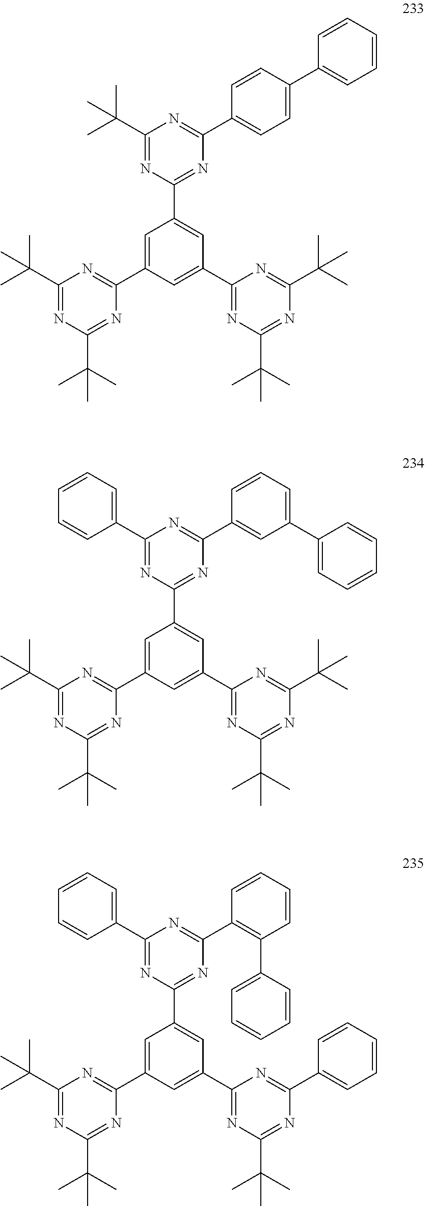 [SMILES: CC(C)(C)c1nc(-c2ccc(-c3ccccc3)cc2)nc(-c2cc(-c3nc(C(C)(C)C)nc(C(C)(C)C)n3)cc(-c3nc(C(C)(C)C)nc(C(C)(C)C)n3)c2)n1 ; CC(C)(C)c1nc(-c2cc(-c3nc(-c4ccccc4)nc(-c4cccc(-c5ccccc5)c4)n3)cc(-c3nc(C(C)(C)C)nc(C(C)(C)C)n3)c2)nc(C(C)(C)C)n1 ; CC(C)(C)c1nc(-c2ccccc2)nc(-c2cc(-c3nc(-c4ccccc4)nc(-c4ccccc4-c4ccccc4)n3)cc(-c3nc(C(C)(C)C)nc(C(C)(C)C)n3)c2)n1]

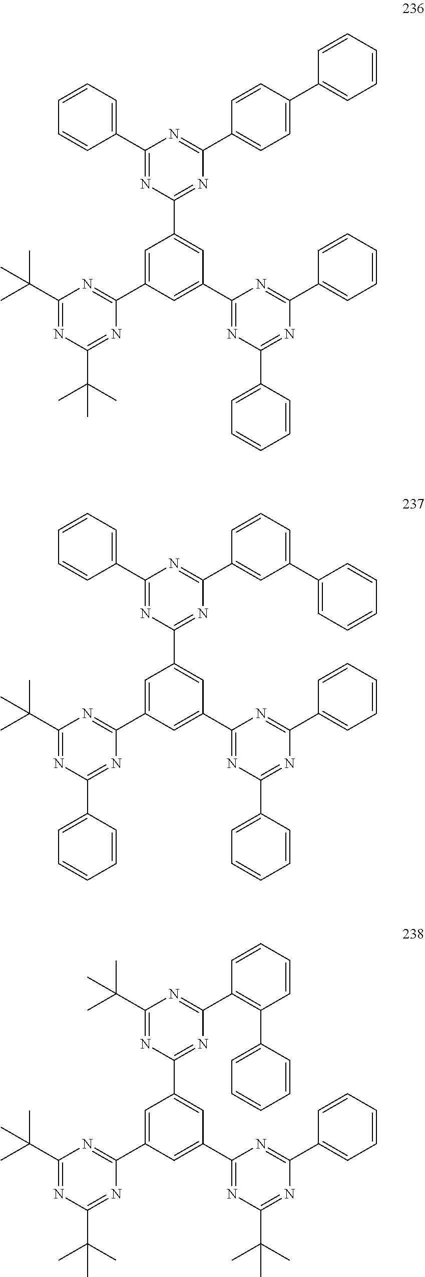 [SMILES: CC(C)(C)c1nc(-c2cc(-c3nc(-c4ccccc4)nc(-c4ccccc4)n3)cc(-c3nc(-c4ccccc4)nc(-c4ccc(-c5ccccc5)cc4)n3)c2)nc(C(C)(C)C)n1 ; CC(C)(C)c1nc(-c2ccccc2)nc(-c2cc(-c3nc(-c4ccccc4)nc(-c4ccccc4)n3)cc(-c3nc(-c4ccccc4)nc(-c4cccc(-c5ccccc5)c4)n3)c2)n1 ; CC(C)(C)c1nc(-c2ccccc2)nc(-c2cc(-c3nc(-c4ccccc4-c4ccccc4)nc(C(C)(C)C)n3)cc(-c3nc(C(C)(C)C)nc(C(C)(C)C)n3)c2)n1]

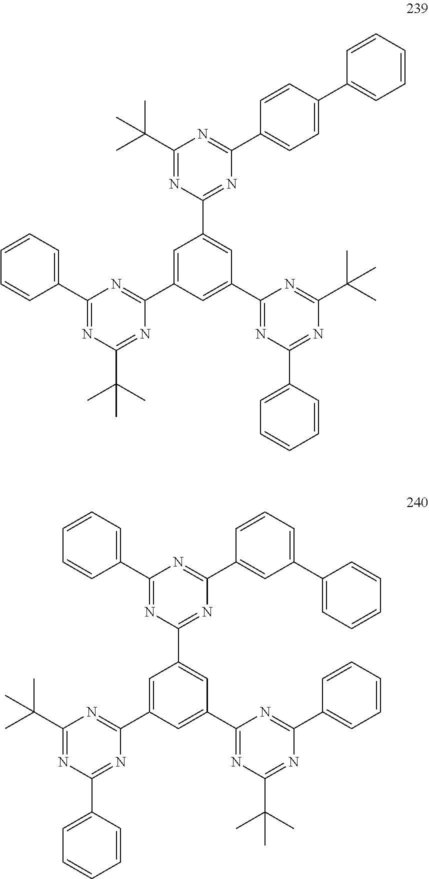 [SMILES: CC(C)(C)c1nc(-c2ccccc2)nc(-c2cc(-c3nc(-c4ccccc4)nc(C(C)(C)C)n3)cc(-c3nc(-c4ccc(-c5ccccc5)cc4)nc(C(C)(C)C)n3)c2)n1 ; CC(C)(C)c1nc(-c2ccccc2)nc(-c2cc(-c3nc(-c4ccccc4)nc(-c4cccc(-c5ccccc5)c4)n3)cc(-c3nc(-c4ccccc4)nc(C(C)(C)C)n3)c2)n1]

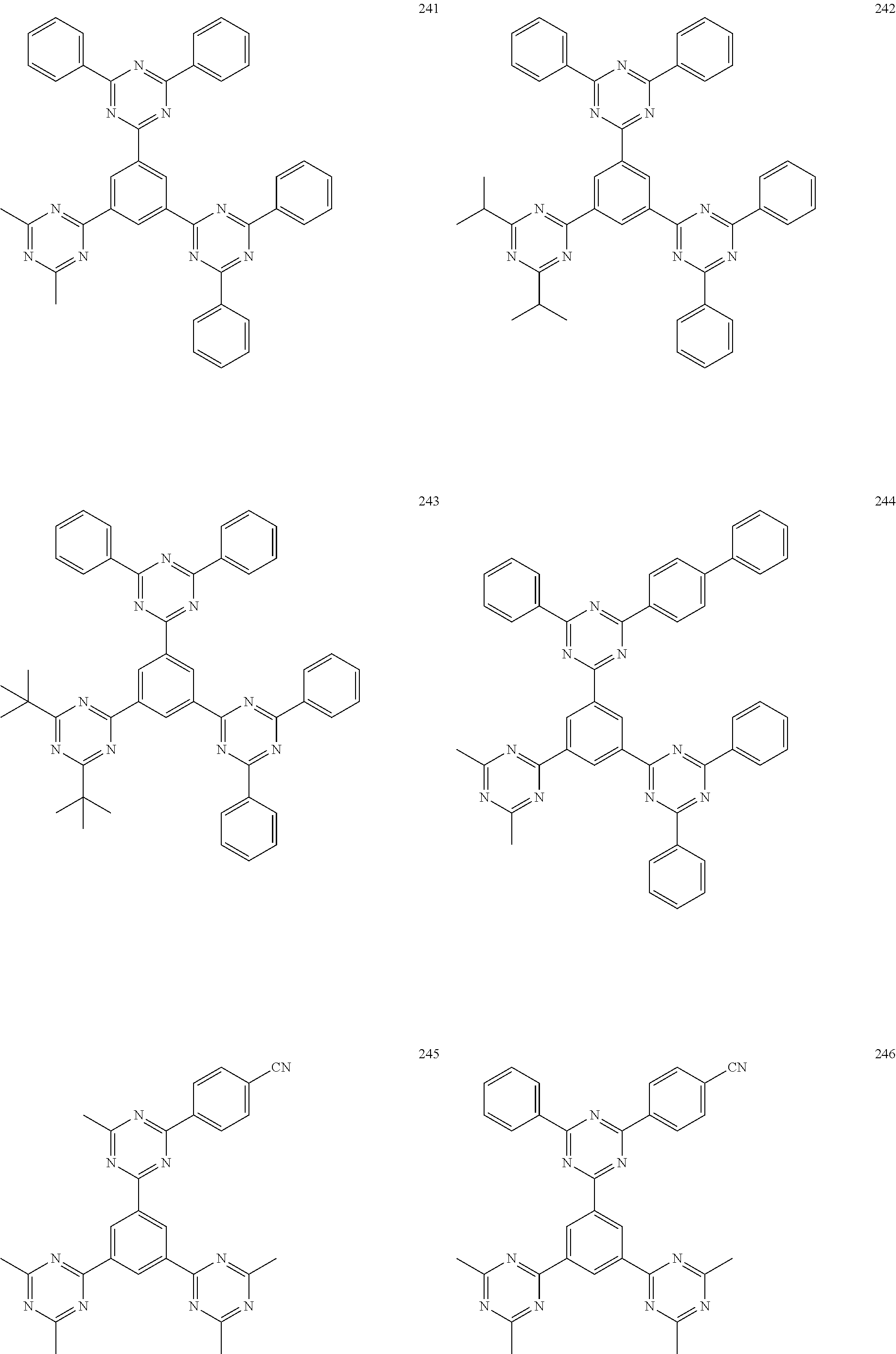

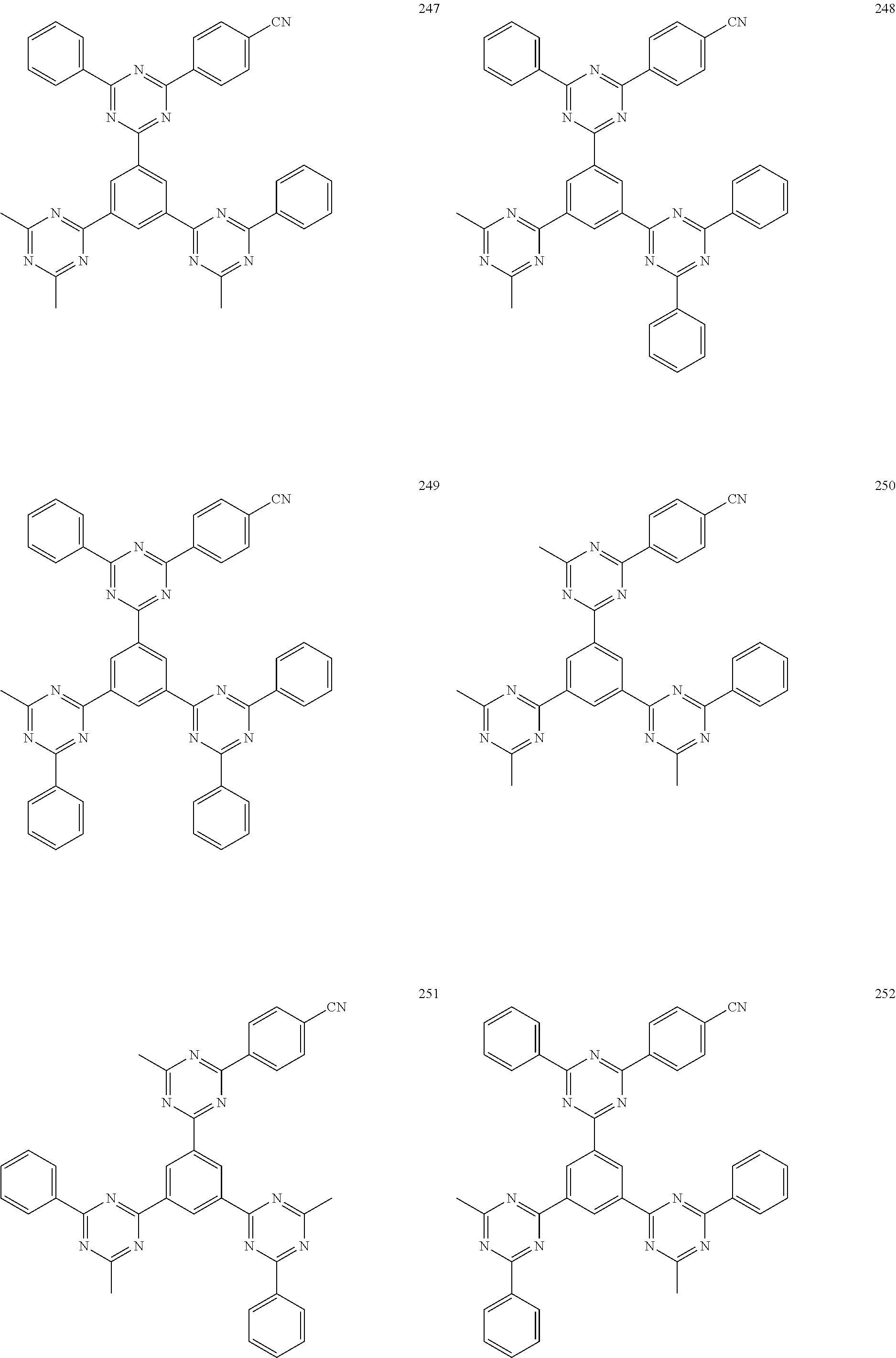

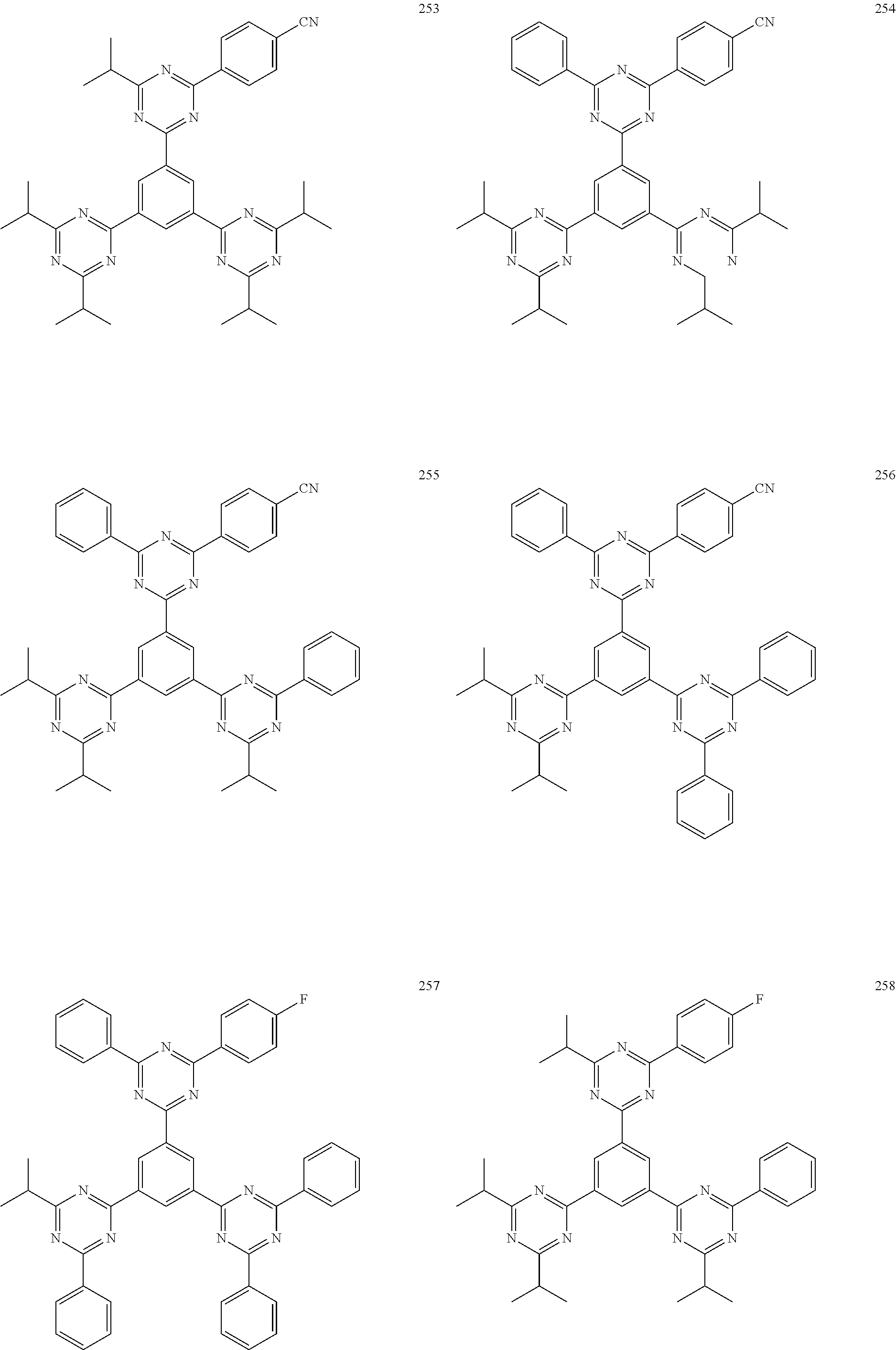

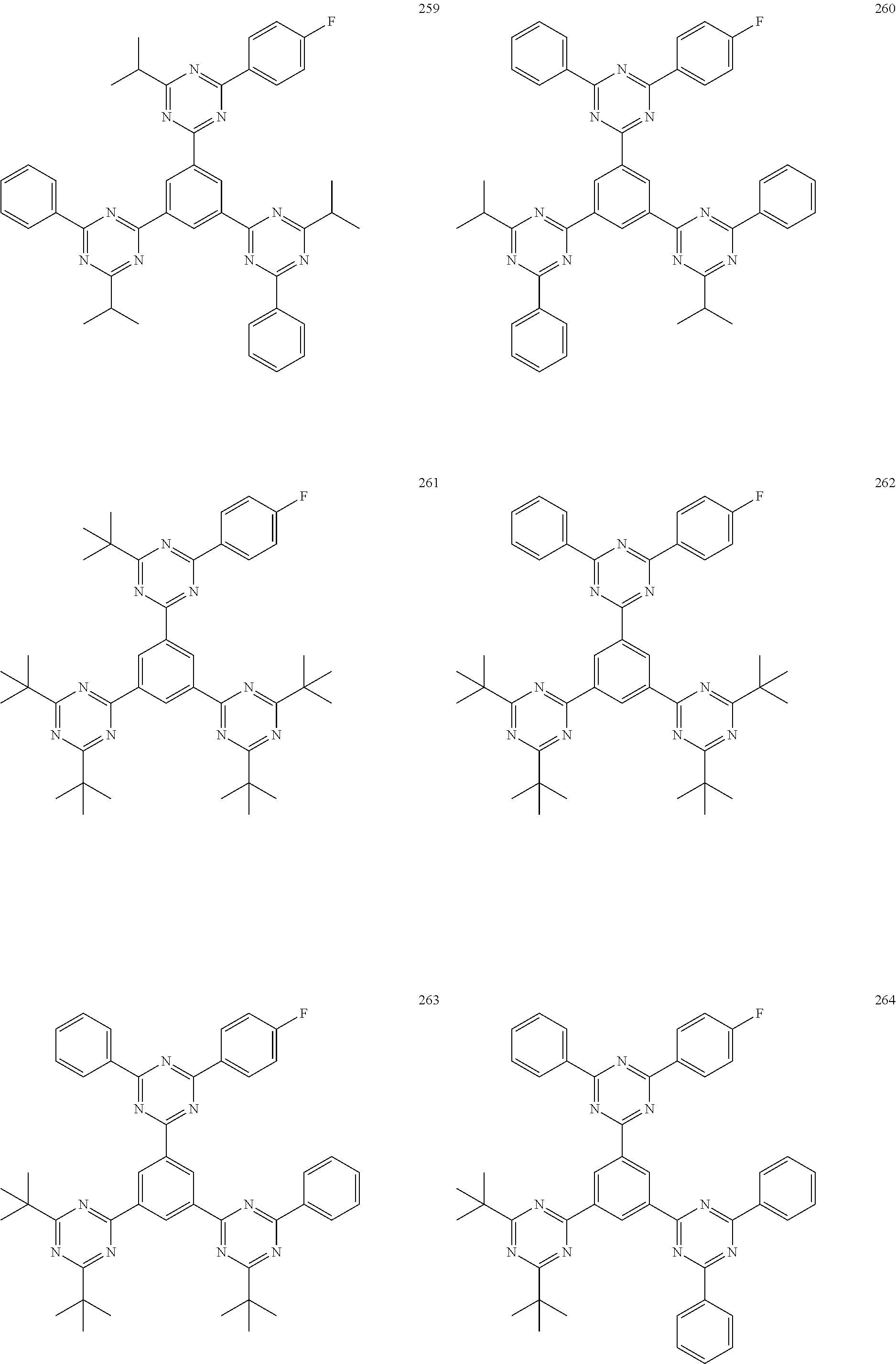

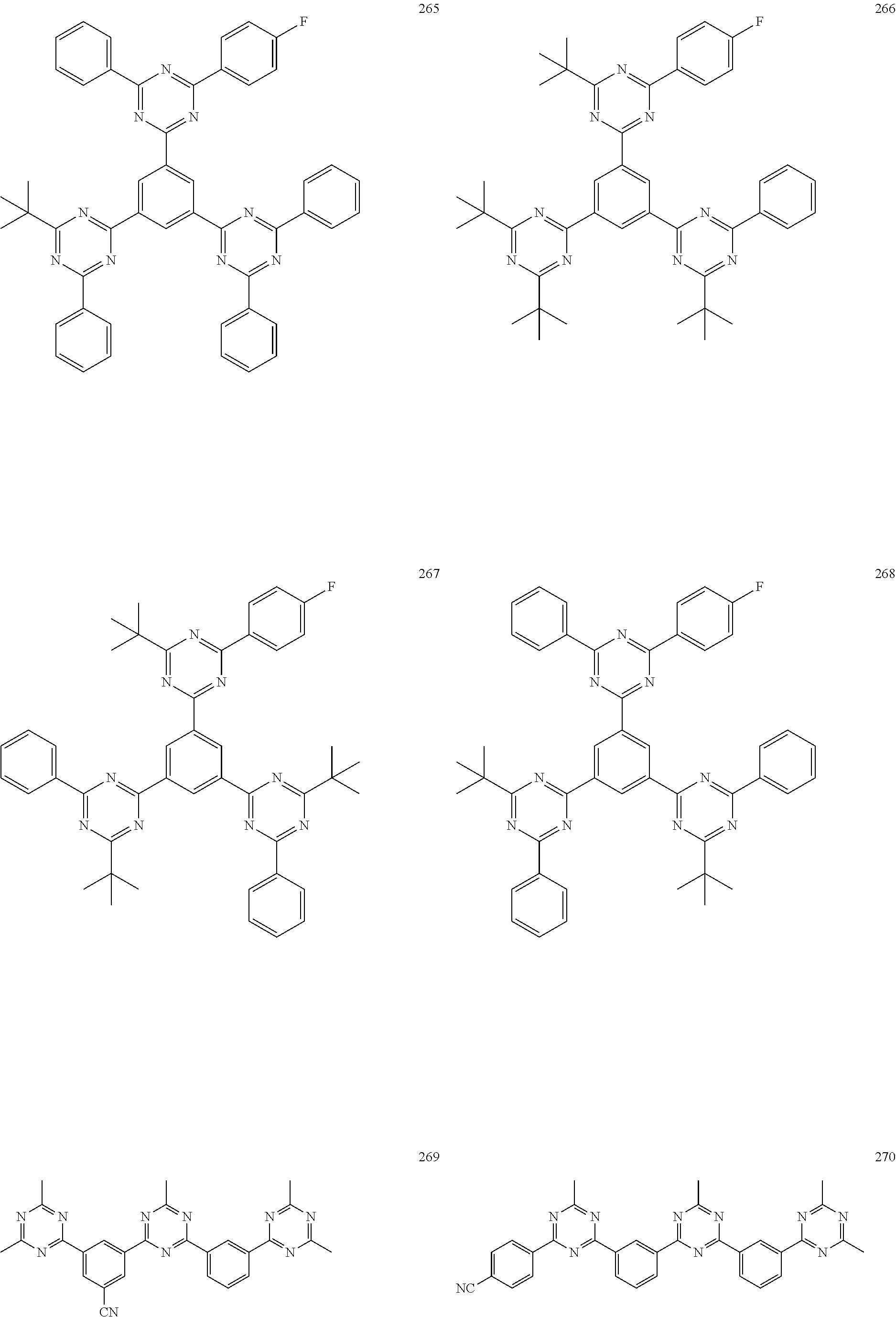

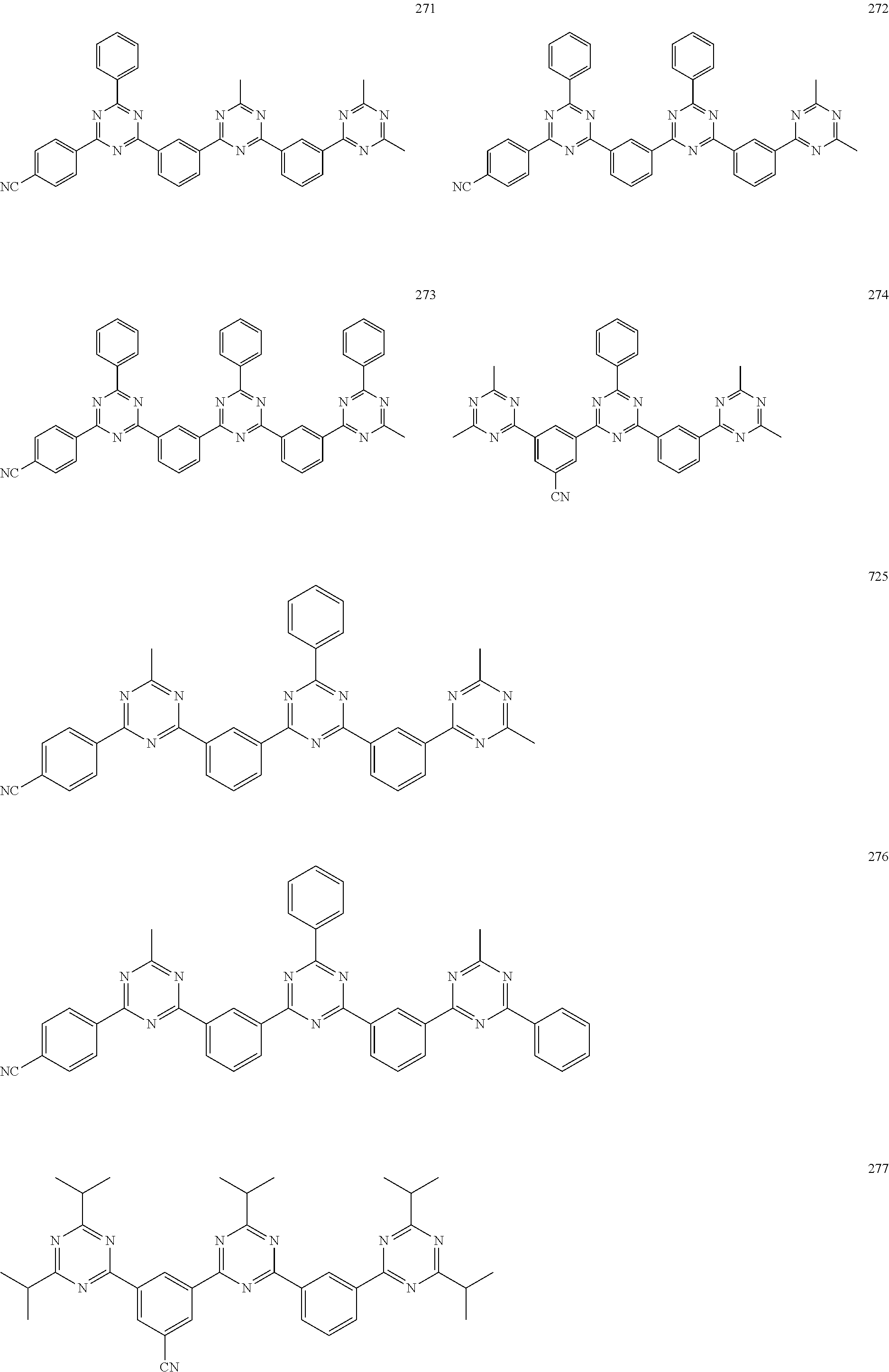

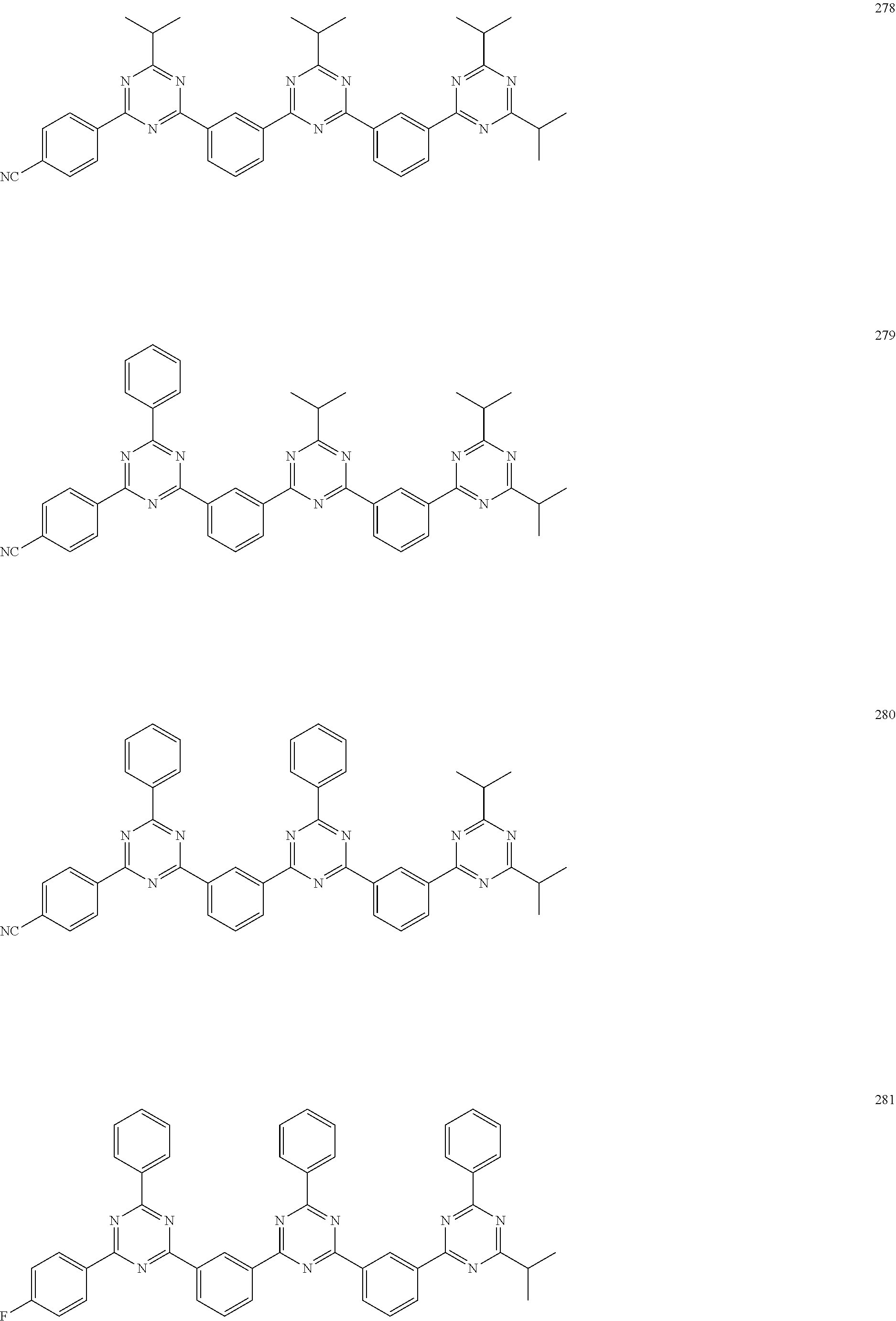 [SMILES: CC(C)c1nc(-c2ccc(C#N)cc2)nc(-c2cccc(-c3nc(-c4cccc(-c5nc(C(C)C)nc(C(C)C)n5)c4)nc(C(C)C)n3)c2)n1 ; CC(C)c1nc(-c2cccc(-c3nc(-c4ccccc4)nc(-c4ccc(C#N)cc4)n3)c2)nc(-c2cccc(-c3nc(C(C)C)nc(C(C)C)n3)c2)n1 ; CC(C)c1nc(-c2cccc(-c3nc(-c4ccccc4)nc(-c4cccc(-c5nc(-c6ccccc6)nc(-c6ccc(C#N)cc6)n5)c4)n3)c2)nc(C(C)C)n1 ; CC(C)c1nc(-c2ccccc2)nc(-c2cccc(-c3nc(-c4ccccc4)nc(-c4cccc(-c5nc(-c6ccccc6)nc(-c6ccc(F)cc6)n5)c4)n3)c2)n1]

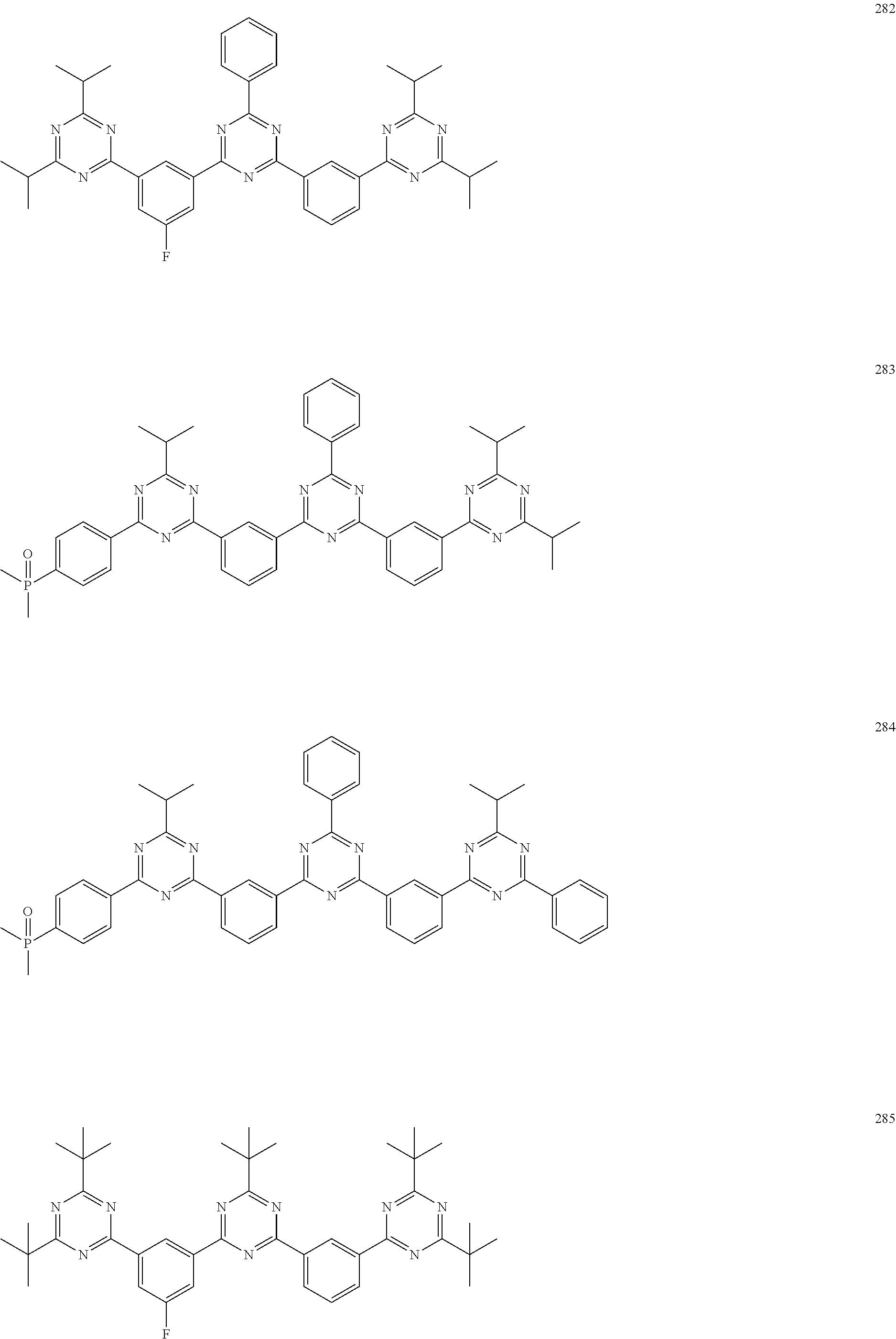

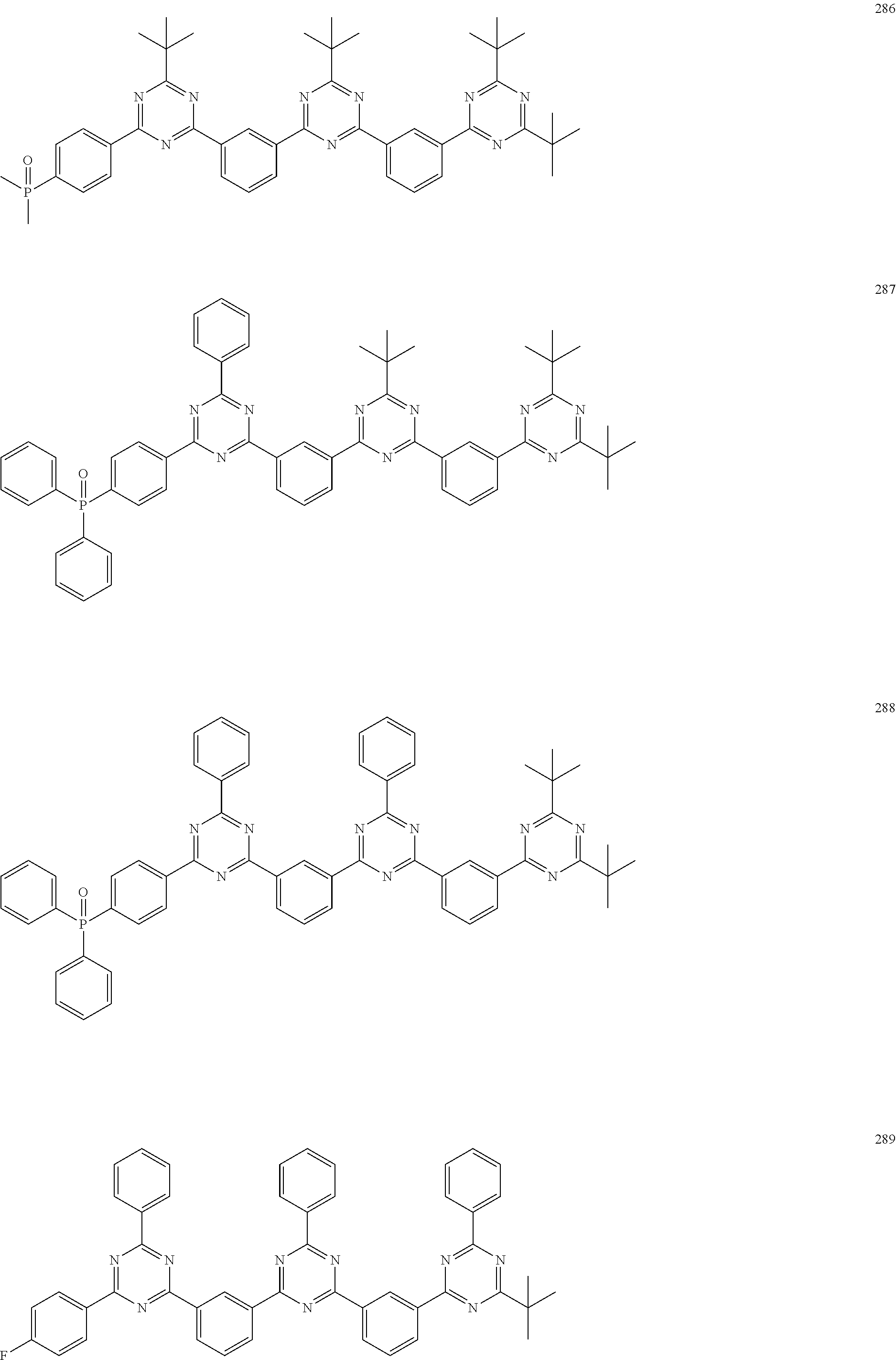 [SMILES: CC(C)(C)c1nc(-c2ccc(P(C)(C)=O)cc2)nc(-c2cccc(-c3nc(-c4cccc(-c5nc(C(C)(C)C)nc(C(C)(C)C)n5)c4)nc(C(C)(C)C)n3)c2)n1 ; CC(C)(C)c1nc(-c2cccc(-c3nc(-c4ccccc4)nc(-c4ccc(P(=O)(c5ccccc5)c5ccccc5)cc4)n3)c2)nc(-c2cccc(-c3nc(C(C)(C)C)nc(C(C)(C)C)n3)c2)n1 ; CC(C)(C)c1nc(-c2cccc(-c3nc(-c4ccccc4)nc(-c4cccc(-c5nc(-c6ccccc6)nc(-c6ccc(P(=O)(c7ccccc7)c7ccccc7)cc6)n5)c4)n3)c2)nc(C(C)(C)C)n1 ; CC(C)(C)c1nc(-c2ccccc2)nc(-c2cccc(-c3nc(-c4ccccc4)nc(-c4cccc(-c5nc(-c6ccccc6)nc(-c6ccc(F)cc6)n5)c4)n3)c2)n1]

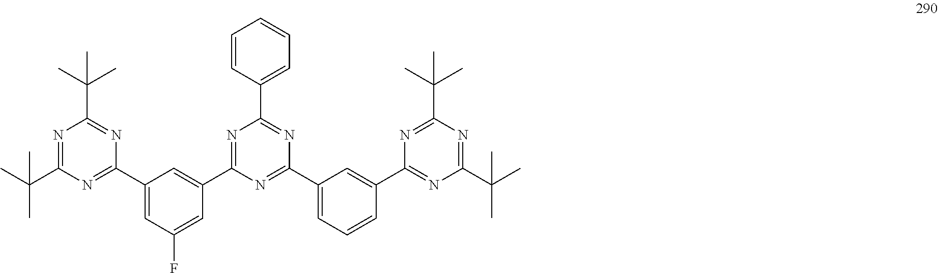 [SMILES: CC(C)(C)c1nc(-c2cccc(-c3nc(-c4ccccc4)nc(-c4cc(F)cc(-c5nc(C(C)(C)C)nc(C(C)(C)C)n5)c4)n3)c2)nc(C(C)(C)C)n1]

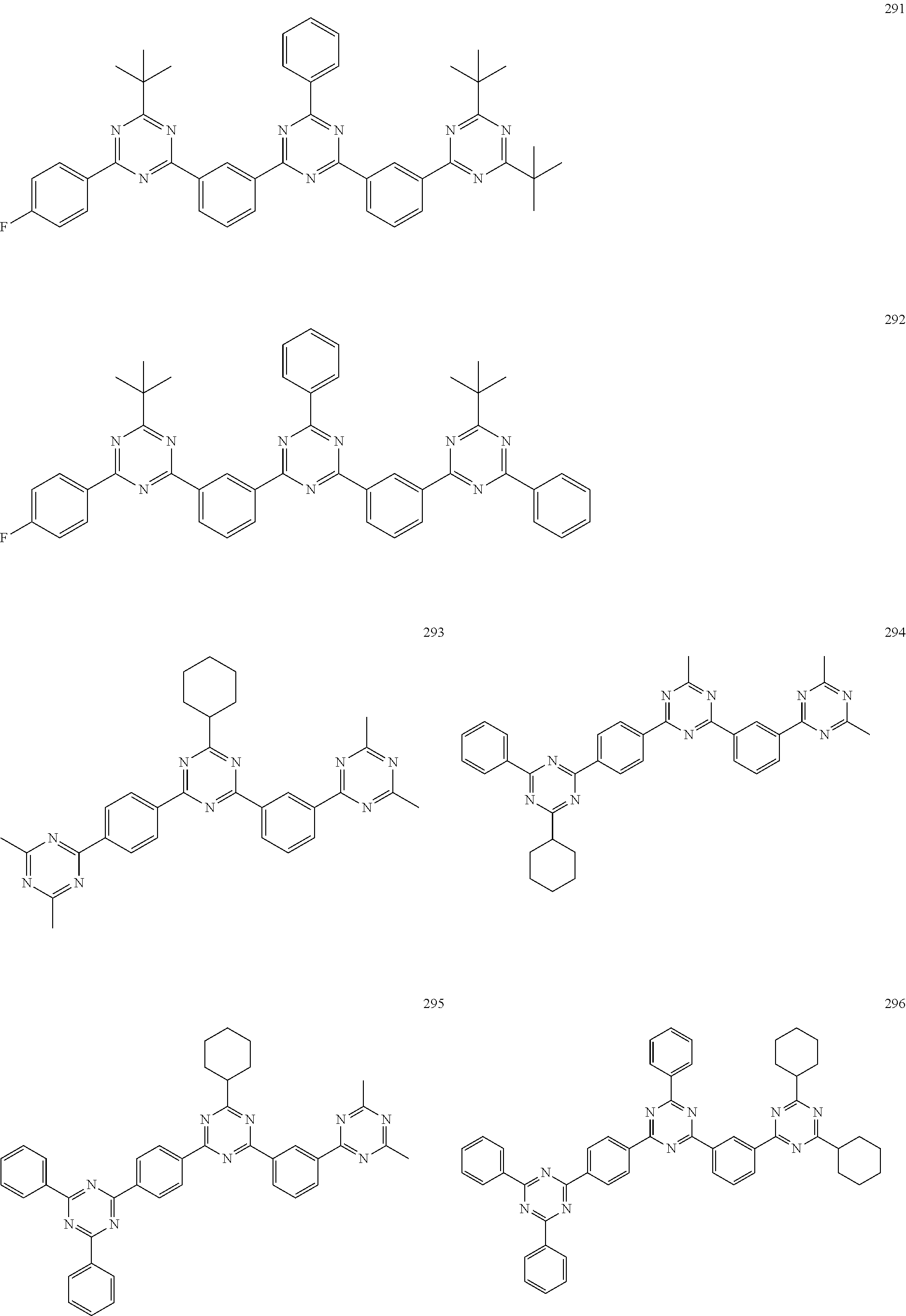

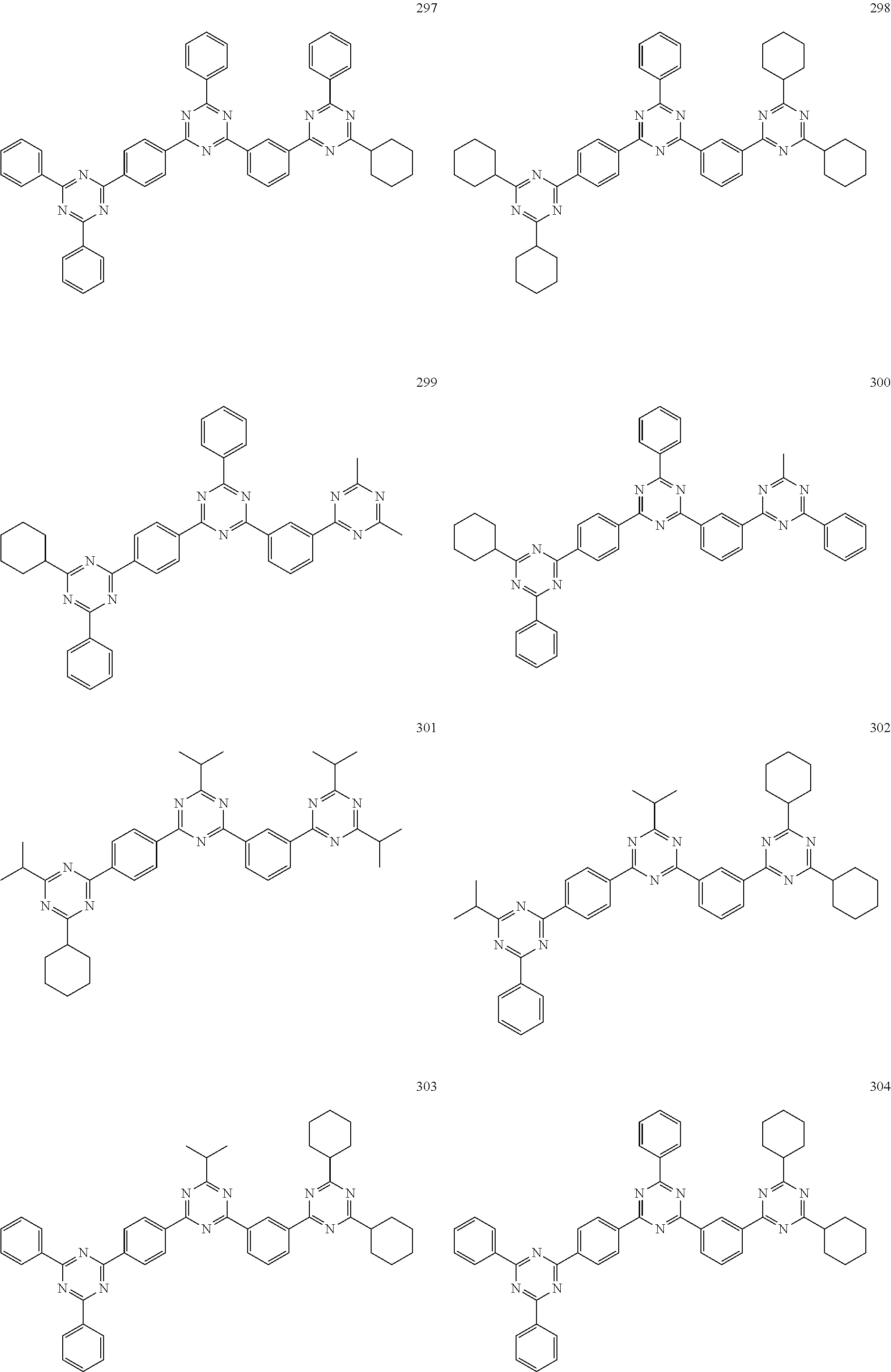

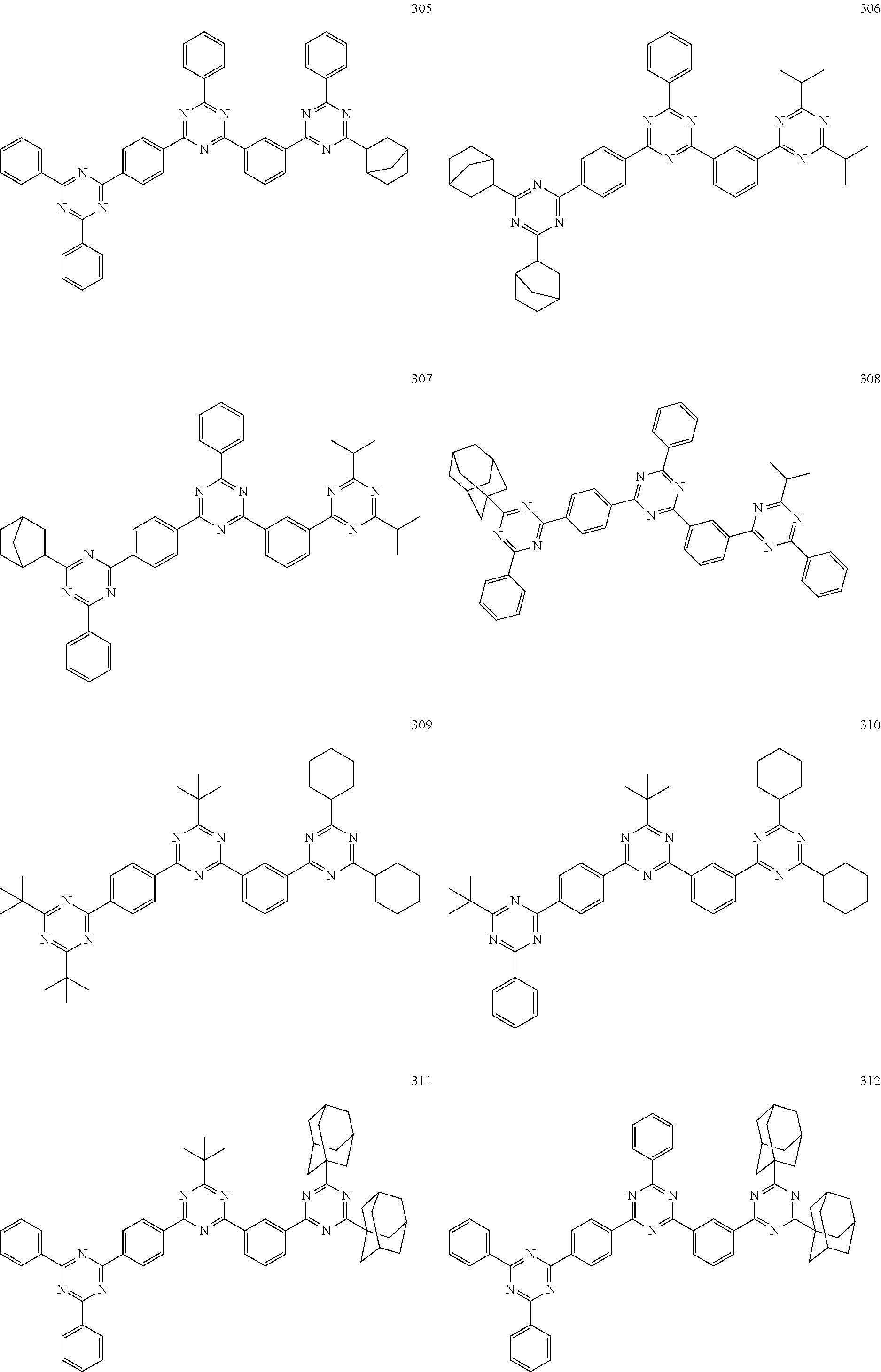

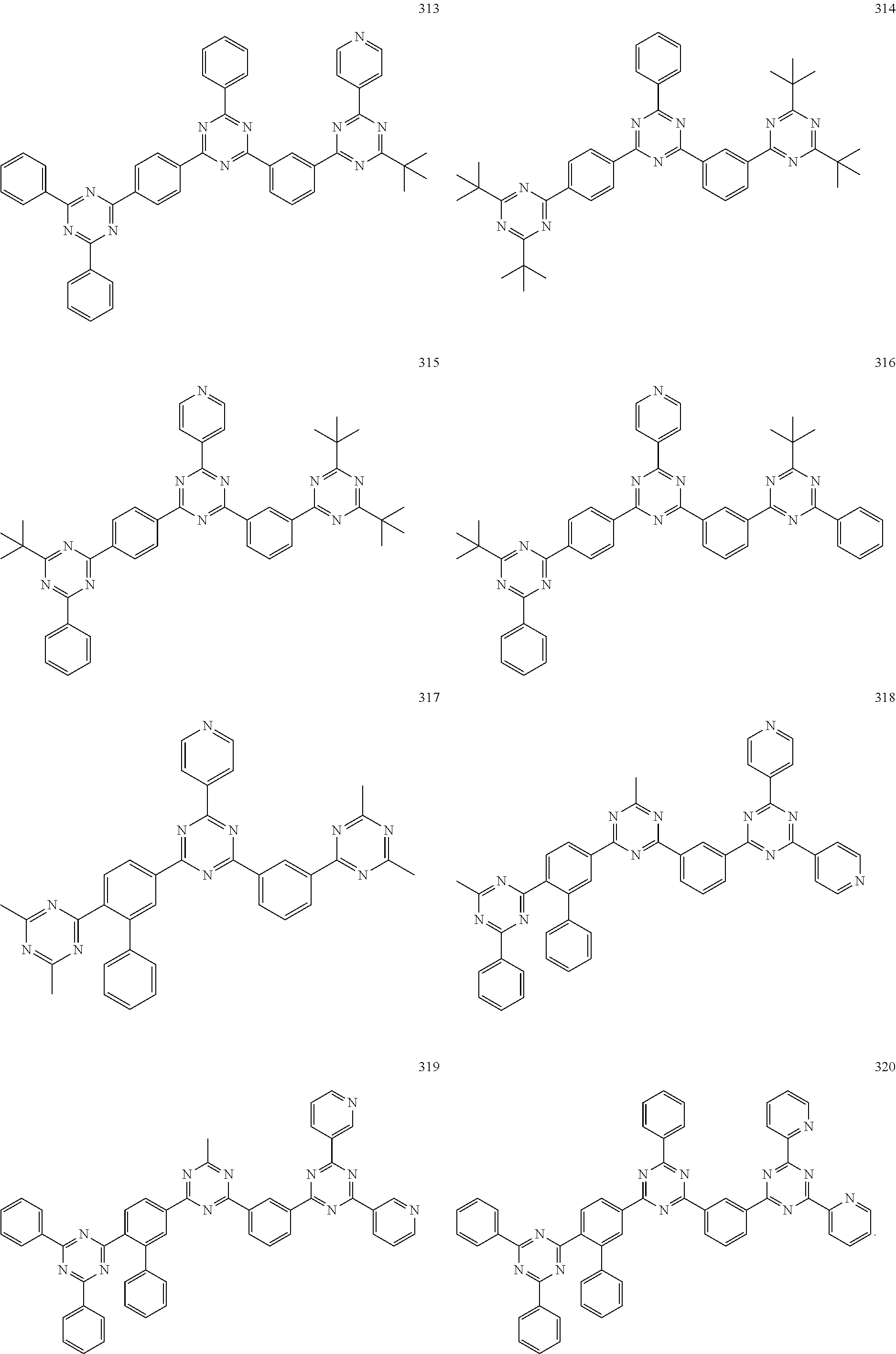

18. A display device comprising:
a base layer;
a circuit layer on the base layer; and
a display element layer on the circuit layer and comprising a light-emitting element,
wherein the light-emitting element comprises a first electrode, a second electrode on the first electrode, an emission layer between the first electrode and the second electrode, and at least one functional layer between the first electrode and the emission layer and comprising a nitrogen-containing compound represented by Formula 1: 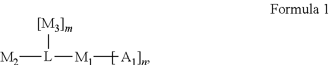
and
wherein, in Formula 1,
A₁ is -(L₁)ₐ₁M₄,
L₁ is a substituted or unsubstituted arylene group having 6 to 60 ring-forming carbons, or a substituted or unsubstituted heteroarylene group having 2 to 60 ring-forming carbons,
M₂ to M₄ are each independently a substituted or unsubstituted triazine group,
a1 is an integer of 1 to 3,
m and n are each independently 0 or 1,
a sum of m and n is 1,
when n is 0, then M₁ is a substituted or unsubstituted triazine group,
when n is 1, then M₁ is a substituted or unsubstituted divalent triazine group,
when m is 0, L is represented by -(L₂)ₐ₂-,
L₂ is a substituted or unsubstituted arylene group having 6 to 60 ring-forming carbons, or a substituted or unsubstituted heteroarylene group having 2 to 60 ring-forming carbons,
a2 is an integer of 1 to 3,
when m is 1, then L is a substituted or unsubstituted trivalent aryl group having 6 to 60 ring-forming carbons, or a substituted or unsubstituted trivalent heteroaryl group having 2 to 60 ring-forming carbons,
at least one selected from among M₁ to M₄ is a substituted or unsubstituted alkyl group having 1 to 30 carbons, or a substituted or unsubstituted cycloalkyl group having 3 to 30 ring-forming carbons, and
each of L, L₁, and L₂ do not comprise a substituted or unsubstituted divalent carbazole group.

19. The display device of claim 18, wherein the light-emitting element further comprises a capping layer on the second electrode, and
a refractive index of the capping layer for light in a wavelength range of about 550 nanometer (nm) to about 660 nm, is at least about 1.6.

20. The display device of claim 18, further comprising an optical control layer on the display element layer and comprising a quantum dot,
the light-emitting element being configured to emit a first color light, and
the optical control layer comprising:
a first optical control part being configured to convert the first color light to a second color light having a wavelength range longer than a wavelength range of the first color light;
a second optical control part being configured to convert the first color light to a third color light having a wavelength range longer than the wavelength range of the first color light and the wavelength range of the second color light; and
a third optical control part being configured to transmit the first color light.
